(19) United States
(12) Patent Application Publication
(10) Pub. No.: US 2009/0270381 A1
(43) Pub. Date: Oct. 29, 2009

(54) Non-steroidal progesterone receptor modulators

(76) Inventors: Wolfgang SCHWEDE, Glienicke (DE); Thomas Andrew Kirkland, American Canyon, CA (US); Carsten Moeller, Berlin (DE); Stefan Baeurle, Berlin (DE); Ralf Wyrwa, Rothenstein (DE); Andrea Rotgeri, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/333,789

(22) Filed: Dec. 12, 2008

Related U.S. Application Data
(60) Provisional application No. 61/006,015, filed on Dec. 14, 2007.

(30) Foreign Application Priority Data
Dec. 14, 2007 (EP) 07076093.9

(51) Int. Cl. A61K 31/277; C07C 255/50; C07D 265/02; C07D 413/06; A61K 31/536; A61P 15/00

(52) U.S. Cl. 514/230.5; 558/414; 544/63; 544/121; 514/522

(57) ABSTRACT

The present invention relates to non-steroidal progesterone receptor modulators of the general formula I,

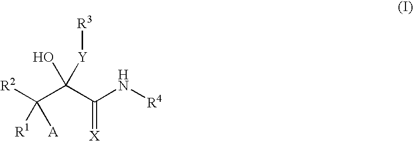

the use of the progesterone receptor modulators for the manufacture of medicaments, and pharmaceutical compositions which comprise these compounds.

The compounds according to the invention are suitable for the therapy and prophylaxis of gynecological disorders such as endometriosis, leiomyomas of the uterus, dysfunctional bleeding and dysmenorrhoea, and for the therapy and prophylaxis of hormone-dependent tumours and for use for female fertility control and for hormone replacement therapy.

This application claims the benefit of the filing date of U.S. Provisional Application Ser. No. 61/006,015 filed Dec. 14, 2007.

The present invention relates to non-steroidal progesterone receptor modulators, a method for their preparation, the use of the progesterone receptor modulators for the manufacture of medicaments, and pharmaceutical compositions which comprise these compounds.

The steroid hormone progesterone controls in a decisive manner the reproductive process in the female body. Progesterone is secreted in large quantities during the cycle and pregnancy respectively by the ovary and the placenta. Progesterone in cooperation with oestrogens brings about cyclic changes in the uterine mucosa (endometrium) during the menstrual cycle. Elevated progesterone levels after ovulation influence the uterine mucosa to convert it into a state permitting nidation of an embryo (blastocyst). During pregnancy, progesterone controls the relaxation of the myometrium and maintains the function of the decidual tissue.

It is further known that progesterone inhibits endometrial proliferation by suppressing oestrogen-mediated mitosis in uterine tissue (K. Chwalisz, R. M. Brenner, U. Fuhrmann, H. Hess-Stumpp, W. Elger, Steroids 65, 2000, 741-751).

Progesterone and progesterone receptors are also known to play a significant part in pathophysiological processes. Progesterone receptors have been detected in the foci of endometriosis, but also in tumours of the uterus, of the breast and of the CNS. It is further known that uterine leiomyomas grow progesterone-dependently.

The effects of progesterone in the tissues of the genital organs and in other tissues occur through interactions with progesterone receptors which are responsible for the cellular effects.

Progesterone receptor modulators are either pure agonists or inhibit the effect of progesterone partly or completely. Accordingly, substances are defined as pure agonists, partial agonists (selective progesterone receptor modulators=SPRMs) and pure antagonists.

In accordance with the ability of progesterone receptor modulators to display their effect via the progesterone receptor, these compounds have a considerable potential as therapeutic agents for gynaecological and oncological indications and for obstetrics and fertility control.

Pure progesterone receptor antagonists completely inhibit the effect of progesterone on the progesterone receptor. They have anti-ovulatory properties and the ability to inhibit oestrogen effects in the endometrium, as far as complete atrophy. They are therefore particularly suitable for intervening in the female reproductive process, e.g. post-ovulation, in order to prevent nidation of a fertilized egg cell, during pregnancy in order to increase the reactivity of the uterus to prostaglandins or oxytocin, or in order to achieve opening and softening (“ripening”) of the cervix, and to induce a great readiness of the myometrium to contract.

A beneficial effect on the pathological event is expected in foci of endometriosis and in tumour tissues which are equipped with progesterone receptors after administration of pure progesterone receptor antagonists. There might be particular advantages for influencing pathological states such as endometriosis or uterine leiomyomas if ovulation inhibition can additionally be achieved by the progesterone receptor antagonists. Ovulation inhibition also dispenses with some of the ovarian hormone production and thus the stimulating effect, deriving from this proportion, on the pathologically altered tissue.

The first progesterone receptor antagonist described, RU 486 (also mifepristone), was followed by the synthesis and characterization of a large number of analogues with progesterone receptor-antagonistic activity of varying strength. Whereas RU 486 also shows an antiglucocorticoid effect in addition to the progesterone receptor-antagonistic effect, compounds synthesized later are notable in particular for a more selective effect as progesterone receptor antagonists.

Besides steroidal compounds such as onapristone or lilopristone, which are notable by comparison with RU 486 for a better dissociation of the progesterone receptor-antagonistic effect and the antiglucocorticoid effect, also known from the literature are various non-steroidal structures whose antagonistic effect on the progesterone receptor is being investigated [see, for example, S. A. Leonhardt and D. P. Edwards, Exp. Biol. Med. 227: 969-980 (2002) and R. Winneker, A. Fensome, J. E. Wrobel, Z. Zhang, P. Zhang, Seminars in Reproductive Medicine, Volume 23: 46-57 (2005)]. However, non-steroidal compounds disclosed to date have only moderate antagonistic activity compared with the activity of known steroidal structures. The most effective non-steroidal compounds are reported to have in vitro activities which are 10% of the activity of RU 486.

The antiglucocorticoid activity is disadvantageous for therapeutic use, where the inhibition of progesterone receptors is at the forefront of the therapy. An antiglucocorticoid activity causes unwanted side effects at the dosages necessary for therapy. This may prevent administration of a therapeutically worthwhile dose or lead to discontinuation of the treatment.

Partial or complete reduction of the antiglucocorticoid properties is therefore an important precondition for therapy with progesterone receptor antagonists, especially for those indications requiring treatment lasting weeks or months.

In contrast to the pure antagonists, partial progesterone receptor agonists (SPRMs) show a residual agonistic property which may vary in strength. This leads to these substances showing agonistic effects on the progesterone receptor in certain organ systems (D. DeManno, W. Elger, R. Garg, R. Lee, B. Schneider, H. Hess-Stumpp, G. Schuber, K. Chwalisz, Steroids 68, 2003, 1019-1032). Such an organ-specific and dissociated effect may be of therapeutic benefit for the described indications.

It is therefore an object of the present invention to provide further non-steroidal progesterone receptor modulators. These compounds are intended to have a reduced antiglucocorticoid effect and therefore be suitable for the therapy and prophylaxis of gynaecological disorders such as endometriosis, leiomyomas of the uterus, dysfunctional bleeding and dysmenorrhoea. The compounds according to the invention are additionally intended to be suitable for the therapy and prophylaxis of hormone-dependent tumours, for example of breast, endometrial, ovarian and prostate carcinomas. The compounds are intended furthermore to be suitable for use in female fertility control and for female hormone replacement therapy.

The object is achieved according to the present invention by the provision of non-steroidal compounds of the general formula I

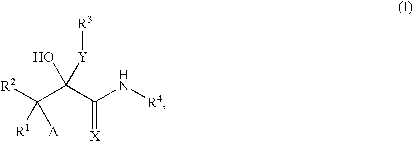

in which
A is hydrogen or a C₁-C₈-alkyl, C₂-C₈-alkenyl, C₂-C₈-alkynyl radical which is optionally mono- or polysubstituted identically or differently by Z, or else is Z itself, where Z is defined as follows:
   cyano, halogen, hydroxyl, nitro, —C(O)R^{b}, —C(O)CH₂R^{b}, —C(O)CF₂R^{b}, CO₂R^{b}, —O—R^{b},
   —S—R^{b}, SO₂NR^{c}R^{d}, —C(O)—NR^{c}R^{d}, —OC(O)—NR^{c}R^{d}, —C═NOR^{b},
   —NR^{c}R^{d}, PO₃(Rb)₂, —NR^{e}COR^{b}, —NR^{e}CSR^{b}, —NR^{e}S(O)R^{b}, —NR^{e}S(O)₂R^{b},
   NR^{e}CONR^{c}R^{d}, —NR^{e}COOR^{b},
   —NR^{e}C(NH)NR^{c}R^{d}, —NR^{e}CSNR^{c}R^{d}, —NR^{e}S(O)N^{c}R^{c}R^{d},
   —NR^{e}S(O)₂NR^{c}R^{d}, —S(O)R^{b}, —S(O)NR^{c}R^{d}, —S(O)₂R^{b}, —S(O)₂CH₂R^{b},
   —S(O)₂CF₂R^{b}, —SO₂OR^{b},
   —CSNR^{c}R^{d}, —CR^{b}(OH)—R^{b} where
R^{b} is hydrogen, a C₁-C₆-alkyl, hydroxy-C₁-C₃-alkyl, C₁-C₃-alkoxy-C₁-C₃-alkyl, C₂-C₈-alkenyl, C₂-C₈-alkynyl, —(CH₂)ₚC(O)₂H, —(CH₂)ₚC(O)₂C₁-C₃-alkyl, a 5- to 6-membered cycloalkyl or heterocycloalkyl radical which is optionally mono- or disubstituted by a halogen, a C₁-C₃-alkyl, C₁-C₃-alkoxy radical or a COOR^{b} radical and has 1, 2 or 3 heteroatoms or
a phenyl or 3-12-membered heteroaryl radical which is optionally mono- or disubstituted by a halogen, a C₁-C₃-alkyl, C₁-C₃-alkoxy radical or a COOR^{b} radical and has 1, 2 or 3 heteroatoms, or a —(CH₂)ₚ—C₆-C₁₂-aryl radical which is optionally mono- or disubstituted by a halogen, a C₁-C₃-alkyl, C₁-C₃-alkoxy radical or a COOR^{b} radical
or a partly or fully fluorinated C₁-C₃-fluoroalkyl radical or W and
R^{c} and R^{d} are each independently hydrogen, a C₁-C₆-alkyl, C₂-C₈-alkenyl, C₂-C₈-alkynyl, C₃-C₁₀-cycloalkyl or C₆-C₁₂-aryl radical or a 5- to 12-membered heteroaryl radical optionally substituted by W, a C(O)R^{b} group with the definition of R^{b} specified above, —S(O)₂C₁-C₃-alkyl, —C(O)C₁-C₃-alkyl or a hydroxyl group, or together including the nitrogen form a 3- to 7-membered ring which is optionally mono- or disubstituted by a trifluoromethyl and/or hydroxyl group and which is optionally extended by O, S or NR^{f} where
   W is —NR^{g}R^{h} where
   R^{g} is hydrogen or C₁-C₃-alkyl and
   R^{h} is hydrogen or C₁-C₃-alkyl or
   R^{g} and R^{h} together including the nitrogen form a 3- to 7-membered ring which is optionally extended by O, S or NR^{f} and
   R^{f} is hydrogen, C₁-C₃-alkyl, C₁-C₃-acyl, C₁-C₃-alkylsulphonyl or C₁-C₃-alkoxycarbonyl,
   and
   where, when
   R^{c} is a hydroxyl group, R^{d} can only be hydrogen, C₁-C₆-alkyl, C₂-C₈-alkenyl, C₂-C₈-alkynyl, C₃-C₁₀-cycloalkyl or C₆-C₁₂-aryl which is optionally substituted by W, and vice versa, and also
   R^{e} is hydrogen, C₁-C₆-alkyl, C₂-C₈-alkenyl, C₂-C₈-alkynyl, C₃-C₁₀-cycloalkyl or C₆-C₁₂-aryl which is optionally substituted by W,
   or
A is a C₃-C₁₀-cycloalkyl radical or 3-12-membered heterocycloalkyl radical which is optionally mono- or polysubstituted identically or differently by M and
   M is C₁-C₆-alkyl or a —COR^{b}, CO₂R^{b}, —O—R^{b} or —NR^{c}R^{d} group, where R^{b}, R^{c} and R^{d} are each as specified above, and
R¹ and R² are each independently an unbranched or branched C₁-C₅-alkyl group which is optionally substituted by Z or, together with the carbon atom of the chain, form a carbocyclic or heterocyclic ring which is optionally substituted by Z and has a total of 3-7 members, where, when
   A is hydrogen and R¹ is a methyl radical, R² cannot be a methyl radical or an ethyl radical,
   A is hydrogen, R¹ and R² cannot together be a ring having 3-4 members,
   A is a methyl radical, R¹ and R² cannot both be a methyl radical or, together with the carbon atom of the chain, form a cyclopropyl ring,
R³ is hydrogen or a C₁-C₈-alkyl, C₂-C₈-alkenyl, C₂-C₈-alkynyl, C₃-C₁₀-cycloalkyl, 3-12-membered heterocycloalkyl radical which is optionally mono- or polysubstituted identically or differently by K, or a C₆-C₁₂-aryl or 3-12-membered heteroaryl radical which is optionally mono-, di or trisubstituted identically or differently by L, and
   K is cyano, halogen, hydroxyl, nitro, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, —C═NOR^{b}, —NR^{c}R^{d} or a C₃-C₁₀-cycloalkyl, 3-12-membered heterocycloalkyl radical which is optionally mono- or polysubstituted identically or differently by M, or a C₆-C₁₂-aryl or 3-12-membered heteroaryl radical which is optionally mono-, di- or trisubstituted identically or differently by L, with the definition of M specified under A, and
   L is C₁-C₈-alkyl, C₂-C₈-alkenyl, C₂-C₈-alkynyl, a partly or fully fluorinated C₁-C₆-fluoroalkyl, a partly or fully fluorinated C₁-C₆-fluoroalkoxy, C₁-C₆-alkoxy-C₁-C₆-alkyl, C₁-C₆-alkoxy-C₁-C₆-alkoxy, a mono- or bicyclic (CH₂)ₚ—C₃-C₁₀-cycloalkyl, a mono- or bicyclic 3-12-membered (CH₂)ₚ-heterocycloalkyl radical, (CH₂)ₚCN, (CH₂)ₚHal, (CH₂)ₚNO₂, a mono- or bicyclic (CH₂)ₚ—C₆-C₁₂-aryl radical which is optionally substituted by V, a mono- or bicyclic 3-12-membered (CH₂)ₚ-heteroaryl radical which is optionally substituted by V, or
      —(CH₂)pPO3(Rb)2, —(CH2)pNRcRd, —(CH2)pNReCORb,
      —(CH2)pNReCSRb, —(CH2)pNReS(O) Rb, —(CH2)pNReS(O)2Rb,
      —(CH2)pNReCONRcRd, —(CH2)pNReCOORb,
      —(CH2)pNReC(NH)NRcRd, —(CH2)pNReCSNRcRd,
      —(CH2)pNReS(O)NRcRd, —(CH2)pNReS(O)₂NRcRd, —(CH2)pCORb,
      —(CH2)pCSRb, —(CH2)pS(O)Rb, —(CH2)pS(O)(NH)Rb,
      —(CH2)pS(O)₂Rb,
      —(CH2)pS(O)₂NRcRd, —(CH2)pSO2ORb, —(CH2)pCO2Rb,
      —(CH2)pCONRcRd, —(CH2)pCSNRcRd, —(CH2)pORb, —(CH2)pSRb,
      —(CH2)pCRb(OH)—Rb, —(CH₂)ₚ—C═NOR^{b},
      —O—(CH₂)ₙ—O—, —O—(CH₂)ₙ—CH₂—, —O—CH═CH— or —(CH₂)ₙ₊₂—, and the terminal oxygen atoms and/or carbon atoms are linked to directly adjacent ring carbon atoms, and
   n is 1 or 2 and
   p is 0, 1, 2, 3, 4, 5 or 6, and
   V is cyano, halogen, nitro, —(CH₂)ₚOR^{b}, —(CH₂)ₚS(O)₂R^{b}, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, —C═NOR^{b}, —(CH₂)ₚNR^{c}R^{d}, partly or fully fluorinated C₁-C₆-fluoroalkyl or partly or fully fluorinated C₁-C₆-fluoroalkoxy,
X is one oxygen atom or two hydrogen atoms
Y is (CH₂)ₘ, —C≡C— or —CH═CH— where
   m=0 or 1,
where, when Y is a CH₂ radical, R³ cannot be hydrogen, and
R⁴ is a mono- or bicyclic C₆-C₁₂-aryl which is optionally substituted identically or differently by 2 L, or one of the following groups mentioned under B or C:

### B: 6-Membered/6-Membered Ring Systems

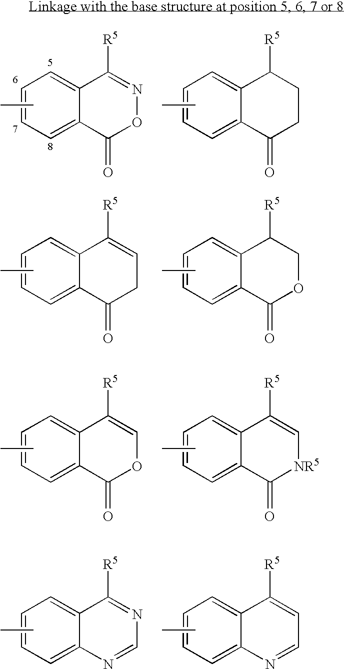

### C: 6-Membered/5-Membered Ring Systems

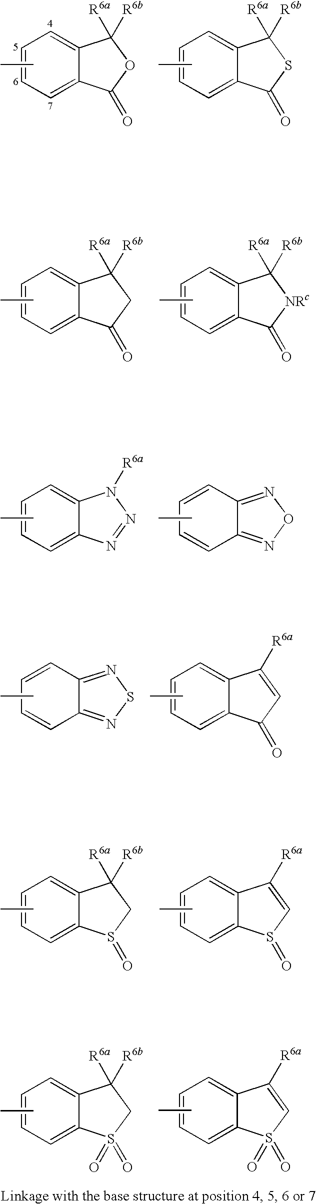 [0067] where
R⁵ is hydrogen or C₁-C₄-alkyl, or a partly or fully fluorinated C₁-C₄-fluoroalkyl,
R^{6a} and R^{6b} are each independently hydrogen, C₁-C₄-alkyl or a partly or fully fluorinated C₁-C₄-fluoroalkyl, or, together with the ring carbon atom, form a 3- to 6-membered ring,
   and the pharmaceutically acceptable salts thereof.

The compounds according to the invention of the general formula I may, owing to the presence of centres of asymmetry, exist as different stereoisomers. Both the racemates and the separate stereoisomers belong to the subject matter of the present invention.

The present invention further includes the novel compounds as active pharmaceutical ingredients, their therapeutic use and pharmaceutical dosage forms which comprise the novel substances.

The compounds according to the invention of the general formula (I) or their pharmaceutically acceptable salts can be used to produce a medicament, in particular for the treatment and prophylaxis of gynaecological disorders such as endometriosis, leiomyomas of the uterus, dysfunctional bleeding and dysmenorrhoea. The compounds according to the invention may further be used for the treatment and prophylaxis of hormone-dependent tumours such as, for example, for breast, prostate and endometrial carcinoma.

The compounds according to the invention of the general formula (I) or their pharmaceutically acceptable salts are also suitable for use for female fertility control or for female hormone replacement therapy.

The non-steroidal compounds according to the invention of the general formula I have strong antagonistic effects on the progesterone receptor with high potency. They show a strong dissociation of effects in relation to their strength of binding to the progesterone receptor and to the glucocorticoid receptor. Whereas known progesterone receptor antagonists such as mifepristone (RU 486) show, besides the desired high binding affinity for the progesterone receptor, likewise a high affinity for the glucocorticoid receptor, the compounds according to the invention are notable for a very low glucocorticoid receptor binding with simultaneously a high progesterone receptor affinity.

The substituents, defined as groups, of the compounds according to the invention of the general formula I may in each case have the following meanings:

C₁-C₃-, C₁-C₄-, C₁-C₅-, C₁-C₆- and C₁-C₈-alkyl group means unbranched or optionally branched alkyl radicals. Examples thereof are a methyl, ethyl, n-propyl, isopropyl, n-, iso-, tert-butyl, an n-pentyl, 2,2-dimethylpropyl, 3-methylbutyl, hexyl, heptyl or octyl group.

In the meaning of R¹, R² and R³, the methyl, ethyl, n-propyl or n-butyl group and an n-pentyl group are preferred.

According to the invention, preference is given to methyl or ethyl for R⁵, and to hydrogen for R^{6a} and R^{6b}.

Alkenyl means unbranched or optionally branched alkenyl radicals. Examples of the meaning of a C₂-C₈-alkenyl group in the context of the invention are the following: vinyl, allyl, 3-buten-1-yl or 2,3-dimethyl-2-propenyl. When the aromatic in R³ is substituted by a C₂-C₈-alkenyl radical, it is preferably a vinyl group.

Alkynyl means unbranched or optionally branched alkynyl radicals. A C₂-C₈-alkynyl radical is intended to be for example an ethynyl, propynyl, butynyl, pentynyl, hexynyl and octynyl group, but preferably an ethynyl or propynyl group.

A C₁-C₃-acyl radical in the context of R^{f} is a formyl, acetyl and an n- or isopropionyl radical. An acetyl radical is preferred for R^{f}.

C₁-C₃-Alkoxy is understood to mean a methoxy, ethoxy and an n- or isopropoxy radical. Methoxy and ethoxy are preferred.

Possible examples of C₁-C₆-alkoxyl-C₁-C₆-alkoxy group are methoxymethoxy, ethoxymethoxy or 2-methoxyethoxy.

A radical OR^{b} in the context of the invention is a hydroxy, methoxy, ethoxy, n-propoxy, isopropoxy, n-, iso-, tert-butoxy or n-pentoxy, 2,2-dimethylpropoxy or 3-methylbutoxy group. Hydroxy, methoxy and ethoxy are preferred.

Suitable for a partly or completely fluorinated C₁-C₃-, C₁-C₄- and C₁-C₆-fluoroalkyl group are in particular the trifluoromethyl or pentafluoroethyl group.

A halogen atom may be a fluorine, chlorine, bromine or iodine atom. Fluorine, chlorine or bromine is preferred here.

Possible examples of a mono- or bicyclic C₆-C₁₂-aryl radical in the meaning of R³, or R^{c}, R^{d}, R^{e}, and also K and L, are, for example, a phenyl or naphthyl radical, preferably a phenyl radical.

Examples of a 3-12-membered heteroaryl radical in the meaning of R³, K and L, and also R^{c} and R^{d}, are the 2-, 3- or 4-pyridinyl, the 2- or 3-furyl, the 2- or 3-thienyl, the 2- or 3-pyrrolyl, the 2-, 4- or 5-imidazolyl, the pyrazinyl, the 2-, 4- or 5-pyrimidinyl or 3- or 4-pyridazinyl radical.

5- to 6-membered C₃-C₁₀-cycloalkyl in the meaning of A, R³, K and L and 3- to 12-membered heterocycloalkyl groups in the meaning of A, R³, K and L are understood to mean both monocyclic and bicyclic groups.

Heteroatoms for 3-12-membered heteroaryls in the meaning of R^{b} and 5-12-membered heteroaryls in the meaning of R^{c} and R^{d} are nitrogen, sulfur or oxygen.

Examples which may be mentioned of monocyclic C₃-C₁₀-cycloalkyl in the meaning of R^{c} and R^{e} are cyclopropane, cyclobutane, cyclopentane and cyclohexane. Cyclopropyl, cyclopentyl and cyclohexyl are preferred.

Examples of monocyclic 3-12-membered or 5-12-membered heterocyclic radicals in the meaning of A, Z, K, R³ or R⁴ are morpholine, tetrahydrofuran, piperidine, pyrrolidine, oxirane, oxetane, aziridine, dioxolane, dioxane, thiophene, furan, pyran, pyrrole, imidazole, pyrazole, pyridine, pyrazine, pyrimidine, pyridazine, piperazine, thiazole, oxazole, furazan, pyrroline, thiazoline, triazole, tetrazole, using any of the chemically possible isomers in relation to the positions of the heteroatoms.

Examples which may be mentioned of bicyclic 3-12-membered or 5-12-membered heterocycles are quinoline, quinazoline and naphthyridine.

For R⁴, according to the invention, the bicyclic ring systems specified under B and C are preferred.

R¹ and R² which, together with the carbon atom of the chain, can form a carbocyclic ring having a total of 3-7 members are understood to mean carbocycles having 3 to 7 carbon atoms, preferably 3 to 6 carbon atoms. Particular preference is given to cyclopropyl, cyclopentyl and cyclohexyl.

Heterocycles in the sense of R¹ and R², which can be formed together with the carbon atom of the chain, may be cyclic ring compounds having at least one heteroatom, preferably oxygen, nitrogen and sulphur. Particular preference is given to tetrahydropyranyl, piperidinyl, tetrahydrothiopyranyl.

The number p for the (CH₂)ₚ radical may be a number 0, 1, 2, 3, 4, 5 or 6, preferably 0, 1 or 2. “Radical” means according to the invention all functional groups which are mentioned under L in connection with (CH₂)ₚ.

In the case where the compounds of the general formula I are in the form of salts, this is possible for example in the form of the hydrochloride, sulphate, nitrate, tartrate, citrate, fumarate, succinate or benzoate.

If the compounds according to the invention are in the form of racemic mixtures, they can be fractionated by methods of racemate resolution familiar to the skilled person into the pure optically active forms. For example, the racemic mixtures can be separated into the pure isomers by chromatography on a support material which is itself optically active (CHIRALPAK AD®). It is also possible to esterify the free hydroxy group in a racemic compound of the general formula I with an optically active acid, and to separate the resulting diastereoisomeric esters by fractional crystallization or chromatography and to hydrolyse the separated esters in each case to the optically pure isomers. It is possible to use as optically active acid for example mandelic acid, camphorsulphonic acid or tartaric acid.

Compounds of the general formula (I) preferred according to the present invention are those in which A is a hydrogen atom.

Further preferred are compounds in which:
Y is a —C≡C-radical, and
R¹ and R² together with the carbon atom of the chain form a carbocyclic or heterocyclic 3-6-membered ring and
R³ is optionally K-substituted C₁-C₈-alkyl, aryl optionally mono- or polysubstituted identically or differently by L, or 3- to 12-membered heteroaryl
or
Y is (CH₂)ₘ and
R³ is aryl optionally mono- or polysubstituted identically or differently by L or 3- to 12-membered heteroaryl, and
R⁴ is mono- or bicyclic aryl disubstituted identically or differently by L, or one of the B groups specified under R⁴ with linkage at position 6 or C with linkage at position 5.

In the case that Y is (CH₂)ₘ, m is preferably 1.

Irrespective of m, in the case that Y is (CH₂)ₘ, R⁴ is a phenyl ring substituted by 2 of the radicals specified under L.

For L, particular preference is given to a cyano radical, a chlorine and/or a trifluoromethyl radical.

In the case that Y is (CH₂)ₘ, R⁴, alternatively to the substituted phenyl ring, may also be defined as follows:

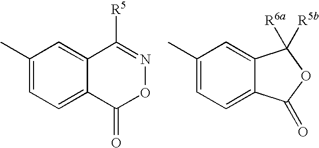

For these, the following substituents are preferred:
R⁵ is methyl or ethyl,
R⁶ is hydrogen.

Preference is additionally given to compounds of the general formula (I) in which
A is hydrogen,
p is 0, 1 or 2,
L is C₁-C₈-alkyl, C₂-C₈-alkenyl, C₂-C₈-alkynyl, a partly or fully fluorinated C₁-C₆-fluoroalkyl, —(CH₂)ₚCN, (CH₂)ₚHal, (CH₂)ₚNO₂, (CH₂)ₚ—C₆-C₁₂-aryl, —(CH₂)ₚ-heteroaryl, —(CH₂)ₚNR^{c}R^{d}, —(CH₂)ₚNR^{e}COR^{b}, —(CH₂)ₚNR^{e}S(O)₂R^{b}, —(CH₂)ₚNR^{e}CONR^{c}R^{d}, —(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚS(O)R^{b}, —(CH₂)ₚS(O)₂R^{b}, —(CH₂)ₚS(O)₂NR^{c}R^{d}, —(CH₂)ₚCO₂R^{b}, —(CH₂)ₚCONR^{c}R^{d}, —(CH₂)ₚOR^{b}, (H₂)ₚCR^{b}(OH)—R^{b} and
Z is cyano, halogen, hydroxyl, nitro, —C(O)R^{b}, CO₂R^{b}, —R^{b}, —SO₂NR^{c}R^{d}, —C(O)—NR^{c}R^{d}, —NR^{c}R^{d}, —NR^{e}COR^{b}, —NR^{e}S(O)R^{b}, —NR^{e}S(O)₂R^{b},
   NR^{e}CONR^{c}R^{d}, —S(O)R^{b}, —S(O)NR^{c}R^{d}, —S(O)₂R^{b}, —CR^{b}(OH)—R^{b} or a C₃-C₁₀-cycloalkyl or heterocycloalkyl optionally mono- or polysubstituted identically or differently by M.

Preference is additionally given to compounds of the general formula (I) in which
A is hydrogen and
R¹ and R² together with the carbon atom of the chain form a cyclopropyl, cyclopentyl or cyclohexyl ring.

Particular preference is given in this context in turn to a tetrahydropyranyl ring, a piperidinyl ring or a tetrahydrothiopyranyl ring.

The compounds mentioned below, and the use thereof, are preferred according to the invention:

| No. | Racemic or enantiomer | R3 |
|---|---|---|
| 1 2 3 | rac + − | 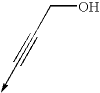 |
| 4 5 6 | rac + − | 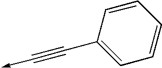 |
| 7 8 9 | rac + − | 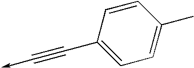 |
| 10 11 12 | rac + − | 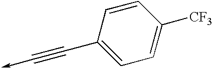 |
| 13 14 15 | rac + − | 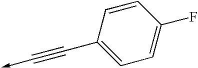 |
| 16 17 18 | rac + − | 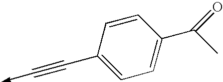 |
| 19 20 21 | rac + − | 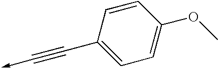 |
| 22 23 24 | rac + − | 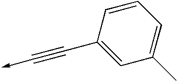 |
| 25 26 27 | rac + − | 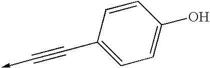 |
| 28 29 30 | rac + − | 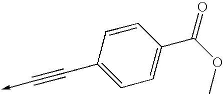 |
| 31 32 33 | rac + − | 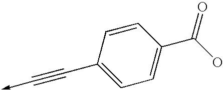 |
| 34 35 36 | rac + − | 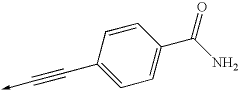 |
| 37 38 39 | rac + − | 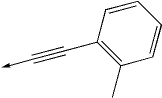 |
| 40 41 42 | rac + − | 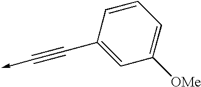 |
| 43 44 45 | rac + − | 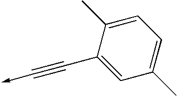 |
| 46 47 48 | rac + − | 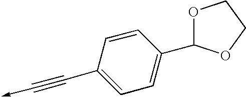 |
| 49 50 51 | rac + − | 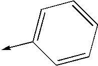 |
| 52 53 54 | rac + − | 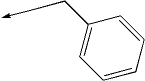 |
| 55 56 57 | rac + − | 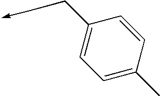 |
| 58 59 60 | rac + − | 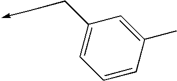 |
| 61 62 63 | rac + − | 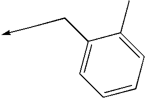 |
| 64 65 66 | rac + − | 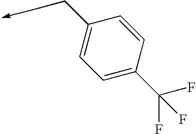 |
| 67 68 69 | rac + − | 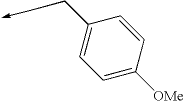 |
| 70 71 72 | rac + − | 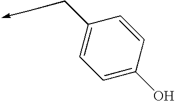 |
| 73 74 75 | rac + − | 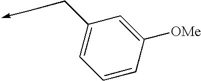 |
| 76 77 78 | rac + − | 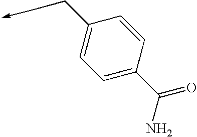 |
| 79 80 81 | rac + − | 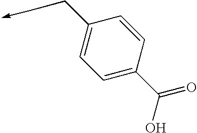 |
| 82 83 84 | rac + − | 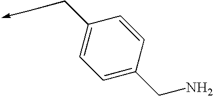 |
| 85 86 87 | rac + − | 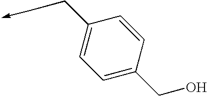 |
| 88 89 90 | rac + − | 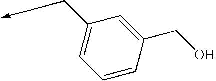 |
| 91 92 93 | rac + − | 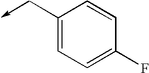 |
| 94 95 96 | rac + − | 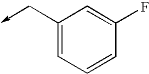 |
| 97 98 99 | rac + − | 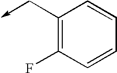 |
| 100 101 102 | rac + − | 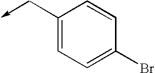 |
| 103 104 105 | rac + − | 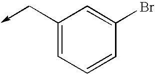 |
| 106 107 108 | rac + − | 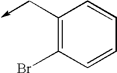 |
| 109 110 111 | rac + − | 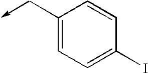 |
| 112 113 114 | rac + − | 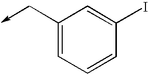 |
| 115 116 117 | rac + − | 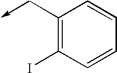 |
| 118 119 120 | rac + − | 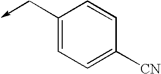 |
| 121 122 123 | rac + − | 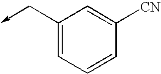 |
| 124 125 126 | rac + − | 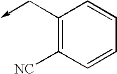 |
| 127 128 129 | rac + − | 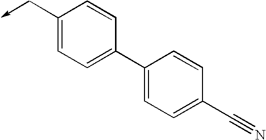 |
| 130 131 132 | rac + − | 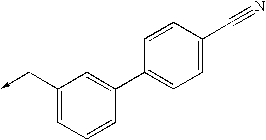 |
| 133 134 135 | rac + − | 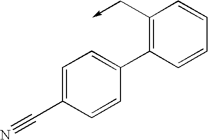 |
| 136 137 138 | rac + − | 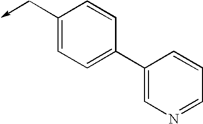 |
| 139 140 141 | rac + − | 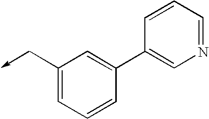 |
| 142 143 144 | rac + − | 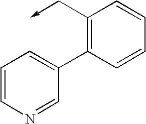 |
| 145 146 147 | rac + − | 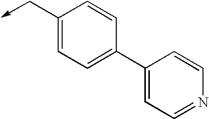 |
| 148 149 150 | rac + − | 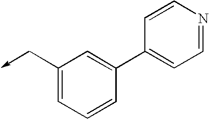 |
| 151 152 153 | rac + − | 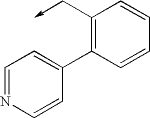 |
| 154 155 156 | rac + − | 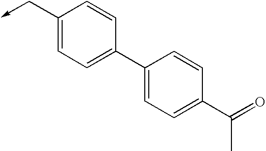 |
| 157 158 159 | rac + − | 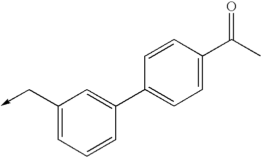 |
| 160 161 162 | rac + − | 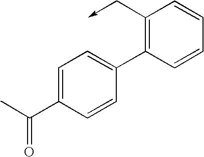 |
| 163 164 165 | rac + − | 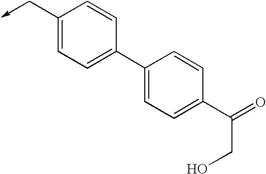 |
| 166 167 168 | rac + − | 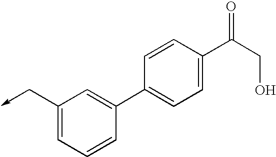 |
| 169 170 171 | rac + − | 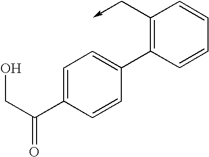 |
| 172 173 174 | rac + − | 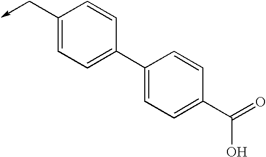 |
| 175 176 177 | rac + − | 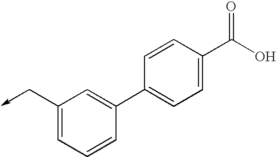 |
| 178 179 180 | rac + − | 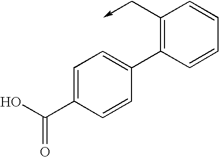 |
| 181 182 183 | rac + − | 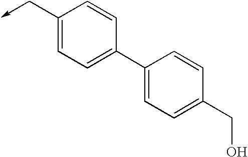 |
| 184 185 186 | rac + − | 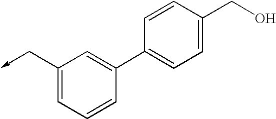 |
| 187 188 189 | rac + − | 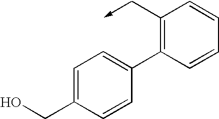 |
| 190 191 192 | rac + − | 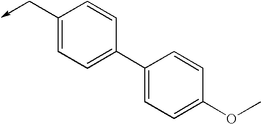 |
| 193 194 195 | rac + − | 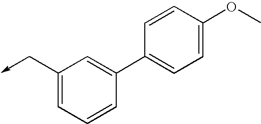 |
| 196 197 198 | rac + − | 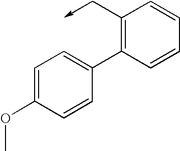 |
| 199 200 201 | rac + − | 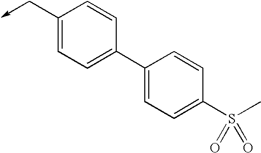 |
| 202 203 204 | rac + − | 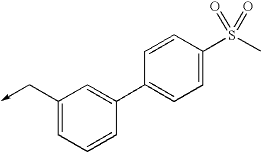 |
| 205 206 207 | rac + − | 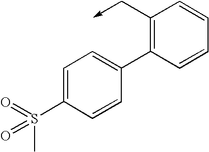 |
| 208 209 210 | rac + − | 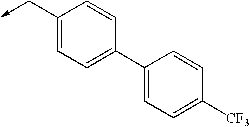 |
| 211 212 213 | rac + − | 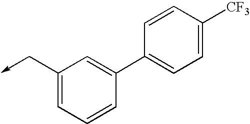 |
| 214 215 216 | rac + − | 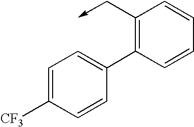 |
| 217 218 219 | rac + − | 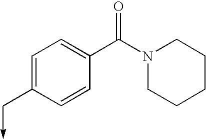 |
| 220 221 222 | rac + − | 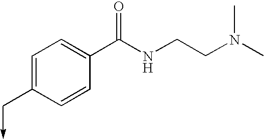 |
| 223 224 225 | rac + − | 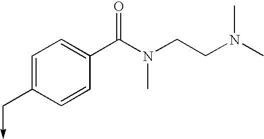 |
| 226 227 228 | rac + − | 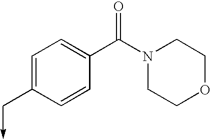 |
| 229 230 231 | rac + − | 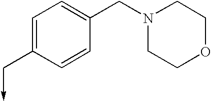 |
| 232 233 234 | rac + − | 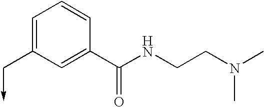 |
| 235 236 237 | rac + − | 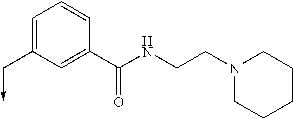 |
| 238 239 240 | rac + − | 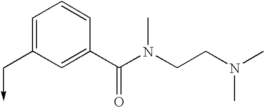 |
| 241 242 243 | rac + − | 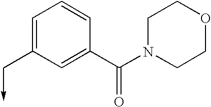 |
| 244 245 246 | rac + − | 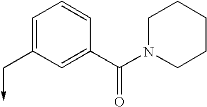 |
| 247 248 249 | rac + − | 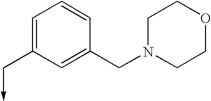 |
| 250 251 252 | rac + − | 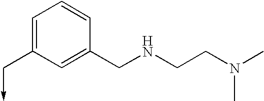 |
| 253 254 255 | rac + − | 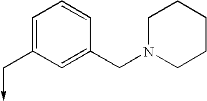 |
| 256 257 258 | rac + − | 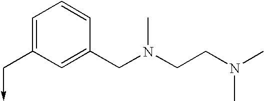 |
| 259 260 261 | rac + − | 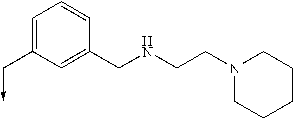 |

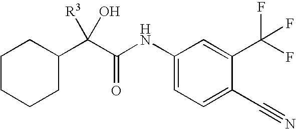

| No. | Racemic or enantiomer | R3 |
|---|---|---|
| 262 263 264 | rac + − | 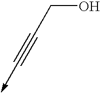 |
| 265 266 267 | rac + − | 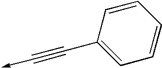 |
| 268 269 270 | rac + − | 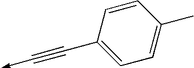 |
| 271 272 273 | rac + − | 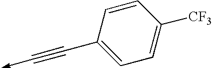 |
| 274 275 276 | rac + − | 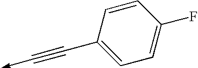 |
| 277 278 279 | rac + − | 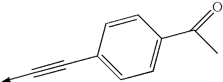 |
| 280 281 282 | rac + − | 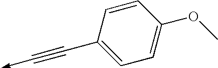 |
| 283 284 285 | rac + − | 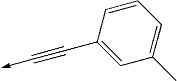 |
| 286 287 288 | rac + − | 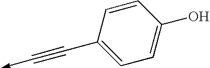 |
| 289 290 291 | rac + − | 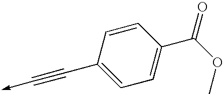 |
| 292 293 294 | rac + − | 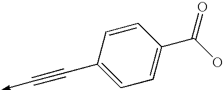 |
| 295 296 297 | rac + − | 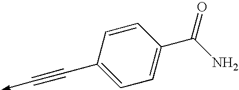 |
| 298 299 300 | rac + − | 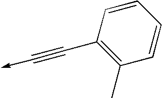 |
| 301 302 303 | rac + − | 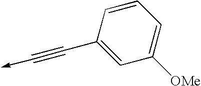 |
| 304 305 306 | rac + − | 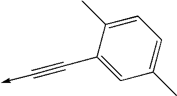 |
| 307 308 309 | rac + − | 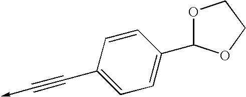 |
| 310 311 312 | rac + − | 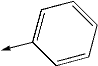 |
| 313 314 315 | rac + − | 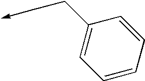 |
| 316 317 318 | rac + − | 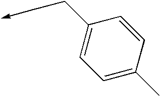 |
| 319 320 321 | rac + − | 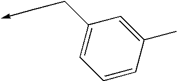 |
| 322 323 324 | rac + − | 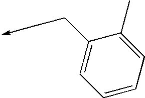 |
| 325 326 327 | rac + − | 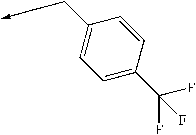 |
| 328 329 330 | rac + − | 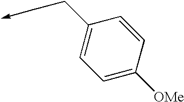 |
| 331 332 333 | rac + − | 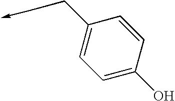 |
| 334 335 336 | rac + − | 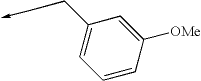 |
| 337 338 339 | rac + − | 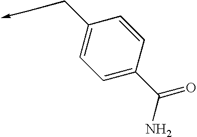 |
| 340 341 342 | rac + − | 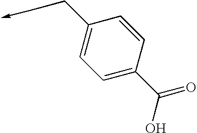 |
| 343 344 345 | rac + − | 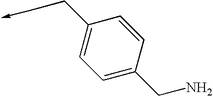 |
| 346 347 348 | rac + − | 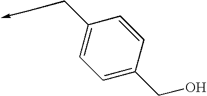 |
| 349 350 351 | rac + − | 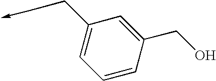 |
| 352 353 354 | rac + − | 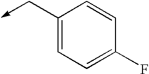 |
| 355 356 357 | rac + − | 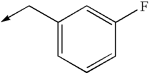 |
| 358 359 360 | rac + − | 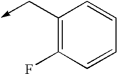 |
| 361 362 363 | rac + − | 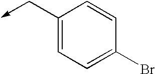 |
| 364 365 366 | rac + − | 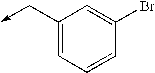 |
| 367 368 369 | rac + − | 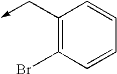 |
| 370 371 372 | rac + − | 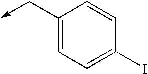 |
| 373 374 375 | rac + − | 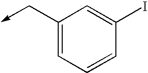 |
| 376 377 378 | rac + − | 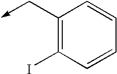 |
| 379 380 381 | rac + − | 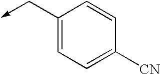 |
| 382 383 384 | rac + − | 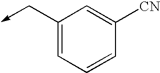 |
| 385 386 387 | rac + − | 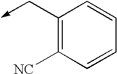 |
| 388 389 390 | rac + − | 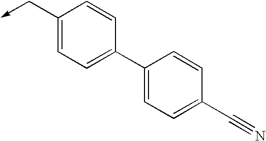 |
| 391 392 393 | rac + − | 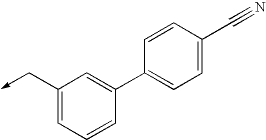 |
| 394 395 396 | rac + − | 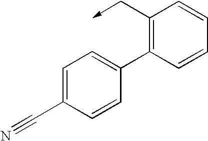 |
| 397 398 399 | rac + − | 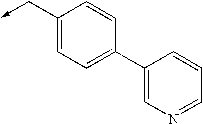 |
| 400 401 402 | rac + − | 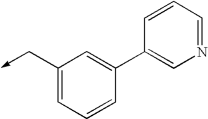 |
| 403 404 405 | rac + − | 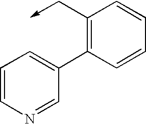 |
| 406 407 408 | rac + − | 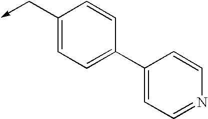 |
| 409 410 411 | rac + − | 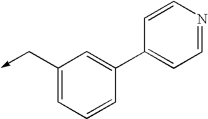 |
| 412 413 414 | rac + − | 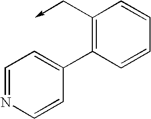 |
| 415 416 417 | rac + − | 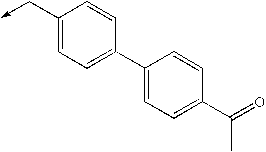 |
| 418 419 420 | rac + − | 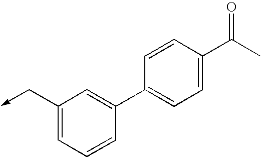 |
| 421 422 423 | rac + − | 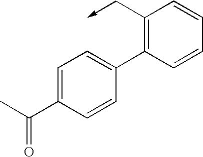 |
| 424 425 426 | rac + − | 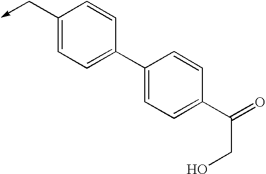 |
| 427 428 429 | rac + − | 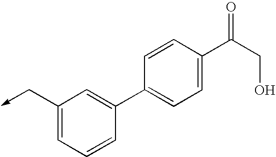 |
| 430 431 432 | rac + − | 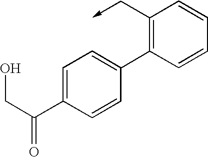 |
| 433 434 435 | rac + − | 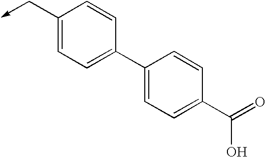 |
| 436 437 438 | rac + − | 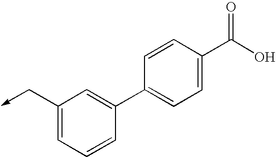 |
| 439 440 441 | rac + − | 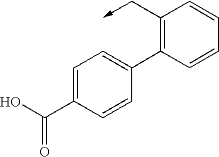 |
| 442 443 444 | rac + − | 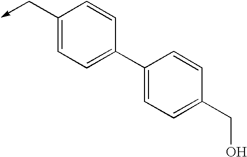 |
| 445 446 447 | rac + − | 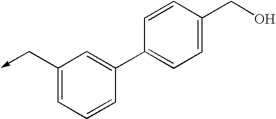 |
| 448 449 450 | rac + − | 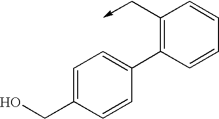 |
| 451 452 453 | rac + − | 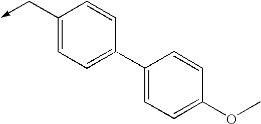 |
| 454 455 456 | rac + − | 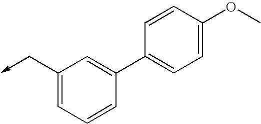 |
| 457 458 459 | rac + − | 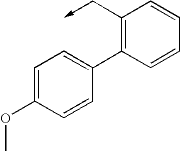 |
| 460 461 462 | rac + − | 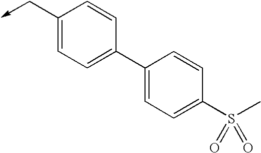 |
| 463 464 465 | rac + − | 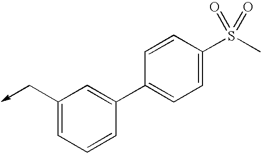 |
| 466 467 468 | rac + − | 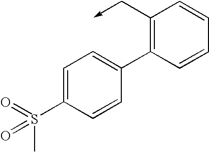 |
| 469 470 471 | rac + − | 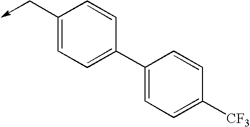 |
| 472 473 474 | rac + − | 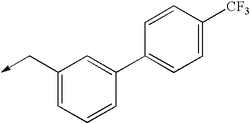 |
| 475 476 477 | rac + − | 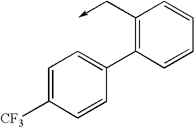 |
| 478 479 480 | rac + − | 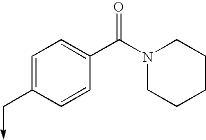 |
| 481 482 483 | rac + − | 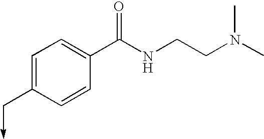 |
| 484 485 486 | rac + − | 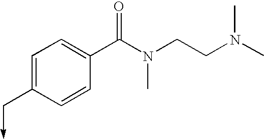 |
| 487 488 489 | rac + − | 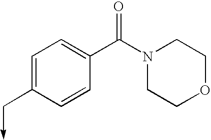 |
| 490 491 492 | rac + − | 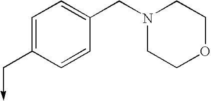 |
| 493 494 495 | rac + − | 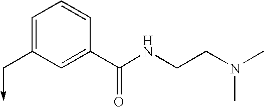 |
| 496 497 498 | rac + − | 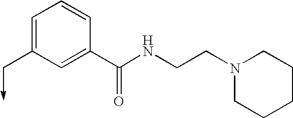 |
| 499 500 501 | rac + − | 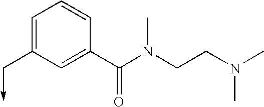 |
| 502 503 504 | rac + − | 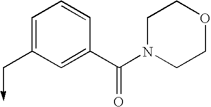 |
| 505 506 507 | rac + − | 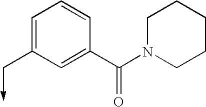 |
| 508 509 510 | rac + − | 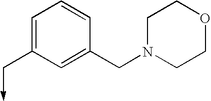 |
| 511 512 513 | rac + − | 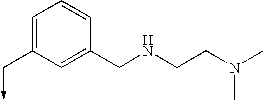 |
| 514 515 516 | rac + − | 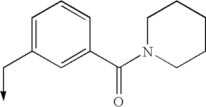 |
| 517 518 519 | rac + − | 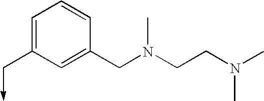 |
| 520 521 522 | rac + − | 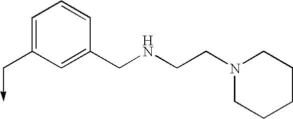 |
| 523 524 525 | rac + − | 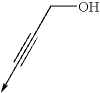 |
| 526 527 528 | rac + − | 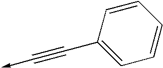 |
| 529 530 531 | rac + − | 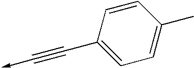 |
| 532 533 534 | rac + − | 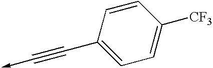 |
| 535 536 537 | rac + − | 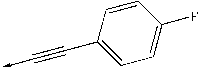 |
| 538 539 540 | rac + − | 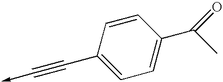 |
| 541 542 543 | rac + − | 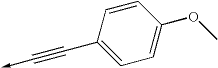 |
| 544 545 546 | rac + − | 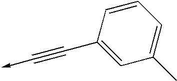 |
| 547 548 549 | rac + − | 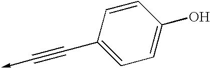 |
| 550 551 552 | rac + − | 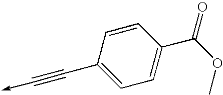 |
| 553 554 555 | rac + − | 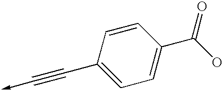 |
| 556 557 558 | rac + − | 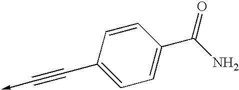 |
| 559 560 561 | rac + − | 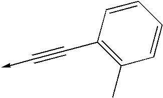 |
| 562 563 564 | rac + − | 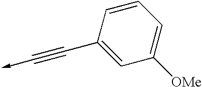 |
| 565 566 567 | rac + − | 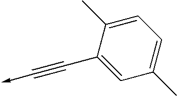 |
| 568 569 570 | rac + − | 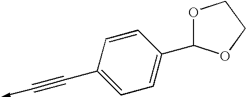 |
| 571 572 573 | rac + − | 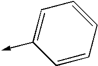 |
| 574 575 576 | rac + − | 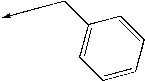 |
| 577 578 579 | rac + − | 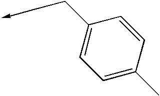 |
| 580 581 582 | rac + − | 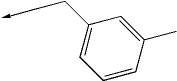 |
| 583 584 585 | rac + − | 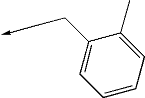 |
| 586 587 588 | rac + − | 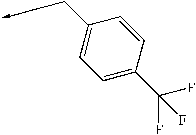 |
| 589 590 591 | rac + − | 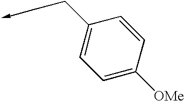 |
| 592 593 594 | rac + − | 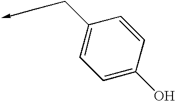 |
| 595 596 597 | rac + − | 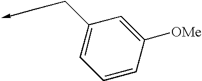 |
| 598 599 600 | rac + − | 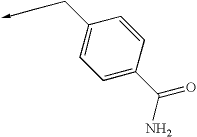 |
| 601 602 603 | rac + − | 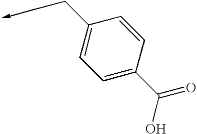 |
| 604 605 606 | rac + − | 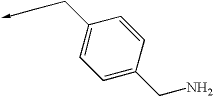 |
| 607 608 609 | rac + − | 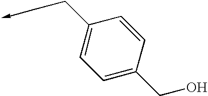 |
| 610 611 612 | rac + − | 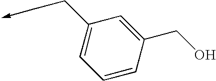 |
| 613 614 615 | rac + − | 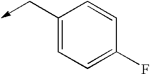 |
| 616 617 618 | rac + − | 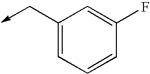 |
| 619 620 621 | rac + − | 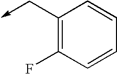 |
| 622 623 624 | rac + − | 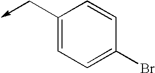 |
| 625 626 627 | rac + − | 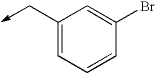 |
| 628 629 630 | rac + − | 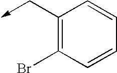 |
| 631 632 633 | rac + − | 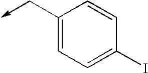 |
| 634 635 636 | rac + − | 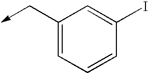 |
| 637 638 639 | rac + − | 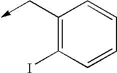 |
| 640 641 642 | rac + − | 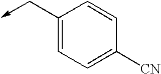 |
| 643 644 645 | rac + − | 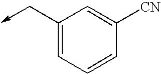 |
| 646 647 648 | rac + − | 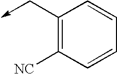 |
| 649 650 651 | rac + − | 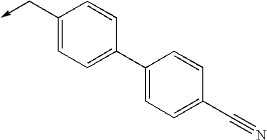 |
| 652 653 654 | rac + − | 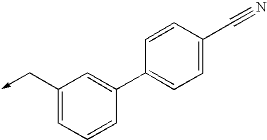 |
| 655 656 657 | rac + − | 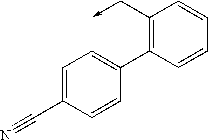 |
| 658 659 660 | rac + − | 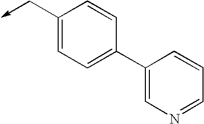 |
| 661 662 663 | rac + − | 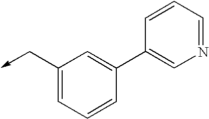 |
| 664 665 666 | rac + − | 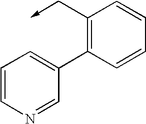 |
| 667 668 669 | rac + − | 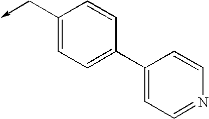 |
| 670 671 672 | rac + − | 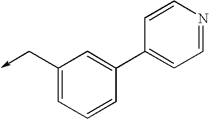 |
| 673 674 675 | rac + − | 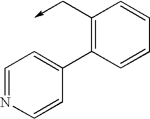 |
| 676 677 678 | rac + − | 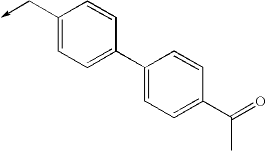 |
| 679 680 681 | rac + − | 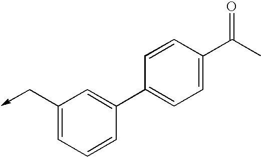 |
| 682 683 684 | rac + − | 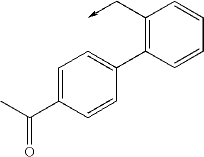 |
| 685 686 687 | rac + − | 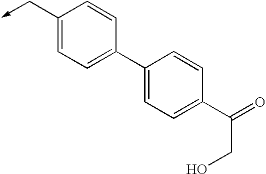 |
| 688 689 690 | rac + − | 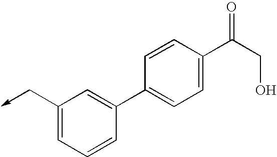 |
| 691 692 693 | rac + − | 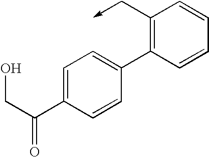 |
| 694 695 696 | rac + − | 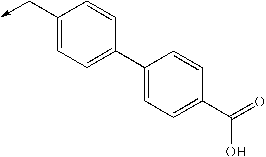 |
| 697 698 699 | rac + − | 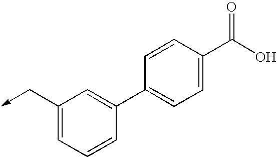 |
| 700 701 702 | rac + − | 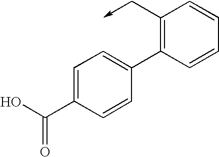 |
| 703 704 705 | rac + − | 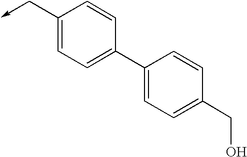 |
| 706 707 708 | rac + − | 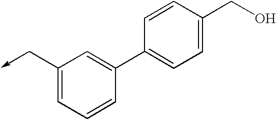 |
| 709 710 711 | rac + − | 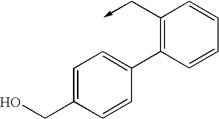 |
| 712 713 714 | rac + − | 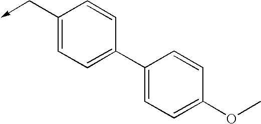 |
| 715 716 717 | rac + − | 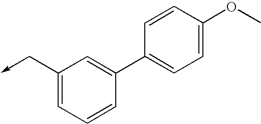 |
| 718 719 720 | rac + − | 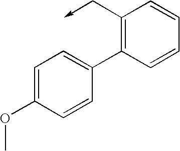 |
| 721 722 723 | rac + − | 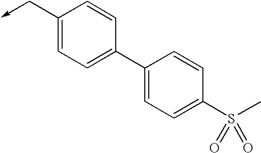 |
| 724 725 726 | rac + − | 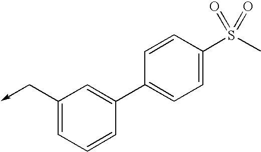 |
| 727 728 729 | rac + − | 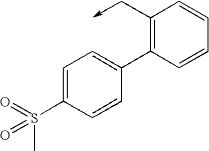 |
| 730 731 732 | rac + − | 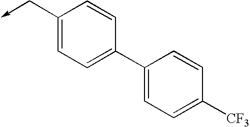 |
| 733 734 735 | rac + − | 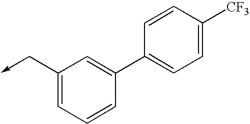 |
| 736 737 738 | rac + − | 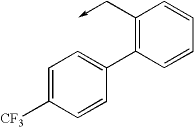 |
| 739 740 741 | rac + − | 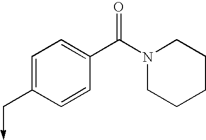 |
| 742 743 744 | rac + − | 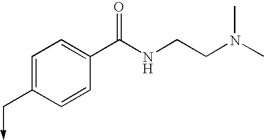 |
| 745 746 747 | rac + − | 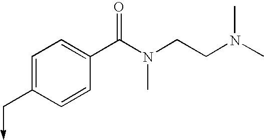 |
| 748 749 750 | rac + − | 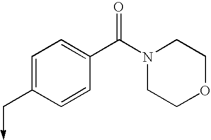 |
| 751 752 753 | rac + − | 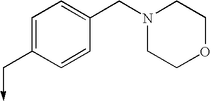 |
| 754 755 756 | rac + − | 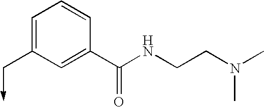 |
| 757 758 759 | rac + − | 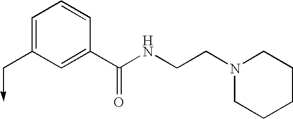 |
| 760 761 762 | rac + − | 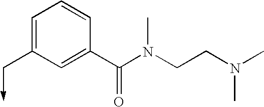 |
| 763 764 765 | rac + − | 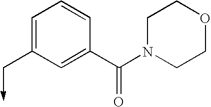 |
| 766 767 768 | rac + − | 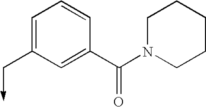 |
| 769 770 771 | rac + − | 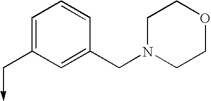 |
| 772 773 774 | rac + − | 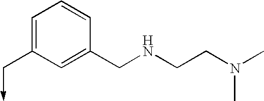 |
| 775 776 777 | rac + − | 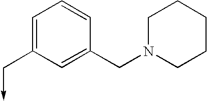 |
| 778 779 780 | rac + − | 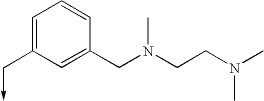 |
| 781 782 783 | rac + − | 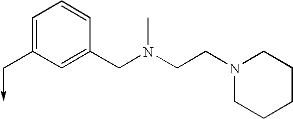 |
| 784 785 786 | rac + − | 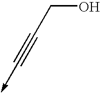 |
| 787 788 789 | rac + − | 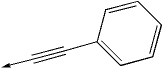 |
| 790 791 792 | rac + − | 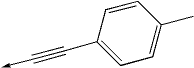 |
| 793 794 795 | rac + − | 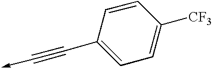 |
| 796 797 798 | rac + − | 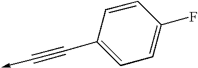 |
| 799 800 801 | rac + − | 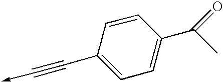 |
| 802 803 804 | rac + − | 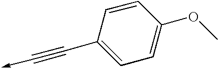 |
| 805 806 807 | rac + − | 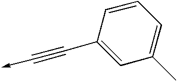 |
| 808 809 810 | rac + − | 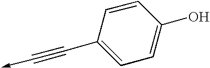 |
| 811 812 813 | rac + − | 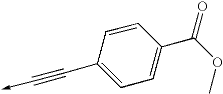 |
| 814 815 816 | rac + − | 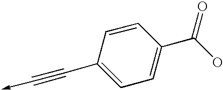 |
| 817 818 819 | rac + − | 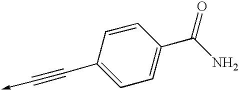 |
| 820 821 822 | rac + − | 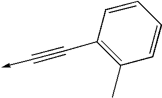 |
| 823 824 825 | rac + − | 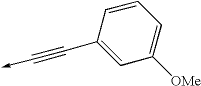 |
| 826 827 828 | rac + − | 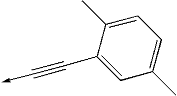 |
| 829 830 831 | rac + − | 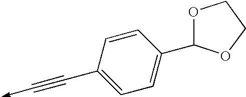 |
| 832 833 834 | rac + − | 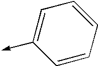 |
| 835 836 837 | rac + − | 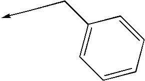 |
| 838 839 840 | rac + − | 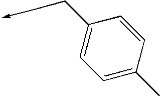 |
| 841 842 843 | rac + − | 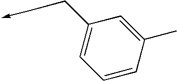 |
| 844 845 846 | rac + − | 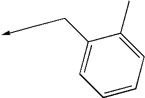 |
| 847 848 849 | rac + − | 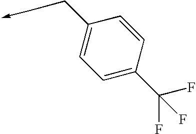 |
| 850 851 852 | rac + − | 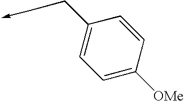 |
| 853 854 855 | rac + − | 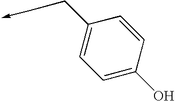 |
| 856 857 858 | rac + − | 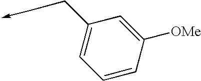 |
| 859 860 861 | rac + − | 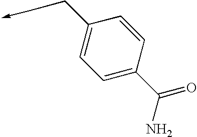 |
| 862 863 864 | rac + − | 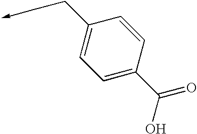 |
| 865 866 867 | rac + − | 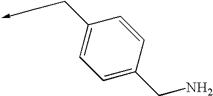 |
| 868 869 870 | rac + − | 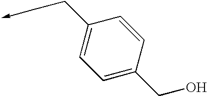 |
| 871 872 873 | rac + − | 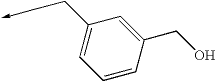 |
| 874 875 876 | rac + − | 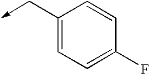 |
| 877 878 879 | rac + − | 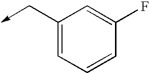 |
| 880 881 882 | rac + − | 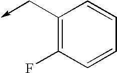 |
| 883 884 885 | rac + − | 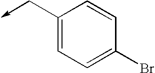 |
| 886 887 888 | rac + − | 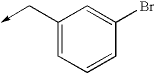 |
| 889 890 891 | rac + − | 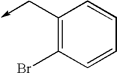 |
| 892 893 894 | rac + − | 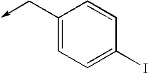 |
| 895 896 897 | rac + − | 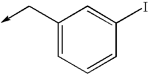 |
| 898 899 900 | rac + − | 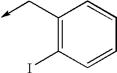 |
| 901 902 903 | rac + − | 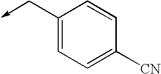 |
| 904 905 906 | rac + − | 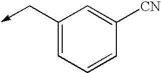 |
| 907 908 909 | rac + − | 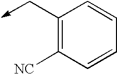 |
| 910 911 912 | rac + − | 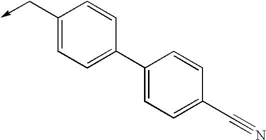 |
| 913 914 915 | rac + − | 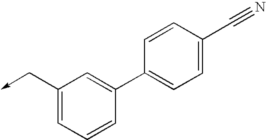 |
| 916 917 918 | rac + − | 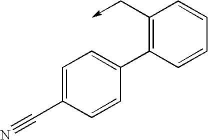 |
| 919 920 921 | rac + − | 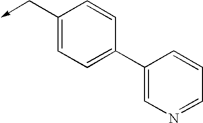 |
| 922 923 924 | rac + − | 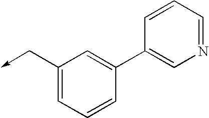 |
| 925 926 927 | rac + − | 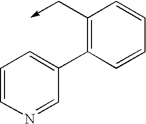 |
| 928 929 930 | rac + − | 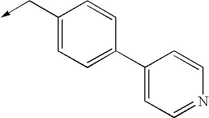 |
| 931 932 933 | rac + − | 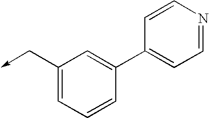 |
| 934 935 936 | rac + − | 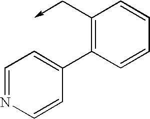 |
| 937 938 939 | rac + − | 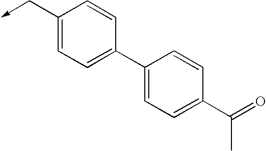 |
| 940 941 942 | rac + − | 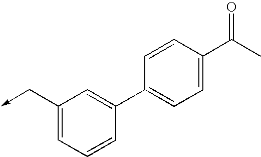 |
| 943 944 945 | rac + − | 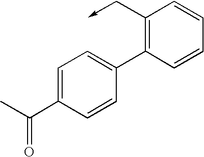 |
| 946 947 948 | rac + − | 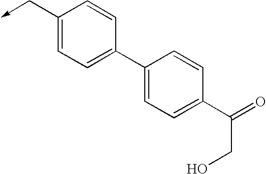 |
| 949 950 951 | rac + − | 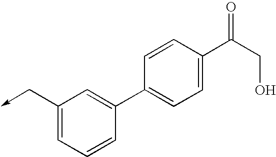 |
| 952 953 954 | rac + − | 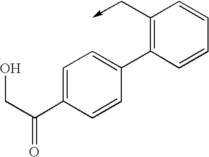 |
| 955 956 957 | rac + − | 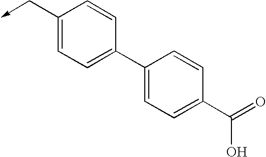 |
| 958 959 960 | rac + − | 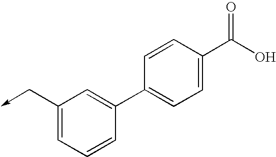 |
| 961 962 963 | rac + − | 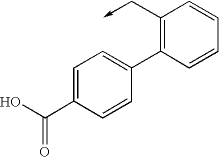 |
| 964 965 966 | rac + − | 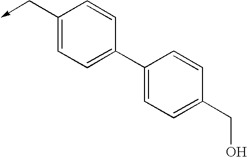 |
| 967 968 969 | rac + − | 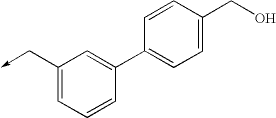 |
| 970 971 972 | rac + − | 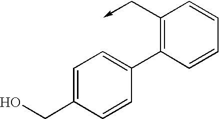 |
| 973 974 975 | rac + − | 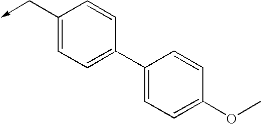 |
| 976 977 978 | rac + − | 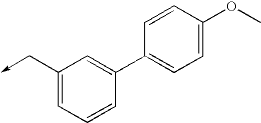 |
| 979 980 981 | rac + − | 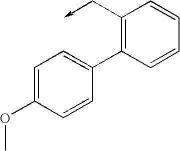 |
| 982 983 984 | rac + − | 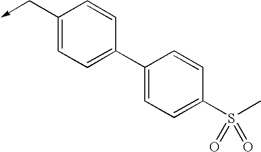 |
| 985 986 987 | rac + − | 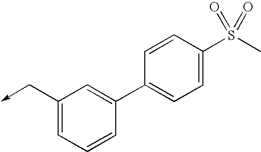 |
| 988 989 990 | rac + − | 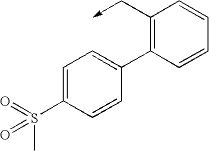 |
| 991 992 993 | rac + − | 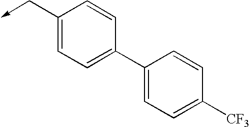 |
| 994 995 996 | rac + − | 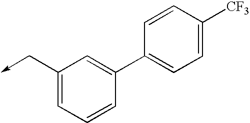 |
| 997 998 999 | rac + − | 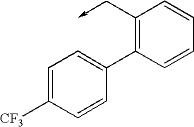 |
| 1000 1001 1002 | rac + − | 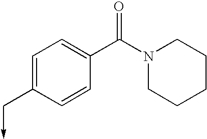 |
| 1003 1004 1005 | rac + − | 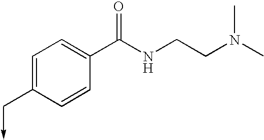 |
| 1006 1007 1008 | rac + − | 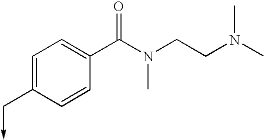 |
| 1009 1010 1011 | rac + − | 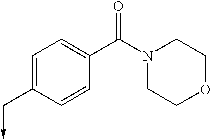 |
| 1012 1013 1014 | rac + − | 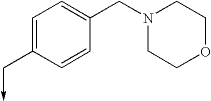 |
| 1015 1016 1017 | rac + − | 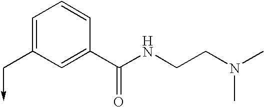 |
| 1018 1019 1020 | rac + − | 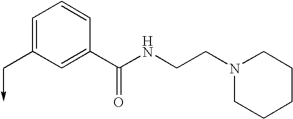 |
| 1021 1022 1023 | rac + − | 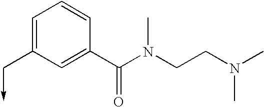 |
| 1024 1025 1026 | rac + − | 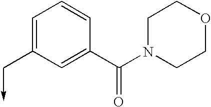 |
| 1027 1028 1029 | rac + − | 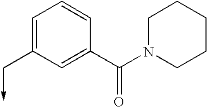 |
| 1030 1031 1032 | rac + − | 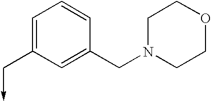 |
| 1033 1034 1035 | rac + − | 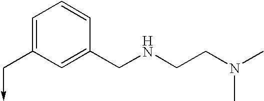 |
| 1036 1037 1038 | rac + − | 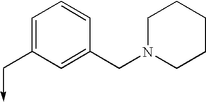 |
| 1039 1040 1041 | rac + − | 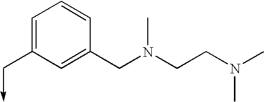 |
| 1042 1043 1044 | rac + − | 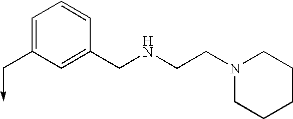 |

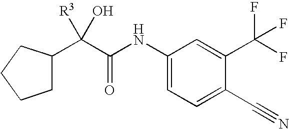

| No. | Racemic or enantiomer | R3 |
|---|---|---|
| 1045 1046 1047 | rac + − | 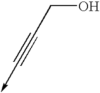 |
| 1048 1049 1050 | rac + − | 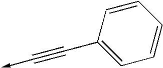 |
| 1051 1052 1053 | rac + − | 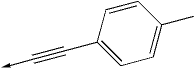 |
| 1054 1055 1056 | rac + − | 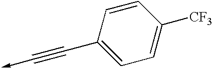 |
| 1057 1058 1059 | rac + − | 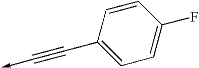 |
| 1060 1061 1062 | rac + − | 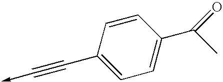 |
| 1063 1064 1065 | rac + − | 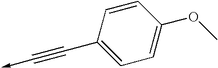 |
| 1066 1067 1068 | rac + − | 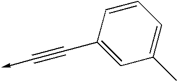 |
| 1069 1070 1071 | rac + − | 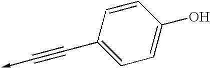 |
| 1072 1073 1074 | rac + − | 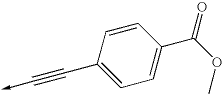 |
| 1075 1076 1077 | rac + − | 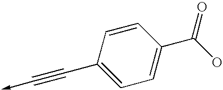 |
| 1078 1079 1080 | rac + − | 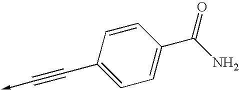 |
| 1081 1082 1083 | rac + − | 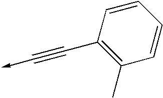 |
| 1084 1085 1086 | rac + − | 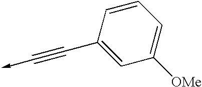 |
| 1087 1088 1089 | rac + − | 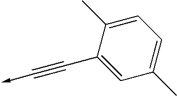 |
| 1090 1091 1092 | rac + − | 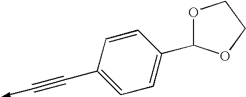 |
| 1093 1094 1095 | rac + − | 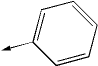 |
| 1096 1097 1098 | rac + − | 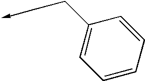 |
| 1099 1100 1101 | rac + − | 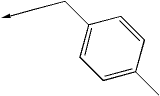 |
| 1102 1103 1104 | rac + − | 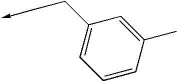 |
| 1105 1106 1107 | rac + − | 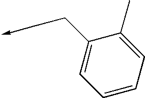 |
| 1108 1109 1110 | rac + − | 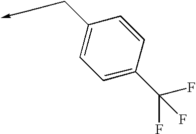 |
| 1111 1112 1113 | rac + − | 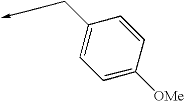 |
| 1114 1115 1116 | rac + − | 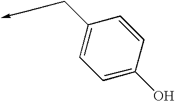 |
| 1117 1118 1119 | rac + − | 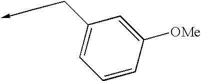 |
| 1120 1121 1122 | rac + − | 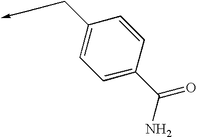 |
| 1123 1124 1125 | rac + − | 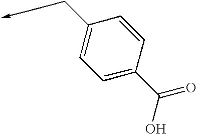 |
| 1126 1127 1128 | rac + − | 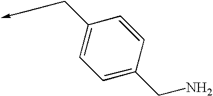 |
| 1129 1130 1131 | rac + − | 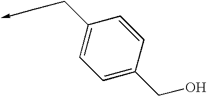 |
| 1132 1133 1134 | rac + − | 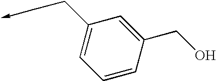 |
| 1135 1136 1137 | rac + − | 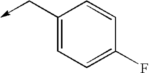 |
| 1138 1139 1140 | rac + − | 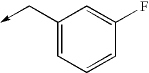 |
| 1141 1142 1143 | rac + − | 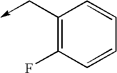 |
| 1144 1145 1146 | rac + − | 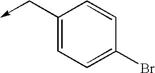 |
| 1147 1148 1149 | rac + − | 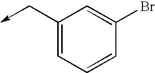 |
| 1150 1151 1152 | rac + − | 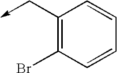 |
| 1153 1154 1155 | rac + − | 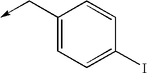 |
| 1156 1157 1158 | rac + − | 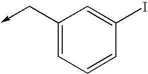 |
| 1159 1160 1161 | rac + − | 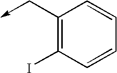 |
| 1162 1163 1164 | rac + − | 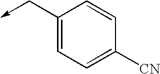 |
| 1165 1166 1167 | rac + − | 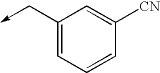 |
| 1168 1169 1170 | rac + − | 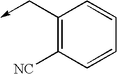 |
| 1171 1172 1173 | rac + − | 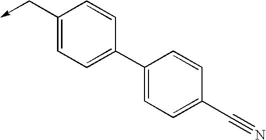 |
| 1174 1175 1176 | rac + − | 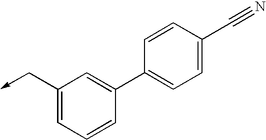 |
| 1177 1178 1179 | rac + − | 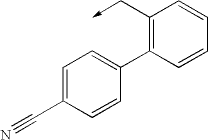 |
| 1180 1181 1182 | rac + − | 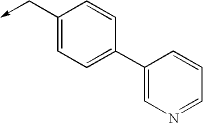 |
| 1183 1184 1185 | rac + − | 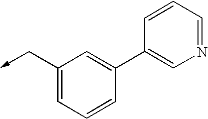 |
| 1186 1187 1188 | rac + − | 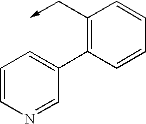 |
| 1189 1190 1191 | rac + − | 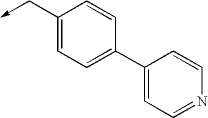 |
| 1192 1193 1194 | rac + − | 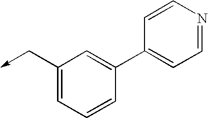 |
| 1195 1196 1197 | rac + − | 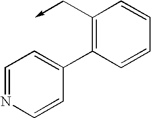 |
| 1198 1199 1200 | rac + − | 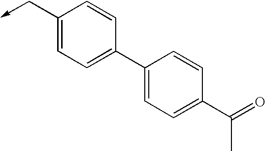 |
| 1201 1202 1203 | rac + − | 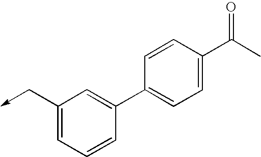 |
| 1204 1205 1206 | rac + − | 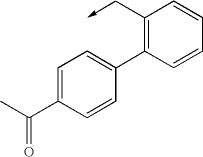 |
| 1207 1208 1209 | rac + − | 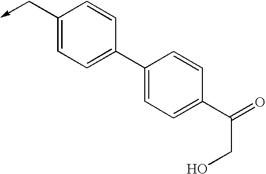 |
| 1210 1211 1212 | rac + − | 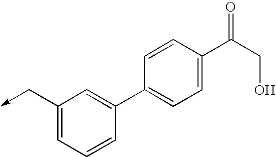 |
| 1213 1214 1215 | rac + − | 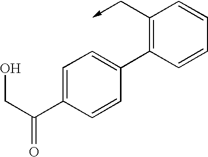 |
| 1216 1217 1218 | rac + − | 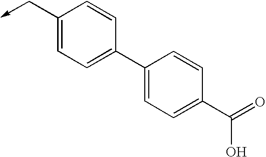 |
| 1219 1220 1221 | rac + − | 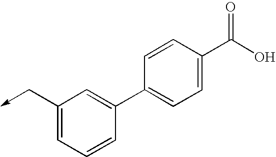 |
| 1222 1223 1224 | rac + − | 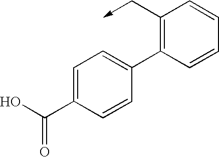 |
| 1225 1226 1227 | rac + − | 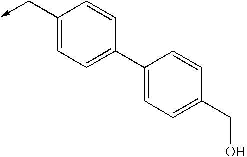 |
| 1228 1229 1230 | rac + − | 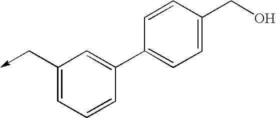 |
| 1231 1232 1233 | rac + − | 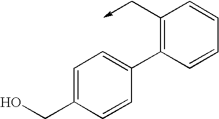 |
| 1234 1235 1236 | rac + − | 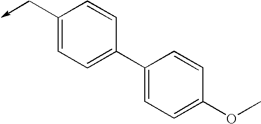 |
| 1237 1238 1239 | rac + − | 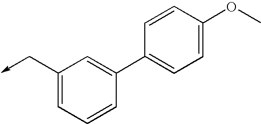 |
| 1240 1241 1242 | rac + − | 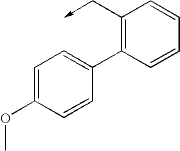 |
| 1243 1244 1245 | rac + − | 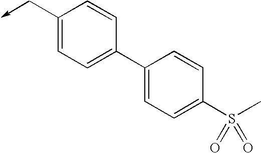 |
| 1246 1247 1248 | rac + − | 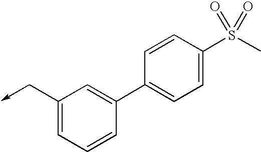 |
| 1249 1250 1251 | rac + − | 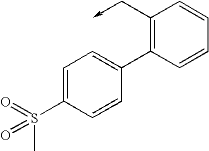 |
| 1252 1253 1254 | rac + − | 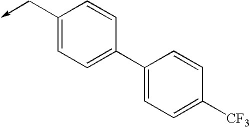 |
| 1255 1256 1257 | rac + − | 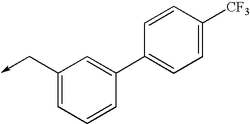 |
| 1258 1259 1260 | rac + − | 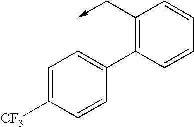 |
| 1261 1262 1263 | rac + − | 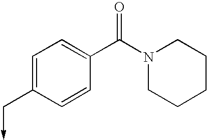 |
| 1264 1265 1266 | rac + − | 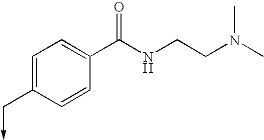 |
| 1267 1268 1269 | rac + − | 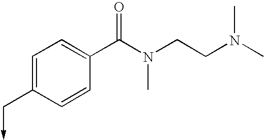 |
| 1270 1271 1272 | rac + − | 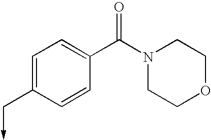 |
| 1273 1274 1275 | rac + − | 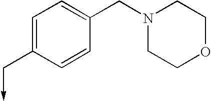 |
| 1276 1277 1278 | rac + − | 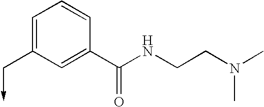 |
| 1279 1280 1281 | rac + − | 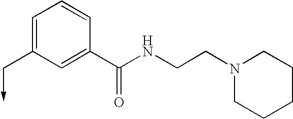 |
| 1282 1283 1284 | rac + − | 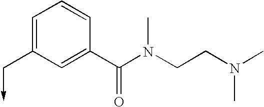 |
| 1285 1286 1287 | rac + − | 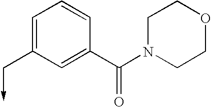 |
| 1288 1289 1290 | rac + − | 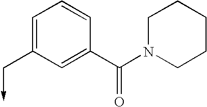 |
| 1291 1292 1293 | rac + − | 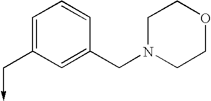 |
| 1294 1295 1296 | rac + − | 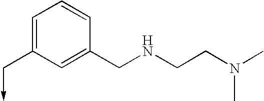 |
| 1297 1298 1299 | rac + − | 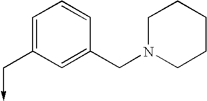 |
| 1300 1301 1302 | rac + − | 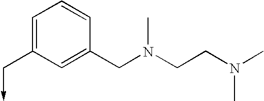 |
| 1303 1304 1305 | rac + − | 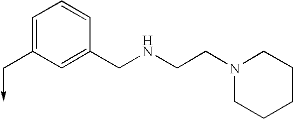 |

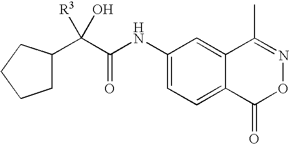

| No. | Racemic or enantiomer | R3 |
|---|---|---|
| 1306 1307 1308 | rac + − | 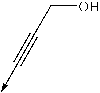 |
| 1309 1310 1311 | rac + − | 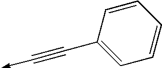 |
| 1312 1313 1314 | rac + − | 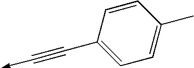 |
| 1315 1316 1317 | rac + − | 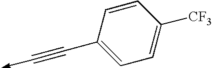 |
| 1318 1319 1320 | rac + − | 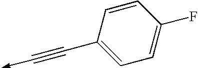 |
| 1321 1322 1323 | rac + − | 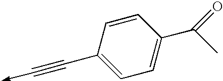 |
| 1324 1325 1326 | rac + − | 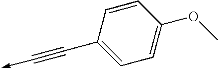 |
| 1327 1328 1329 | rac + − | 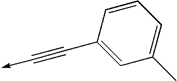 |
| 1330 1331 1332 | rac + − | 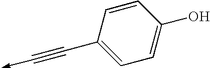 |
| 1333 1334 1335 | rac + − | 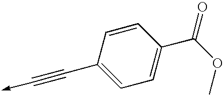 |
| 1336 1337 1338 | rac + − | 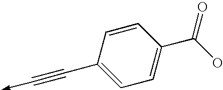 |
| 1339 1340 1341 | rac + − | 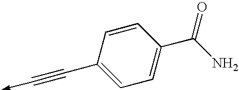 |
| 1342 1343 1344 | rac + − | 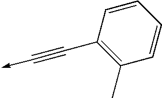 |
| 1345 1346 1347 | rac + − | 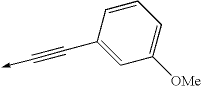 |
| 1348 1349 1350 | rac + − | 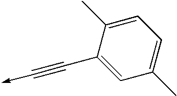 |
| 1351 1352 1353 | rac + − | 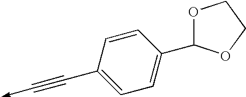 |
| 1354 1355 1356 | rac + − | 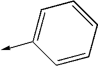 |
| 1357 1358 1359 | rac + − | 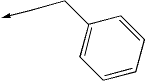 |
| 1360 1361 1362 | rac + − | 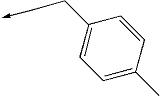 |
| 1363 1364 1365 | rac + − | 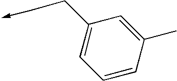 |
| 1366 1367 1368 | rac + − | 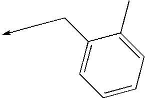 |
| 1369 1370 1371 | rac + − | 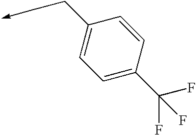 |
| 1372 1373 1374 | rac + − | 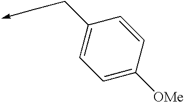 |
| 1375 1376 1377 | rac + − | 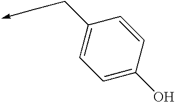 |
| 1378 1379 1380 | rac + − | 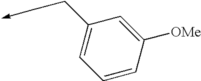 |
| 1381 1382 1383 | rac + − | 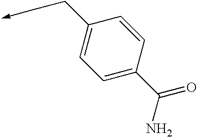 |
| 1384 1385 1386 | rac + − | 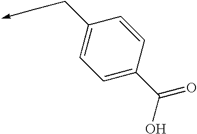 |
| 1387 1388 1389 | rac + − | 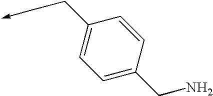 |
| 1390 1391 1392 | rac + − | 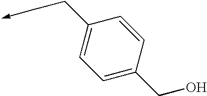 |
| 1393 1394 1395 | rac + − | 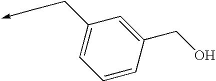 |
| 1396 1397 1398 | rac + − | 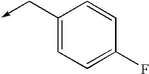 |
| 1399 1400 1401 | rac + − | 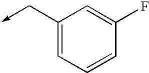 |
| 1402 1403 1404 | rac + − | 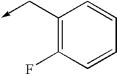 |
| 1405 1406 1407 | rac + − | 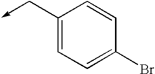 |
| 1408 1409 1410 | rac + − | 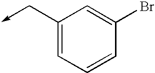 |
| 1411 1412 1413 | rac + − | 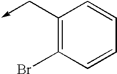 |
| 1414 1415 1416 | rac + − | 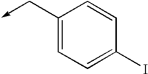 |
| 1417 1418 1419 | rac + − | 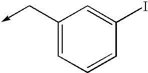 |
| 1420 1421 1422 | rac + − | 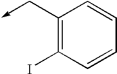 |
| 1423 1424 1425 | rac + − | 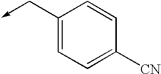 |
| 1426 1427 1428 | rac + − | 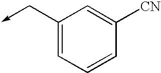 |
| 1429 1430 1431 | rac + − | 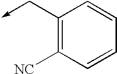 |
| 1432 1433 1434 | rac + − | 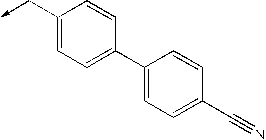 |
| 1435 1436 1437 | rac + − | 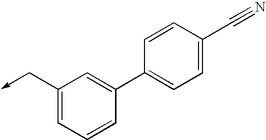 |
| 1438 1439 1440 | rac + − | 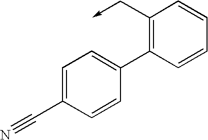 |
| 1441 1442 1443 | rac + − | 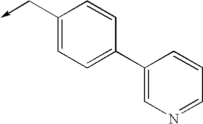 |
| 1444 1445 1446 | rac + − | 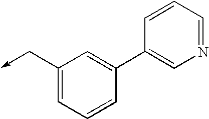 |
| 1447 1448 1449 | rac + − | 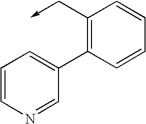 |
| 1450 1451 1452 | rac + − | 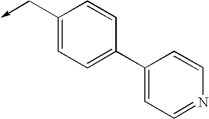 |
| 1453 1454 1455 | rac + − | 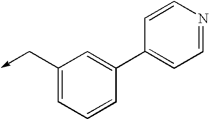 |
| 1456 1457 1458 | rac + − | 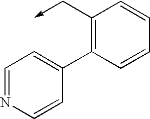 |
| 1459 1460 1461 | rac + − | 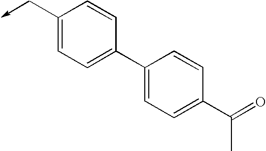 |
| 1462 1463 1464 | rac + − | 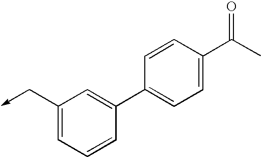 |
| 1465 1466 1467 | rac + − | 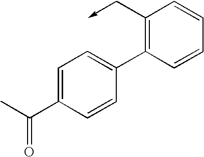 |
| 1468 1469 1470 | rac + − | 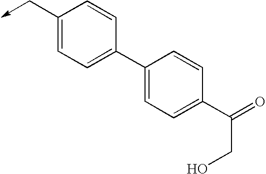 |
| 1471 1472 1473 | rac + − | 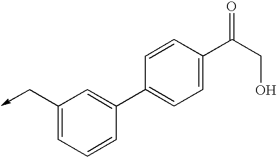 |
| 1474 1475 1476 | rac + − | 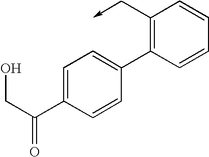 |
| 1477 1478 1479 | rac + − | 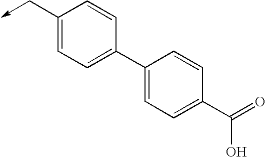 |
| 1480 1481 1482 | rac + − | 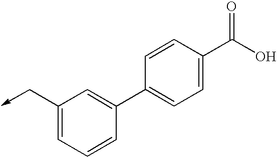 |
| 1483 1484 1485 | rac + − | 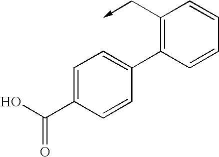 |
| 1486 1487 1488 | rac + − | 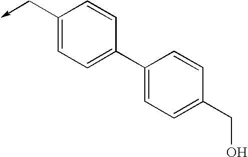 |
| 1489 1490 1491 | rac + − | 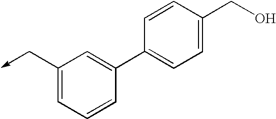 |
| 1492 1493 1494 | rac + − | 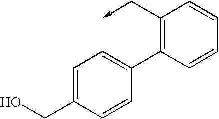 |
| 1495 1496 1497 | rac + − | 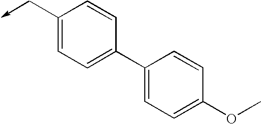 |
| 1498 1499 1500 | rac + − | 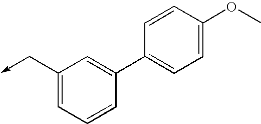 |
| 1501 1502 1503 | rac + − | 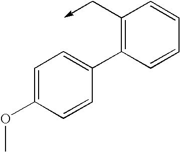 |
| 1504 1505 1506 | rac + − | 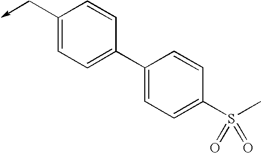 |
| 1507 1508 1509 | rac + − | 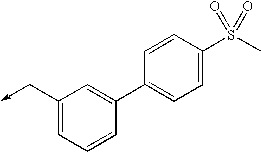 |
| 1510 1511 1512 | rac + − | 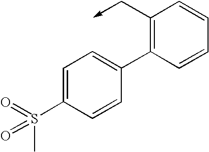 |
| 1513 1514 1515 | rac + − | 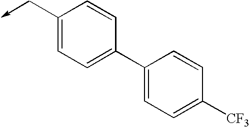 |
| 1516 1517 1518 | rac + − | 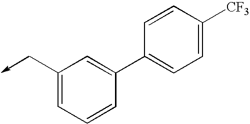 |
| 1519 1520 1521 | rac + − | 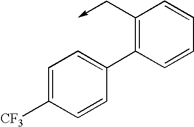 |
| 1522 1523 1524 | rac + − | 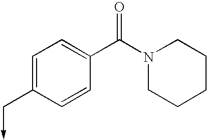 |
| 1525 1526 1527 | rac + − | 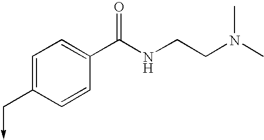 |
| 1528 1529 1530 | rac + − | 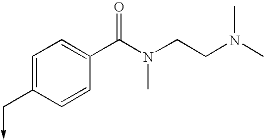 |
| 1531 1532 1533 | rac + − | 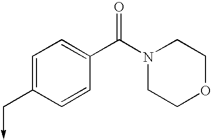 |
| 1534 1535 1536 | rac + − | 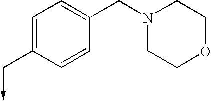 |
| 1537 1538 1539 | rac + − | 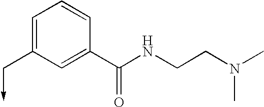 |
| 1540 1541 1542 | rac + − | 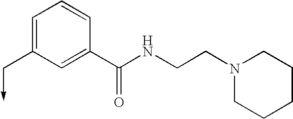 |
| 1543 1544 1545 | rac + − | 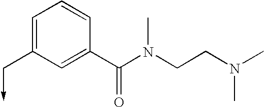 |
| 1546 1547 1548 | rac + − | 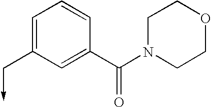 |
| 1549 1550 1551 | rac + − | 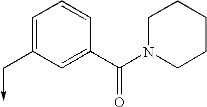 |
| 1552 1553 1554 | rac + − | 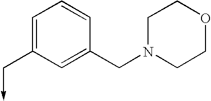 |
| 1555 1556 1557 | rac + − | 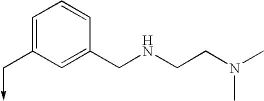 |
| 1558 1559 1560 | rac + − | 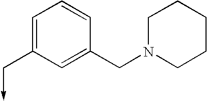 |
| 1561 1562 1563 | rac + − | 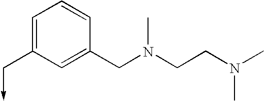 |
| 1564 1565 1566 | rac + − | 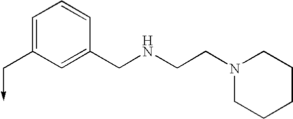 |

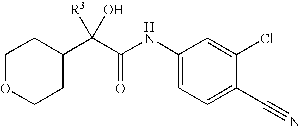

| No. | Racemic or enantiomer | R3 |
|---|---|---|
| 1567 1568 1569 | rac + − | 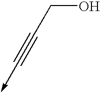 |
| 1570 1571 1572 | rac + − | 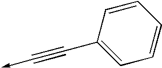 |
| 1573 1574 1575 | rac + − | 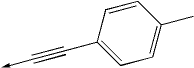 |
| 1576 1577 1578 | rac + − | 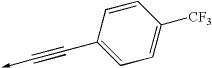 |
| 1579 1580 1581 | rac + − | 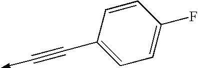 |
| 1582 1583 1584 | rac + − | 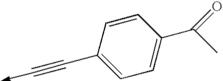 |
| 1585 1586 1587 | rac + − | 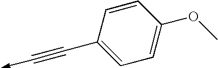 |
| 1588 1589 1590 | rac + − | 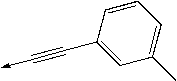 |
| 1591 1592 1593 | rac + − | 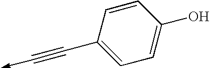 |
| 1594 1595 1596 | rac + − | 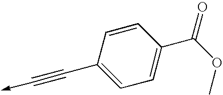 |
| 1597 1598 1599 | rac + − | 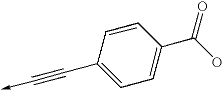 |
| 1600 1601 1602 | rac + − | 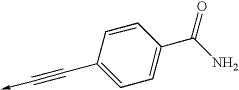 |
| 1603 1604 1605 | rac + − | 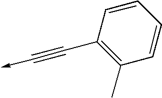 |
| 1606 1607 1608 | rac + − | 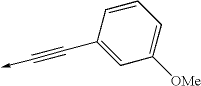 |
| 1609 1610 1611 | rac + − | 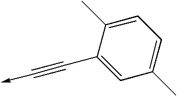 |
| 1612 1613 1614 | rac + − | 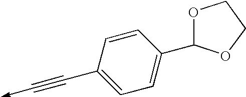 |
| 1615 1616 1617 | rac + − | 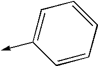 |
| 1618 1619 1620 | rac + − | 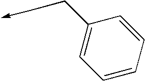 |
| 1621 1622 1623 | rac + − | 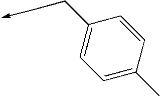 |
| 1624 1625 1626 | rac + − | 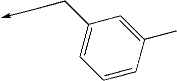 |
| 1627 1628 1629 | rac + − | 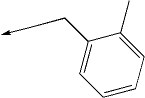 |
| 1630 1631 1632 | rac + − | 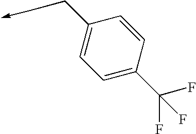 |
| 1633 1634 1635 | rac + − | 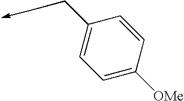 |
| 1636 1637 1638 | rac + − | 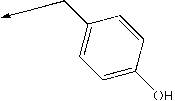 |
| 1639 1640 1641 | rac + − | 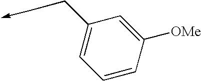 |
| 1642 1643 1644 | rac + − | 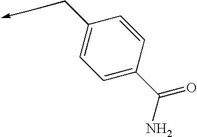 |
| 1645 1646 1647 | rac + − | 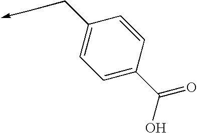 |
| 1648 1649 1650 | rac + − | 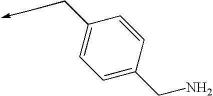 |
| 1651 1652 1653 | rac + − | 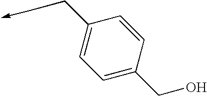 |
| 1654 1655 1656 | rac + − | 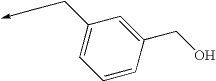 |
| 1657 1658 1659 | rac + − | 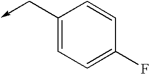 |
| 1660 1661 1662 | rac + − | 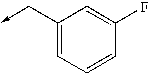 |
| 1663 1664 1665 | rac + − | 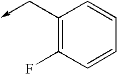 |
| 1666 1667 1668 | rac + − | 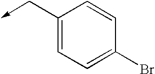 |
| 1669 1670 1671 | rac + − | 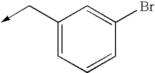 |
| 1672 1673 1674 | rac + − | 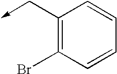 |
| 1675 1676 1677 | rac + − | 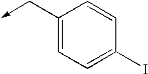 |
| 1678 1679 1680 | rac + − | 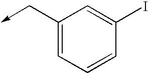 |
| 1681 1682 1683 | rac + − | 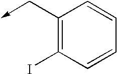 |
| 1684 1685 1686 | rac + − | 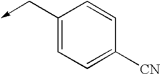 |
| 1687 1688 1689 | rac + − | 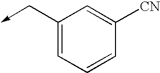 |
| 1690 1691 1692 | rac + − | 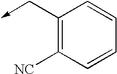 |
| 1693 1694 1695 | rac + − | 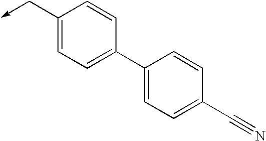 |
| 1696 1697 1698 | rac + − | 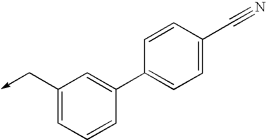 |
| 1699 1700 1701 | rac + − | 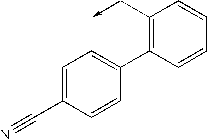 |
| 1702 1703 1704 | rac + − | 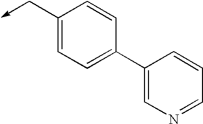 |
| 1705 1706 1707 | rac + − | 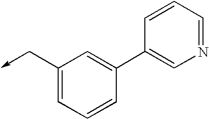 |
| 1708 1709 1710 | rac + − | 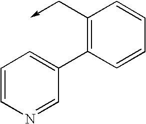 |
| 1711 1712 1713 | rac + − | 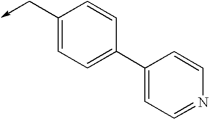 |
| 1714 1715 1716 | rac + − | 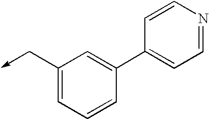 |
| 1717 1718 1719 | rac + − | 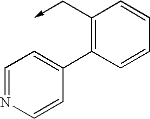 |
| 1720 1721 1722 | rac + − | 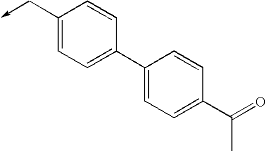 |
| 1723 1724 1725 | rac + − | 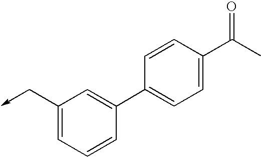 |
| 1726 1727 1728 | rac + − | 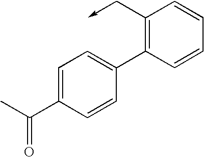 |
| 1729 1730 1731 | rac + − | 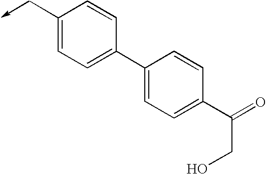 |
| 1732 1733 1734 | rac + − | 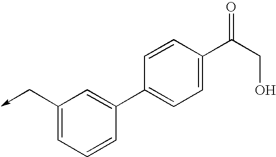 |
| 1735 1736 1737 | rac + − | 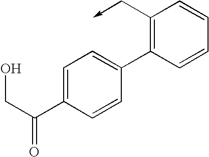 |
| 1738 1739 1740 | rac + − | 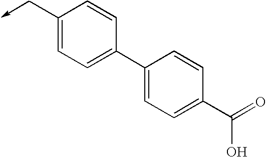 |
| 1741 1742 1743 | rac + − | 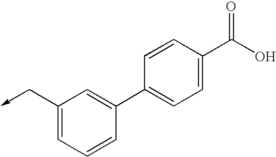 |
| 1744 1745 1746 | rac + − | 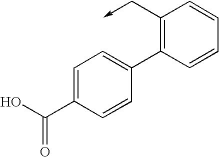 |
| 1747 1748 1749 | rac + − | 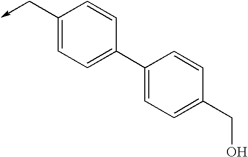 |
| 1750 1751 1752 | rac + − | 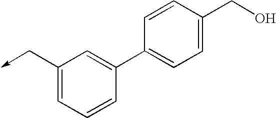 |
| 1753 1754 1755 | rac + − | 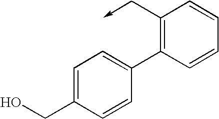 |
| 1756 1757 1758 | rac + − | 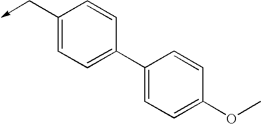 |
| 1759 1760 1761 | rac + − | 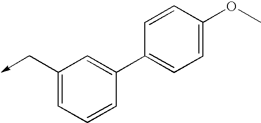 |
| 1762 1763 1764 | rac + − | 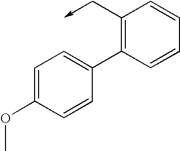 |
| 1765 1766 1767 | rac + − | 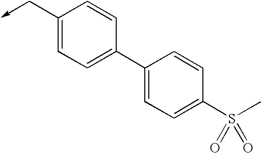 |
| 1768 1769 1770 | rac + − | 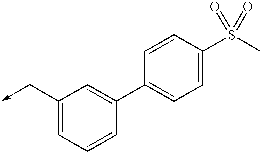 |
| 1771 1772 1773 | rac + − | 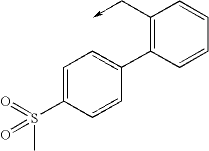 |
| 1774 1775 1776 | rac + − | 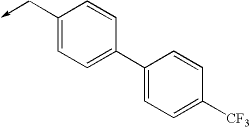 |
| 1777 1778 1779 | rac + − | 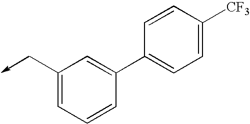 |
| 1780 1781 1782 | rac + − | 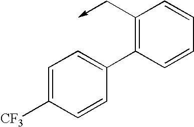 |
| 1783 1784 1785 | rac + − | 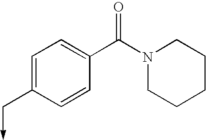 |
| 1786 1787 1788 | rac + − | 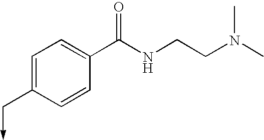 |
| 1789 1790 1791 | rac + − | 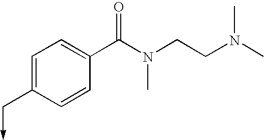 |
| 1792 1793 1794 | rac + − | 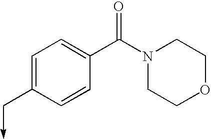 |
| 1795 1796 1797 | rac + − | 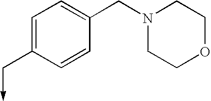 |
| 1798 1799 1800 | rac + − | 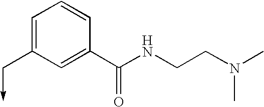 |
| 1801 1802 1803 | rac + − | 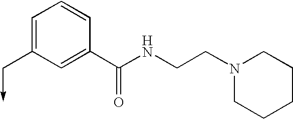 |
| 1804 1805 1806 | rac + − | 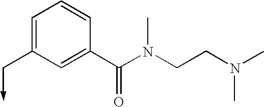 |
| 1807 1808 1809 | rac + − | 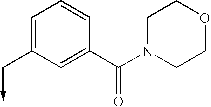 |
| 1810 1811 1812 | rac + − | 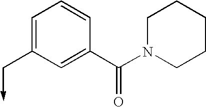 |
| 1813 1814 1815 | rac + − | 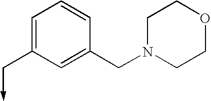 |
| 1816 1817 1818 | rac + − | 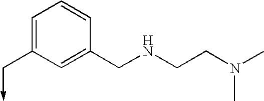 |
| 1819 1820 1821 | rac + − | 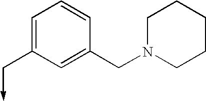 |
| 1822 1823 1824 | rac + − | 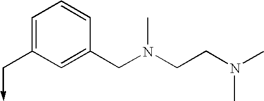 |
| 1825 1826 1827 | rac + − | 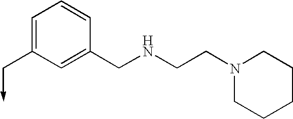 |

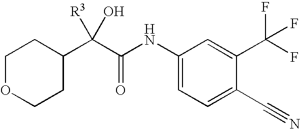

| No. | Racemic or enantiomer | R3 |
|---|---|---|
| 1828 1829 1830 | rac + − | 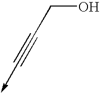 |
| 1831 1832 1833 | rac + − | 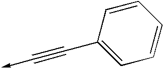 |
| 1834 1835 1836 | rac + − | 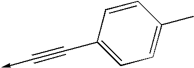 |
| 1837 1838 1839 | rac + − | 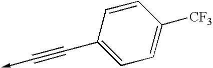 |
| 1840 1841 1842 | rac + − | 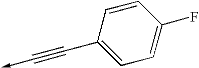 |
| 1843 1844 1845 | rac + − | 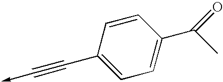 |
| 1846 1847 1848 | rac + − | 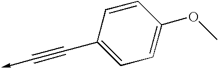 |
| 1849 1850 1851 | rac + − | 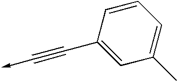 |
| 1852 1853 1854 | rac + − | 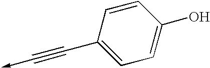 |
| 1855 1856 1857 | rac + − | 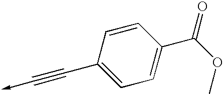 |
| 1858 1859 1860 | rac + − | 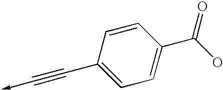 |
| 1861 1862 1863 | rac + − | 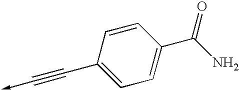 |
| 1864 1865 1866 | rac + − | 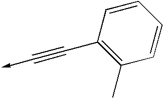 |
| 1867 1868 1869 | rac + − | 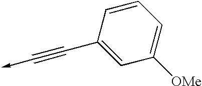 |
| 1870 1871 1872 | rac + − | 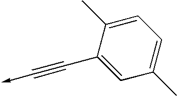 |
| 1873 1874 1875 | rac + − | 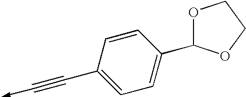 |
| 1876 1877 1878 | rac + − | 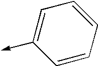 |
| 1879 1880 1881 | rac + − | 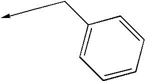 |
| 1882 1883 1884 | rac + − | 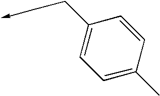 |
| 1885 1886 1887 | rac + − | 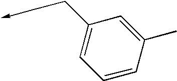 |
| 1888 1889 1890 | rac + − | 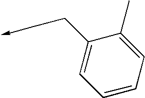 |
| 1891 1892 1893 | rac + − | 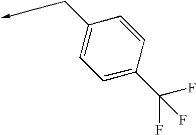 |
| 1894 1895 1896 | rac + − | 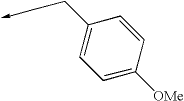 |
| 1897 1898 1899 | rac + − | 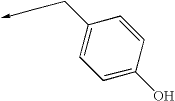 |
| 1900 1901 1902 | rac + − | 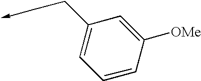 |
| 1903 1904 1905 | rac + − | 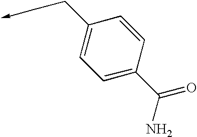 |
| 1906 1907 1908 | rac + − | 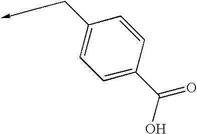 |
| 1909 1910 1911 | rac + − | 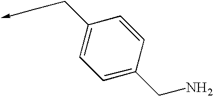 |
| 1912 1913 1914 | rac + − | 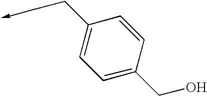 |
| 1915 1916 1917 | rac + − | 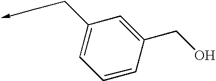 |
| 1918 1919 1920 | rac + − | 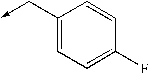 |
| 1921 1922 1923 | rac + − | 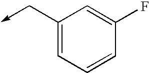 |
| 1924 1925 1926 | rac + − | 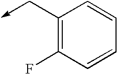 |
| 1927 1928 1929 | rac + − | 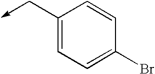 |
| 1930 1931 1932 | rac + − | 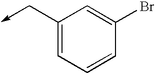 |
| 1933 1934 1935 | rac + − | 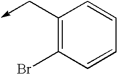 |
| 1936 1937 1938 | rac + − | 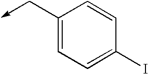 |
| 1939 1940 1941 | rac + − | 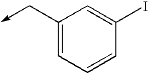 |
| 1942 1943 1944 | rac + − | 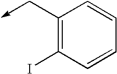 |
| 1945 1946 1947 | rac + − | 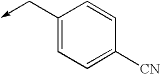 |
| 1948 1949 1950 | rac + − | 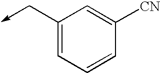 |
| 1951 1952 1953 | rac + − | 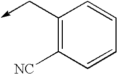 |
| 1954 1955 1956 | rac + − | 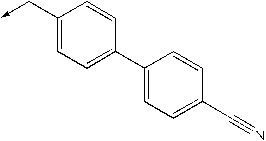 |
| 1957 1958 1959 | rac + − | 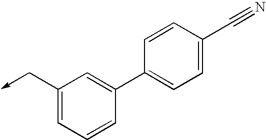 |
| 1960 1961 1962 | rac + − | 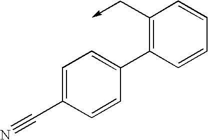 |
| 1963 1964 1965 | rac + − | 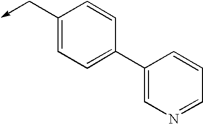 |
| 1966 1967 1968 | rac + − | 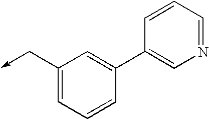 |
| 1969 1970 1971 | rac + − | 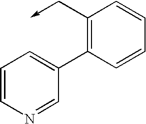 |
| 1972 1973 1974 | rac + − | 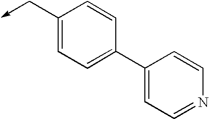 |
| 1975 1976 1977 | rac + − | 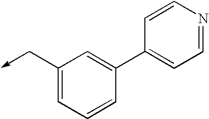 |
| 1978 1979 1980 | rac + − | 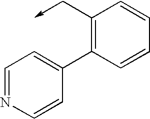 |
| 1981 1982 1983 | rac + − | 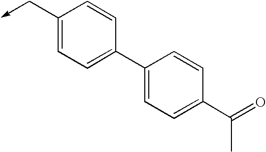 |
| 1984 1985 1986 | rac + − | 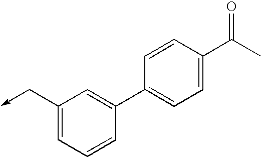 |
| 1987 1988 1989 | rac + − | 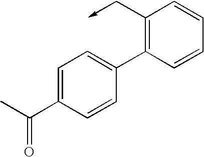 |
| 1990 1991 1992 | rac + − | 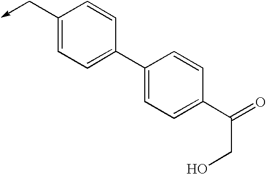 |
| 1993 1994 1995 | rac + − | 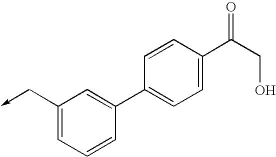 |
| 1996 1997 1998 | rac + − | 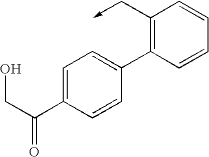 |
| 1999 2000 2001 | rac + − | 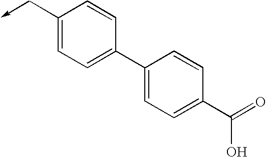 |
| 2002 2003 2004 | rac + − | 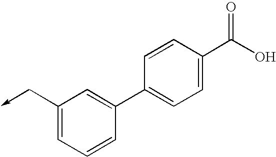 |
| 2005 2006 2007 | rac + − | 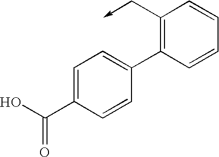 |
| 2008 2009 2010 | rac + − | 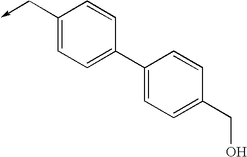 |
| 2011 2012 2013 | rac + − | 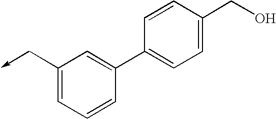 |
| 2014 2015 2016 | rac + − | 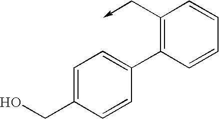 |
| 2017 2018 2019 | rac + − | 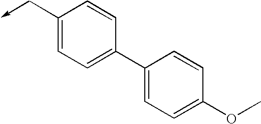 |
| 2020 2021 2022 | rac + − | 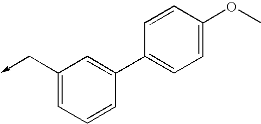 |
| 2023 2024 2025 | rac + − | 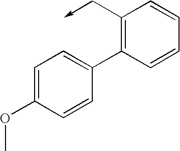 |
| 2026 2027 2028 | rac + − | 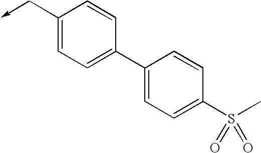 |
| 2029 2030 2031 | rac + − | 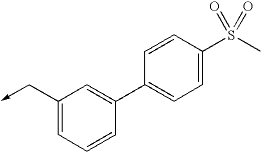 |
| 2032 2033 2034 | rac + − | 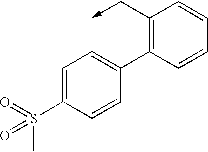 |
| 2035 2036 2037 | rac + − | 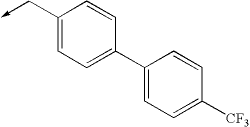 |
| 2038 2039 2040 | rac + − | 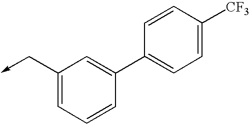 |
| 2041 2042 2043 | rac + − | 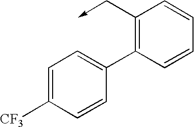 |
| 2044 2045 2046 | rac + − | 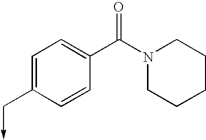 |
| 2047 2048 2049 | rac + − | 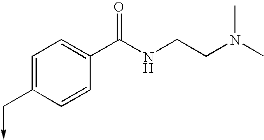 |
| 2050 2051 2052 | rac + − | 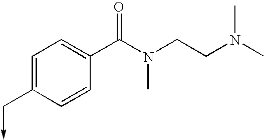 |
| 2053 2054 2055 | rac + − | 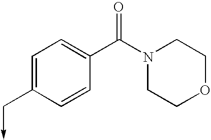 |
| 2056 2057 2058 | rac + − | 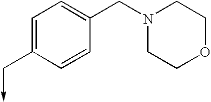 |
| 2059 2060 2061 | rac + − | 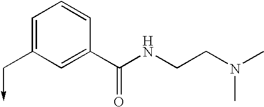 |
| 2062 2063 2064 | rac + − | 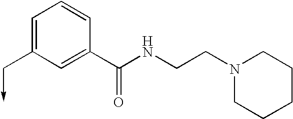 |
| 2065 2066 2067 | rac + − | 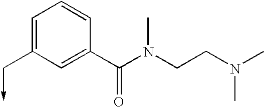 |
| 2068 2069 2070 | rac + − | 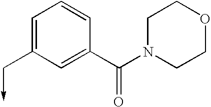 |
| 2071 2072 2073 | rac + − | 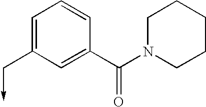 |
| 2074 2075 2076 | rac + − | 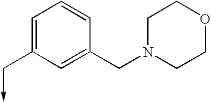 |
| 2077 2078 2079 | rac + − | 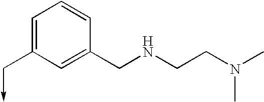 |
| 2080 2081 2082 | rac + − | 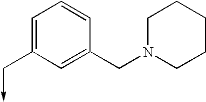 |
| 2083 2084 2085 | rac + − | 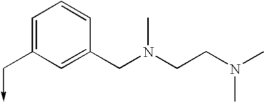 |
| 2086 2087 2088 | rac + − | 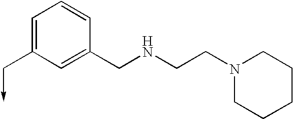 |

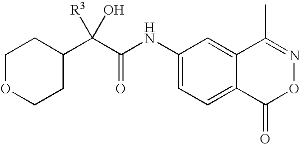

| No. | Racemic or enantiomer | R3 |
|---|---|---|
| 2089 2090 2091 | rac + − | 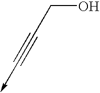 |
| 2092 2093 2094 | rac + − | 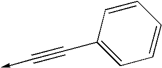 |
| 2095 2096 2097 | rac + − | 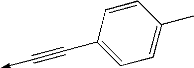 |
| 2098 2099 2100 | rac + − | 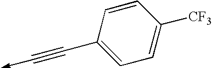 |
| 2101 2102 2103 | rac + − | 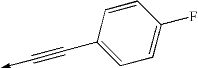 |
| 2104 2105 2106 | rac + − | 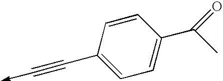 |
| 2107 2108 2109 | rac + − | 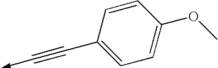 |
| 2110 2111 2112 | rac + − | 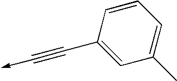 |
| 2113 2114 2115 | rac + − | 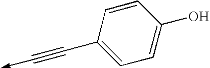 |
| 2116 2117 2118 | rac + − | 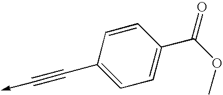 |
| 2119 2120 2121 | rac + − | 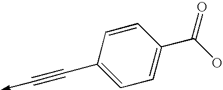 |
| 2122 2123 2124 | rac + − | 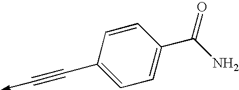 |
| 2125 2126 2127 | rac + − | 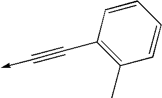 |
| 2128 2129 2130 | rac + − | 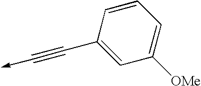 |
| 2131 2132 2133 | rac + − | 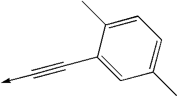 |
| 2134 2135 2136 | rac + − | 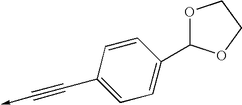 |
| 2137 2138 2139 | rac + − | 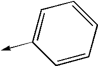 |
| 2140 2141 2142 | rac + − | 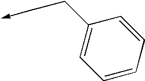 |
| 2143 2144 2145 | rac + − | 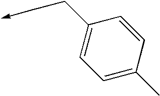 |
| 2146 2147 2148 | rac + − | 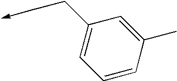 |
| 2149 2150 2151 | rac + − | 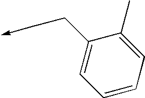 |
| 2152 2153 2154 | rac + − | 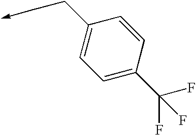 |
| 2155 2156 2157 | rac + − | 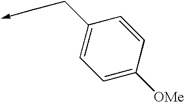 |
| 2158 2159 2160 | rac + − | 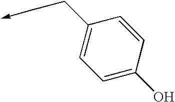 |
| 2161 2162 2163 | rac + − | 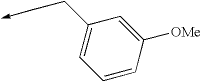 |
| 2164 2165 2166 | rac + − | 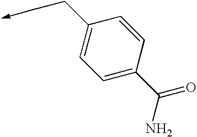 |
| 2167 2168 2169 | rac + − | 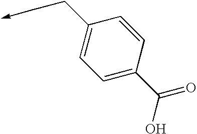 |
| 2170 2171 2172 | rac + − | 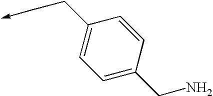 |
| 2173 2174 2175 | rac + − | 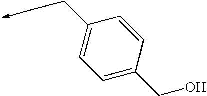 |
| 2176 2177 2178 | rac + − | 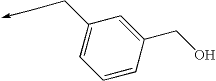 |
| 2179 2180 2181 | rac + − | 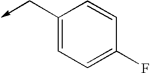 |
| 2182 2183 2184 | rac + − | 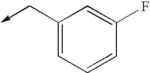 |
| 2185 2186 2187 | rac + − | 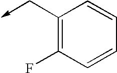 |
| 2188 2189 2190 | rac + − | 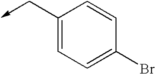 |
| 2191 2192 2193 | rac + − | 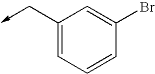 |
| 2194 2195 2196 | rac + − | 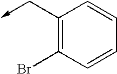 |
| 2197 2198 2199 | rac + − | 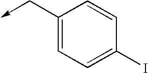 |
| 2200 2201 2202 | rac + − | 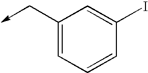 |
| 2203 2204 2205 | rac + − | 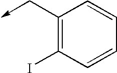 |
| 2206 2207 2208 | rac + − | 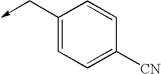 |
| 2209 2210 2211 | rac + − | 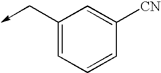 |
| 2212 2213 2214 | rac + − | 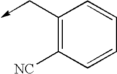 |
| 2215 2216 2217 | rac + − | 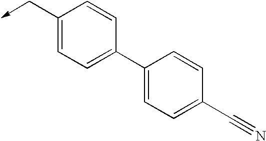 |
| 2218 2219 2220 | rac + − | 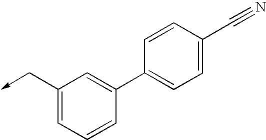 |
| 2221 2222 2223 | rac + − | 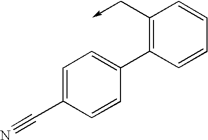 |
| 2224 2225 2226 | rac + − | 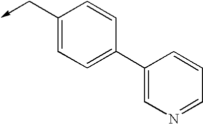 |
| 2227 2228 2229 | rac + − | 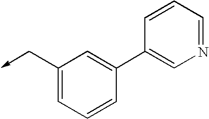 |
| 2230 2231 2232 | rac + − | 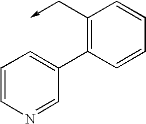 |
| 2233 2234 2235 | rac + − | 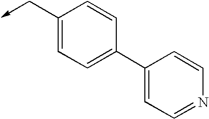 |
| 2236 2237 2238 | rac + − | 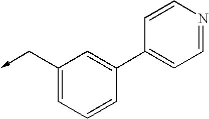 |
| 2239 2240 2241 | rac + − | 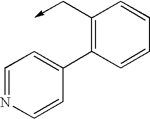 |
| 2242 2243 2244 | rac + − | 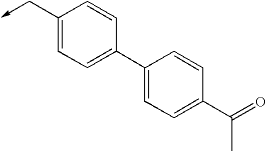 |
| 2245 2246 2247 | rac + − | 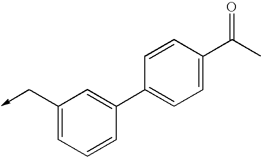 |
| 2248 2249 2250 | rac + − | 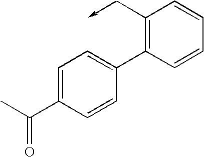 |
| 2251 2252 2253 | rac + − | 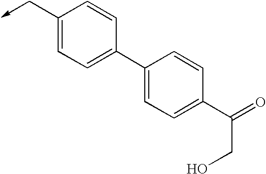 |
| 2254 2255 2256 | rac + − | 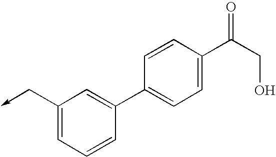 |
| 2257 2258 2259 | rac + − | 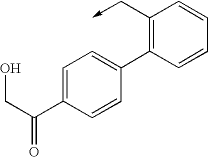 |
| 2260 2261 2262 | rac + − | 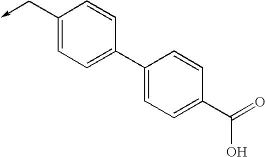 |
| 2263 2264 2265 | rac + − | 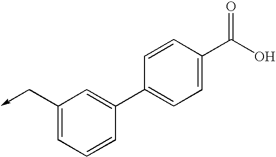 |
| 2266 2267 2268 | rac + − | 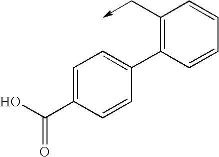 |
| 2269 2270 2271 | rac + − | 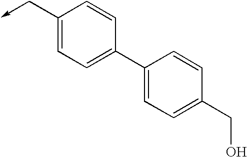 |
| 2272 2273 2274 | rac + − | 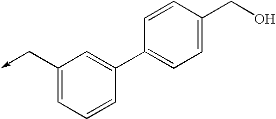 |
| 2275 2276 2277 | rac + − | 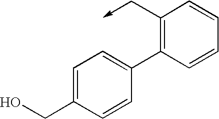 |
| 2278 2279 2280 | rac + − | 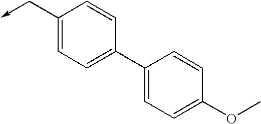 |
| 2281 2282 2283 | rac + − | 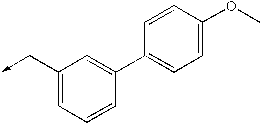 |
| 2284 2285 2286 | rac + − | 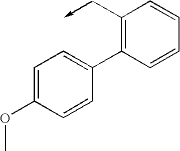 |
| 2287 2288 2289 | rac + − | 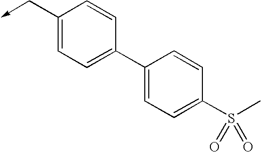 |
| 2290 2291 2292 | rac + − | 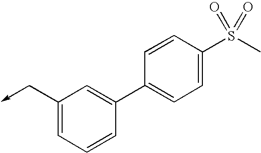 |
| 2293 2294 2295 | rac + − | 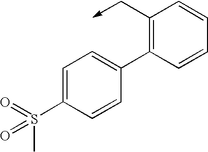 |
| 2296 2297 2298 | rac + − | 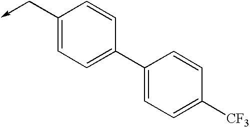 |
| 2299 2300 2301 | rac + − | 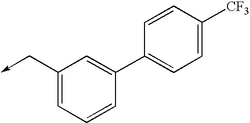 |
| 2302 2303 2304 | rac + − | 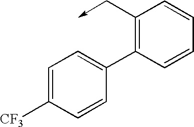 |
| 2305 2306 2307 | rac + − | 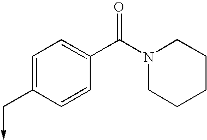 |
| 2308 2309 2310 | rac + − | 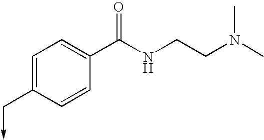 |
| 2311 2312 2313 | rac + − | 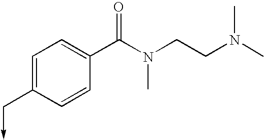 |
| 2314 2315 2316 | rac + − | 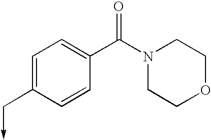 |
| 2317 2318 2319 | rac + − | 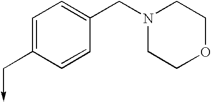 |
| 2320 2321 2322 | rac + − | 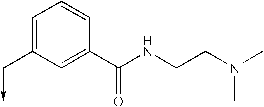 |
| 2323 2324 2325 | rac + − | 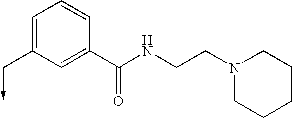 |
| 2326 2327 2328 | rac + − | 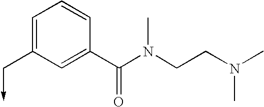 |
| 2329 2330 2331 | rac + − | 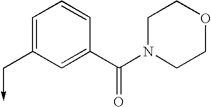 |
| 2332 2333 2334 | rac + − | 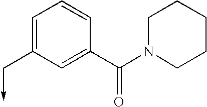 |
| 2335 2336 2337 | rac + − | 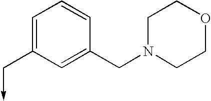 |
| 2338 2339 2340 | rac + − | 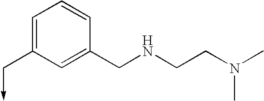 |
| 2341 2342 2343 | rac + − | 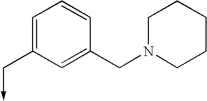 |
| 2344 2345 2346 | rac + − | 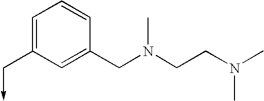 |
| 2347 2348 2349 | rac + − | 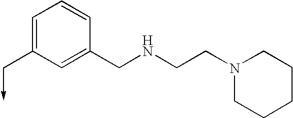 |

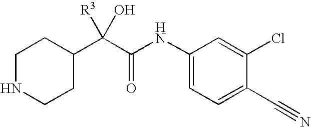

| No. | Racemic or enantiomer | R3 |
|---|---|---|
| 2350 2351 2352 | rac + − | 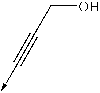 |
| 2353 2354 2355 | rac + − | 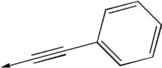 |
| 2356 2357 2358 | rac + − | 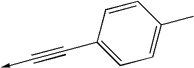 |
| 2359 2360 2361 | rac + − | 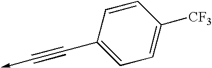 |
| 2362 2363 2364 | rac + − | 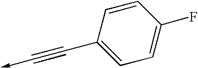 |
| 2365 2366 2367 | rac + − | 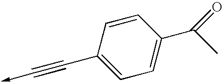 |
| 2368 2369 2370 | rac + − | 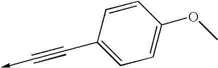 |
| 2371 2372 2373 | rac + − | 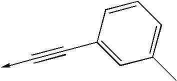 |
| 2374 2375 2376 | rac + − | 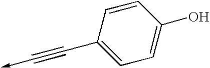 |
| 2377 2378 2379 | rac + − | 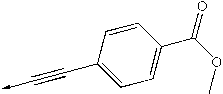 |
| 2380 2381 2382 | rac + − | 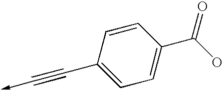 |
| 2383 2384 2385 | rac + − | 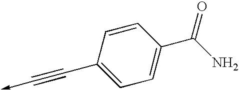 |
| 2386 2387 2388 | rac + − | 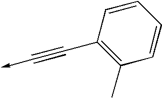 |
| 2389 2390 2391 | rac + − | 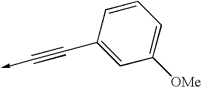 |
| 2392 2393 2394 | rac + − | 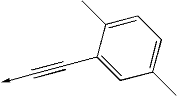 |
| 2395 2396 2397 | rac + − | 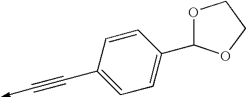 |
| 2398 2399 2400 | rac + − | 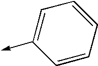 |
| 2401 2402 2403 | rac + − | 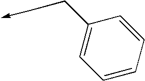 |
| 2404 2405 2406 | rac + − | 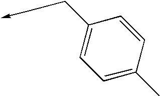 |
| 2407 2408 2409 | rac + − | 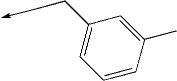 |
| 2410 2411 2412 | rac + − | 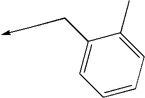 |
| 2413 2414 2415 | rac + − | 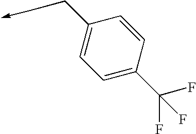 |
| 2416 2417 2418 | rac + − | 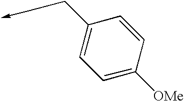 |
| 2419 2420 2421 | rac + − | 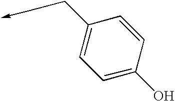 |
| 2422 2423 2424 | rac + − | 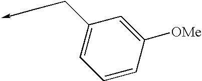 |
| 2425 2426 2427 | rac + − | 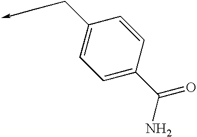 |
| 2428 2429 2430 | rac + − | 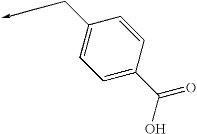 |
| 2431 2432 2433 | rac + − | 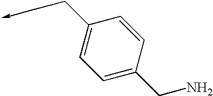 |
| 2434 2435 2436 | rac + − | 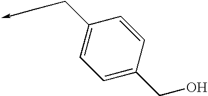 |
| 2437 2438 2439 | rac + − | 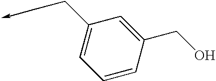 |
| 2440 2441 2442 | rac + − | 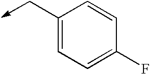 |
| 2443 2444 2445 | rac + − | 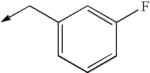 |
| 2446 2447 2448 | rac + − | 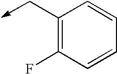 |
| 2449 2450 2451 | rac + − | 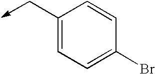 |
| 2452 2453 2454 | rac + − | 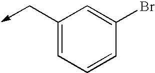 |
| 2455 2456 2457 | rac + − | 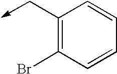 |
| 2458 2459 2460 | rac + − | 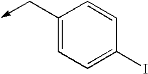 |
| 2461 2462 2463 | rac + − | 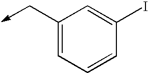 |
| 2464 2465 2466 | rac + − | 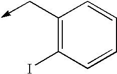 |
| 2467 2468 2469 | rac + − | 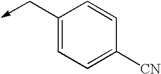 |
| 2470 2471 2472 | rac + − | 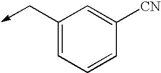 |
| 2473 2474 2475 | rac + − | 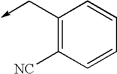 |
| 2476 2477 2478 | rac + − | 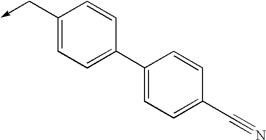 |
| 2479 2480 2481 | rac + − | 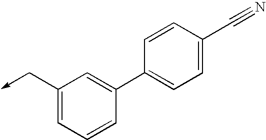 |
| 2482 2483 2484 | rac + − | 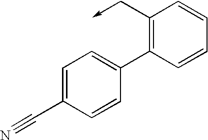 |
| 2485 2486 2487 | rac + − | 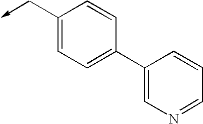 |
| 2488 2489 2490 | rac + − | 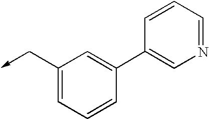 |
| 2491 2492 2493 | rac + − | 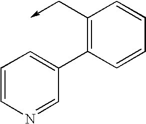 |
| 2494 2495 2496 | rac + − | 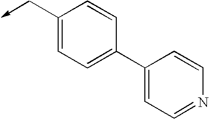 |
| 2497 2498 2499 | rac + − | 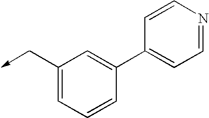 |
| 2500 2501 2502 | rac + − | 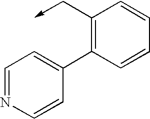 |
| 2503 2504 2505 | rac + − | 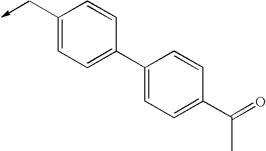 |
| 2506 2507 2508 | rac + − | 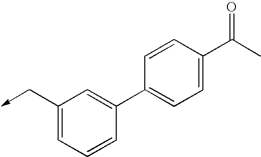 |
| 2509 2510 2511 | rac + − | 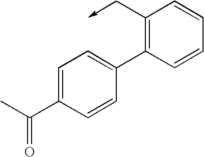 |
| 2512 2513 2514 | rac + − | 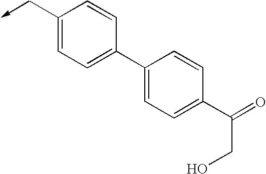 |
| 2515 2516 2517 | rac + − | 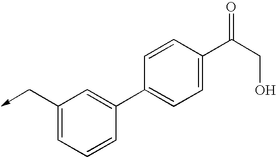 |
| 2518 2519 2520 | rac + − | 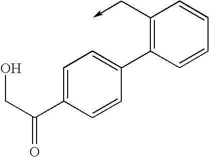 |
| 2521 2522 2523 | rac + − | 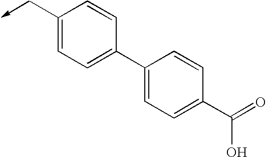 |
| 2524 2525 2526 | rac + − | 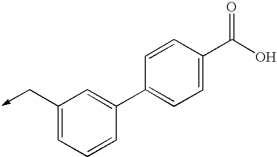 |
| 2527 2528 2529 | rac + − | 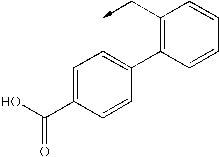 |
| 2530 2531 2532 | rac + − | 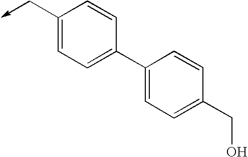 |
| 2533 2534 2535 | rac + − | 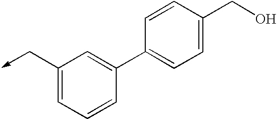 |
| 2536 2537 2538 | rac + − | 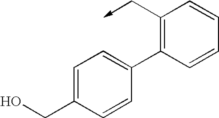 |
| 2539 2540 2541 | rac + − | 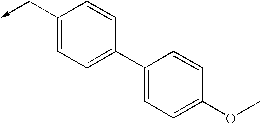 |
| 2542 2543 2544 | rac + − | 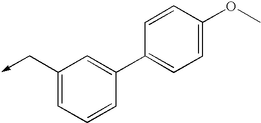 |
| 2545 2546 2547 | rac + − | 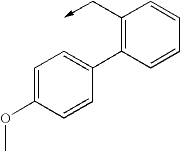 |
| 2548 2549 2550 | rac + − | 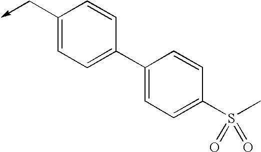 |
| 2551 2552 2553 | rac + − | 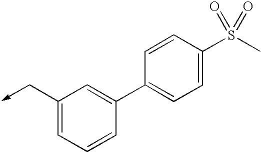 |
| 2554 2555 2556 | rac + − | 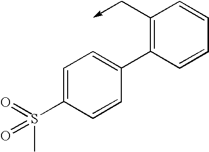 |
| 2557 2558 2559 | rac + − | 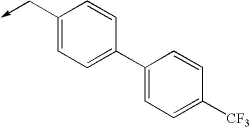 |
| 2560 2561 2562 | rac + − | 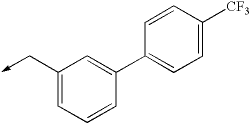 |
| 2563 2564 2565 | rac + − | 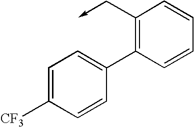 |
| 2566 2567 2568 | rac + − | 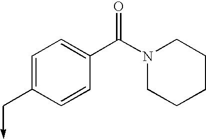 |
| 2569 2570 2571 | rac + − | 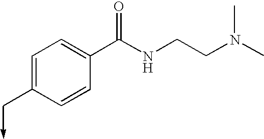 |
| 2572 2573 2574 | rac + − | 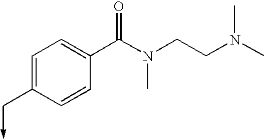 |
| 2575 2576 2577 | rac + − | 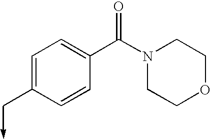 |
| 2578 2579 2580 | rac + − | 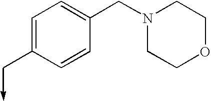 |
| 2581 2582 2583 | rac + − | 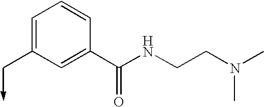 |
| 2584 2585 2586 | rac + − | 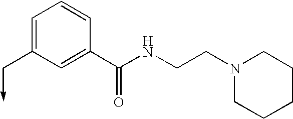 |
| 2587 2588 2589 | rac + − | 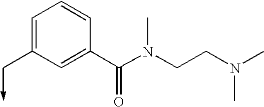 |
| 2590 2591 2592 | rac + − | 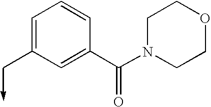 |
| 2593 2594 2595 | rac + − | 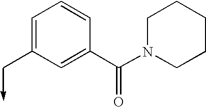 |
| 2596 2597 2598 | rac + − | 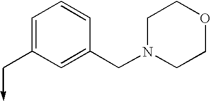 |
| 2599 2600 2601 | rac + − | 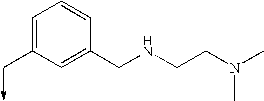 |
| 2602 2603 2604 | rac + − | 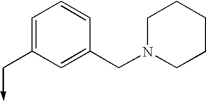 |
| 2605 2606 2607 | rac + − | 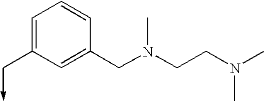 |
| 2608 2609 2610 | rac + − | 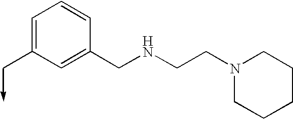 |

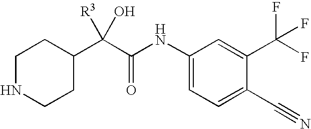

| No. | Racemic or enantiomer | R3 |
|---|---|---|
| 2611 2612 2613 | rac + − | 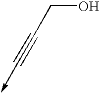 |
| 2614 2615 2616 | rac + − | 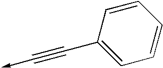 |
| 2617 2618 2619 | rac + − | 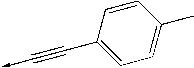 |
| 2620 2621 2622 | rac + − | 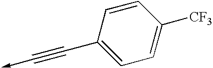 |
| 2623 2624 2625 | rac + − | 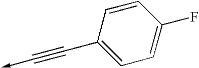 |
| 2626 2627 2628 | rac + − | 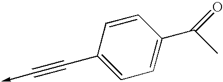 |
| 2629 2630 2631 | rac + − | 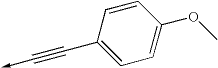 |
| 2632 2633 2634 | rac + − | 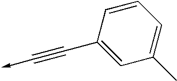 |
| 2635 2636 2637 | rac + − | 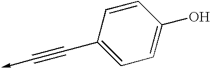 |
| 2638 2639 2640 | rac + − | 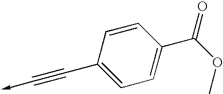 |
| 2641 2642 2643 | rac + − | 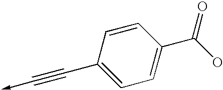 |
| 2644 2645 2646 | rac + − | 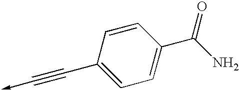 |
| 2647 2648 2649 | rac + − | 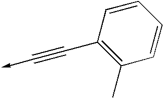 |
| 2650 2651 2652 | rac + − | 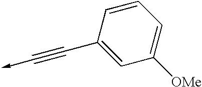 |
| 2653 2654 2655 | rac + − | 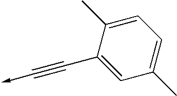 |
| 2656 2657 2658 | rac + − | 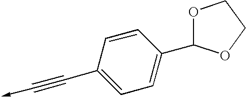 |
| 2659 2660 2661 | rac + − | 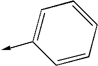 |
| 2662 2663 2664 | rac + − | 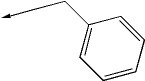 |
| 2665 2666 2667 | rac + − | 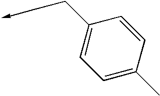 |
| 2668 2669 2670 | rac + − | 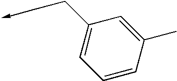 |
| 2671 2672 2673 | rac + − | 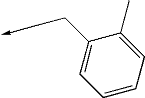 |
| 2674 2675 2676 | rac + − | 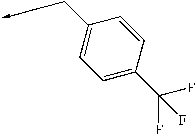 |
| 2677 2678 2679 | rac + − | 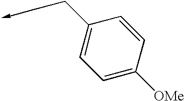 |
| 2680 2681 2682 | rac + − | 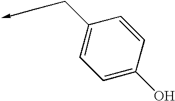 |
| 2683 2684 2685 | rac + − | 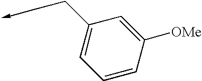 |
| 2686 2687 2688 | rac + − | 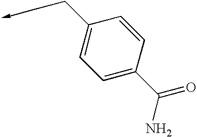 |
| 2689 2690 2691 | rac + − | 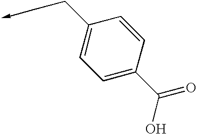 |
| 2692 2693 2694 | rac + − | 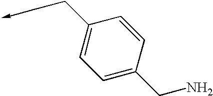 |
| 2695 2696 2697 | rac + − | 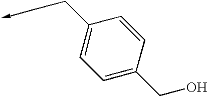 |
| 2698 2699 2700 | rac + − | 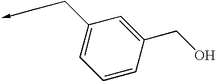 |
| 2701 2702 2703 | rac + − | 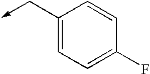 |
| 2704 2705 2706 | rac + − | 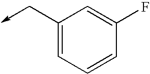 |
| 2707 2708 2709 | rac + − | 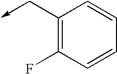 |
| 2710 2711 2712 | rac + − | 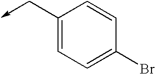 |
| 2713 2714 2715 | rac + − | 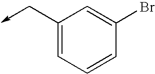 |
| 2716 2717 2718 | rac + − | 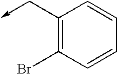 |
| 2719 2720 2721 | rac + − | 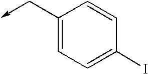 |
| 2722 2723 2724 | rac + − | 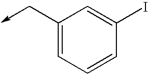 |
| 2725 2726 2727 | rac + − | 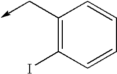 |
| 2728 2729 2730 | rac + − | 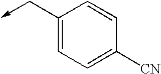 |
| 2731 2732 2733 | rac + − | 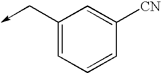 |
| 2734 2735 2736 | rac + − | 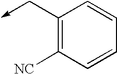 |
| 2737 2738 2739 | rac + − | 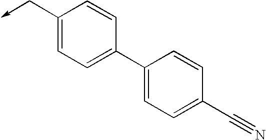 |
| 2740 2741 2742 | rac + − | 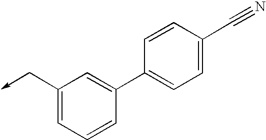 |
| 2743 2744 2745 | rac + − | 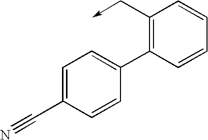 |
| 2746 2747 2748 | rac + − | 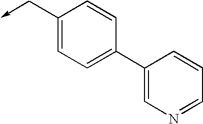 |
| 2749 2750 2751 | rac + − | 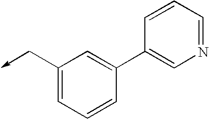 |
| 2752 2753 2754 | rac + − | 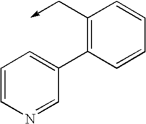 |
| 2755 2756 2757 | rac + − | 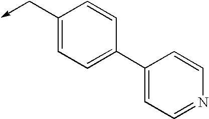 |
| 2758 2759 2760 | rac + − | 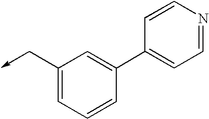 |
| 2761 2762 2763 | rac + − | 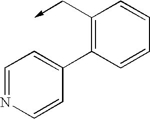 |
| 2764 2765 2766 | rac + − | 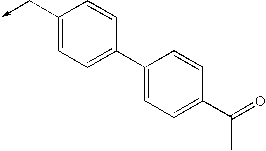 |
| 2767 2768 2769 | rac + − | 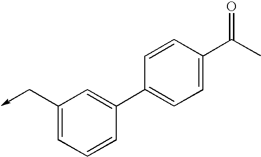 |
| 2770 2771 2772 | rac + − | 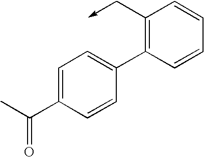 |
| 2773 2774 2775 | rac + − | 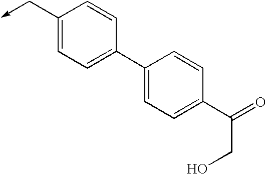 |
| 2776 2777 2778 | rac + − | 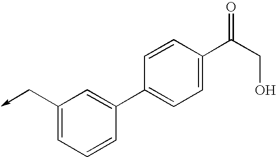 |
| 2779 2780 2781 | rac + − | 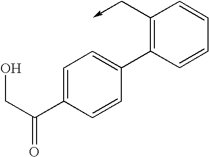 |
| 2782 2783 2784 | rac + − | 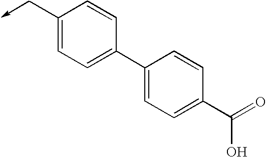 |
| 2785 2786 2787 | rac + − | 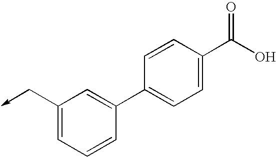 |
| 2788 2789 2790 | rac + − | 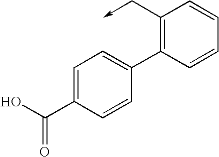 |
| 2791 2792 2793 | rac + − | 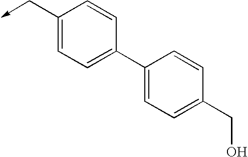 |
| 2794 2795 2796 | rac + − | 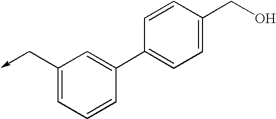 |
| 2797 2798 2799 | rac + − | 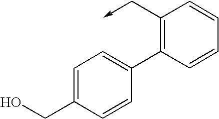 |
| 2800 2801 2802 | rac + − | 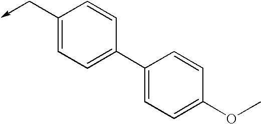 |
| 2803 2804 2805 | rac + − | 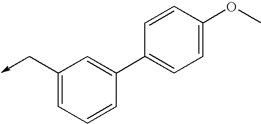 |
| 2806 2807 2808 | rac + − | 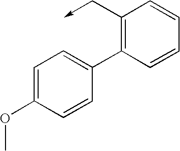 |
| 2809 2810 2811 | rac + − | 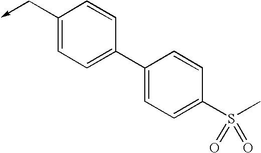 |
| 2812 2813 2814 | rac + − | 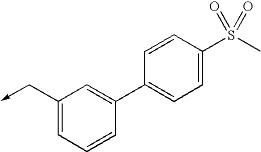 |
| 2815 2816 2817 | rac + − | 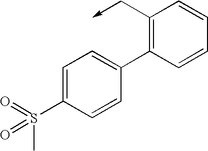 |
| 2818 2819 2820 | rac + − | 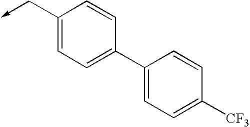 |
| 2821 2822 2823 | rac + − | 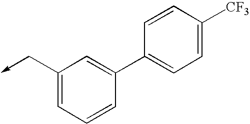 |
| 2824 2825 2826 | rac + − | 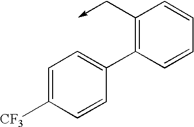 |
| 2827 2828 2829 | rac + − | 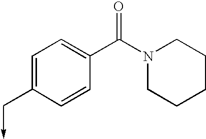 |
| 2830 2831 2832 | rac + − | 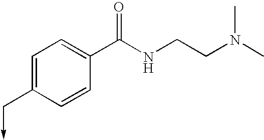 |
| 2833 2834 2835 | rac + − | 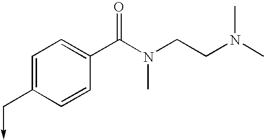 |
| 2836 2837 2838 | rac + − | 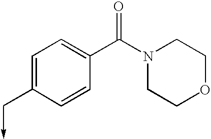 |
| 2839 2840 2841 | rac + − | 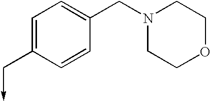 |
| 2842 2843 2844 | rac + − | 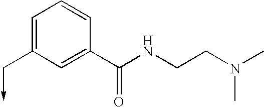 |
| 2845 2846 2847 | rac + − | 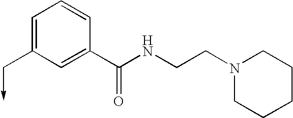 |
| 2848 2849 2850 | rac + − | 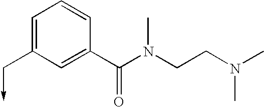 |
| 2851 2852 2853 | rac + − | 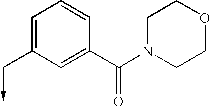 |
| 2854 2855 2856 | rac + − | 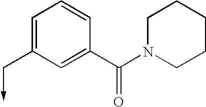 |
| 2857 2858 2859 | rac + − | 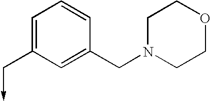 |
| 2860 2861 2862 | rac + − | 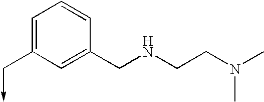 |
| 2863 2864 2865 | rac + − | 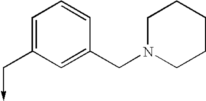 |
| 2866 2867 2868 | rac + − | 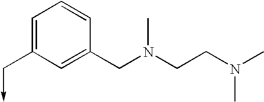 |
| 2869 2870 2871 | rac + − | 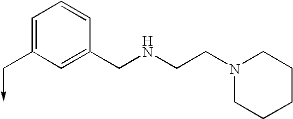 |

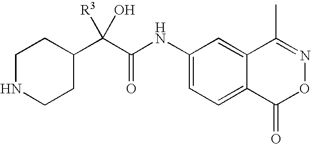

| No. | Racemic or enantiomer | R3 |
|---|---|---|
| 2872 2873 2874 | rac + − | 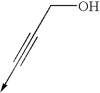 |
| 2875 2876 2877 | rac + − | 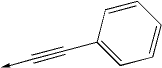 |
| 2878 2879 2880 | rac + − | 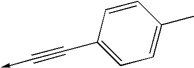 |
| 2881 2882 2883 | rac + − | 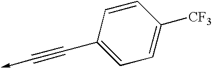 |
| 2884 2885 2886 | rac + − | 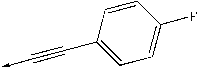 |
| 2887 2888 2889 | rac + − | 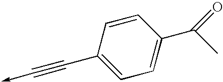 |
| 2890 2891 2892 | rac + − | 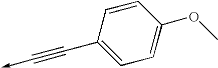 |
| 2893 2894 2895 | rac + − | 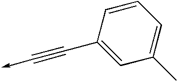 |
| 2896 2897 2898 | rac + − | 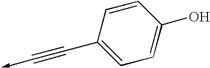 |
| 2899 2900 2901 | rac + − | 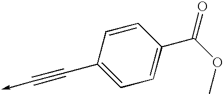 |
| 2902 2903 2904 | rac + − | 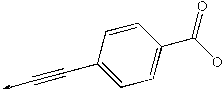 |
| 2905 2906 2907 | rac + − | 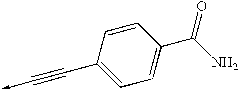 |
| 2908 2909 2910 | rac + − | 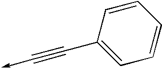 |
| 2911 2912 2913 | rac + − | 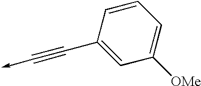 |
| 2914 2915 2916 | rac + − | 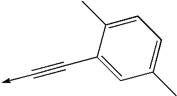 |
| 2917 2918 2919 | rac + − | 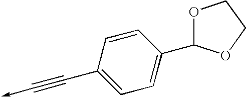 |
| 2920 2921 2922 | rac + − | 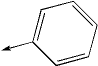 |
| 2923 2924 2925 | rac + − | 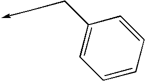 |
| 2926 2927 2928 | rac + − | 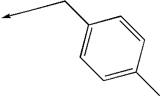 |
| 2929 2930 2931 | rac + − | 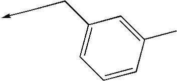 |
| 2932 2933 2934 | rac + − | 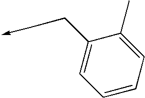 |
| 2935 2936 2937 | rac + − | 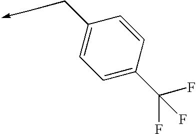 |
| 2938 2939 2940 | rac + − | 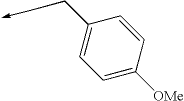 |
| 2941 2942 2943 | rac + − | 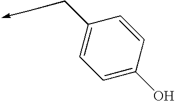 |
| 2944 2945 2946 | rac + − | 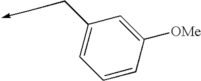 |
| 2947 2948 2949 | rac + − | 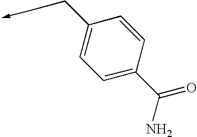 |
| 2950 2951 2952 | rac + − | 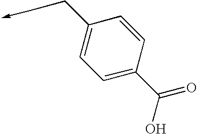 |
| 2953 2954 2955 | rac + − | 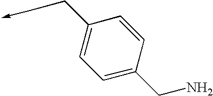 |
| 2956 2957 2958 | rac + − | 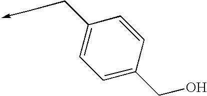 |
| 2959 2960 2961 | rac + − | 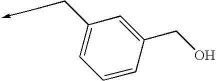 |
| 2962 2963 2964 | rac + − | 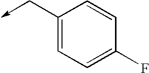 |
| 2965 2966 2967 | rac + − | 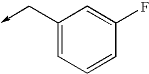 |
| 2968 2969 2970 | rac + − | 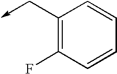 |
| 2971 2972 2973 | rac + − | 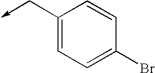 |
| 2974 2975 2976 | rac + − | 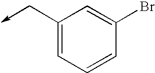 |
| 2977 2978 2979 | rac + − | 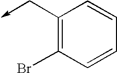 |
| 2980 2981 2982 | rac + − | 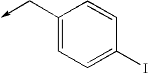 |
| 2983 2984 2985 | rac + − | 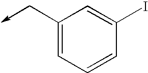 |
| 2986 2987 2988 | rac + − | 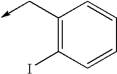 |
| 2989 2990 2991 | rac + − | 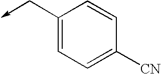 |
| 2992 2993 2994 | rac + − | 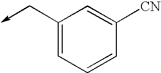 |
| 2995 2996 2997 | rac + − | 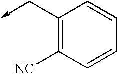 |
| 2998 2999 3000 | rac + − | 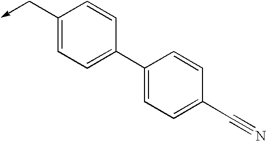 |
| 3001 3002 3003 | rac + − | 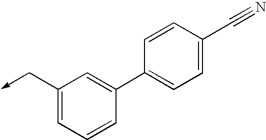 |
| 3004 3005 3006 | rac + − | 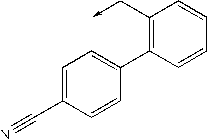 |
| 3007 3008 3009 | rac + − | 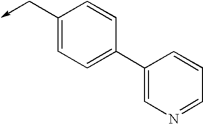 |
| 3010 3011 3012 | rac + − | 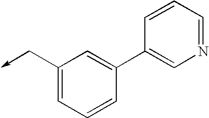 |
| 3013 3014 3015 | rac + − | 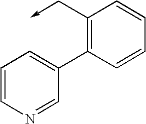 |
| 3016 3017 3018 | rac + − | 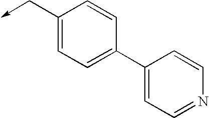 |
| 3019 3020 3021 | rac + − | 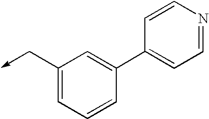 |
| 3022 3023 3024 | rac + − | 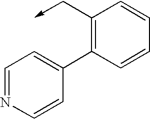 |
| 3025 3026 3027 | rac + − | 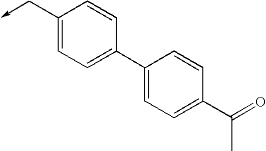 |
| 3028 3029 3030 | rac + − | 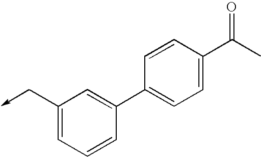 |
| 3031 3032 3033 | rac + − | 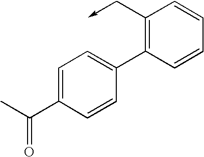 |
| 3034 3035 3036 | rac + − | 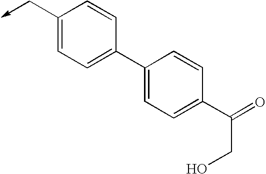 |
| 3037 3038 3039 | rac + − | 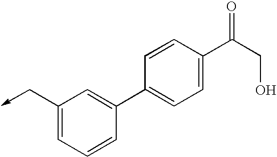 |
| 3040 3041 3042 | rac + − | 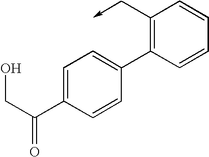 |
| 3043 3044 3045 | rac + − | 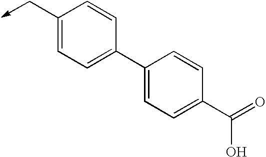 |
| 3046 3047 3048 | rac + − | 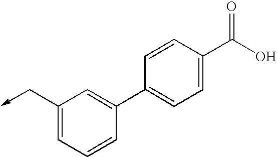 |
| 3049 3050 3051 | rac + − | 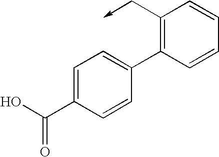 |
| 3052 3053 3054 | rac + − | 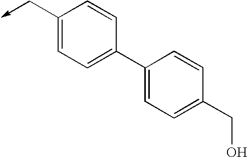 |
| 3055 3056 3057 | rac + − | 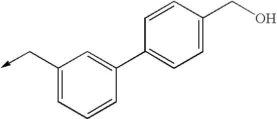 |
| 3058 3059 3060 | rac + − | 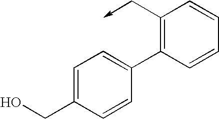 |
| 3061 3062 3063 | rac + − | 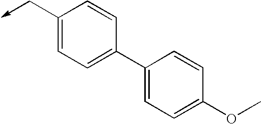 |
| 3064 3065 3066 | rac + − | 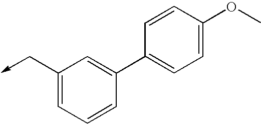 |
| 3067 3068 3069 | rac + − | 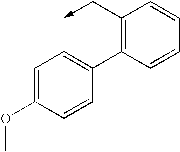 |
| 3070 3071 3072 | rac + − | 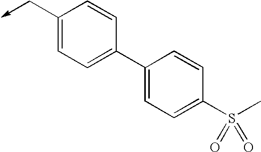 |
| 3073 3074 3075 | rac + − | 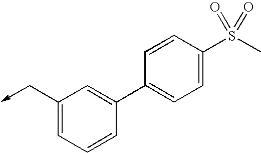 |
| 3076 3077 3078 | rac + − | 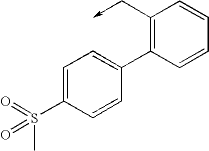 |
| 3079 3080 3081 | rac + − | 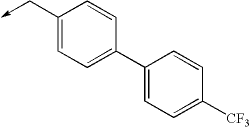 |
| 3082 3083 3084 | rac + − | 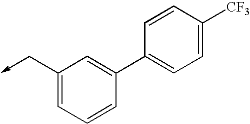 |
| 3085 3086 3087 | rac + − | 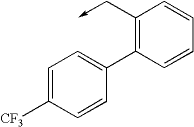 |
| 3088 3089 3090 | rac + − | 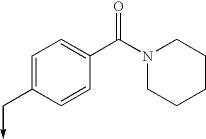 |
| 3091 3092 3093 | rac + − | 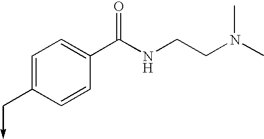 |
| 3094 3095 3096 | rac + − | 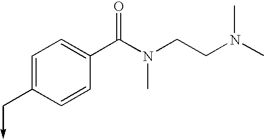 |
| 3097 3098 3099 | rac + − | 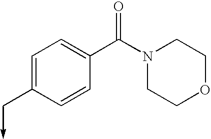 |
| 3100 3101 3102 | rac + − | 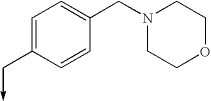 |
| 3103 3104 3105 | rac + − | 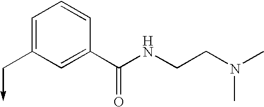 |
| 3106 3107 3108 | rac + − | 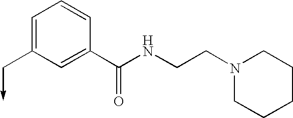 |
| 3109 3110 3111 | rac + − | 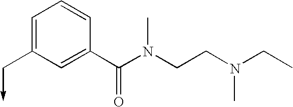 |
| 3112 3113 3114 | rac + − | 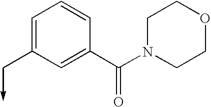 |
| 3115 3116 3117 | rac + − | 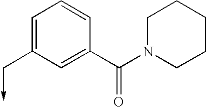 |
| 3118 3119 3120 | rac + − | 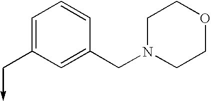 |
| 3121 3122 3123 | rac + − | 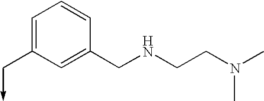 |
| 3124 3125 3126 | rac + − | 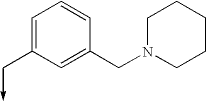 |
| 3127 3128 3129 | rac + − | 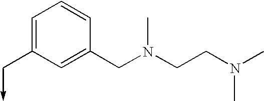 |
| 3130 3131 3132 | rac + − | 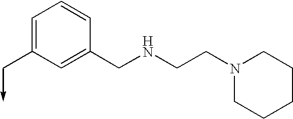 |

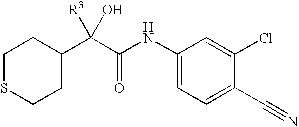

| No. | Racemic or enantiomer | R3 |
|---|---|---|
| 3133 3134 3135 | rac + − | 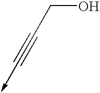 |
| 3136 3137 3138 | rac + − | 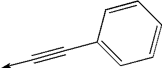 |
| 3139 3140 3141 | rac + − | 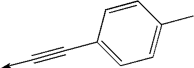 |
| 3142 3143 3144 | rac + − | 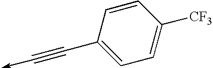 |
| 3145 3146 3147 | rac + − | 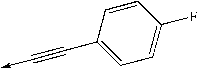 |
| 3148 3149 3150 | rac + − | 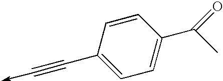 |
| 3151 3152 3153 | rac + − | 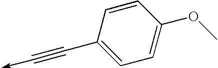 |
| 3154 3155 3156 | rac + − | 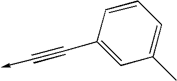 |
| 3157 3158 3159 | rac + − | 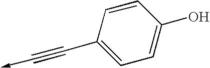 |
| 3160 3161 3162 | rac + − | 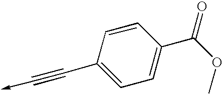 |
| 3163 3164 3165 | rac + − | 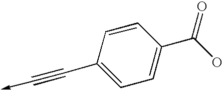 |
| 3166 3167 3168 | rac + − | 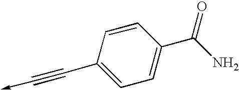 |
| 3169 3170 3171 | rac + − | 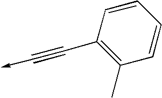 |
| 3172 3173 3174 | rac + − | 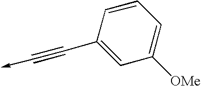 |
| 3175 3176 3177 | rac + − | 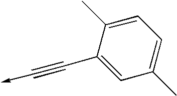 |
| 3178 3179 3180 | rac + − | 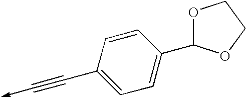 |
| 3181 3182 3183 | rac + − | 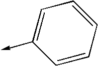 |
| 3184 3185 3186 | rac + − | 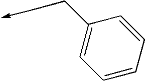 |
| 3187 3188 3189 | rac + − | 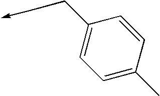 |
| 3190 3191 3192 | rac + − | 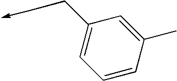 |
| 3193 3194 3195 | rac + − | 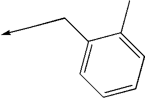 |
| 3196 3197 3198 | rac + − | 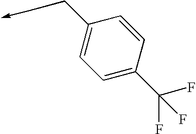 |
| 3199 3200 3201 | rac + − | 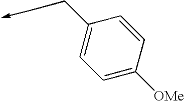 |
| 3202 3203 3204 | rac + − | 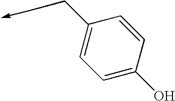 |
| 3205 3206 3207 | rac + − | 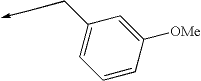 |
| 3208 3209 3210 | rac + − | 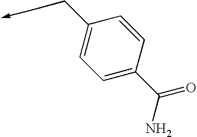 |
| 3211 3212 3213 | rac + − | 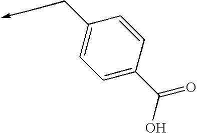 |
| 3214 3215 3216 | rac + − | 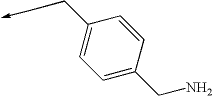 |
| 3217 3218 3219 | rac + − | 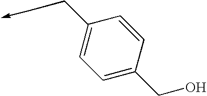 |
| 3220 3221 3222 | rac + − | 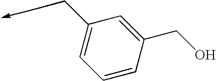 |
| 3223 3224 3225 | rac + − | 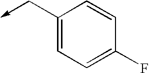 |
| 3226 3227 3228 | rac + − | 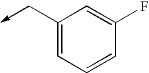 |
| 3229 3230 3231 | rac + − | 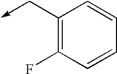 |
| 3232 3233 3234 | rac + − | 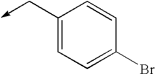 |
| 3235 3236 3237 | rac + − | 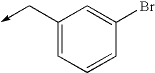 |
| 3238 3239 3240 | rac + − | 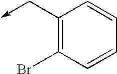 |
| 3241 3242 3243 | rac + − | 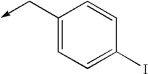 |
| 3244 3245 3246 | rac + − | 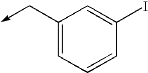 |
| 3247 3248 3249 | rac + − | 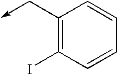 |
| 3250 3251 3252 | rac + − | 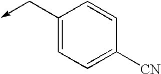 |
| 3253 3254 3255 | rac + − | 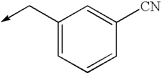 |
| 3256 3257 3258 | rac + − | 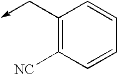 |
| 3259 3260 3261 | rac + − | 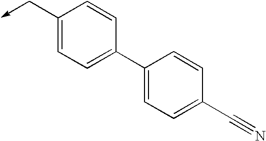 |
| 3262 3263 3264 | rac + − | 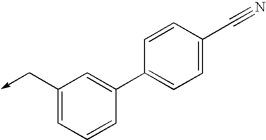 |
| 3265 3266 3267 | rac + − | 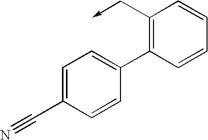 |
| 3268 3269 3270 | rac + − | 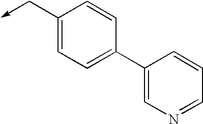 |
| 3271 3272 3273 | rac + − | 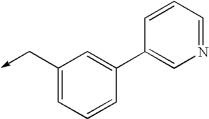 |
| 3274 3275 3276 | rac + − | 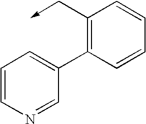 |
| 3277 3278 3279 | rac + − | 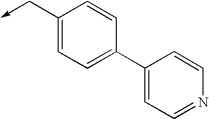 |
| 3280 3281 3282 | rac + − | 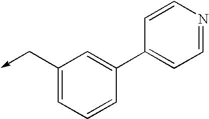 |
| 3283 3284 3285 | rac + − | 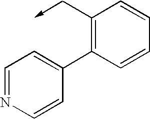 |
| 3286 3287 3288 | rac + − | 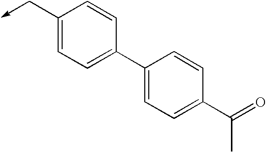 |
| 3289 3290 3291 | rac + − | 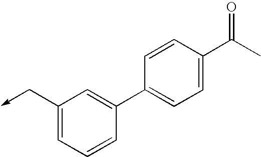 |
| 3292 3293 3294 | rac + − | 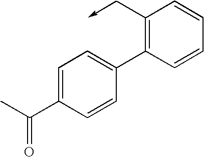 |
| 3295 3296 3297 | rac + − | 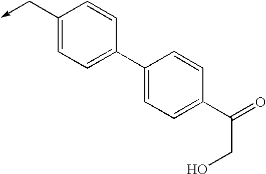 |
| 3298 3299 3300 | rac + − | 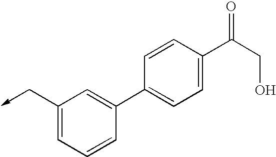 |
| 3301 3302 3303 | rac + − | 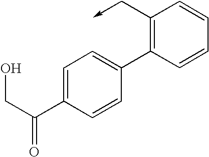 |
| 3304 3305 3306 | rac + − | 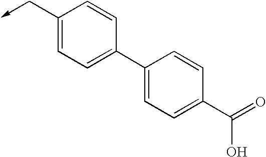 |
| 3307 3308 3309 | rac + − | 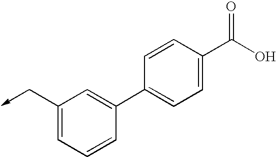 |
| 3310 3311 3312 | rac + − | 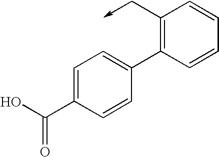 |
| 3313 3314 3315 | rac + − | 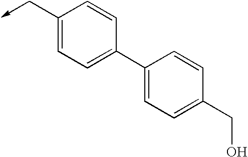 |
| 3316 3317 3318 | rac + − | 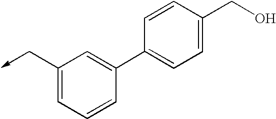 |
| 3319 3320 3321 | rac + − | 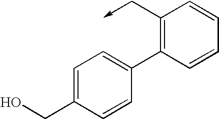 |
| 3322 3323 3324 | rac + − | 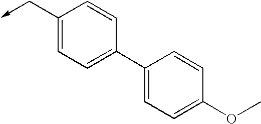 |
| 3325 3326 3327 | rac + − | 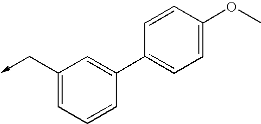 |
| 3328 3329 3330 | rac + − | 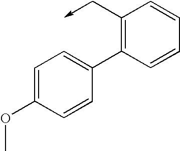 |
| 3331 3332 3333 | rac + − | 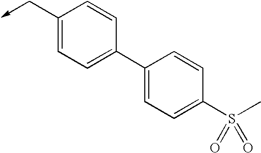 |
| 3334 3335 3336 | rac + − | 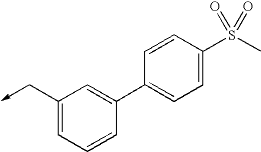 |
| 3337 3338 3339 | rac + − | 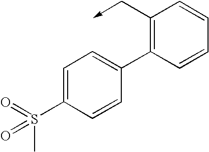 |
| 3340 3341 3342 | rac + − | 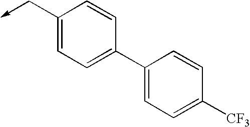 |
| 3343 3344 3345 | rac + − | 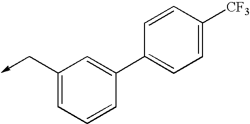 |
| 3346 3347 3348 | rac + − | 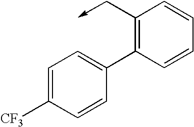 |
| 3349 3350 3351 | rac + − | 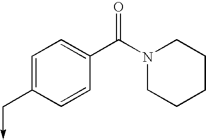 |
| 3352 3353 3354 | rac + − | 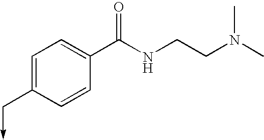 |
| 3355 3356 3357 | rac + − | 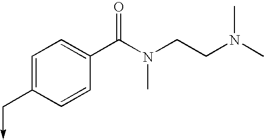 |
| 3358 3359 3360 | rac + − | 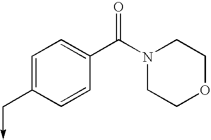 |
| 3361 3362 3363 | rac + − | 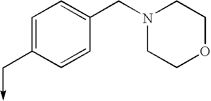 |
| 3364 3365 3366 | rac + − | 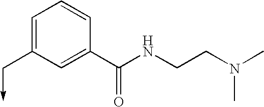 |
| 3367 3368 3369 | rac + − | 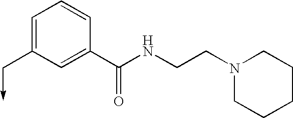 |
| 3370 3371 3372 | rac + − | 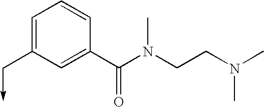 |
| 3373 3374 3375 | rac + − | 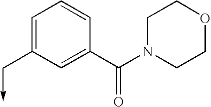 |
| 3376 3377 3378 | rac + − | 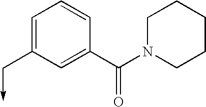 |
| 3379 3380 3381 | rac + − | 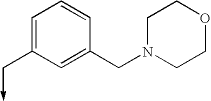 |
| 3382 3383 3384 | rac + − | 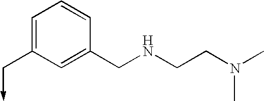 |
| 3385 3386 3387 | rac + − | 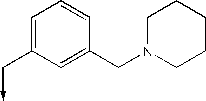 |
| 3388 3389 3390 | rac + − | 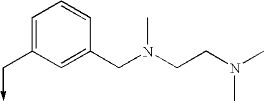 |
| 3391 3392 3393 | rac + − | 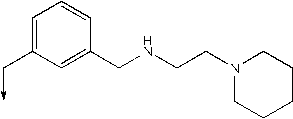 |

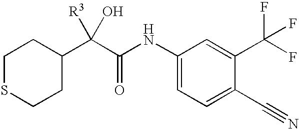

| No. | Racemic or enantiomer | R3 |
|---|---|---|
| 3394 3395 3396 | rac + − | 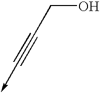 |
| 3397 3398 3399 | rac + − | 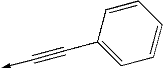 |
| 3400 3401 3402 | rac + − | 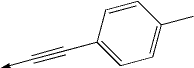 |
| 3403 3404 3405 | rac + − | 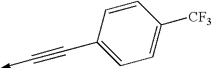 |
| 3406 3407 3408 | rac + − | 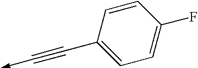 |
| 3409 3410 3411 | rac + − | 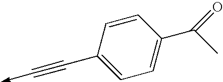 |
| 3412 3413 3414 | rac + − | 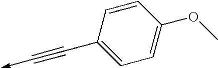 |
| 3415 3416 3417 | rac + − | 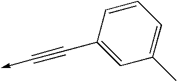 |
| 3418 3419 3420 | rac + − | 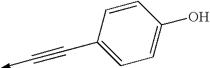 |
| 3421 3422 3423 | rac + − | 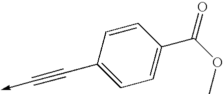 |
| 3424 3425 3426 | rac + − | 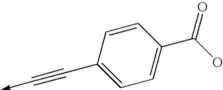 |
| 3427 3428 3429 | rac + − | 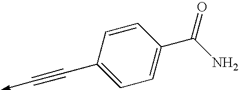 |
| 3430 3431 3432 | rac + − | 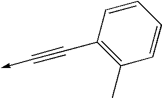 |
| 3433 3434 3435 | rac + − | 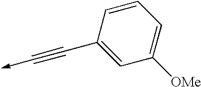 |
| 3436 3437 3438 | rac + − | 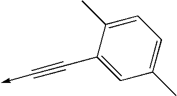 |
| 3439 3440 3441 | rac + − | 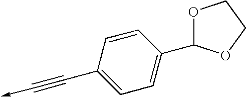 |
| 3442 3443 3444 | rac + − | 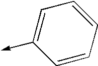 |
| 3445 3446 3447 | rac + − | 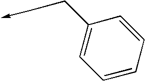 |
| 3448 3449 3450 | rac + − | 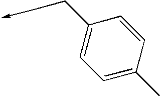 |
| 3451 3452 3453 | rac + − | 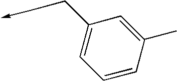 |
| 3454 3455 3456 | rac + − | 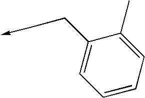 |
| 3457 3458 3459 | rac + − | 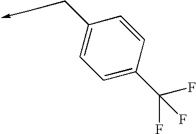 |
| 3460 3461 3462 | rac + − | 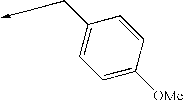 |
| 3463 3464 3465 | rac + − | 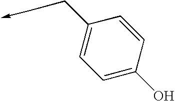 |
| 3466 3467 3468 | rac + − | 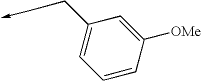 |
| 3469 3470 3471 | rac + − | 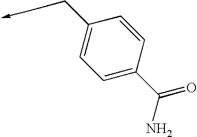 |
| 3472 3473 3474 | rac + − | 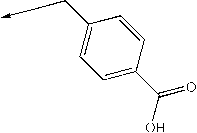 |
| 3475 3476 3477 | rac + − | 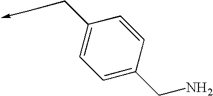 |
| 3478 3479 3480 | rac + − | 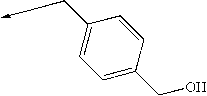 |
| 3481 3482 3483 | rac + − | 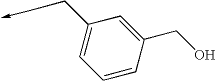 |
| 3484 3485 3486 | rac + − | 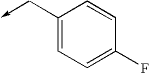 |
| 3487 3488 3489 | rac + − | 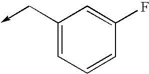 |
| 3490 3491 3492 | rac + − | 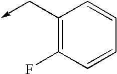 |
| 3493 3494 3495 | rac + − | 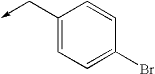 |
| 3496 3497 3498 | rac + − | 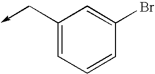 |
| 3499 3500 3501 | rac + − | 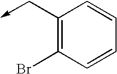 |
| 3502 3503 3504 | rac + − | 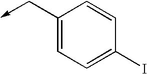 |
| 3505 3506 3507 | rac + − | 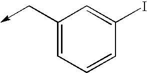 |
| 3508 3509 3510 | rac + − | 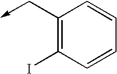 |
| 3511 3512 3513 | rac + − | 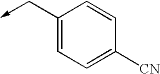 |
| 3514 3515 3516 | rac + − | 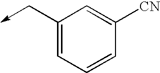 |
| 3517 3518 3519 | rac + − | 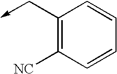 |
| 3520 3521 3522 | rac + − | 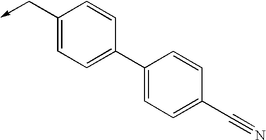 |
| 3523 3524 3525 | rac + − | 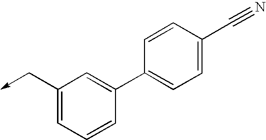 |
| 3526 3527 3528 | rac + − | 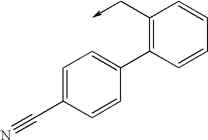 |
| 3529 3530 3531 | rac + − | 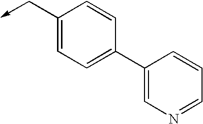 |
| 3532 3533 3534 | rac + − | 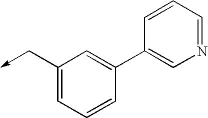 |
| 3535 3536 3537 | rac + − | 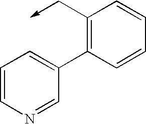 |
| 3538 3539 3540 | rac + − | 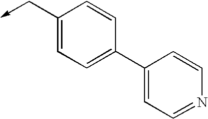 |
| 3541 3542 3543 | rac + − | 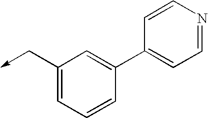 |
| 3544 3545 3546 | rac + − | 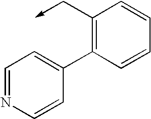 |
| 3547 3548 3549 | rac + − | 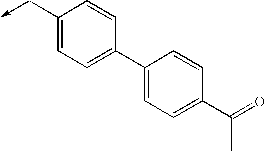 |
| 3550 3551 3552 | rac + − | 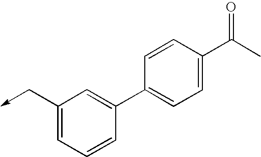 |
| 3553 3554 3555 | rac + − | 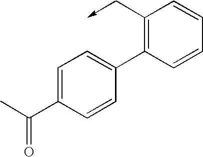 |
| 3556 3557 3558 | rac + − | 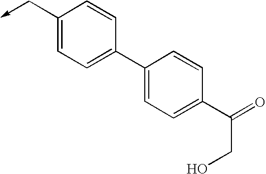 |
| 3559 3560 3561 | rac + − | 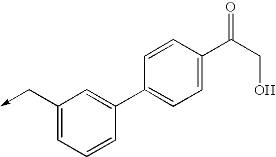 |
| 3562 3563 3564 | rac + − | 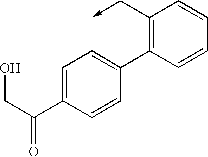 |
| 3565 3566 3567 | rac + − | 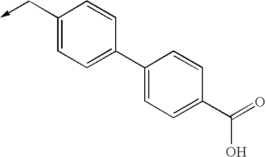 |
| 3568 3569 3570 | rac + − | 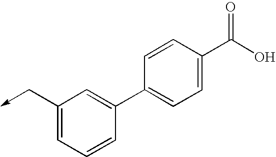 |
| 3571 3572 3573 | rac + − | 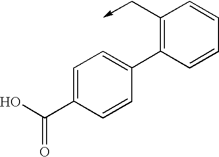 |
| 3574 3575 3576 | rac + − | 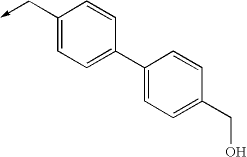 |
| 3577 3578 3579 | rac + − | 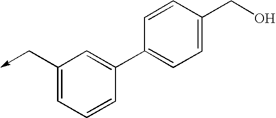 |
| 3580 3581 3582 | rac + − | 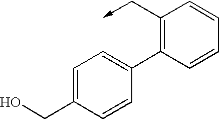 |
| 3583 3584 3585 | rac + − | 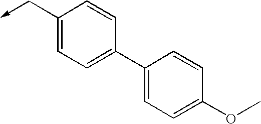 |
| 3586 3587 3588 | rac + − | 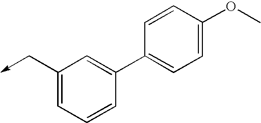 |
| 3589 3590 3591 | rac + − | 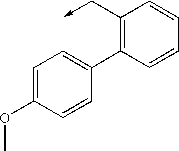 |
| 3592 3593 3594 | rac + − | 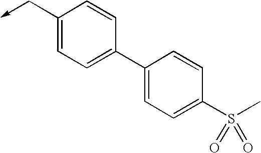 |
| 3595 3596 3597 | rac + − | 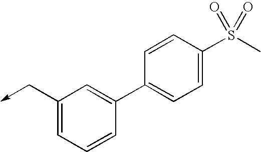 |
| 3598 3599 3600 | rac + − | 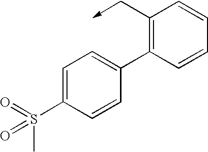 |
| 3601 3602 3603 | rac + − | 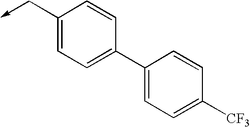 |
| 3604 3605 3606 | rac + − | 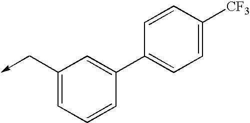 |
| 3607 3608 3609 | rac + − | 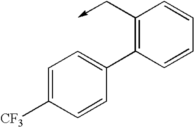 |
| 3610 3611 3612 | rac + − | 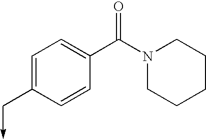 |
| 3613 3614 3615 | rac + − | 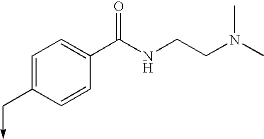 |
| 3616 3617 3618 | rac + − | 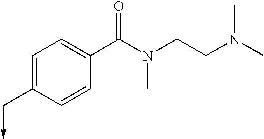 |
| 3619 3620 3621 | rac + − | 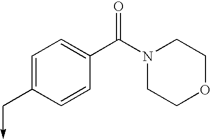 |
| 3622 3623 3624 | rac + − | 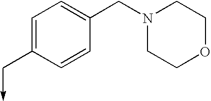 |
| 3625 3626 3627 | rac + − | 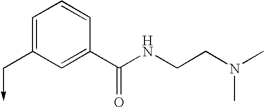 |
| 3628 3629 3630 | rac + − | 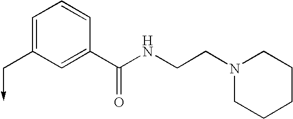 |
| 3631 3632 3633 | rac + − | 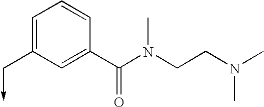 |
| 3634 3635 3636 | rac + − | 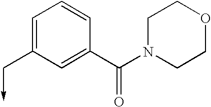 |
| 3637 3638 3639 | rac + − | 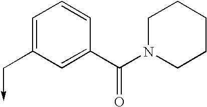 |
| 3640 3641 3642 | rac + − | 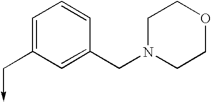 |
| 3643 3644 3645 | rac + − | 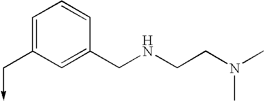 |
| 3646 3647 3648 | rac + − | 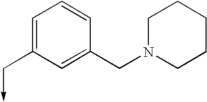 |
| 3649 3650 3651 | rac + − | 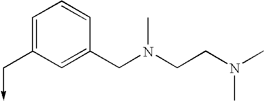 |
| 3652 3653 3654 | rac + − | 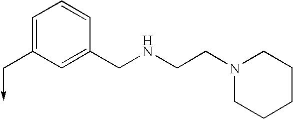 |

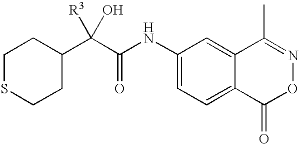

| No. | Racemic or enantiomer | R3 |
|---|---|---|
| 3655 3656 3657 | rac + − | 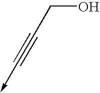 |
| 3658 3659 3660 | rac + − | 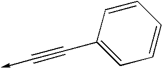 |
| 3661 3662 3663 | rac + − | 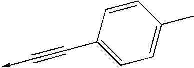 |
| 3664 3665 3666 | rac + − | 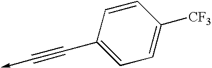 |
| 3667 3668 3669 | rac + − | 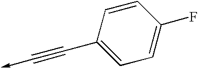 |
| 3670 3671 3672 | rac + − | 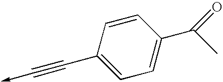 |
| 3673 3674 3675 | rac + − | 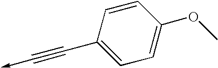 |
| 3676 3677 3678 | rac + − | 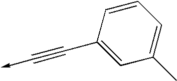 |
| 3679 3680 3681 | rac + − | 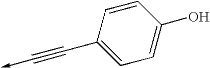 |
| 3682 3683 3684 | rac + − | 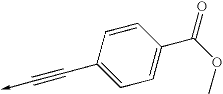 |
| 3685 3686 3687 | rac + − | 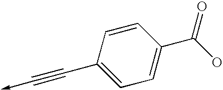 |
| 3688 3689 3690 | rac + − | 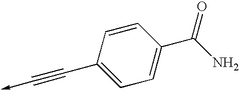 |
| 3691 3692 3693 | rac + − | 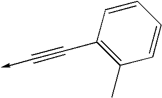 |
| 3694 3695 3696 | rac + − | 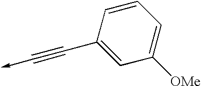 |
| 3697 3698 3699 | rac + − | 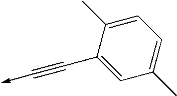 |
| 3700 3701 3702 | rac + − | 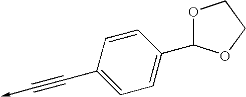 |
| 3703 3704 3705 | rac + − | 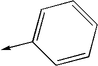 |
| 3706 3707 3708 | rac + − | 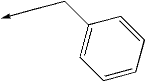 |
| 3709 3710 3711 | rac + − | 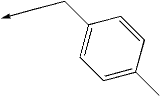 |
| 3712 3713 3714 | rac + − | 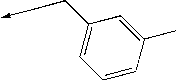 |
| 3715 3716 3717 | rac + − | 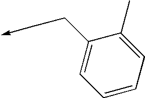 |
| 3718 3719 3720 | rac + − | 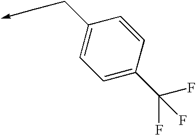 |
| 3721 3722 3723 | rac + − | 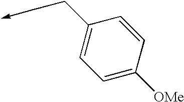 |
| 3724 3725 3726 | rac + − | 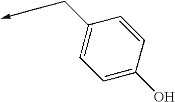 |
| 3727 3728 3729 | rac + − | 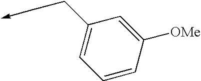 |
| 3730 3731 3732 | rac + − | 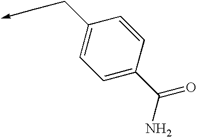 |
| 3733 3734 3735 | rac + − | 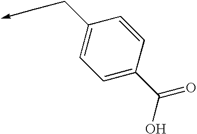 |
| 3736 3737 3738 | rac + − | 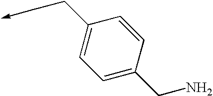 |
| 3739 3740 3741 | rac + − | 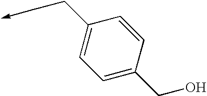 |
| 3742 3743 3744 | rac + − | 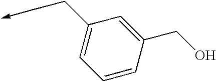 |
| 3745 3746 3747 | rac + − | 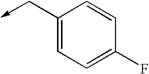 |
| 3748 3749 3750 | rac + − | 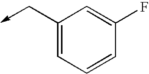 |
| 3751 3752 3753 | rac + − | 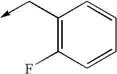 |
| 3754 3755 3756 | rac + − | 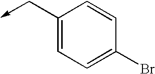 |
| 3757 3758 3759 | rac + − | 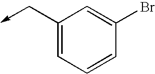 |
| 3760 3761 3762 | rac + − | 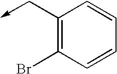 |
| 3763 3764 3765 | rac + − | 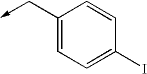 |
| 3766 3767 3768 | rac + − | 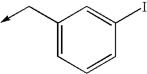 |
| 3769 3770 3771 | rac + − | 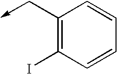 |
| 3772 3773 3774 | rac + − | 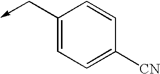 |
| 3775 3776 3777 | rac + − | 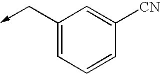 |
| 3778 3779 3780 | rac + − | 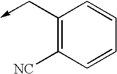 |
| 3781 3782 3783 | rac + − | 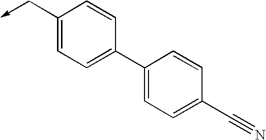 |
| 3784 3785 3786 | rac + − | 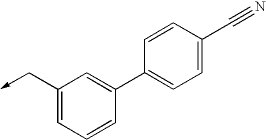 |
| 3787 3788 3789 | rac + − | 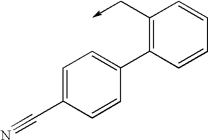 |
| 3790 3791 3792 | rac + − | 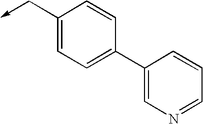 |
| 3793 3794 3795 | rac + − | 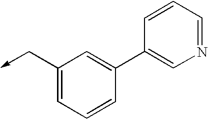 |
| 3796 3797 3798 | rac + − | 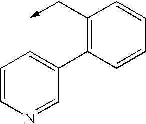 |
| 3799 3800 3801 | rac + − | 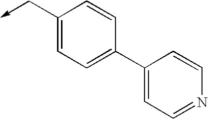 |
| 3802 3803 3804 | rac + − | 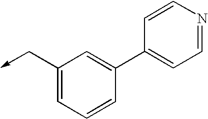 |
| 3805 3806 3807 | rac + − | 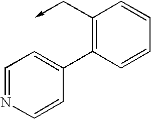 |
| 3808 3809 3810 | rac + − | 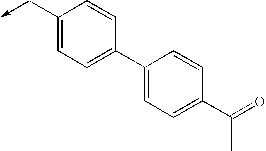 |
| 3811 3812 3813 | rac + − | 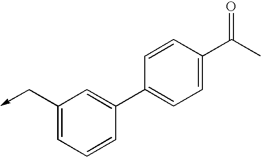 |
| 3814 3815 3816 | rac + − | 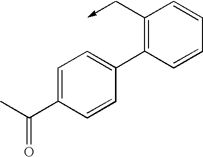 |
| 3817 3818 3819 | rac + − | 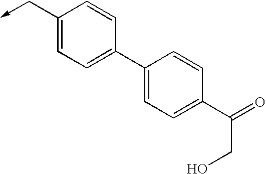 |
| 3820 3821 3822 | rac + − | 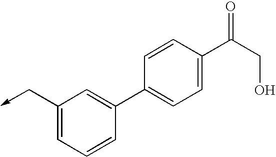 |
| 3823 3824 3825 | rac + − | 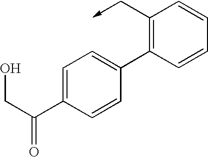 |
| 3826 3827 3828 | rac + − | 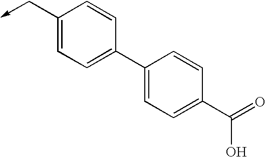 |
| 3829 3830 3831 | rac + − | 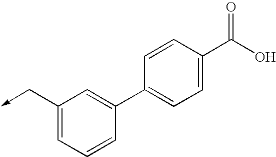 |
| 3832 3833 3834 | rac + − | 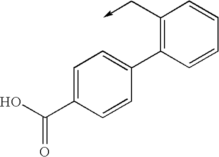 |
| 3835 3836 3837 | rac + − | 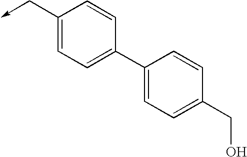 |
| 3838 3839 3840 | rac + − | 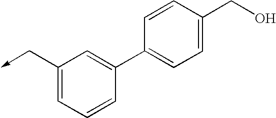 |
| 3841 3842 3843 | rac + − | 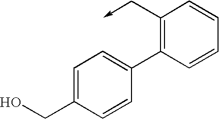 |
| 3844 3845 3846 | rac + − | 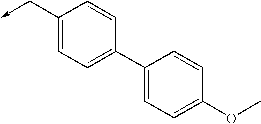 |
| 3847 3848 3849 | rac + − | 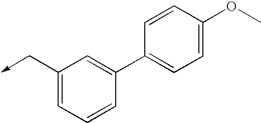 |
| 3850 3851 3852 | rac + − | 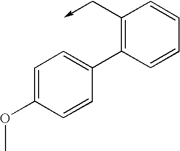 |
| 3853 3854 3855 | rac + − | 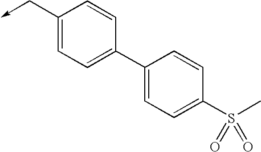 |
| 3856 3857 3858 | rac + − | 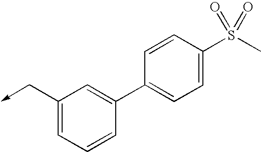 |
| 3859 3860 3861 | rac + − | 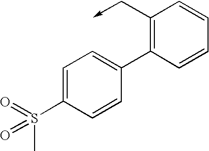 |
| 3862 3863 3864 | rac + − | 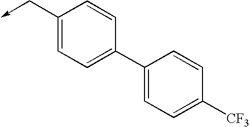 |
| 3865 3866 3867 | rac + − | 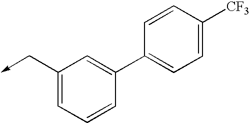 |
| 3868 3869 3870 | rac + − | 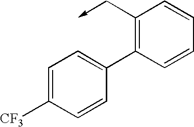 |
| 3871 3872 3873 | rac + − | 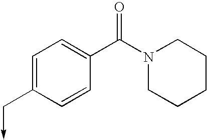 |
| 3874 3875 3876 | rac + − | 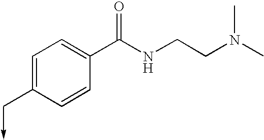 |
| 3877 3878 3879 | rac + − | 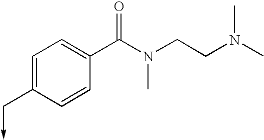 |
| 3880 3881 3882 | rac + − | 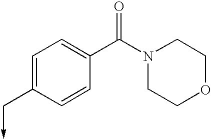 |
| 3883 3884 3885 | rac + − | 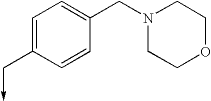 |
| 3886 3887 3888 | rac + − | 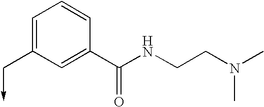 |
| 3889 3890 3891 | rac + − | 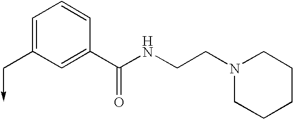 |
| 3892 3893 3894 | rac + − | 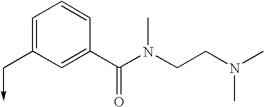 |
| 3895 3896 3897 | rac + − | 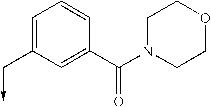 |
| 3898 3899 3900 | rac + − | 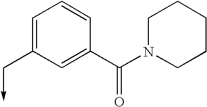 |
| 3901 3902 3903 | rac + − | 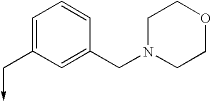 |
| 3904 3905 3906 | rac + − | 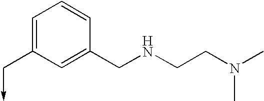 |
| 3907 3908 3909 | rac + − | 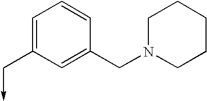 |
| 3910 3911 3912 | rac + − | 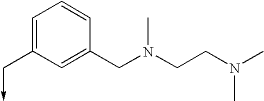 |
| 3913 3914 3915 | rac + − | 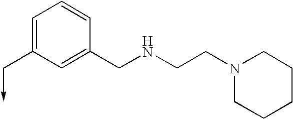 |
| No. | racemic or enantiomer or stereoisomer | structure |
| 3916 3917 3918 | rac + − | 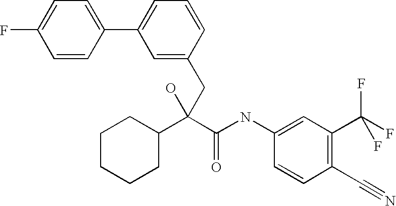 |
| 3919 3920 3921 3922 3923 | stereoisomer mixture (R, R) (S, S) (R, S) (S, R) | 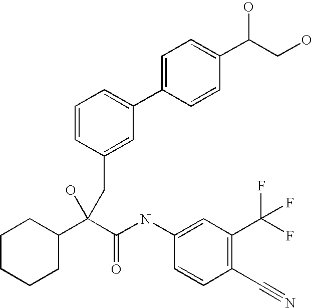 |
| 3924 3925 3926 | rac + − | 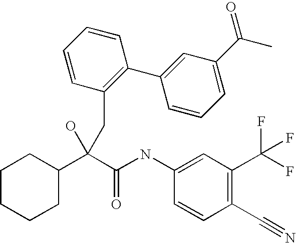 |
| 3927 3928 3929 | rac + − | 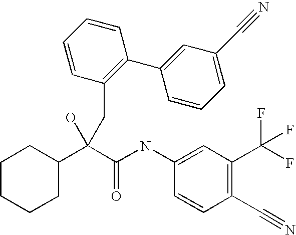 |
| 3930 3931 3932 | rac + − | 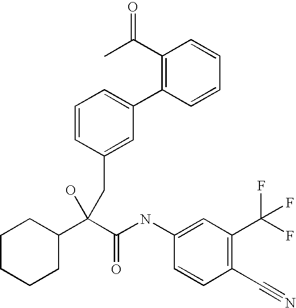 |
| 3933 3934 3935 | rac + − | 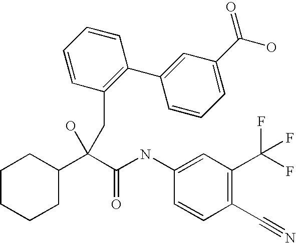 |
| 3936 3937 3938 | rac + − | 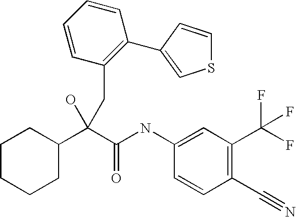 |
| 3939 3940 3941 | rac + − | 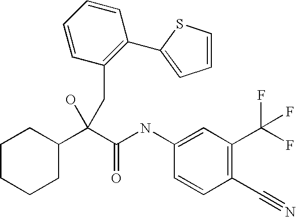 |
| 3942 3943 3944 | rac + − | 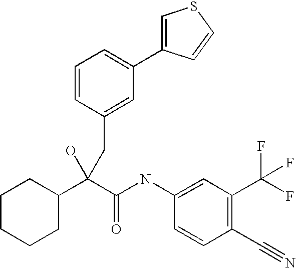 |
| 3945 3946 3947 | rac + − | 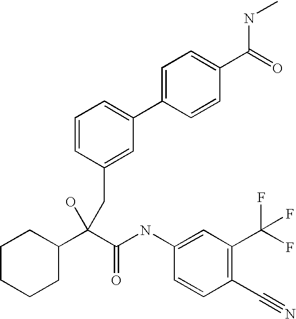 |
| 3948 3949 3950 | rac + − | 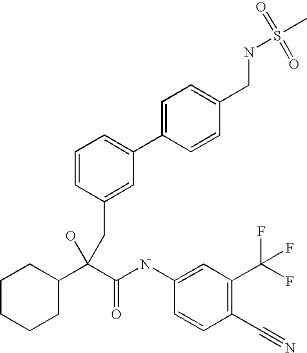 |
| 3951 3952 3953 | rac + − | 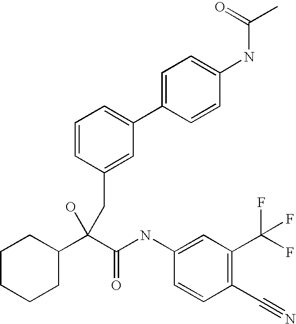 |
| 3954 3955 3956 | rac + − | 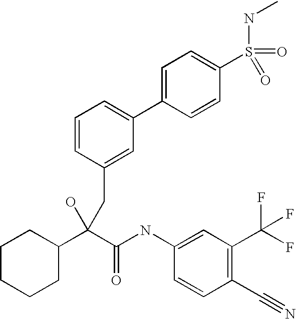 |
| 3957 3958 3959 | rac + − | 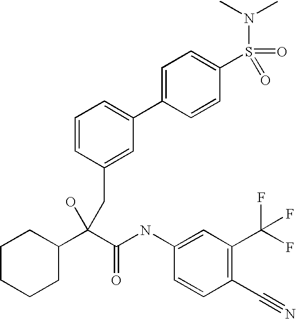 |
| 3960 3961 3962 | rac + − | 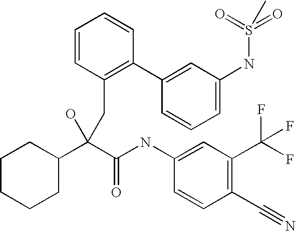 |
| 3963 3964 3965 | rac + − | 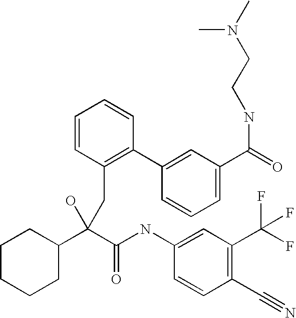 |
| 3966 3967 3968 3969 3970 | stereoisomer mixture (R, R) (S, S) (R, S) (S, R) | 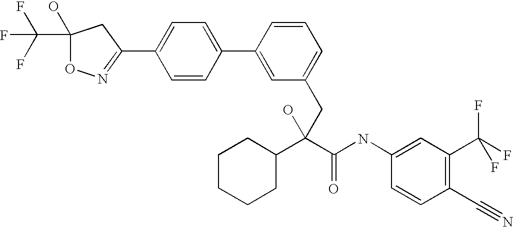 |
| 3971 3972 3973 | rac + − | 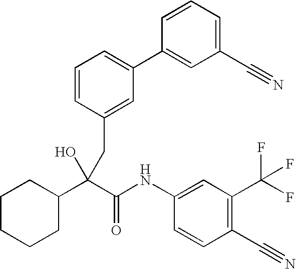 |
| 3974 3975 3976 | rac + − | 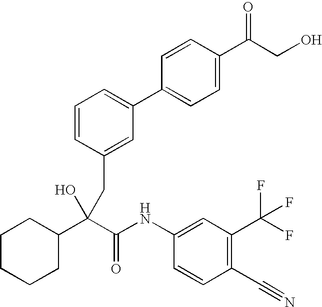 |
| 3977 3978 3979 | rac + − | 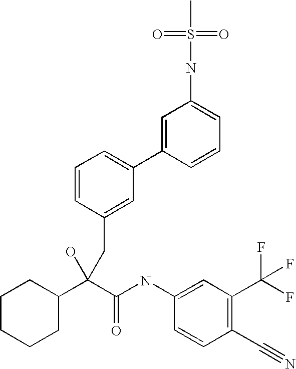 |
| 3980 3981 3982 | rac + − | 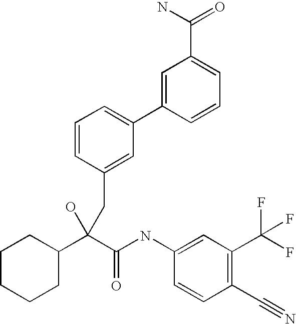 |
| 3983 3984 3985 | rac + − | 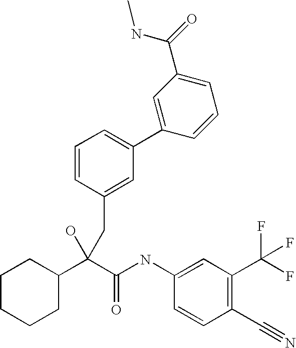 |
| 3986 3987 3988 | rac + − | 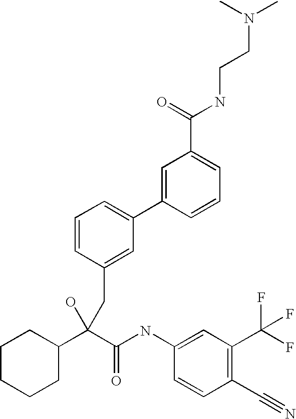 |
| 3989 3990 3991 | rac + − | 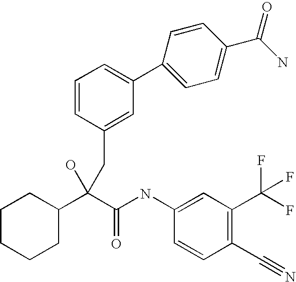 |
| 3992 3993 3994 | rac + − | 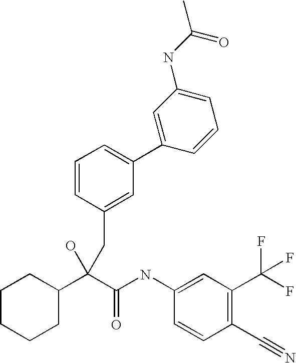 |
| 3995 3996 3997 | rac + − | 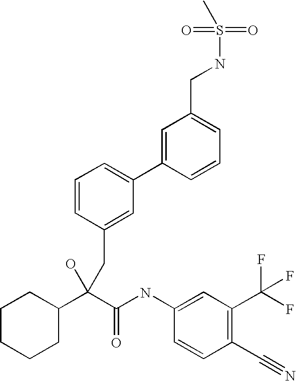 |
| 3998 3999 4000 4001 4002 | stereoisomer mixture (R, R) (S, S) (R, S) (S, R) | 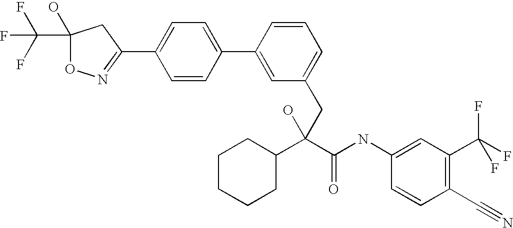 |
| 4003 4004 4005 | rac + − | 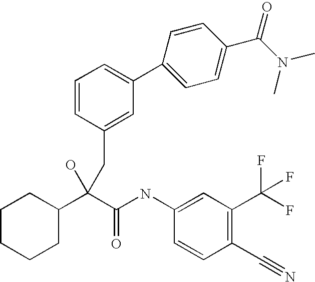 |
| 4006 4007 4008 | rac + − | 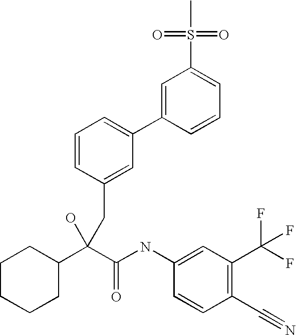 |
| 4009 4010 4011 | rac + − | 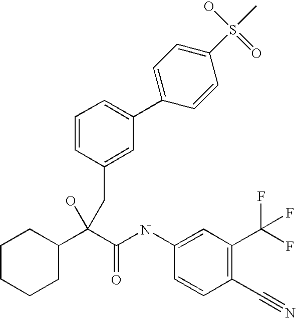 |
| 4012 4013 4014 | rac + − | 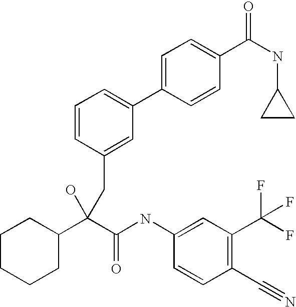 |
| 4015 4016 4017 | rac + − | 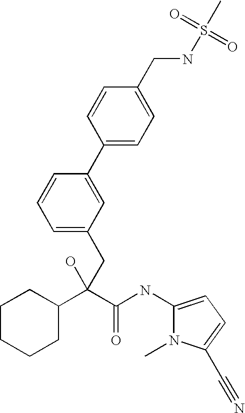 |
| 4018 4019 4020 | rac + − | 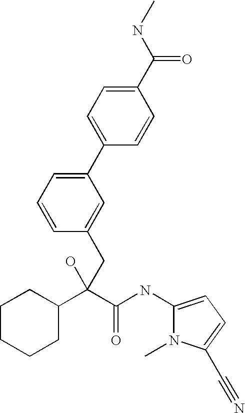 |
| 4021 4022 4023 | rac + − | 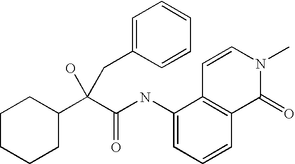 |
| 4024 4025 4026 | rac + − | 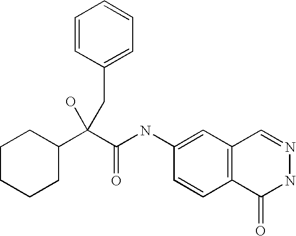 |
| 4027 4028 4029 | rac + − | 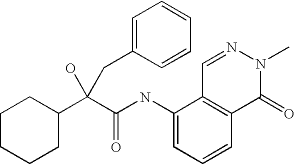 |
| 4030 4031 4032 | rac + − | 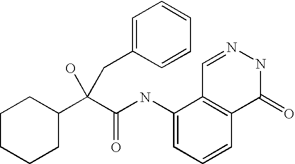 |
| 4033 4034 4035 | rac + − | 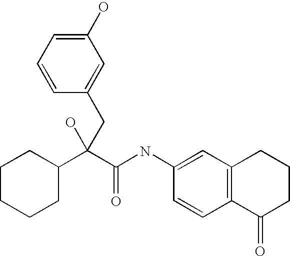 |
| 4036 4037 4038 | rac + − | 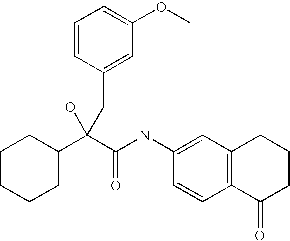 |
| 4039 4040 4041 | rac + − | 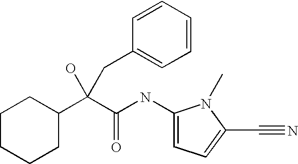 |
| 4042 4043 4044 | rac + − | 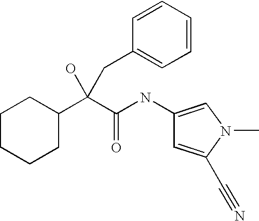 |
| 4045 4046 4047 | rac + − | 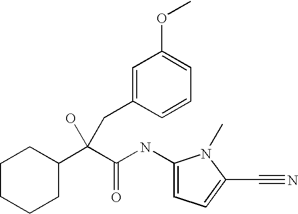 |
| 4048 4049 4050 | rac + − | 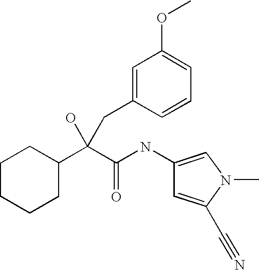 |
| 4051 4052 4053 | rac + − | 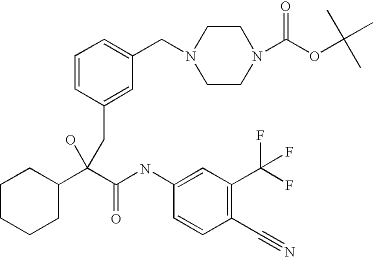 |
| 4054 4055 4056 | rac + − | 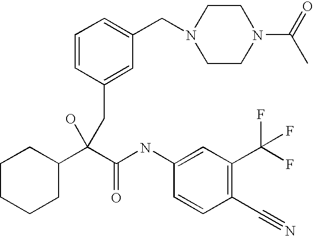 |
| 4057 4058 4059 | rac + − | 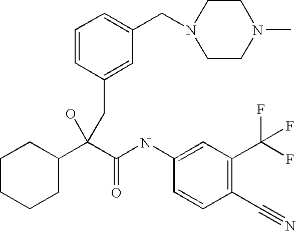 |
| 4060 4061 4062 | rac + − | 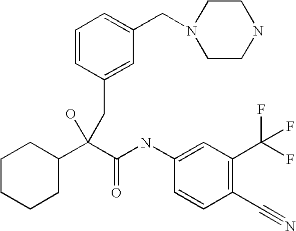 |
| 4063 4064 4065 | rac + − | 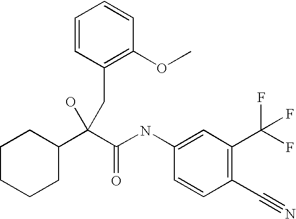 |
| 4066 4067 4068 | rac + − | 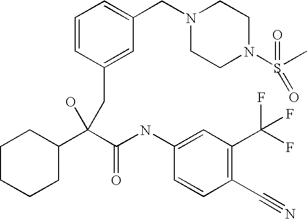 |
| 4069 4070 4071 | rac + − | 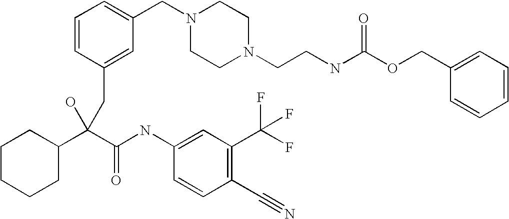 |
| 4072 4073 4074 | rac + − | 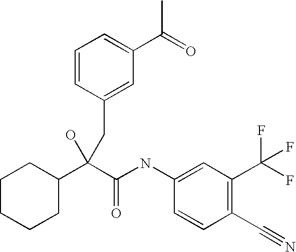 |
| 4075 4076 4077 | rac + − | 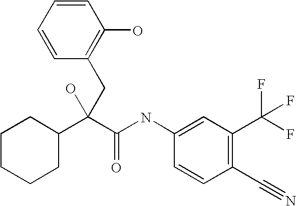 |
| 4078 4079 4080 | rac + − | 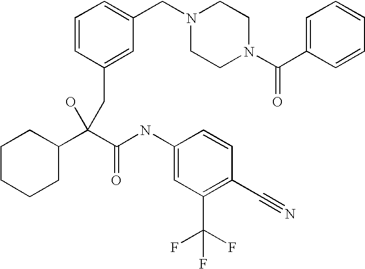 |
| 4081 4082 4083 | rac + − | 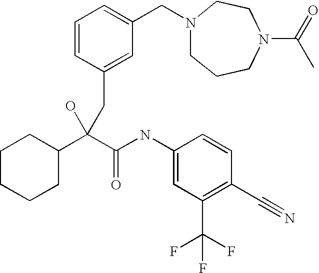 |
| 4084 4085 4086 | rac + − | 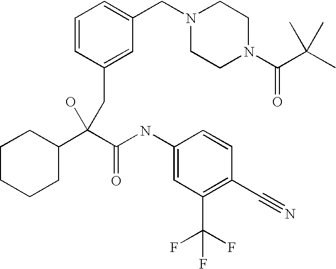 |
| 4087 4088 4089 | rac + − | 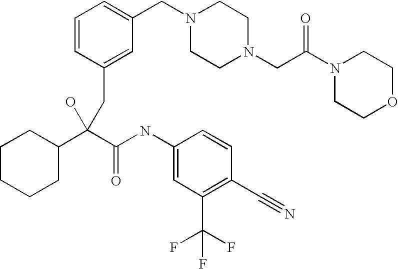 |
| 4090 4091 4092 | rac + − | 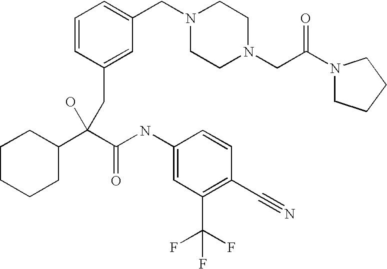 |
| 4093 4094 4095 | rac + − | 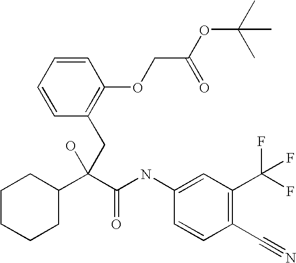 |
| 4096 4097 4098 | rac + − | 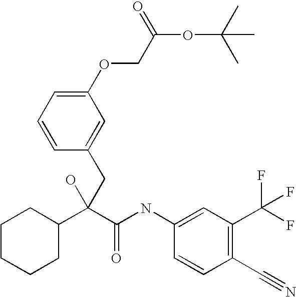 |
| 4099 4100 4101 | rac + − | 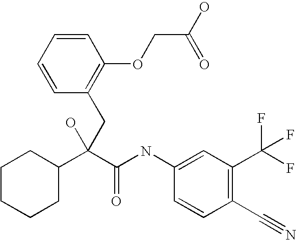 |
| 4102 4103 4104 | rac + − | 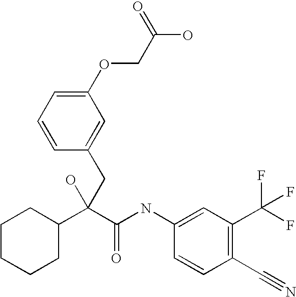 |
| 4105 4106 4107 | rac + − | 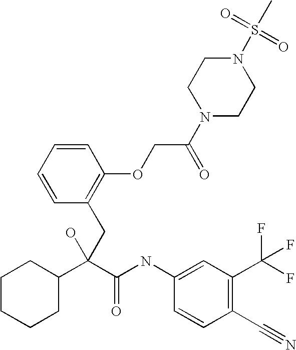 |
| 4108 4109 4110 | rac + − | 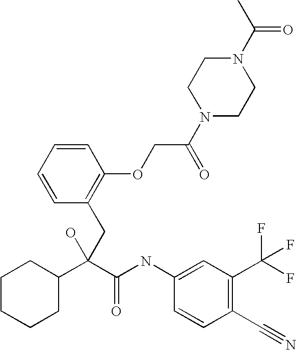 |
| 4111 4112 4113 | rac + − | 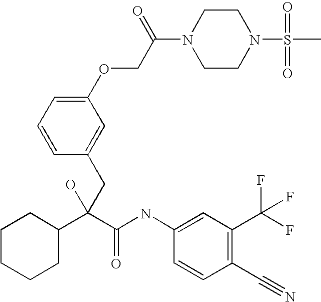 |
| 4114 4115 4116 | rac + − | 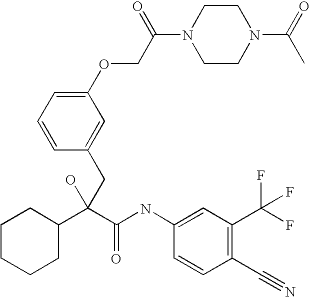 |
| 4117 4118 4119 | rac + − | 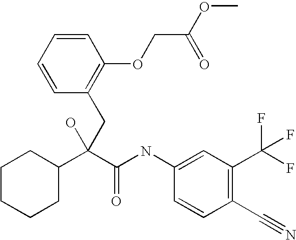 |
| 4120 4121 4122 | rac + − | 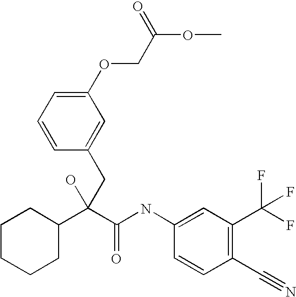 |
| 4123 4124 4125 | rac + − | 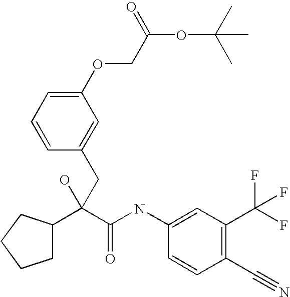 |
| 4126 4127 4128 | rac + − | 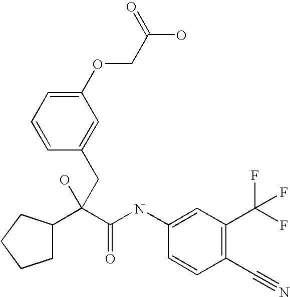 |
| 4129 4130 4131 | rac + − | 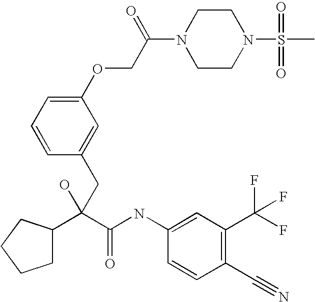 |
| 4132 4133 4134 | rac + − | 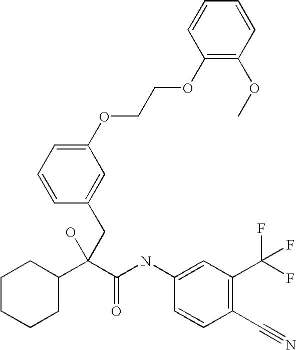 |
| 4135 4136 4137 | rac + − | 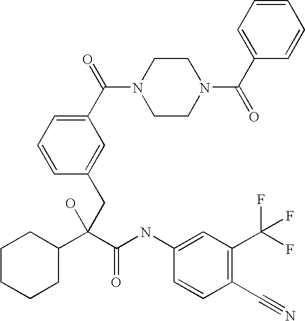 |
| 4138 4139 4140 | rac + − | 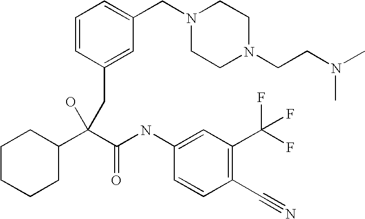 |
| 4141 4142 4143 | rac + − | 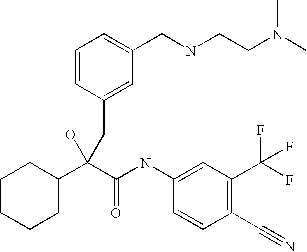 |
| 4144 4145 4146 | rac + − | 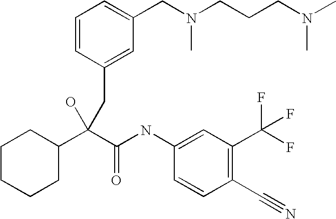 |
| 4147 4148 4149 | rac + − | 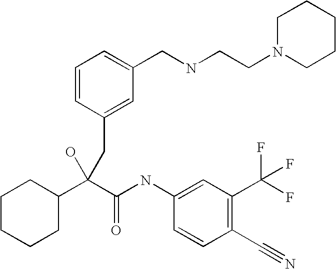 |
| 4150 4151 4152 | rac + − | 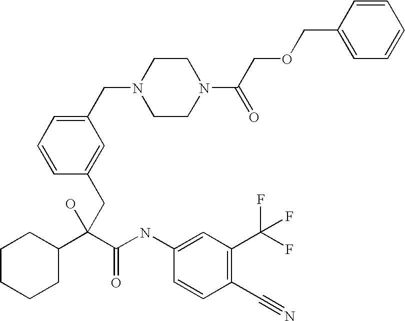 |
| 4153 4154 4155 | rac + − | 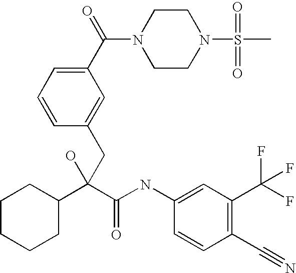 |
| 4156 4157 4158 | rac + − | 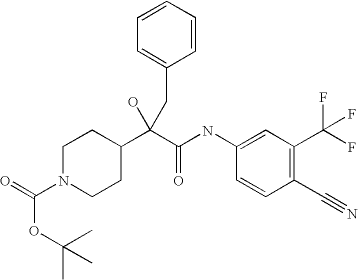 |
| 4159 4160 4161 | rac + − | 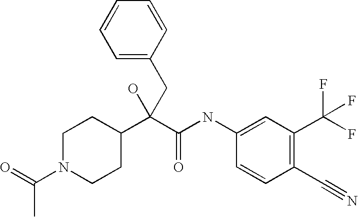 |
| 4162 4163 4164 | rac + − | 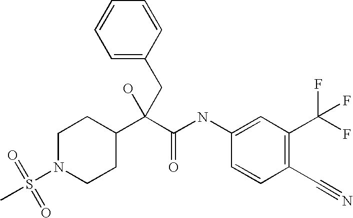 |
| 4165 4166 4167 | rac + − | 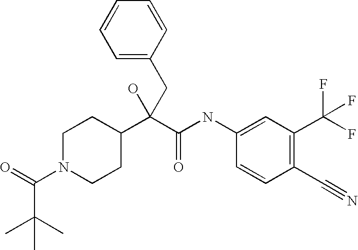 |
| 4168 4169 4170 | rac + − | 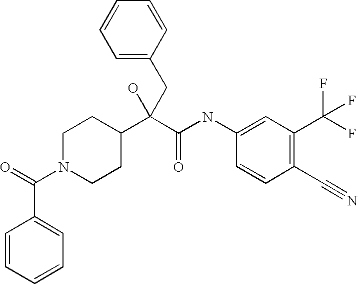 |
| 4171 4172 4173 | rac + − | 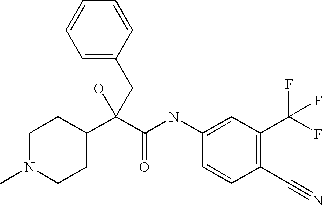 |
| 4174 4175 4176 | rac + − | 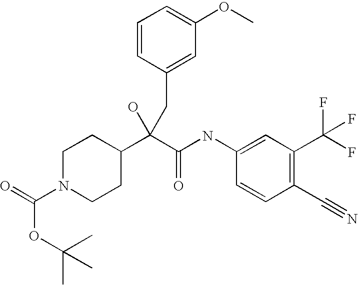 |
| 4177 4178 4179 | rac + − | 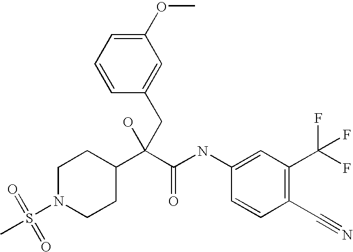 |
| 4180 4181 4182 | rac + − | 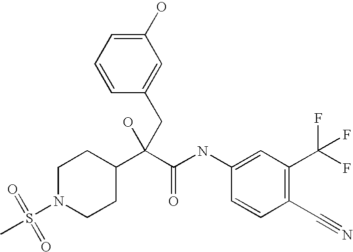 |
| 4183 4184 4185 | rac + − | 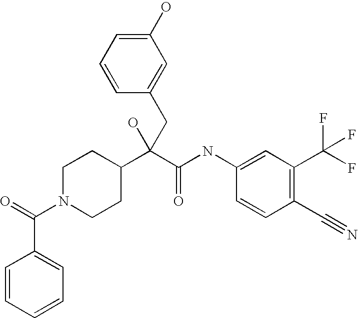 |
| 4186 4187 4188 | rac + − | 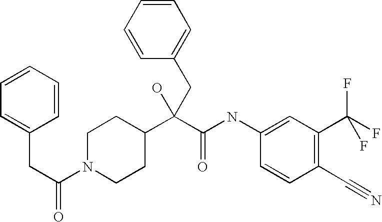 |
| 4189 4190 4191 | rac + − | 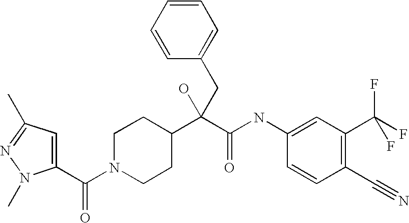 |
| 4192 4193 4194 | rac + − | 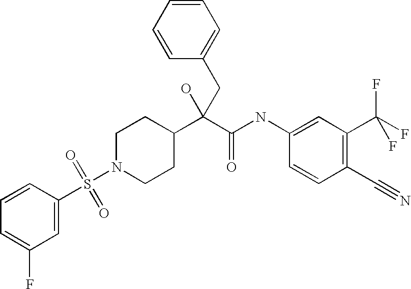 |
| 4195 4196 4197 | rac + − | 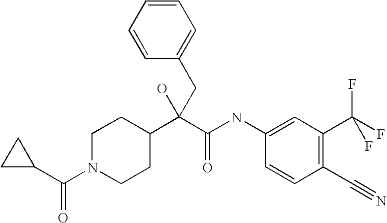 |
| 4198 4199 4200 | rac + − | 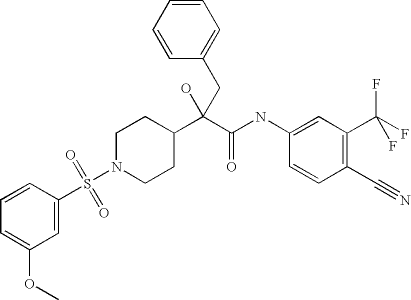 |
| 4201 4202 4203 | rac + − | 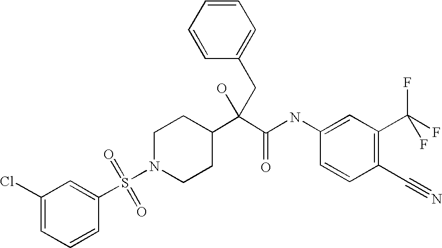 |
| 4204 4205 4206 | rac + − | 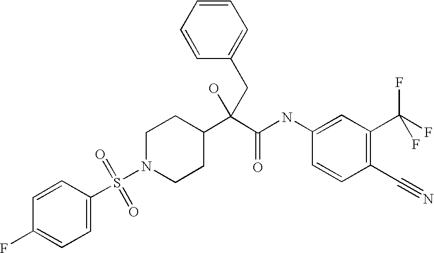 |
| 4207 4208 4209 | rac + − | 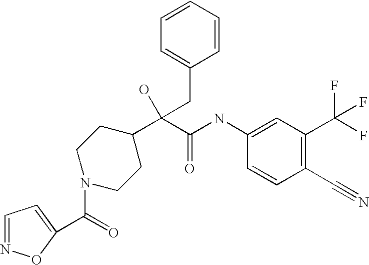 |
| 4210 4211 4212 | rac + − | 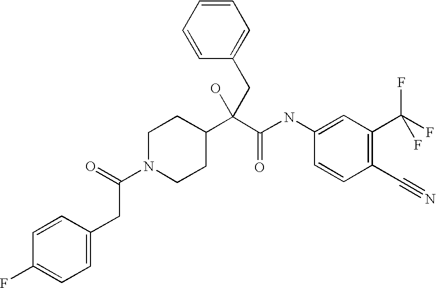 |
| 4213 4214 4215 | rac + − | 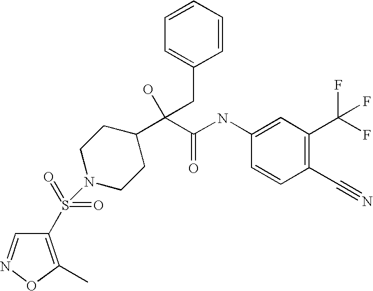 |
| 4216 4217 4218 | rac + − | 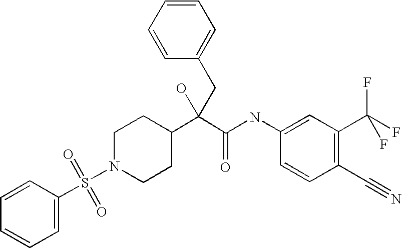 |
| 4219 4220 4221 | rac + − | 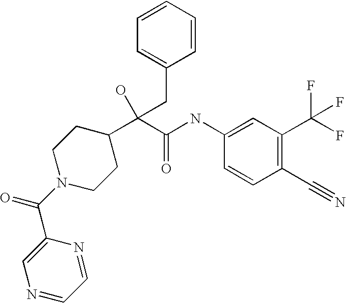 |
| 4222 4223 4224 | rac + − | 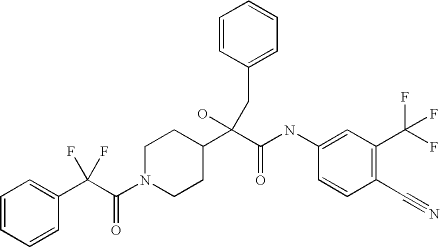 |
| 4225 4226 4227 | rac + − | 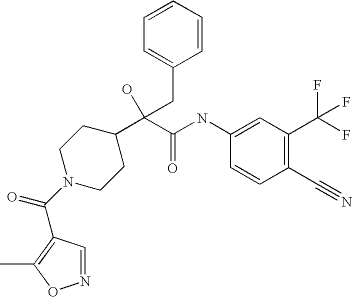 |
| 4228 4229 4230 | rac + − | 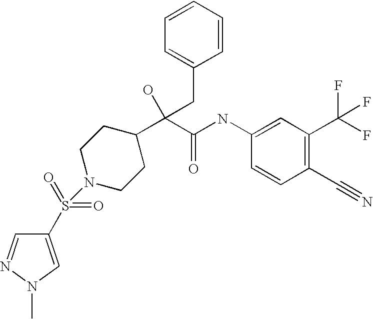 |
| 4231 4232 4233 | rac + − | 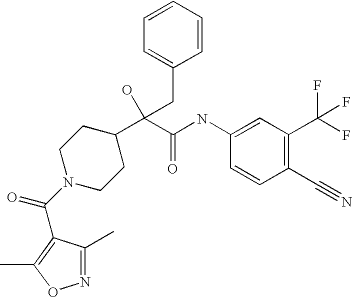 |
| 4234 4235 4236 | rac + − | 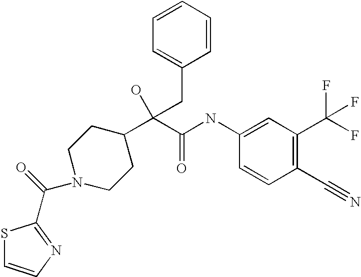 |
| 4237 4238 4239 | rac + − | 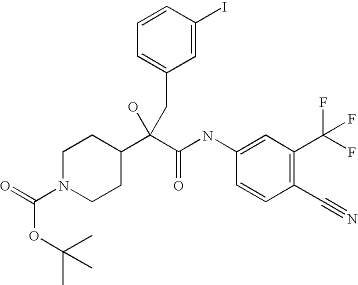 |
| 4240 4241 4242 | rac + − | 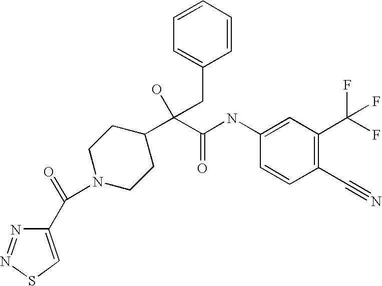 |
| 4243 4244 4245 | rac + − | 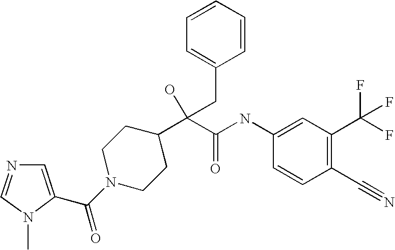 |
| 4246 4247 4248 | rac + − | 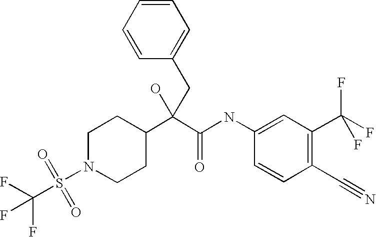 |
| 4249 4250 4251 | rac + − | 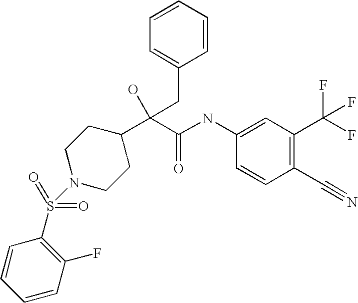 |
| 4252 4253 4254 | rac + − | 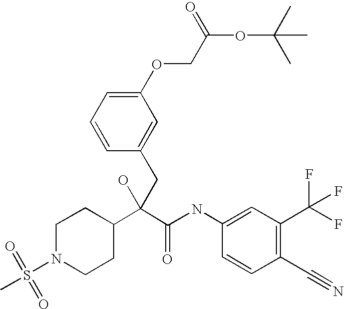 |
| 4255 4256 4257 | rac + − | 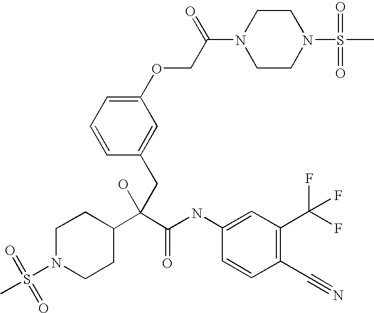 |
| 4258 4259 4260 | rac + − | 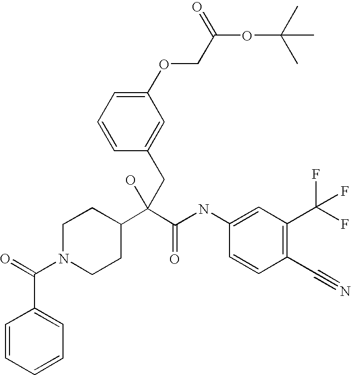 |
| 4261 4262 4263 | rac + − | 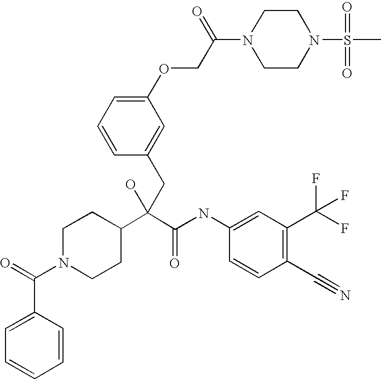 |
| 4264 4265 4266 | rac + − | 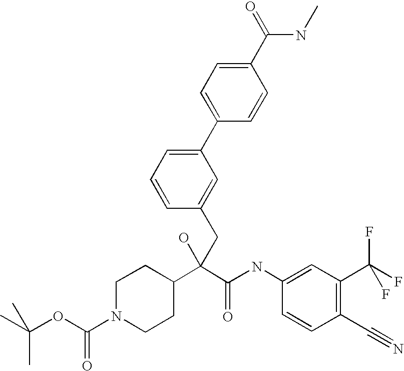 |
| 4267 4268 4269 | rac + − | 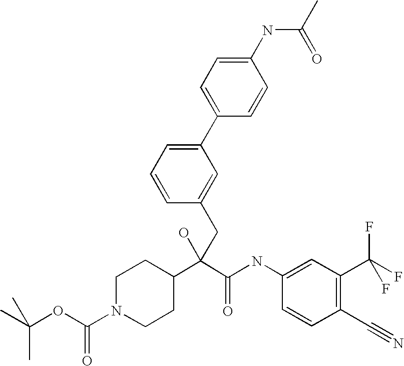 |
| 4270 4271 4272 | rac + − | 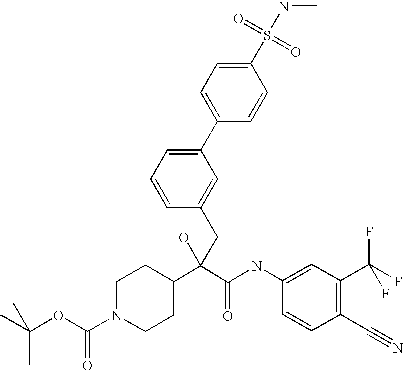 |
| 4273 4274 4275 | rac + − | 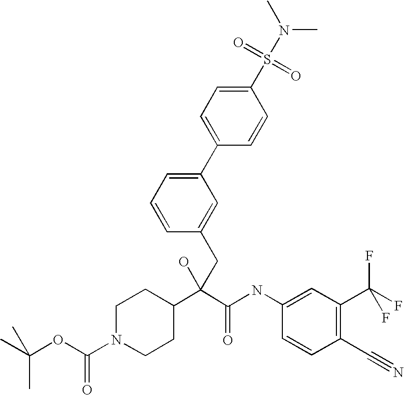 |
| 4276 4277 4278 | rac + − | 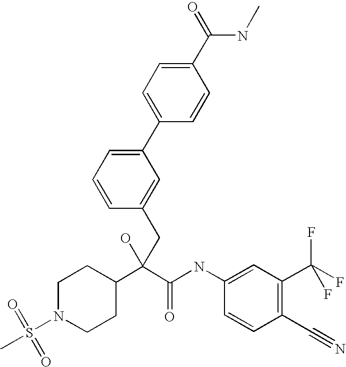 |
| 4279 4280 4281 | rac + − | 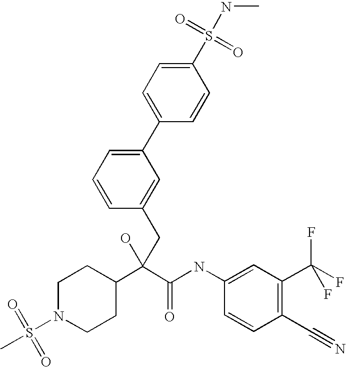 |
| 4282 4283 4284 | rac + − | 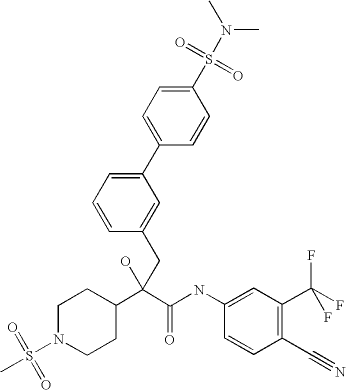 |
| 4285 4286 4287 4288 4289 | stereoisomer mixture (R, R) (S, S) (R, S) (S, R) | 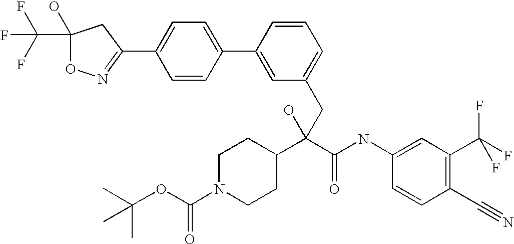 |
| 4290 4291 4292 4293 4294 | stereoisomer mixture (R, R) (S, S) (R, S) (S, R) | 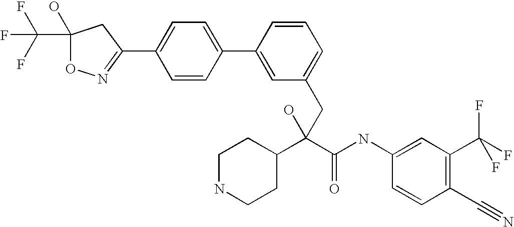 |
| 4295 4296 4297 | rac + − | 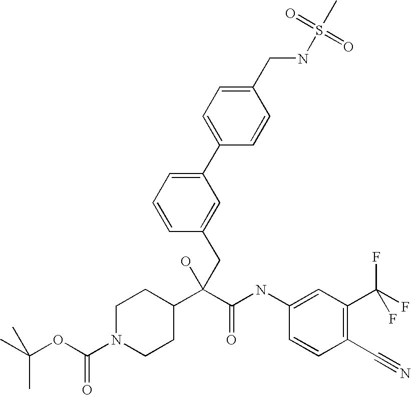 |
| 4298 4299 4300 | rac + − | 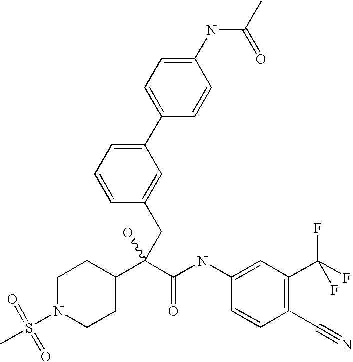 |
| 4301 4302 4303 4304 4305 | stereoisomer mixture (R, R) (S, S) (R, S) (S, R) | 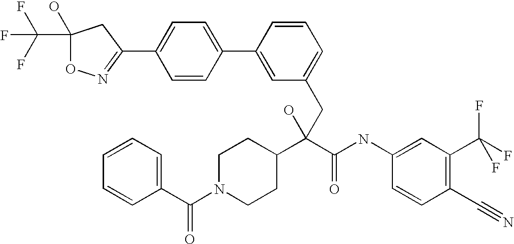 |
| 4306 4307 4308 | rac + − | 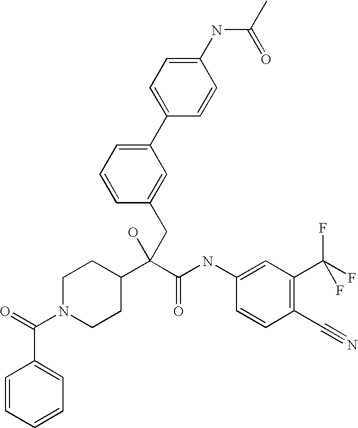 |
| 4309 4310 4311 | rac + − | 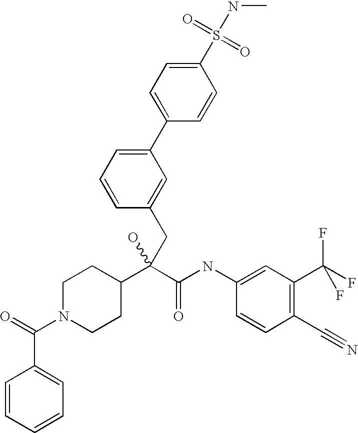 |
| 4312 4313 4314 | rac + − | 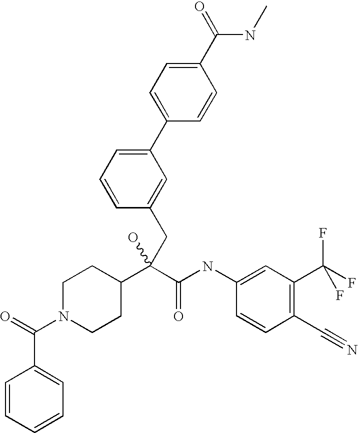 |
| 4315 4316 4317 | rac + − | 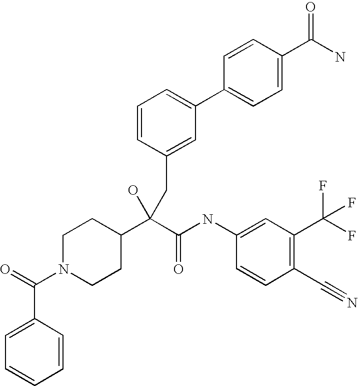 |
| 4318 4319 4320 | rac + − | 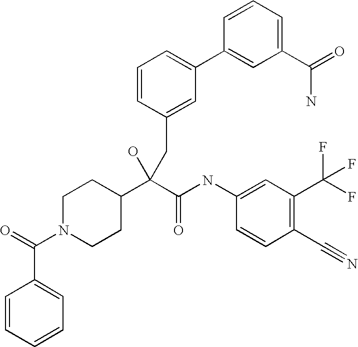 |
| 4321 4322 4323 | rac + − | 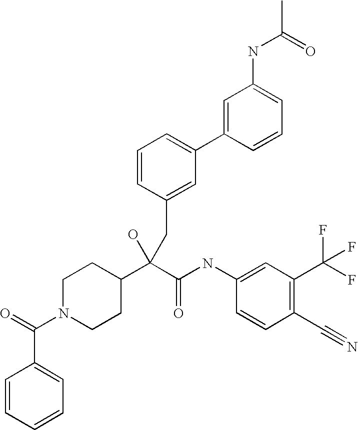 |
| 4324 4325 4326 | rac + − | 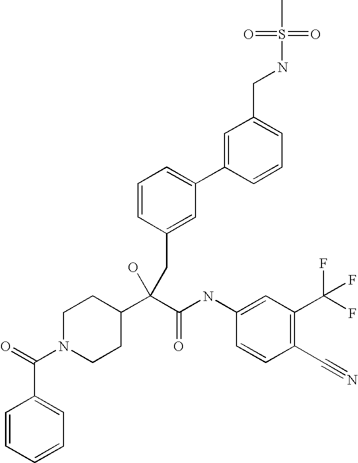 |
| 4327 4328 4329 | rac + − | 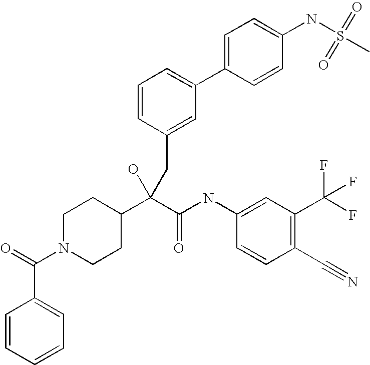 |
| 4330 4331 4332 | rac + − | 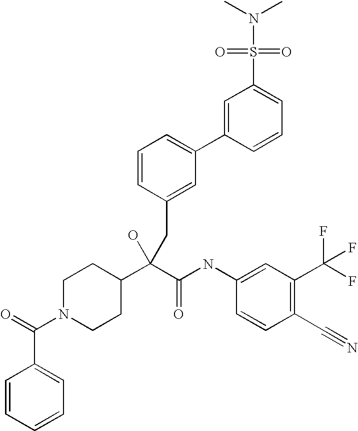 |
| 4333 4334 4335 | rac + − | 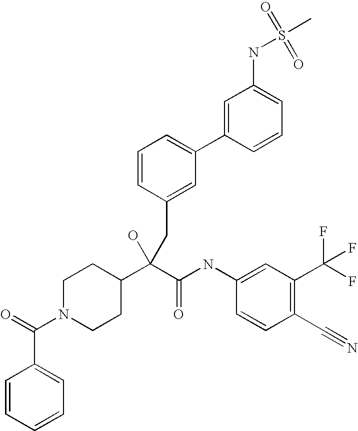 |
| 4336 4337 4338 | rac + − | 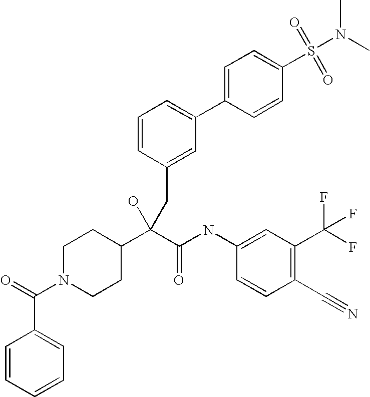 |
| 4339 4340 4341 | rac + − | 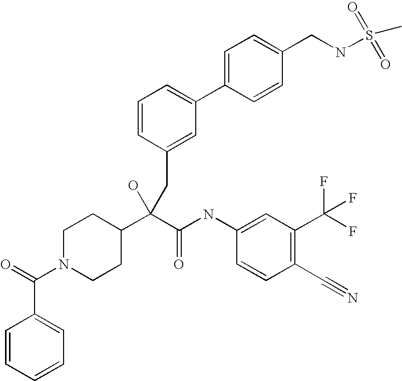 |
| 4342 4343 4344 | rac + − | 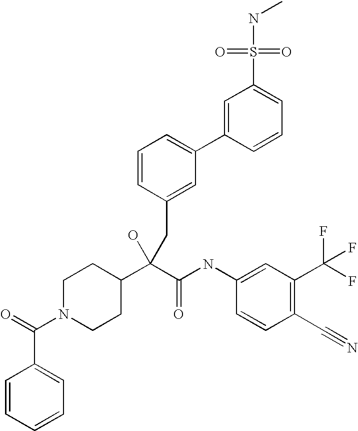 |
| 4345 4346 4347 | rac + − | 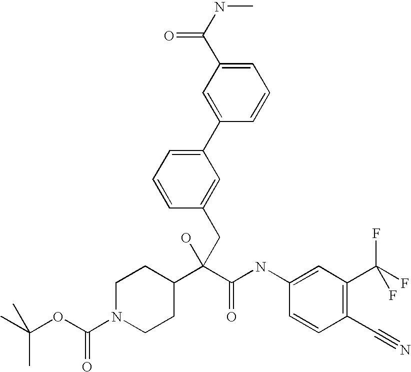 |
| 4348 4349 4350 | rac + − | 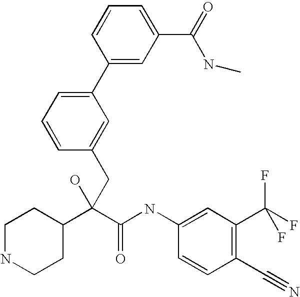 |
| 4351 4352 4353 | rac + − | 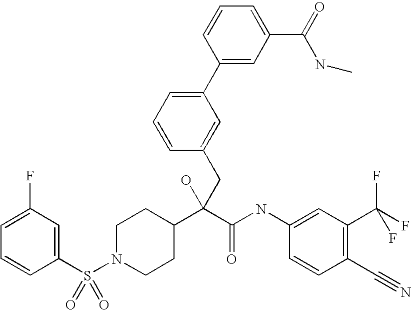 |
| 4354 4355 4356 | rac + − | 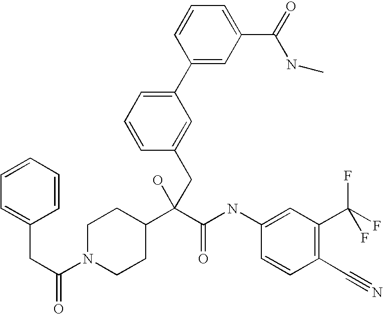 |
| 4357 4358 4359 | rac + − | 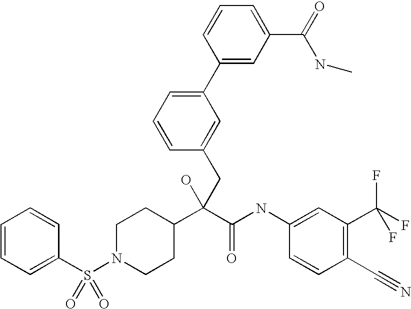 |
| 4360 4361 4362 | rac + − | 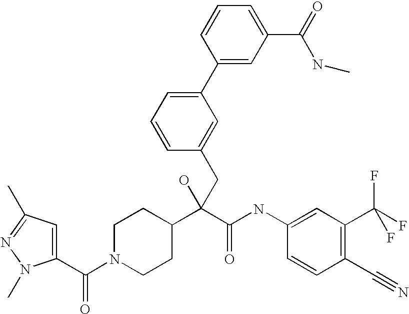 |
| 4363 4364 4365 | rac + − | 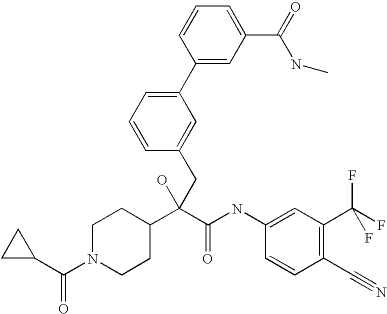 |
| 4366 4367 4368 | rac + − | 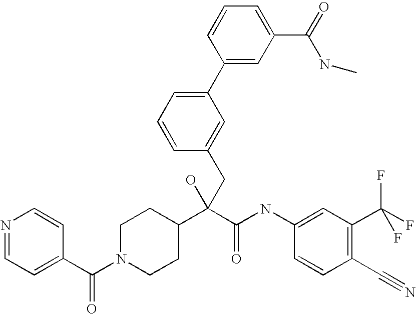 |
| 4369 4370 4371 4372 4373 | stereoisomer mixture (R, R) (S, S) (R, S) (S, R) | 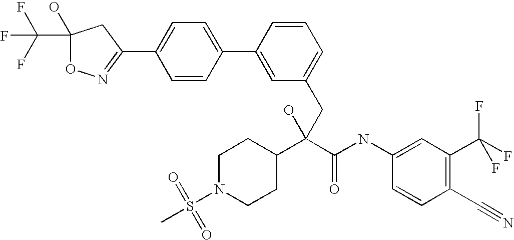 |
| 4374 4375 4376 | rac + − | 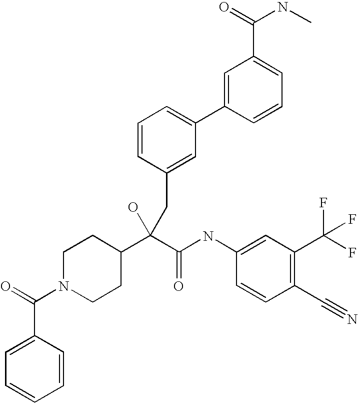 |
| 4377 4378 4379 | rac + − | 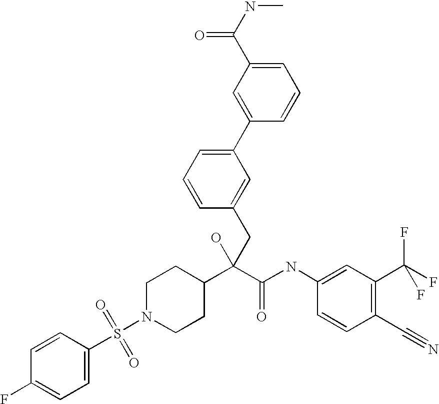 |
| 4380 4381 4382 | rac + − | 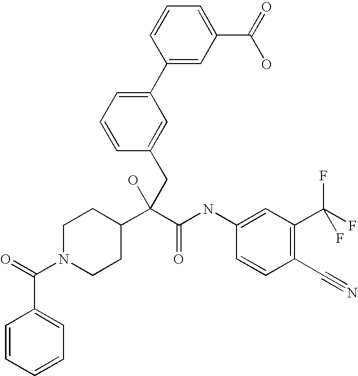 |
| 4383 4384 4385 | rac + − | 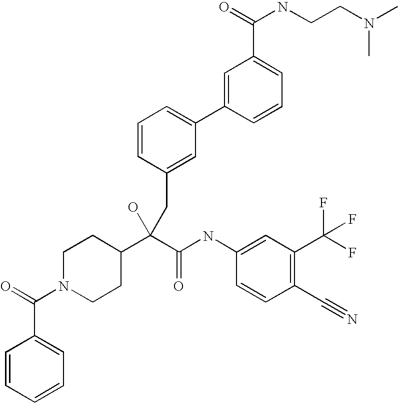 |
| 4386 4387 4388 | rac + − | 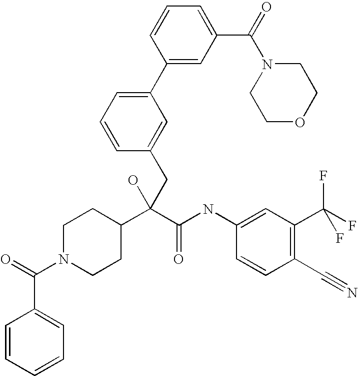 |
| 4389 4390 4391 | rac + − | 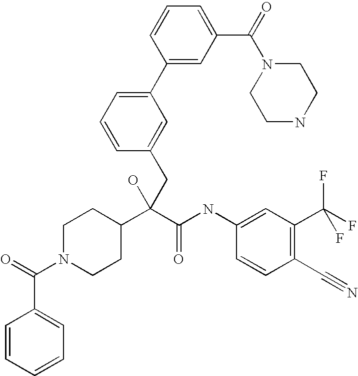 |
| 4392 4393 4394 | rac + − | 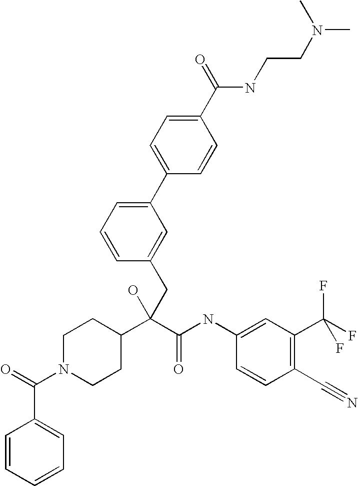 |
| 4395 4396 4397 | rac + − | 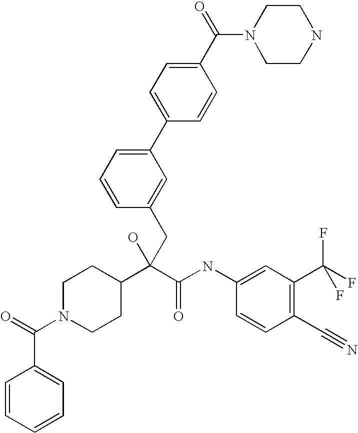 |
| 4398 4399 4400 | rac + − | 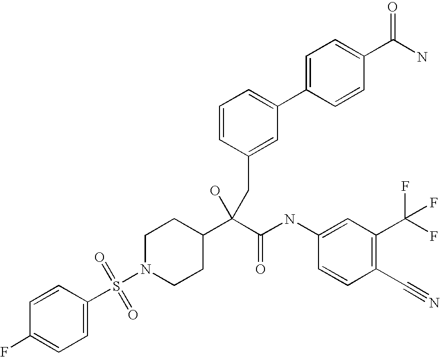 |
| 4401 4402 4403 | rac + − | 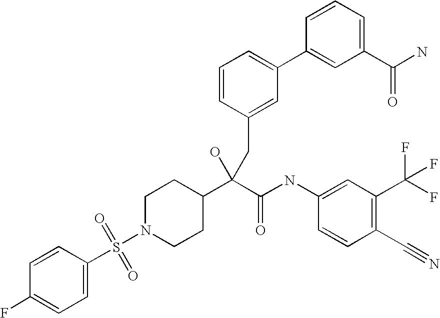 |
| 4404 4405 4406 | rac + − | 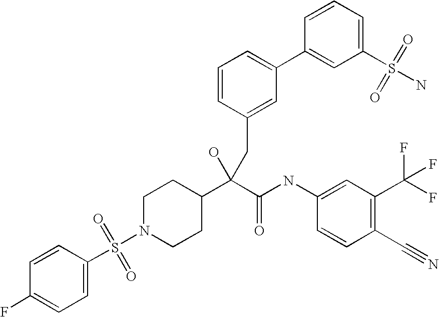 |
| 4407 4408 4409 | rac + − | 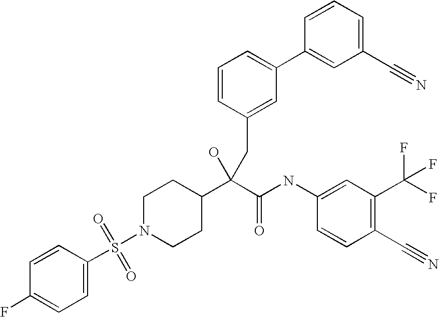 |
| 4410 4411 4412 | rac + − | 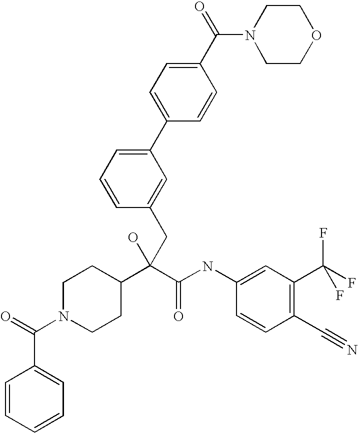 |
| 4413 4414 4415 | rac + − | 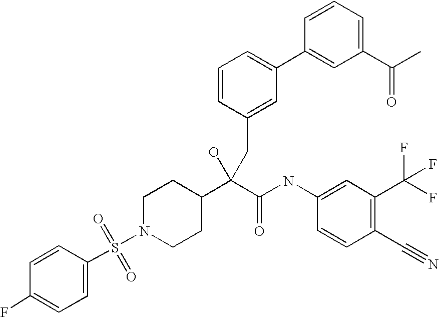 |
| 4416 4417 4418 | rac + − | 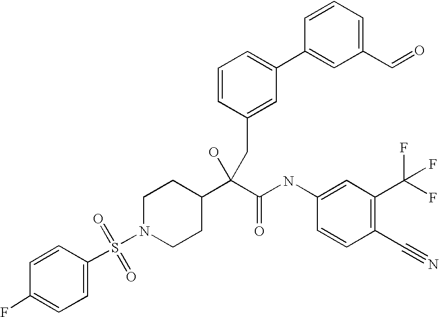 |
| 4419 4420 4421 | rac + − | 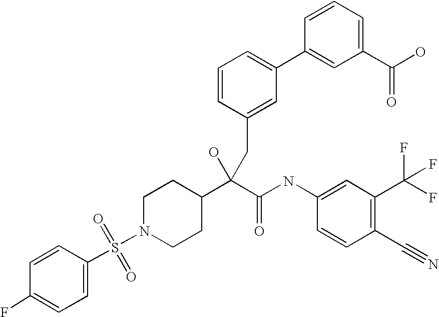 |
| 4422 4423 4424 | rac + − | 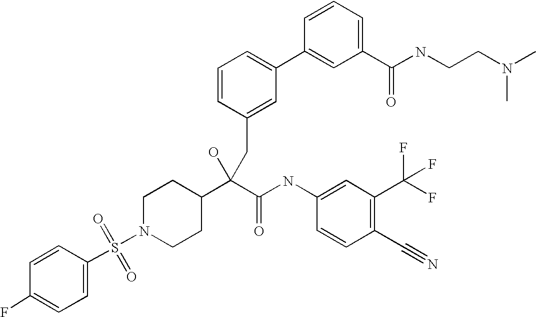 |
| 4425 4426 4427 | rac + − | 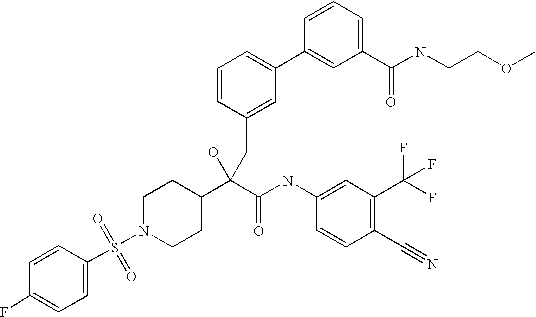 |
| 4428 4429 4430 | rac + − | 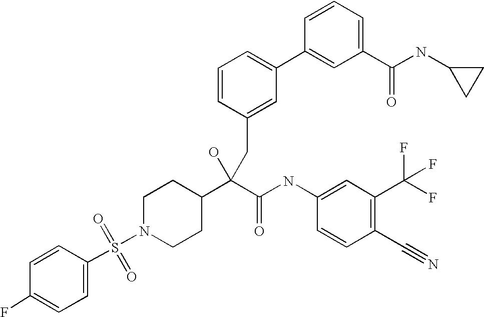 |
| 4431 4432 4433 | rac + − | 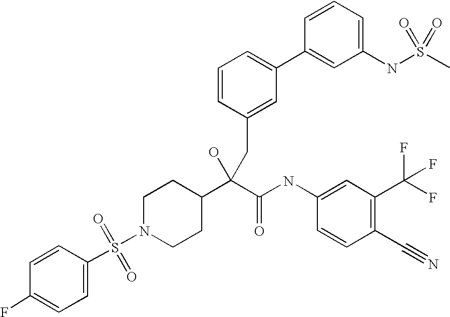 |
| 4434 4435 4436 4437 4438 | stereoisomer mixture (R, R) (S, S) (R, S) (S, R) | 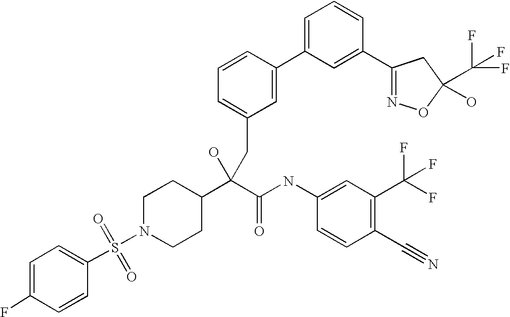 |
| 4439 4440 4441 | rac + − | 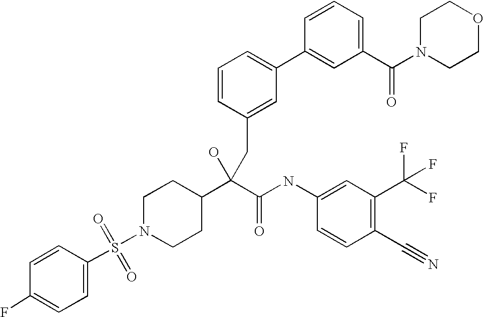 |
| 4442 4443 4444 | rac + − | 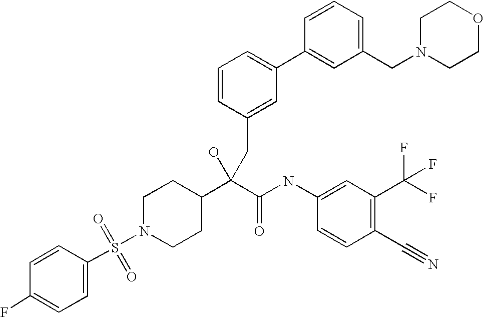 |
| 4445 4446 4447 | rac + − | 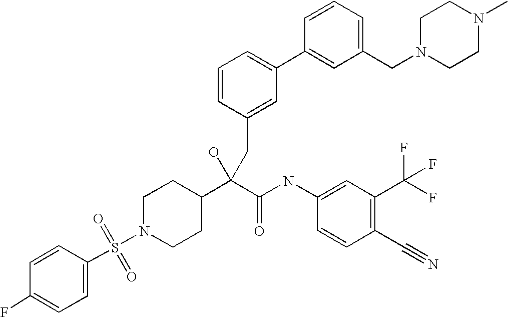 |
| 4448 4449 4450 | rac + − | 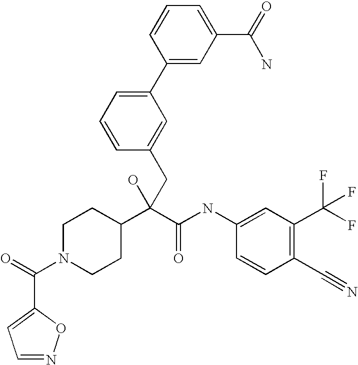 |
| 4451 4452 4453 | rac + − | 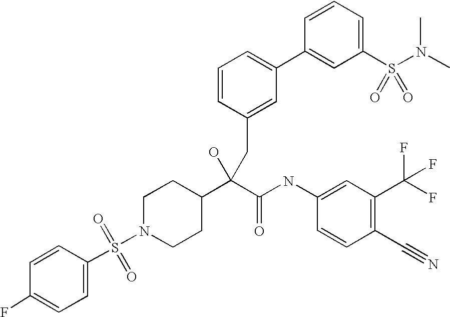 |
| 4454 4455 4456 | rac + − | 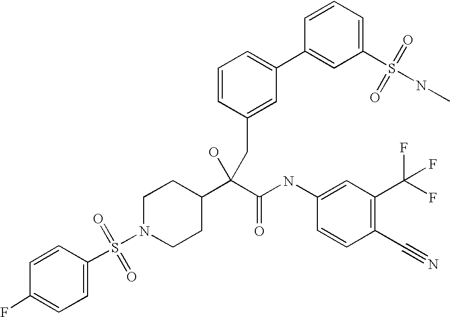 |
| 4457 4458 4459 | rac + − | 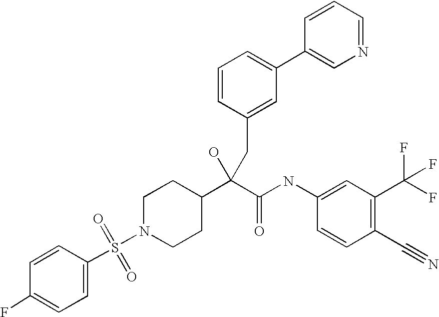 |
| 4460 4461 4462 | rac + − | 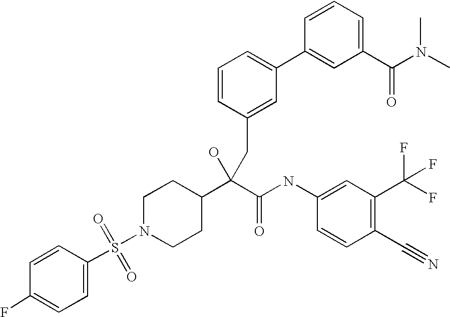 |
| 4463 4464 4465 | rac + − | 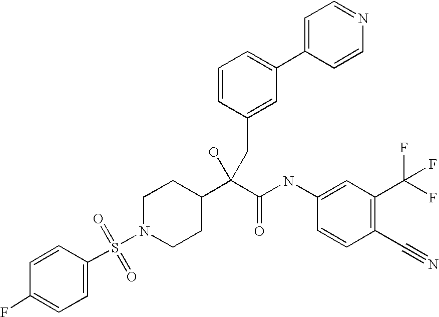 |
| 4466 4467 4468 | rac + − | 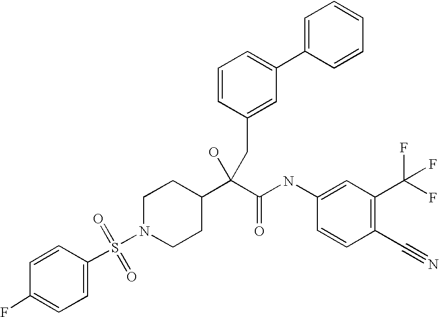 |
| 4469 4470 4471 4472 4473 | stereoisomer mixture (R, R) (S, S) (R, S) (S, R) | 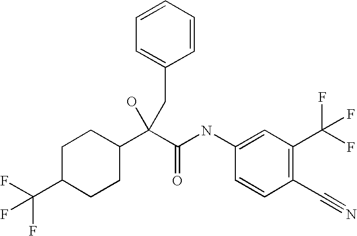 |
| 4474 4475 4476 4477 4478 4479 4480 4481 4482 | stereoisomer mixture (R, R, R) (S, S, S) (R, R, S) (R, S, S) (R, S, R) (S, S, R) (S, R, R) (S, R, S) | 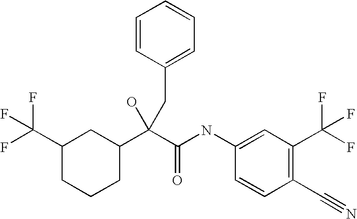 |
| 4483 4484 4485 4486 4487 | stereoisomer mixture (R, R) (S, S) (R, S) (S, R) | 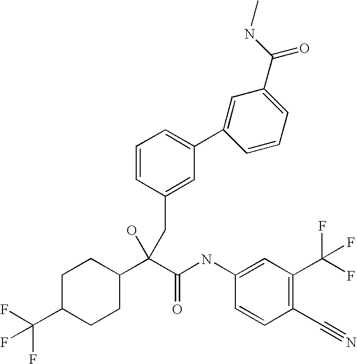 |
| 4488 4489 4490 | rac + − | 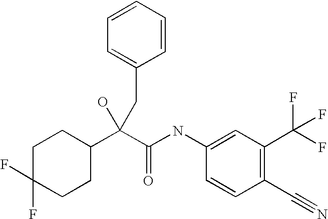 |
| 4491 4492 4493 | rac + − | 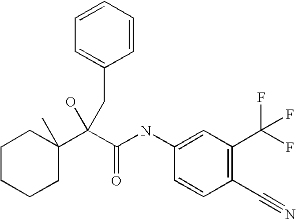 |

### Biological Characterization of the Compounds According to the Invention

Progesterone receptor modulators can be identified with the aid of simple methods, test programmes known to the skilled person. It is possible for this purpose for example to incubate a compound to be tested together with a progestogen in a test system for progesterone receptor ligands and to check whether the effect mediated by progesterone is altered in the presence of the modulator in this test system.

The substances according to the invention of the general formula I were tested in the following models:

### Progesterone Receptor-Binding Assay

### Measurement of the Receptor Binding Affinity:

The receptor binding affinity was determined by competitive binding of a specifically binding ³H-labelled hormone (tracer) and of the compound to be tested on receptors in the cytosol from animal target organs. The aim in this case was receptor saturation and reaction equilibrium.

The tracer and increasing concentrations of the compound to be tested (competitor) were coincubated at 0-4° C. for 18 h with the receptor-containing cytosol fraction. After removal of unbound tracer with carbon-dextran suspension, the receptor-bound tracer content was measured for each concentration, and the IC₅₀ was determined from the concentration series. The relative molar binding affinity (RBA) was calculated as ratio of the IC₅₀ values for reference substance and compound to be tested (×100%) (RBA of the reference substance=100%).

The following incubation conditions were chosen for the receptor types:

### Progesterone Receptor:

Uterus cytosol of the estradiol-primed rabbit, homogenized in TED buffer (20 mMTris/HCl, pH 7.4; 1 mM ethylenediamine tetraacetate, 2 mM dithiothreitol) with 250 mM sucrose; stored at −30° C. Tracer: ³H-ORG 2058, 5 nM; reference substance: progesterone.

### Glucocorticoid Receptor:

Thymus cytosol from the adrenalectomized rat, thymi stored at −30° C.; buffer: TED. Tracer: ³H-dexamethasone, 20 nM; reference substance: dexamethasone.

The competition factors (CF values) for the compounds according to the invention of the general formula (I) on the progesterone receptor are between 0.2 and 35 relative to progesterone. The CF values on the glucocorticoid receptor are in the range from 3 to 35 relative to dexamethasone.

The compounds according to the invention accordingly have a high affinity for the progesterone receptor, but only a low affinity for the glucocorticoid receptor.

### Antagonism on the Progesterone Receptor PR

The transactivation assay is carried out as described in WO 02/054064.

The IC₅₀ values are in the range from 0.1 to 150 nM.

### Agonism on the Progesterone Receptor PR

The transactivation assay is carried out as described in Fuhrmann et al. (Fuhrmann U., Hess-Stump H., Cleve A., Neef G., Schwede W., Hoffmann J., Fritzemeier K.-H., Chwalisz K., Journal of Medicinal Chemistry, 43, 26, 2000, 5010-5016).

The table which follows shows, by way of example, results from the transactivation test on antagonistic activity on (PR-B).

TABLE 1

| Example No. | IC₅₀ mol |
|---|---|
| 2b | 1.8E−9 |
| 3 | 5.6E−9 |
| 4 | 2.0E−9 |
| 5 | 7.9E−9 |
| 6 | 5.0E−9 |
| 10 | 7.0E−9 |
| 12 | 1.8E−9 |
| 16 | 1.7E−8 |
| 32 | 2.7E−8 |
| 39 | 1.7E−8 |
| 44 | 8.0E−9 |

Table 2 shows results from the transactivation test on antagonistic activity at PR-A. Both the efficacy and the IC50 are shown. All substances exhibit significant antagonism. The IC₅₀ values of the inventive compounds shown by way of example in table 2 are in the range of 1-60 nM.

TABLE 2

| | | Efficacy | IC50 |
|---|---|---|---|
| Example | | (antagonism at | (antagonism at |
| No. | Structure | PR-A) [%] | PR-A) [mol/l] |
| 19a | 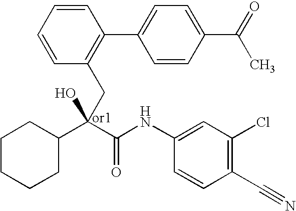 | 107.57 | 6.26E-9 |
| 59 | 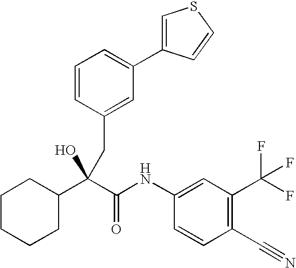 | 103.21 | 4.68E-8 |
| 63 | 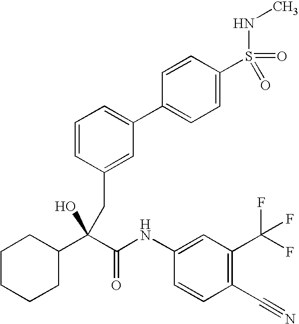 | 103.64 | 2.63E-8 |
| 68 | 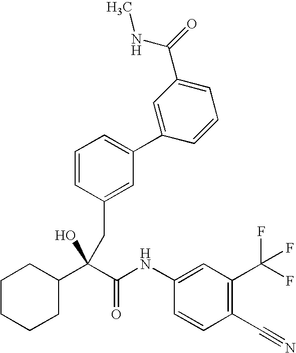 | 101.96 | 5.13E-9 |
| 71 | 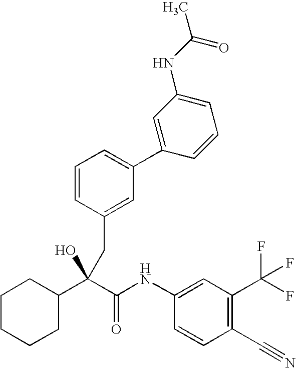 | 100.46 | 1.73E-8 |
| 78 | 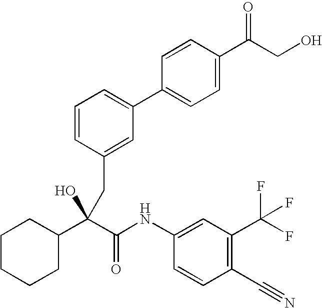 | 100.76 | 5.57E-8 |
| 84 | 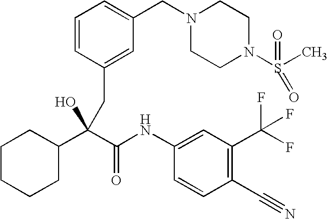 | 101.51 | 5.12E-8 |
| 97 | 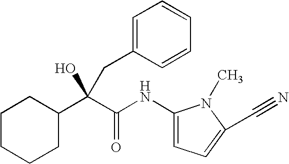 | 105.36 | 5.56E-8 |
| 101a | 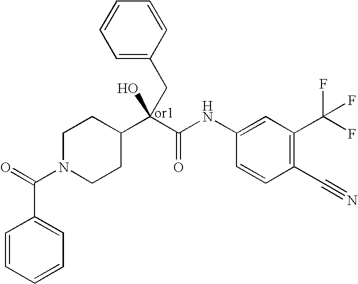 | 103.44 | 1.47E-8 |
| 103 | 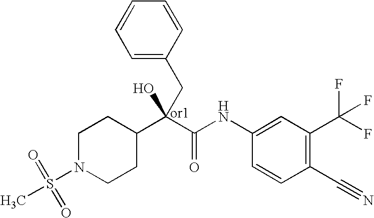 | 98.48 | 4.88E-8 |
| 108 | 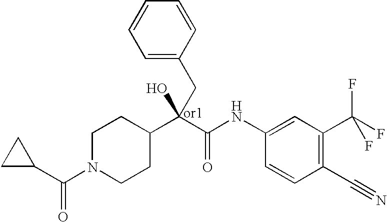 | 95.98 | 3.61E-8 |
| 111 | 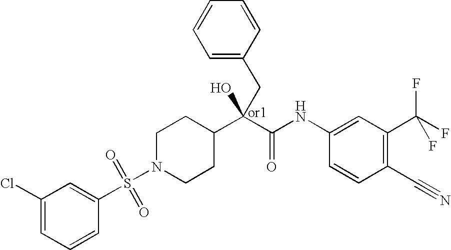 | 103.18 | 4.17E-9 |
| 113 | 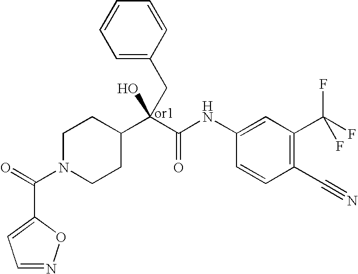 | 97.94 | 2.17E-8 |
| 159 | 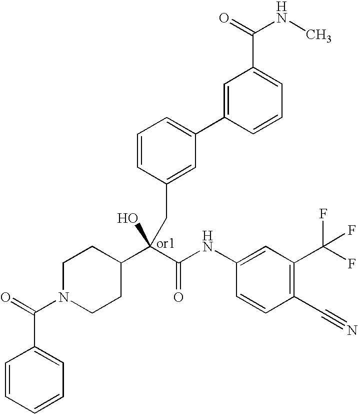 | 96.21 | 1.59E-8 |
| 162 | 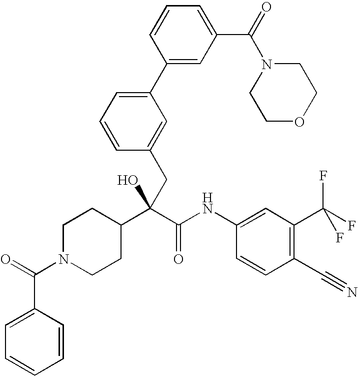 | 99.01 | 4.41E-8 |
| 169 | 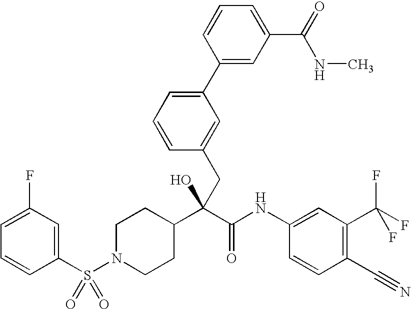 | 103.21 | 1.7E-9 |
| 172 | 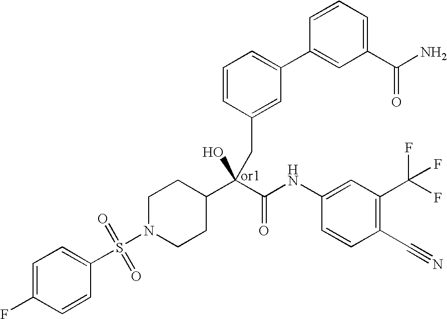 | 100.63 | 1.2E-9 |
| 199 | 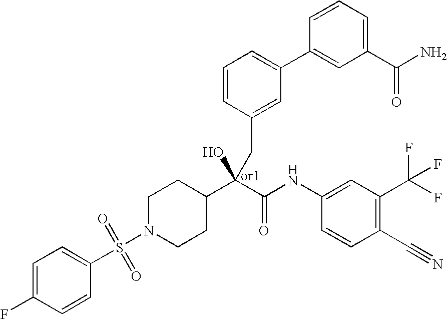 | 96.41 | 3.77E-8 |
| 200b | 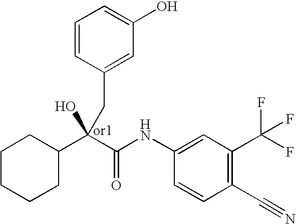 | 98.5 | 5.66E-9 |

### Dosage

The progesterone receptor modulators can be administered orally, enterally, parenterally or transdermally for the use according to the invention.

Satisfactory results are generally to be expected in the treatment of the indications mentioned hereinbefore when the daily doses cover a range from 1 μg to 1000 mg of the compound according to the invention for gynaecological indications such as the treatment of endometriosis, leiomyomas of the uterus and dysfunctional bleeding, and for use in fertility control and for hormone replacement therapy. For oncological indications, daily dosages in the range from 1 μg to 2000 mg of the compound according to the invention are to be administered.

Suitable dosages of the compounds according to the invention in humans for the treatment of endometriosis, of leiomyomas of the uterus and dysfunctional bleeding and for use in fertility control and for hormone replacement therapy are from 50 μg to 500 mg per day, depending on the age and constitution of the patient, it being possible to administer the necessary daily dose by single or multiple administration.

The dosage range for the compounds according to the invention for the treatment of breast carcinomas is 10 mg to 2000 mg per day.

The pharmaceutical products based on the novel compounds are formulated in a manner known per se by processing the active ingredient with the carrier substances, fillers, substances influencing disintegration, binders, humectants, lubricants, absorbents, diluents, masking flavours, colourants, etc. which are used in pharmaceutical technology, and converting into the desired administration form. Reference should be made in this connection to Remington's Pharmaceutical Science, 15^{th} ed. Mack Publishing Company, East Pennsylvania (1980).

Suitable for oral administration are in particular tablets, film-coated tablets, sugar-coated tablets, capsules, pills, powders, granules, pastilles, suspensions, emulsions or solutions.

Preparations for injection and infusion are possible for parenteral administration.

Appropriately prepared crystal suspensions can be used for intraarticular injection.

Aqueous and oily solutions for injection or suspensions and corresponding depot preparations can be used for intramuscular injection.

For rectal administration, the novel compounds can be used in the form of suppositories, capsules, solutions (e.g. in the form of enemas) and ointments, both for systemic and for local therapy.

Furthermore, compositions for vaginal use may also be mentioned as preparation.

For pulmonary administration of the novel compounds, they can be used in the form of aerosols and inhalants.

Patches are possible for transdermal administration, and formulations in gels, ointments, fatty ointments, creams, pastes, dusting powders, milk and tinctures are possible for topical application. The dosage of the compounds of the general formula I in these preparations should be 0.01%-20% in order to achieve an adequate pharmacological effect.

Corresponding tablets can be obtained for example by mixing the active ingredient with known excipients, for example inert diluents such as dextrose, sugar, sorbitol, mannitol, polyvinylpyrrolidone, disintegrants such as maize starch or alginic acid, binders such as starch or gelatin, lubricants such as magnesium stearate or talc and/or means to achieve a depot effect such as carboxypolymethylene, carboxymethylcellulose, cellulose acetate phthalate or polyvinyl acetate. The tablets may also consist of a plurality of layers.

Correspondingly, coated tablets can be produced by coating cores produced in analogy to the tablets with compositions normally used in tablet coatings, for example polyvinylpyrrolidone or shellac, gum arabic, talc, titanium oxide or sugar. The tablet covering may in this case also consist of a plurality of layers, it being possible to use the excipients mentioned above for tablets.

Solutions or suspensions of the compounds according to the invention of the general formula I may additionally comprise taste-improving agents such as saccharin, cyclamate or sugar, and, for example, flavourings such as vanillin or orange extract. They may additionally comprise suspending excipients such as sodium carboxymethylcellulose or preservatives such as p-hydroxybenzoates.

Capsules comprising the compounds of the general formula I can be produced for example by mixing the compound(s) of the general formula I with an inert carrier such as lactose or sorbitol and encapsulating it in gelatin capsules.

Suitable suppositories can be produced for example by mixing with carriers intended for this purpose, such as neutral fats or polyethylene glycol or derivatives thereof.

The compounds according to the invention of the general formula (I) or their pharmaceutically acceptable salts can be used, because of their antagonistic or partial agonistic activity, for the manufacture of a medicament, in particular for the treatment and prophylaxis of gynaecological disorders such as endometriosis, leiomyomas of the uterus, dysfunctional bleeding and dysmenorrhoea. They can furthermore be employed to counteract hormonal irregularities, for inducing menstruation and alone or in combination with prostaglandins and/or oxytocin to induce labour.

The compounds according to the invention of the general formula (I) or their pharmaceutically acceptable salts are furthermore suitable for the manufacture of products for female contraception (see also WO 93/23020, WO 93/21927).

The compounds according to the invention or their pharmaceutically acceptable salts can additionally be employed alone or in combination with a selective oestrogen receptor modulator (SERM) for female hormone replacement therapy.

In addition, the said compounds have an antiproliferative effect in hormone-dependent tumours. They are therefore suitable for the therapy of hormone-dependent carcinomas such as, for example, for breast, prostate and endometrial carcinomas.

The compounds according to the invention or their pharmaceutically acceptable salts can be employed for the treatment of hormone-dependent carcinomas both in first-line therapy and in second-line therapy, especially after tamoxifen failure.

The compounds according to the invention, having antagonistic or partially agonistic activity, of the general formula (I) or their pharmaceutically acceptable salts can also be used in combination with compounds having antioestrogenic activity (oestrogen receptor antagonists or aromatase inhibitors) or selective oestrogen receptor modulators (SERM) for producing pharmaceutical products for the treatment of hormone-dependent tumours. The compounds according to the invention can likewise be used in combination with SERMs or an antioestrogen (oestrogen receptor antagonist or aromatase inhibitor) for the treatment of endometriosis or of leiomyomas of the uterus.

Suitable for combination with the non-steroidal progesterone receptor modulators according to the invention in this connection are for example the following antioestrogens (oestrogen receptor antagonists or aromatase inhibitors) or SERMs: tamoxifen, 5-(4-{5-[(RS)-(4,4,5,5,5-pentafluoropentyl)sulphinyl]pentyloxy}phenyl)-6-phenyl-8,9-dihydro-7H-benzocyclohepten-2-ol (WO 00/03979), ICI 182 780 (7alpha-[9-(4,4,5,5-pentafluoropentylsulphinyl)nonyl]estra-1,3,5(10)-triene-3,17beta-diol), 11beta-fluoro-7alpha-[5-(methyl{3-[(4,4,5,5,5-pentafluoropentyl)sulphanyl]propyl}amino)pentyl]-estra-1,3,5(10)-triene-3,17beta-diol (WO98/07740), 11beta-fluoro-7alpha-{5-[methyl(7,7,8,8,9,9,10,10,10-nonafluorodecyl)amino]pentyl}estra-1,3,5(110)-triene-3,17-beta-diol (WO 99/33855), 11beta-fluoro-17alpha-methyl-7alpha-{5-[methyl(8,8,9,9,9-pentafluorononyl)amino]pentyl}estra-1,3,5(10)-triene-3,17beta-diol (WO 03/045972), clomifene, raloxifene, and further compounds having antioestrogenic activity, and aromatase inhibitors such as, for example, fadrozole, formestane, letrozole, anastrozole or atamestane.

Finally, the present invention also relates to the use of the compounds of the general formula I, where appropriate together with an antioestrogen or SERM, for the manufacture of a medicament.

The present invention further relates to pharmaceutical compositions which comprise at least one compound according to the invention, where appropriate in the form of a pharmaceutically/pharmacologically acceptable salt.

These pharmaceutical compositions and medicaments may be intended for oral, rectal, vaginal, subcutaneous, percutaneous, intravenous or intramuscular administration. Besides conventional carriers and/or diluents, they comprise at least one compound according to the invention.

The medicaments of the invention are manufactured with the conventional solid or liquid carriers or diluents and the excipients normally used in pharmaceutical technology appropriate for the desired mode of administration with a suitable dosage in a known manner. The preferred preparations consist of a dosage form suitable for oral administration. Examples of such dosage forms are tablets, film-coated tablets, sugar-coated tablets, capsules, pills, powders, solutions or suspensions, where appropriate as depot form.

The pharmaceutical compositions comprising at least one of the compounds according to the invention are preferably administered orally.

Also suitable are parenteral preparations such as solutions for injection. Further preparations which may also be mentioned are for example suppositories and compositions for vaginal use.

### Preparation of the Inventive Compounds:

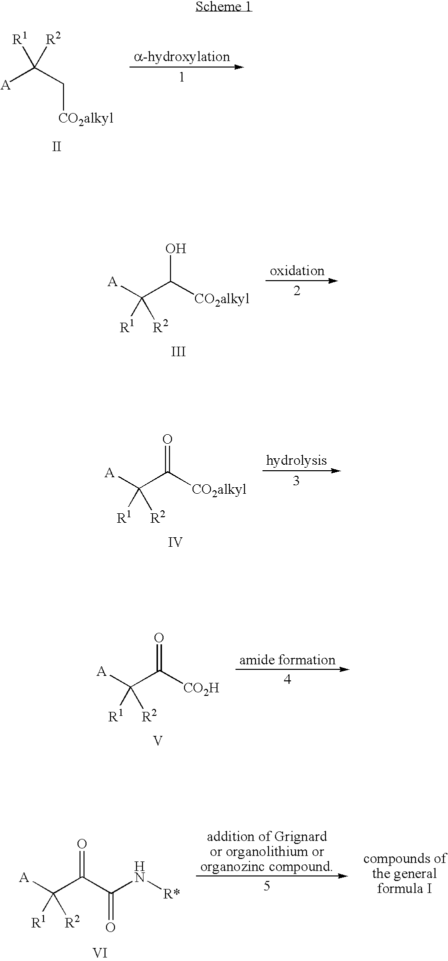

The compounds of the general formula I may be synthesized, for example, as shown in scheme 1. An α-hydroxylation of the ester of the general formula II and subsequent oxidation of the alcohol III formed to the ketone gives rise to compounds of the general formula IV.

For the preparation of compounds of the general formula III by α-hydroxylation of esters, various processes known from the literature are useful (for example Davis et al. in J. Org. Chem., 1984, 49, 3241 using 2-sulphonyloxaziridine). The oxidation to compounds of the general formula IV can then be effected by known standard methods. The amides of the general formula VI are prepared, for example, via the formation of the acid chlorides and subsequent reaction with the corresponding amines. Alternatively, for this purpose, it is also possible to utilize other methods of amide formation according to the amine to be introduced. The compounds of the general formula I are then prepared from the amides of the general formula VI by addition of organometallic compounds such as magnesium, lithium or organozinc compounds. Steps 1-5 can, though, also be performed in an altered sequence.

Some intermediate compounds of the general formulae III-V are commercially available. The substituents A, X, Y, R¹, R², R³ and R⁴ may optionally be modified further after they have been introduced. Possible reactions for this purpose include, for example, oxidation, reduction, alkylations, acylations, nucleophilic additions or else transition metal-catalysed coupling reactions.

Functional groups in compounds of the general formulae II-VI are optionally provided intermediately with protective groups which are then detached again at a suitable stage.

The examples which follow serve to illustrate the subject-matter of the invention in detail without any intention to restrict it to them.

The preparation of 6-amino-4-methyl-2,3-benzoxazin-1-one is described in WO 199854159.

### Preparation of 6-(4,4-dimethyl-2-oxovaleroylamino)-4-methyl-2,3-benzoxazin-1-one

### a) 2-Hydroxy-2-cyclohexylacetic acid ethyl ester

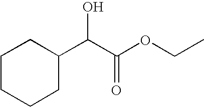

To a solution of 2-cyclohexylacetic acid ethyl ester (1.2 g) in tetrahydrofuran (25 ml) was added, at −70° C., a solution of potassium hexamethyldisilizide (20 ml, 0.5 M in toluene). The mixture was left to stir at −70° C. for a further 30 minutes and then a solution of 3-phenyl-2-phenylsulphonyloxaziridine (2.6 g) in tetrahydrofuran (25 ml) was added. The mixture was left to stir at −70° C. for one hour. The reaction mixture was then poured onto saturated aqueous ammonium chloride solution. The mixture was stirred for a further 30 minutes and the phases were separated. The organic phase was washed with saturated aqueous sodium chloride solution, dried over sodium sulphate and concentrated under reduced pressure. The resulting crude product was chromatographed on silica gel. 1.3 g of product were obtained.

¹H NMR (ppm, CDCl₃, 400 MHz): 1.11-1.27 (6H), 1.31 (3H), 1.44 (1H), 1.61-1.79 (4H), 2.73 (1H), 4.01 (1H), 4.25 (2H).

### b) 2-Oxo-2-cyclohexylacetic acid ethyl ester

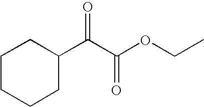

To a solution of the compounds described under a) in 20 ml of dichloromethane were added 20 ml of a 0.35 molar solution of 1,1-dihydro-1,1,1-triacetoxy-1,2-benzodioxol-3(1H)-one (Dess-Martin periodane) in dichloromethane. The mixture was left to stir at 23° C. for 14 hours. Subsequently, the mixture was diluted with 500 ml of methyl tert-butyl ether and then poured onto 1 l of an aqueous solution of 34 g of sodium hydrogencarbonate and 100 g of sodium thiosulphate. The mixture was left to stir for 30 minutes, then the phases were separated and the aqueous phase was extracted with methyl tert-butyl ether. The combined organic phases were washed with saturated aqueous sodium hydrogencarbonate solution and saturated aqueous sodium chloride solution and dried over sodium sulphate. The crude product was chromatographed on silica gel. 0.8 g of product was obtained.

¹H NMR (ppm, CDCl₃, 400 MHz): 1.15-1.29 (5H), 1.36 (3H), 1.67-1.91 (5H), 3.03 (1H), 4.32 (2H).

### c) 2-Oxo-2-cyclohexylacetic acid

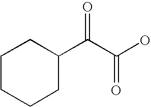

To a solution of the compound described under b) in 13 ml of ethanol was added a solution of 0.5 g of sodium hydroxide in 8 ml of water. The mixture was left to stir at 23° C. for a further 14 hours, then diluted with water and extracted with ethyl acetate. Subsequently, the aqueous phase was acidified with 2 normal hydrochloric acid (pH 4). The mixture was then extracted with ethyl acetate and the organic phase was washed with saturated aqueous sodium chloride solution. It was then dried over sodium sulphate and concentrated under reduced pressure. The resulting crude product (0.7 g) was used in the next stage without purification.

### d) N-(3-Chloro-4-cyanophenyl)-2-oxo-2-cyclohexylacetamide

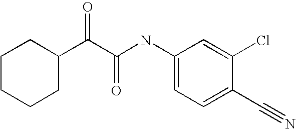

The carboxylic acid described under c) was dissolved in 20 ml of N,N-dimethylacetamide. 0.38 ml of thionyl chloride was added at −10° C. and the mixture was left to stir at −10° C. for one hour. Subsequently, 0.92 g of 3-chloro-4-cyanoaniline was added in portions. The mixture was then stirred for a further 3 hours (−10° C. to 0° C.). Subsequently, the reaction mixture was poured on ice-water. It was extracted with ethyl acetate. The organic phase was washed with saturated aqueous sodium chloride solution, dried over sodium sulphate and concentrated under reduced pressure. The crude product was purified by column chromatography on silica gel with a mixture of hexane/ethyl acetate. 1.2 g of product were obtained.

¹H NMR (ppm, CDCl₃, 400 MHz): 1.22-1.46 (5H), 1.72-1.96 (5H), 3.42-3.50 (1H), 7.59 (1H), 7.67 (1H), 8.02 (1H), 8.96 (1H).

### EXAMPLE 1

### rac-N-(3-Chloro-4-cyanophenyl)-2-oxo-2-cyclohexyl-2-phenylacetamide

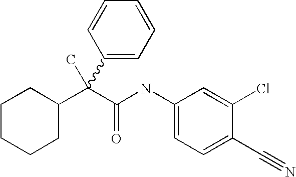

A 1 molar solution of phenylmagnesium bromide in tetrahydrofuran (0.9 ml) was diluted with 10 ml of absolute tetrahydrofuran. The mixture was cooled to −70° C. and then a solution of 100 mg of N-(3-chloro-4-cyanophenyl)-2-oxo-2-cyclohexylacetamide in 6 ml of tetrahydrofuran was added. Subsequently, the mixture was left to stir at −70° C. for a further 2.5 hours. The reaction mixture was then poured onto ice-cold saturated ammonium chloride solution. It was extracted with ethyl acetate. The combined organic phases were washed with saturated sodium chloride solution and dried over sodium sulphate. The crude product was chromatographed on silica gel. 130 mg of product were obtained.

¹H NMR (ppm, CDCl₃, 400 MHz): 0.84 (1H), 1.10-1.43 (5H), 1.71-1.84 (4H), 2.60 (2H), 7.29-7.42 (3H), 7.48 (1H), 7.57 (1H), 7.65 (2H), 7.96 (1H), 8.95 (1H).

### EXAMPLE 2

### rac-N-(3-Chloro-4-cyanophenyl)-2-hydroxy-2-cyclohexyl-2-phenylethynyl-acetamide

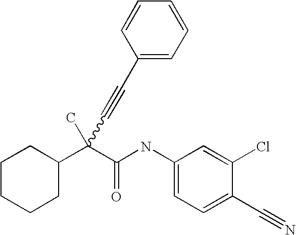

To a solution of 94 μl of phenylacetylene in tetrahydrofuran (5 ml) was added, at −78° C., n-butyllithium (540 μl, 1.6 M in hexane). The mixture was left to stir at this temperature for a further 30 minutes and then a solution of N-(3-chloro-4-cyanophenyl)-2-oxo-2-cyclohexylacetamide (100 mg) in 5 ml of tetrahydrofuran was added dropwise. Subsequently, the mixture was allowed to come to 23° C. over approx. 3 h and then stirred for a further 10 h. The reaction mixture was then poured onto ice-cold saturated ammonium chloride solution. The mixture was extracted with ethyl acetate. The combined organic phases were washed with saturated sodium chloride solution and dried over sodium sulphate. The crude product was chromatographed on silica gel. 110 mg of product were obtained.

¹H NMR (ppm, DMSO-d₆, 400 MHz): 0.84 (1H), 1.14-1.28 (5H), 1.60-1.98 (6H), 6.86 (1H), 7.36-7.46 (5H), 7.94 (2H), 8.27 (1H), 10.41 (1H).

### EXAMPLE 2A AND 2B

### (+)-N-(3-Chloro-4-cyanophenyl)-2-hydroxy-2-cyclohexyl-2-phenylethynyl-acetamide 2a and (−)-N-(3-chloro-4-cyanophenyl)-2-hydroxy-2-cyclohexyl-2-phenylethynylacetamide 2b

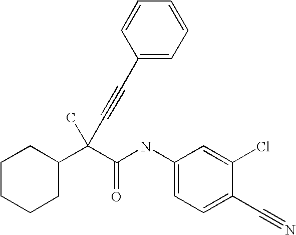

The racemic mixture obtained in Example 2 was separated by preparative chiral HPLC (column: Chiralpak AD 250×10 mm) into enantiomers 2a and 2b.

2a: [α]^{D}₂₀: +2.30 (CHCl₃, 11.3 mg/l ml; λ=589 nM)

2b: [α]^{D}₂₀: −2.00 (CHCl₃, 11.0 mg/1 ml; λ=589 nM)

### EXAMPLE 3

### rac-N-(3-Chloro-4-cyanophenyl)-2-hydroxy-2-cyclohexyl-3-phenylpropanamide

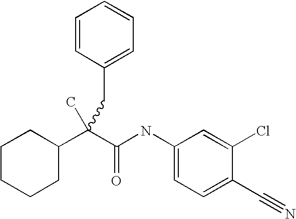

A 2 molar solution of benzylmagnesium chloride in tetrahydrofuran (430 μl) was diluted with 4 ml of absolute tetrahydrofuran. The mixture was cooled to −70° C. and then a solution of 100 mg of N-(3-chloro-4-cyanophenyl)-2-oxo-2-cyclohexylacetamide in 6 ml of tetrahydrofuran was added. Subsequently, the mixture was left to stir at −70° C. for a further 2 hours. The reaction mixture was then poured onto ice-cold saturated ammonium chloride solution. The mixture was extracted with ethyl acetate. The combined organic phases were washed with saturated sodium chloride solution and dried over sodium sulphate. The crude product was chromatographed on silica gel. 78 mg of product were obtained.

¹H NMR (ppm, DMSO-d₆, 400 MHz): 1.01-1.76 (10H), 1.93 (1H), 2.87 (1H), 3.02 (1H), 5.46 (1H), 7.05-7.28 (5H), 7.69-7.78 (2H), 8.01 (1H), 9.69 (1H).

### EXAMPLE 4

### rac-N-(3-Chloro-4-cyanophenyl)-2-hydroxy-2-cyclohexyl-2-(4-methyl phenyl)-ethynylacetamide

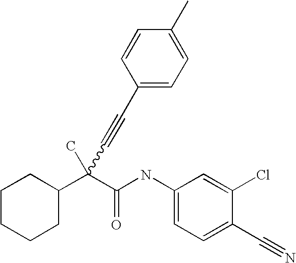

N-(3-Chloro-4-cyanophenyl)-2-oxo-2-cyclohexylacetamide (100 mg) was reacted with 4-methylphenylethyne and n-butyllithium in tetrahydrofuran in analogy to Example 2. After column chromatography, 45 mg of product were obtained.

¹H NMR (ppm, CDCl₃, 400 MHz): 1.16-1.35 (5H), 1.66-1.88 (4H), 2.05-2.14 (2H), 2.35 (3H), 3.02 (1H), 7.13 (2H), 7.35 (2H), 7.56 (1H), 7.62 (1H), 7.98 (1H), 8.80 (1H).

### EXAMPLE 5

### rac-N-(3-Chloro-4-cyanophenyl)-2-hydroxy-2-cyclohexyl-2-(4-trifluoromethyl-phenyl)ethynylacetamide

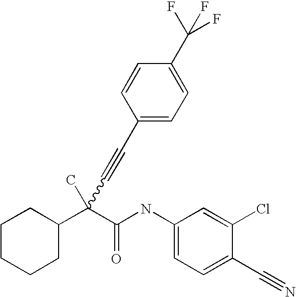

N-(3-Chloro-4-cyanophenyl)-2-oxo-2-cyclohexylacetamide (150 mg) was reacted with 4-trifluoromethylphenylethyne and n-butyllithium in tetrahydrofuran in analogy to Example 2. After column chromatography, 140 mg of product were obtained.

¹H NMR (ppm, CDCl₃, 400 MHz): 1.12-1.39 (5H), 1.64-1.90 (4H), 2.08-2.16 (2H), 3.08 (1H), 7.54-7.65 (6H), 7.99 (1H), 8.80 (1H).

### EXAMPLE 6

### rac-N-(3-Chloro-4-cyanophenyl)-2-hydroxy-2-cyclohexyl-2-(4-fluorophenyl)-ethynylacetamide

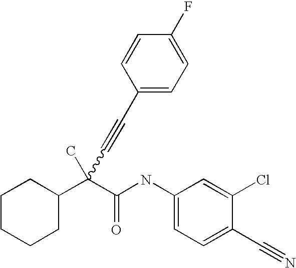

N-(3-Chloro-4-cyanophenyl)-2-oxo-2-cyclohexylacetamide (150 mg) was reacted with 4-fluorophenylethyne and n-butyllithium in tetrahydrofuran in analogy to Example 2. After column chromatography, 140 mg of product were obtained.

¹H NMR (ppm, CDCl₃, 400 MHz): 1.21-1.37 (5H), 1.64-1.91 (4H), 2.05-2.16 (2H), 2.95 (1H), 7.02 (2H), 7.45 (2H), 7.56 (1H), 7.64 (1H), 7.98 (1H), 8.79 (1H).

### EXAMPLE 7

### rac-N-(3-Chloro-4-cyanophenyl)-2-hydroxy-2-cyclohexyl-2-(4-formylphenyl)-ethynylacetamide

### 7a) rac-N-(3-Chloro-4-cyanophenyl)-2-hydroxy-2-cyclohexyl-2-(4-(1,3-dioxolanyl)-phenyl)ethynylacetamide

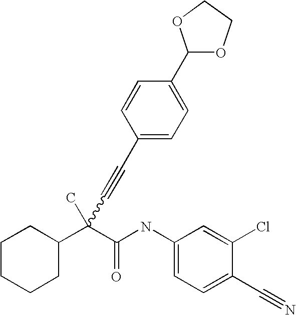

N-(3-Chloro-4-cyanophenyl)-2-oxo-2-cyclohexylacetamide (150 mg) was reacted with 4-(1-3-dioxolanyl)phenylethyne and n-butyllithium in tetrahydrofuran in analogy to Example 2. After column chromatography, 180 mg of product were obtained.

¹H NMR (ppm, DMSO-d₆, 400 MHz): 1.07-1.25 (5H), 1.56-1.95 (6H), 3.88-4.03 (4H), 5.70 (1H), 6.86 (1H), 7.42 (4H), 7.91 (2H), 8.24 (1H), 10.38 (1H).

### 7b) rac-N-(3-Chloro-4-cyanophenyl)-2-hydroxy-2-cyclohexyl-2-(4-formylphenyl)ethynyl-acetamide

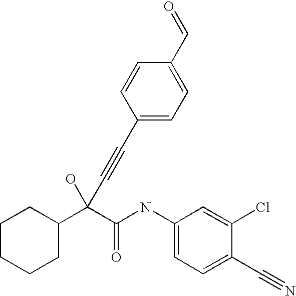

To a solution of the compound described under 7a (50 mg) in methanol (2 ml) was added HCl (1 M in water, 0.4 ml) at 23° C. The mixture was left to stir at 23° C. for a further 24 hours. The reaction mixture was then poured onto saturated sodium hydrogen carbonate solution. It was extracted with ethyl acetate. The combined organic phases were washed with saturated sodium chloride solution and dried over sodium sulphate. The crude product was chromatographed on silica gel. 20 mg of product 7b and 23 mg of the following product 7c were obtained.

¹H NMR (ppm, DMSO-d₆, 400 MHz): 1.10-1.30 (5H), 1.64-1.79 (4H), 1.94-1.99 (2H), 7.66 (2H), 7.89-7.97 (4H), 8.26 (1H), 10.02 (1H).

### 7c) rac-N-(3-Chloro-4-cyanophenyl)-2-hydroxy-2-cyclohexyl-2-(4-(1,1-dimethoxyacetyl)-phenyl)ethynylacetamide

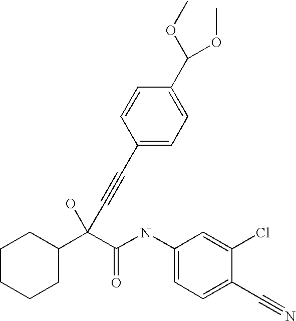

Compound 7c was obtained as a by-product in the purification of 7b.

¹H NMR (ppm, DMSO-d₆, 400 MHz): 1.11-1.29 (5H), 1.62-1.78 (4H), 1.92-1.99 (2H), 3.24 (6H), 5.40 (1H), 7.39 (2H), 7.46 (2H), 7.80 (1H), 7.96 (1H), 8.26 (1H).

### EXAMPLE 8

### rac-N-(3-Chloro-4-cyanophenyl)-2-hydroxy-2-cyclohexyl-2-(3-methyl phenyl)-ethynylacetamide

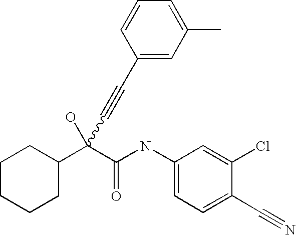

N-(3-Chloro-4-cyanophenyl)-2-oxo-2-cyclohexylacetamide (150 mg) was reacted with 3-methylphenylethyne and n-butyllithium in tetrahydrofuran in analogy to Example 2. After column chromatography, 140 mg of product were obtained.

¹H NMR (ppm, CDCl₃, 400 MHz): 1.22-1.42 (5H), 1.73-1.94 (4H), 2.09-2.19 (2H), 2.37 (3H), 3.07 (1H), 7.20-7.33 (4H), 7.60 (1H), 7.67 (1H), 8.02 (1H), 8.84 (1H).

### EXAMPLE 9

### rac-N-(3-Chloro-4-cyanophenyl)-2-hydroxy-2-cyclohexyl-2-(2-methyl phenyl)-ethynylacetamide

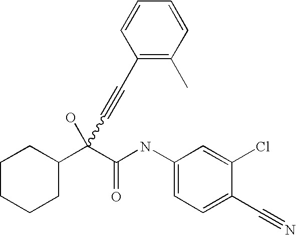

N-(3-Chloro-4-cyanophenyl)-2-oxo-2-cyclohexylacetamide (150 mg) was reacted with 2-methylphenylethyne and n-butyllithium in tetrahydrofuran in analogy to Example 2. After column chromatography, 160 mg of product were obtained.

¹H NMR (ppm, CDCl₃, 400 MHz): 1.22-1.37 (5H), 1.70-1.90 (4H), 2.05-2.16 (2H), 2.45 (3H), 3.07 (1H), 7.15 (1H), 7.20-7.29 (3H), 7.43 (1H), 7.56 (1H), 7.64 (1H), 7.98 (1H), 8.81 (1H).

### EXAMPLE 10

### rac-N-(3-Chloro-4-cyanophenyl)-2-hydroxy-2-cyclohexyl-2-(4-hydroxyphenyl)-ethynylacetamide

### 10a) rac-N-(3-Chloro-4-cyanophenyl)-2-hydroxy-2-cyclohexyl-2-(4-methoxyphenyl)-ethynylacetamide

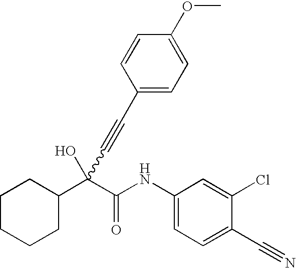

N-(3-Chloro-4-cyanophenyl)-2-oxo-2-cyclohexylacetamide (750 mg) was reacted with 4-methoxyphenylethyne and n-butyllithium in tetrahydrofuran in analogy to Example 2. After column chromatography, 1 g of product was obtained.

¹H NMR (ppm, CDCl₃, 400 MHz): 1.13-1.37 (5H), 1.66-1.89 (4H), 2.05-2.16 (2H), 3.08 (1H), 3.82 (3H), 6.85 (2H), 7.40 (2H), 7.56 (1H), 7.63 (1H), 7.98 (1H), 8.83 (1H).

### 10b) rac-N-(3-Chloro-4-cyanophenyl)-2-hydroxy-2-cyclohexyl-2-(4-hydroxyphenyl)-ethynylacetamide

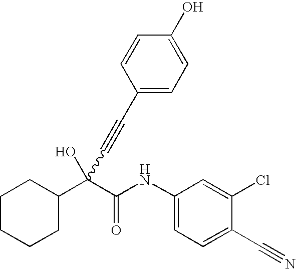

To a solution of the compound described in 10a (200 mg) in dichloromethane (6 ml) was added boron tribromide (1 M in dichloromethane, 2.8 ml) at −15° C. The reaction mixture was left to warm at 23° C. over 3 hours and left to stir over a further 24 hours. The reaction mixture was then poured onto ice-cold saturated sodium hydrogencarbonate solution. It was extracted with ethyl acetate. The combined organic phases were washed with saturated sodium chloride solution and dried over sodium sulphate. The crude product was chromatographed on silica gel. 81 mg of product were obtained.

¹H NMR (ppm, CDCl₃, 400 MHz): 1.16-1.43 (5H), 1.65-1.98 (5H), 2.44 (1H), 3.20 (1H), 5.22 (1H), 6.55 (1H), 6.78 (2H), 7.19 (2H), 7.44 (1H), 7.70 (1H), 8.14 (1H).

### EXAMPLE 11

### rac-N-(3-Chloro-4-cyanophenyl)-2-hydroxy-2-cyclohexyl-2-(1-hydroxypropynyl)-acetamide

### 11a) rac-N-(3-Chloro-4-cyanophenyl)-2-hydroxy-2-cyclohexyl-2-(1-(2-tetrahydro-pyranyloxy)propynyl)acetamide

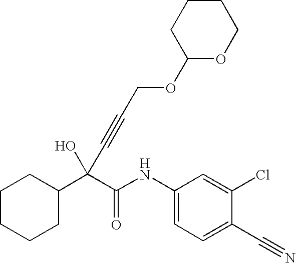

N-(3-Chloro-4-cyanophenyl)-2-oxo-2-cyclohexylacetamide (150 mg) was reacted with tetrahydro-2-(2-propynyloxy)-2H-pyran and n-butyllithium in tetrahydrofuran in analogy to Example 2. After column chromatography, 190 mg of product were obtained.

¹H NMR (ppm, DMSO-d₆, 400 MHz): 1.06-1.20 (5H), 1.43-1.87 (12H), 3.39-3.44 (1H), 3.66-3.73 (1H), 4.25 (2H), 4.74 (1H), 7.89 (2H), 8.19 (1H).

### 11b) rac-N-(3-Chloro-4-cyanophenyl)-2-hydroxy-2-cyclohexyl-2-(1-hydroxypropynyl)-acetamide

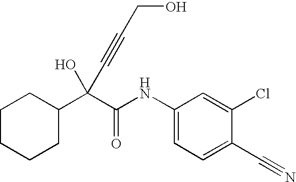

To a solution of the compound described in 11a (50 mg) in acetone (2 ml) was added HCl (1 M in water, 0.4 ml) at 23° C. The mixture was left to stir at 23° C. for a further 24 hours. Thereafter, the reaction mixture was poured onto saturated sodium hydrogencarbonate solution. It was extracted with ethyl acetate. The combined organic phases were washed with saturated sodium chloride solution and dried over sodium sulphate. The crude product was chromatographed on silica gel. 40 mg of product were obtained.

¹H NMR (ppm, DMSO-d₆, 400 MHz): 1.05-1.23 (5H), 1.49-1.93 (6H), 4.13 (2H), 5.22 (1H), 6.66 (1H), 7.89-7.97 (2H), 8.24 (1H), 10.30 (1H).

### EXAMPLE 12

### rac-N-(3-Chloro-4-cyanophenyl)-2-hydroxy-2-cyclohexyl-2-(4-carboxyphenyl)-ethynylacetamide

### 12a) rac-N-(3-Chloro-4-cyanophenyl)-2-hydroxy-2-cyclohexyl-2-(4-carbomethoxy-phenyl)ethynylacetamide

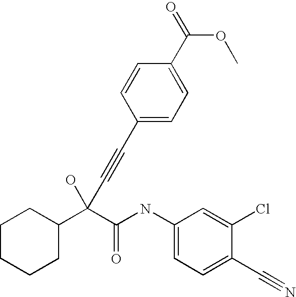

N-(3-Chloro-4-cyanophenyl)-2-oxo-2-cyclohexylacetamide (140 mg) was reacted with 4-ethynylbenzoic acid methyl ester and lithium diisopropylamide in tetrahydrofuran in analogy to Example 2. After column chromatography, 130 mg of product were obtained.

¹H NMR (ppm, DMSO-d₆, 400 MHz): 1.07-1.26 (5H), 1.58-1.72 (4H), 1.87-1.95 (2H), 3.82 (3H), 6.93 (1H), 7.55 (2H), 7.86-7.95 (4H), 8.23 (1H), 10.41 (1H).

### 12b) rac-N-(3-Chloro-4-cyanophenyl)-2-hydroxy-2-cyclohexyl-2-(4-carboxyphenyl)-ethynylacetamide

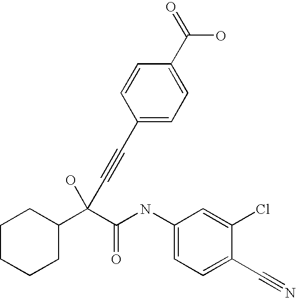

To a solution of the compound described in 12a (50 mg) in methanol (1.5 ml) was added a solution of potassium carbonate (100 mg) in water (50 μl) at 23° C. The mixture was left to stir at 23° C. for a further 4 days. The reaction mixture was then warmed to 40° C. and left to stir for a further 4 hours. The reaction mixture was then poured onto saturated ammonium chloride solution. It was extracted with ethyl acetate. The combined organic phases were washed with saturated sodium chloride solution and dried over sodium sulphate. The crude product was chromatographed on silica gel. 28 mg of product were obtained.

¹H NMR (ppm, DMSO-d₆, 400 MHz): 1.02-1.31 (5H), 1.58-1.76 (4H), 1.87-1.95 (2H), 6.93 (1H), 7.53 (2H), 7.86-7.95 (4H), 8.23 (1H), 10.41 (1H), 13.11 (1H).

### EXAMPLE 13

### rac-N-(3-Chloro-4-cyanophenyl)-2-hydroxy-2-cyclohexyl-2-(2,5-dimethylphenyl)-ethynylacetamide

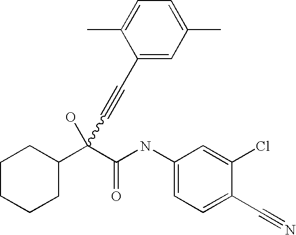

N-(3-Chloro-4-cyanophenyl)-2-oxo-2-cyclohexylacetamide (150 mg) were reacted with 2,5-dimethylphenylethyne and n-butyllithium in tetrahydrofuran in analogy to Example 2. After column chromatography, 190 mg of product were obtained.

¹H NMR (ppm, CDCl₃, 400 MHz): 1.14-1.38 (5H), 1.70-1.90 (4H), 2.05-2.16 (2H), 2.29 (3H), 2.40 (3H), 3.07 (1H), 7.09 (2H), 7.25 (1H), 7.56 (1H), 7.63 (1H), 7.98 (1H), 8.82 (1H).

### EXAMPLE 14

### rac-N-(3-Chloro-4-cyanophenyl)-2-hydroxy-2-cyclohexyl-2-(3-methoxyphenyl)-ethynylacetamide

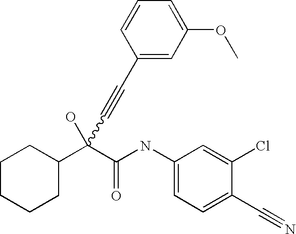

N-(3-Chloro-4-cyanophenyl)-2-oxo-2-cyclohexylacetamide (750 mg) was reacted with 3-methoxyphenylethyne and n-butyllithium in tetrahydrofuran in analogy to Example 2. After column chromatography, 1.0 g of product was obtained.

¹H NMR (ppm, CDCl₃, 400 MHz): 1.14-1.38 (5H), 1.66-1.90 (4H), 2.10-2.18 (2H), 3.15 (1H), 3.80 (3H), 6.92 (1H), 6.98 (1H), 7.06 (1H), 7.22 (1H), 7.56 (1H), 7.63 (1H), 7.98 (1H), 8.83 (1H).

### EXAMPLE 15

### rac-N-(3-Chloro-4-cyanophenyl)-2-cyclohexyl-2-hydroxy-3-m-tolylpropionamide

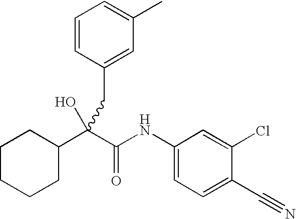

The preparation was effected analogously to Example 3 with 3-methylbenzylmagnesium chloride. ¹H NMR (ppm, CDCl₃, 400 MHz): 1.0-2.2 (m, 11H), 2.20 (s, 1H), 2.29 (s, 3H), 2.90 (d, 1H), 3.45 (d, 1H), 6.97-7.33 (m, 4H), 7.41 (dd, 1H), 7.60 (d, 1H), 7.85 (d, 1H), 8.56 (s, 1H).

### EXAMPLE 16

### rac-N-(3-Chloro-4-cyanophenyl)-2-cyclohexyl-2-hydroxy-3-(4-methoxyphenyl)-propionamide

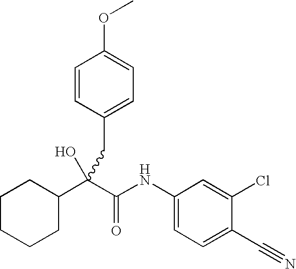

The preparation was effected analogously to Example 3 with 4-methoxybenzyl-magnesium chloride. ¹H NMR (ppm, CDCl₃, 400 MHz): 1.09-1.40 (m, 5H), 1.63-2.04 (m, 6H), 2.11 (s, 1H), 2.83 (d, 1H), 3.38 (d, 1H), 3.75 (s, 3H), 6.80 (d, 2H), 7.07 (d, 2H), 7.37 (dd, 1H), 7.55 (d, 1H), 7.84 (d, 1H), 8.54 (s, 1H).

### EXAMPLE 17

### rac-N-(3-Chloro-4-cyanophenyl)-2-cyclohexyl-2-hydroxy-3-(3-methoxyphenyl)-propionamide

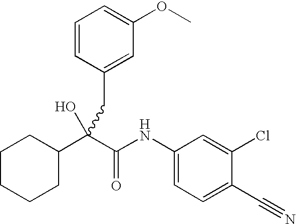

The preparation was effected analogously to Example 3 with 3-methoxybenzyl-magnesium chloride. ¹H NMR (ppm, CDCl₃, 400 MHz): 1.05-1.40 (m, 5H), 1.60-2.05 (m, 6H), 2.88 (d, 1H), 3.30 (d, 1H), 3.70 (s, 3H), 6.75 (m, 3H), 7.16 (dd, 1H), 7.40 (dd, 1H), 7.55 (d, 1H), 7.82 (d, 1H), 8.75 (s, 1H).

### EXAMPLE 18

### rac-N-(3-Chloro-4-cyanophenyl)-2-cyclohexyl-2-hydroxy-3-(3-iodophenyl)-propionamide

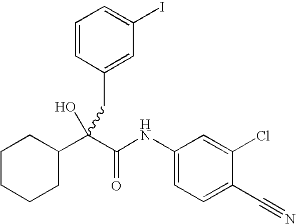

35 ml of 3-iodobenzylzinc bromide solution (0.5M in THF) were initially charged in 15 ml of THF and cooled to −75° C. 1.0 g of N-(3-chloro-4-cyanophenyl)-2-cyclohexyl-2-oxoacetamide, dissolved in 15 ml of THF, was added dropwise. The mixture was stirred at −75° C. for 4 h and at room temperature for a further hour, then added to sat. ammonium chloride solution and extracted with ethyl acetate. The organic phases were washed with sat. NaCl solution and dried over sodium sulphate. The crude product was purified by chromatography and then recrystallized from hexane/diisopropyl ether. 338 mg of the desired product were obtained as a colourless solid. ¹H NMR (ppm, CDCl₃, 400 MHz): 1.0-1.4 (m, 5H), 1.6-2.0 (m, 6H), 2.03 (s, 1H), 2.85 (d, 1H), 3.27 (d, 1H), 6.97 (dd, 1H), 7.12 (d, 1H), 7.36 (dd, 1H), 7.55 (m, 3H), 7.79 (s, 1H), 8.46 (s, 1H).

### EXAMPLE 19

### rac-3-(4′-Acetylbiphenyl-2-yl)-N-(3-chloro-4-cyanophenyl)-2-cyclohexyl-2-hydroxy-propionamide

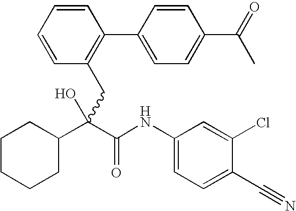

rac-N-(3-Chloro-4-cyanophenyl)-2-cyclohexyl-2-hydroxy-3-(2-iodophenyl)propionamide was prepared analogously to Example 18.

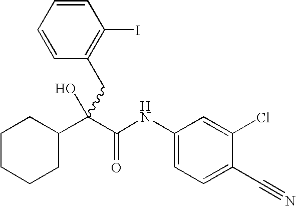

390 mg of the iodine compound and 197 mg of 4-acetylphenylboronic acid were initially charged in 6 ml of 1:1 toluene/ethanol, and 1.5 ml of 1M sodium carbonate solution and 90 mg of tetrakis(triphenylphosphine)palladium were added. The mixture was heated at 120° C. in a microwave for 25 min, then filtered through Celite and rinsed with ethyl acetate. The solution was washed with sat. NaCl solution, dried over sodium sulphate and concentrated. The crude product was purified by chromatography. 137 mg of the desired product were obtained as a colourless foam. ¹H NMR (ppm, DMSO-D₆, 400 MHz): 0.95-1.35 (m, 5H), 1.50-1.80 (m, 6H), 2.63 (s, 3H), 3.03 (d, 1H), 3.16 (d, 1H), 7.09 (m, 1H), 7.23 (m, 2H), 7.46 (d, 2H), 7.59 (m, 1H), 7.76 (dd, 1H), 7.84 (d, 1H), 7.97 (d, 2H), 8.05 (d, 1H), 9.91 (s, 1H).

The racemic mixture obtained was separated into the enantiomers 19a and 19b by preparation chiral HPLC (column Chiralpak 1A 5μ 250×200 mm eluent hexane/ethanol 85:15).

### EXAMPLE 19B

Rₜ=−11.6 min.

### EXAMPLE 20

### rac-N-(4-Cyano-3-trifluoromethyl phenyl)-2-cyclohexyl-2-hydroxy-3-phenyl-propionamide

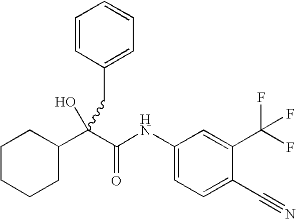

The preparation was effected analogously to Example 3 from N-(4-cyano-3-trifluoromethylphenyl)-2-cyclohexyl-2-oxoacetamide (prepared analogously to N-(3-chloro-4-cyanophenyl)-2-cyclohexyl-2-oxoacetamide, see above) and benzylmagnesium chloride. ¹H NMR (ppm, CDCl₃, 400 MHz): 1.05-1.40 (m, 5H), 1.65-2.05 (m, 6H), 2.15 (s, 1H), 2.92 (d, 1H), 3.41 (d, 1H), 7.16 (m, 2H), 7.24 (m, 3H), 7.73 (d, 1H), 7.81 (dd, 1H), 7.86 (d, 1H), 8.63 (s, 1H).

The racemic mixture obtained was separated into the enantiomers 20a and 20b by preparative chiral HPLC (column: Chiralpak AD 250×10 mm).

Example 20a: [α]^{D}₂₀=−129.4° (MeOH, c=1.01) Example 20b: [α]^{D}₂₀=+132.7° (MeOH, c=1.00)

### EXAMPLE 21

### rac-N-(4-Cyano-3-trifluoromethyl phenyl)-2-cyclohexyl-2-hydroxy-3-m-tolyl-propionamide

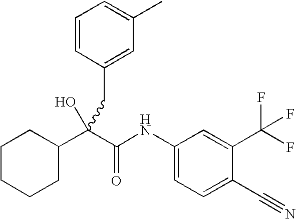

The preparation was effected analogously to Example 20 with 3-methylbenzyl-magnesium chloride. ¹H NMR (ppm, CDCl₃, 400 MHz): 1.2-1.5 (m, 5H), 1.6-2.1 (m, 6H), 2.20 (s, 1H), 2.28 (s, 3H), 2.94 (d, 1H), 3.42 (d, 1H), 7.0-7.3 (m, 4H), 7.85 (m, 3H), 8.68 (s, 1H).

### EXAMPLE 22

### rac-N-(4-Cyano-3-trifluoromethyl phenyl)-2-cyclohexyl-2-hydroxy-3-p-tolyl-propionamide

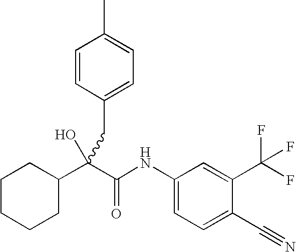

The preparation was effected analogously to Example 20 with 4-methylbenzyl-magnesium chloride. ¹H NMR (ppm, DMSO-D₆, 400 MHz): 1.0-1.3 (m, 5H), 1.4-1.8 (m, 6H), 2.12 (s, 1H), 2.23 (s, 3H), 2.84 (d, 1H), 2.98 (d, 1H), 7.0-7.3 (m, 4H), 7.97 (d, 1H), 8.11 (dd, 1H), 8.23 (d, 1H), 9.90 (s, 1H).

### EXAMPLE 23

### rac-N-(4-Cyano-3-trifluoromethyl phenyl)-2-cyclohexyl-2-hydroxy-3-(4-methoxy-phenyl)propionamide

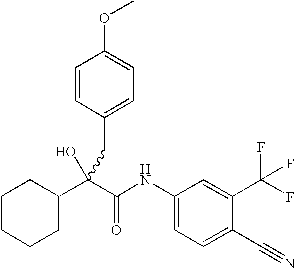

The preparation was effected analogously to Example 20 with 4-methoxybenzyl-magnesium chloride. ¹H NMR (ppm, CDCl₃, 400 MHz): 1.10-1.45 (m, 5H), 1.65-2.05 (m, 6H), 2.19 (s, 1H), 2.90 (d, 1H), 3.42 (d, 1H), 3.79 (s, 3H), 6.84 (d, 2H), 7.12 (d, 2H), 7.79 (d, 1H), 7.88 (dd, 1H), 7.94 (d, 1H), 8.71 (s, 1H).

The resulting racemic mixture was separated by preparative chiral HPLC (column: Chiralpak AD 250×10 mm) into enantiomers 23a and 23b.

Example 23a: Rₜ=5.41 min

(Chiralpak IA 5μ 150×4.6, 80% hexane/20% 2-propanol, 1 ml/min)

Example 23b: Rₜ, =6.36 min

(Chiralpak Ia 5μ 150×4.6, 80% Hexane/20% 2-Propanol, 1 ml/min)

### EXAMPLE 24

### rac-N-(4-Cyano-3-trifluoromethyl phenyl)-2-cyclohexyl-2-hydroxy-3-(3-methoxy-phenyl)propionamide

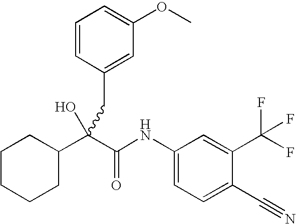

The preparation was effected analogously to Example 3 with 4-methoxybenzyl-magnesium chloride. ¹H NMR (ppm, CDCl₃, 400 MHz): 1.0-2.0 (m, 11H), 2.88 (d, 1H), 2.97 (d, 1H), 3.56 (s, 3H), 5.44 (s, 1H), 6.58 (m, 1H), 6.74 (m, 2H), 7.00 (dd, 1H), 8.17 (dd, 1H), 8.27 (d, 1H), 9.92 (s, 1H).

### EXAMPLE 25

### rac-N-(4-Cyano-3-trifluoromethyl phenyl)-2-cyclohexyl-2-hydroxy-3-(2-iodophenyl)propionamide

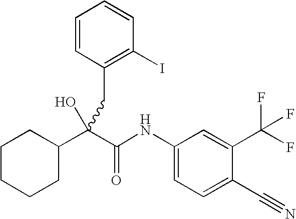

The preparation was effected analogously to Example 18 using 2-iodobenzylzinc bromide. ¹H NMR (ppm, CDCl₃, 400 MHz): 1.0-1.4 (m, 5H), 1.6-2.0 (m, 6H), 2.03 (s, 1H), 2.85 (d, 1H), 3.27 (d, 1H), 6.97 (dd, 1H), 7.12 (d, 1H), 7.36 (dd, 1H), 7.55 (m, 3H), 7.79 (s, 1H), 8.46 (s, 1H).

### EXAMPLE 26

### rac-N-(4-Cyano-3-trifluoromethyl phenyl)-2-cyclohexyl-2-hydroxy-3-(3-iodophenyl)propionamide

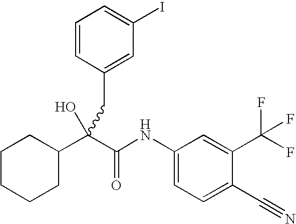

The preparation was effected analogously to Example 25 using 3-iodobenzylzinc bromide. ¹H NMR (ppm, CDCl₃, 400 MHz): 1.05-1.40 (m, 5H), 1.60-2.05 (m, 6H), 2.06 (s, 1H), 2.86 (d, 1H), 3.27 (d, 1H), 6.96 (m, 1H), 7.12 (m, 1H), 7.55 (d, 2H), 7.76 (m, 2H), 7.87 (s, 1H), 8.58 (s, 1H).

The racemic mixture obtained was separated into the enantimers 26a and 26b by preparation chiral HPLC (column Chiralpak 1A 5μ 250×20 mm, eluent hexane/ethanol 95:5).

Example 26a: Rₜ, =15.1-17.4 min

Example 26b:

### EXAMPLE 27

### rac-3-(4′-Acetylbiphenyl-2-yl)-N-(4-cyano-3-trifluoromethyl phenyl)-2-cyclohexyl-2-hydroxypropionamide

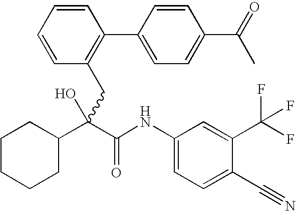

The preparation was effected analogously to Example 19 using the compound prepared in Example 25. ¹H NMR (ppm, DMSO-D₆, 400 MHz): 0.90-1.25 (m, 5H), 1.45-1.80 (m, 6H), 2.57 (s, 3H), 3.01 (d, 1H), 3.13 (d, 1H), 5.49 (s, 1H), 7.04 (dd, 1H), 7.17 (m, 2H), 7.39 (d, 2H), 7.91 (d, 2H), 8.00 (m, 2H), 8.05 (m, 1H), 8.26 (d, 1H), 10.07 (s, 1H).

The racemic mixture obtained was separated into the enantimers 27a and 27b by preparation chiral HPLC (column Chiralpak OD-H5μ 250×20 mm, eluent hexane/2-propanoll 98:15).

Example 27a: Rt=8.1-10.4 min

Example 27b: Rt=10.8-13.4 min

### EXAMPLE 28

### rac-3-(4′-Acetylbiphenyl-3-yl)-N-(4-cyano-3-trifluoromethyl phenyl)-2-cyclohexyl-2-hydroxypropionamide

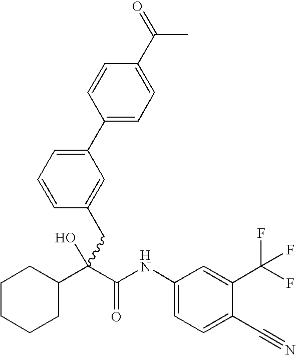

The preparation was effected analogously to Example 19 using the compound prepared in Example 26. ¹H NMR (ppm, CDCl₃, 400 MHz): 1.05-1.45 (m, 5H), 1.65-2.10 (m, 6H), 2.22 (s, 1H), 2.63 (s, 3H), 3.01 (d, 1H), 3.44 (d, 1H), 7.20 (m, 1H), 7.35 (m, 1H), 7.44 (m, 1H), 7.50 (d, 2H), 7.69 (m, 3H), 7.90 (m, 1H), 7.95 (d, 2H), 8.64 (s, 1H).

The racemic mixture obtained was separated into the enantimers 28a and 28b by preparation chiral HPLC (column Chiralpak 1A5μ 250×20 mm, hexane/ethanol 8:2).

Example 28a: [α]^{D}₂₀=−49.6° (MeOH, c=1.0)

Example 28b: [α]^{D}₂₀=???° (MeOH, c=1.0)

### EXAMPLE 29

### rac-6-[2-Cyclohexyl-2-hydroxy-2-(phenylethynyl)ethanoylamino]-4-methyl-2,3-benzoxazin-1-one

### 29a) rac-6-[2-Cyclohexyl-2-oxoethanoylamino]-4-methyl-2,3-benzoxazin-1-one

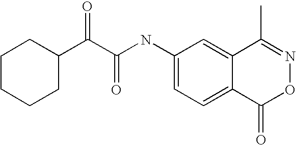

2-Oxo-2-cyclohexylacetic acid (200 mg) was reacted with 6-amino-4-methyl-2,3-benzoxazin-1-one and thionyl chloride in N,N-dimethylacetamide in analogy to Example d. After column chromatography, 360 mg of product were obtained.

¹H NMR (ppm, CDCl₃, 400 MHz): 1.23-1.51 (5H), 1.73-1.98 (5H), 2.61 (3H), 3.45-3.53 (1H), 7.84 (1H), 8.36 (1H), 8.39 (1H), 9.19 (1H).

### 29b) rac-6-[2-Cyclohexyl-2-hydroxy-2-(phenylethynyl)ethanoylamino]-4-methyl-2,3-benzoxazin-1-one

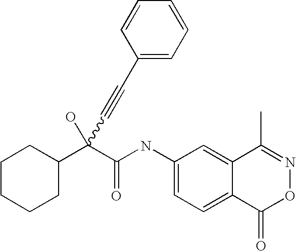

6-[2-Cyclohexyl-2-oxoethanoylamino]-4-methyl-2,3-benzoxazin-1-one (200 mg) was reacted with phenylethyne and n-butyllithium in tetrahydrofuran in analogy to Example 2. After column chromatography, 160 mg of product were obtained.

¹H NMR (ppm, DMSO-d₆, 400 MHz): 1.00-1.32 (5H), 1.58-1.77 (4H), 1.93-2.02 (2H), 2.47 (3H), 6.86 (1H), 7.34-7.43 (5H), 8.19 (1H), 8.37-8.41 (2H), 10.50 (1H).

### EXAMPLE 30

### rac-2-Cyclohexyl-2-hydroxy-N-(4-methyl-1-oxo-1H-benzo[d][1,2]oxazin-6-yl)-3-phenylpropionamide

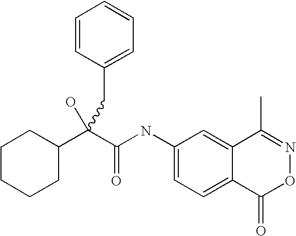

The preparation was effected analogously to Example 3 using the keto amide described in Example 29a and benzylmagnesium chloride. ¹H NMR (ppm, DMSO-D₆, 400 MHz): 1.00-1.35 (m, 5H), 1.45-1.85 (m, 5H), 1.95 (m, 1H), 2.40 (s, 3H), 2.90 (d, 1H), 3.06 (d, 1H), 5.48 (s, 1H), 7.00-7.25 (m, 5H), 8.06 (m, 1H), 8.10 (s, 1H), 8.25 (dd, 1H), 9.81 (s, 1H).

The resulting racemic mixture was separated by preparative chiral HPLC (column: Chiralpak AD 250×10 mm) into enantiomers 30a and 30b.

Example 30a: [α]^{D}₂₀=−119.4° (DMSO, c=0.54)

Example 30b: [α]^{D}₂₀=+113.5° (DMSO, c=0.57)

### EXAMPLE 31

### rac-2-Cyclohexyl-2-hydroxy-3-(4-methoxyphenyl)-N-(4-methyl-1-oxo-1H-benzo[d][1,2]oxazin-6-yl)propionamide

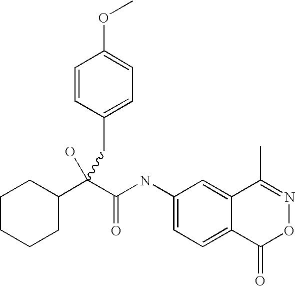

The preparation was effected analogously to Example 30 using 4-methoxybenzyl-magnesium chloride. ¹H NMR (ppm, CDCl₃, 400 MHz): 1.12-1.47 (m, 5H), 1.55-2.14 (m, 6H), 2.23 (s, 1H), 2.64 (s, 3H), 2.92 (d, 1H), 3.44 (d, 1H), 3.86 (s, 3H), 6.83 (d, 2H), 7.14 (d, 2H), 7.62 (dd, 1H), 8.25 (d, 1H), 8.31 (d, 1H), 8.81 (s, 1H).

### EXAMPLE 32

### rac-N-(3-Chloro-4-cyanophenyl)-2-cyclopentyl-2-hydroxy-3-phenylpropionamide

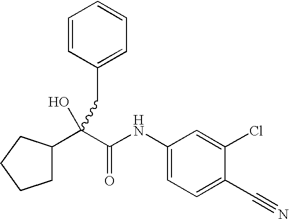

The compound was obtained analogously to Example 3 by reacting N-(3-chloro-4-cyanophenyl)-2-cyclopentyl-2-oxoacetamide (obtained analogously to the corresponding cyclohexyl compound, ¹H NMR (ppm, CDCl₃, 400 MHz): 1.71 (m, 4H), 1.82 (m, 2H), 2.01 (m, 2H), 3.85 (m, 1H), 7.60 (dd, 1H), 7.68 (d, 1H), 8.03 (d, 1H), 9.01 (s, 1H)) with benzylmagnesium chloride. ¹H NMR (ppm, CDCl₃, 400 MHz): 1.45-1.95 (m, 8H), 2.50 (m, 1H), 2.84 (d, 1H), 3.50 (d, 1H), 7.16 (m, 2H), 7.27 (m, 3H), 7.37 (dd, 1H), 7.55 (d, 1H), 7.82 (d, 1H), 8.52 (s, 1H).

### EXAMPLE 33

### rac-N-(3-Chloro-4-cyanophenyl)-2-cyclopentyl-2-hydroxy-3-p-tolylpropionamide

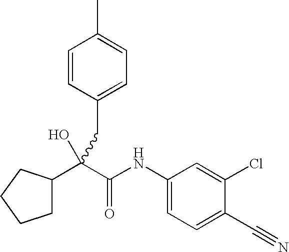

The preparation was effected analogously to Example 32 with 4-methylbenzyl-magnesium chloride. ¹H NMR (ppm, CDCl₃, 400 MHz): 1.45-1.75 (m, 7H), 1.87 (m, 1H), 2.15 (s, 1H), 2.29 (s, 3H), 2.48 (m, 1H), 2.79 (d, 1H), 3.48 (d, 1H), 7.06 (m, 4H), 7.39 (dd, 1H), 7.56 (d, 1H), 7.83 (d, 1H), 8.55 (s, 1H).

### EXAMPLE 34

### rac-N-(3-Chloro-4-cyanophenyl)-2-cyclopentyl-2-hydroxy-3-m-tolylpropionamide

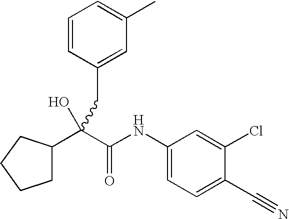

The preparation was effected analogously to Example 32 with 3-methylbenzyl-magnesium chloride. ¹H NMR (ppm, CDCl₃, 400 MHz): 1.45-1.73 (m, 7H), 1.85 (m, 1H), 2.17 (s, 1H), 2.25 (s, 3H), 2.49 (m, 1H), 2.79 (d, 1H), 3.48 (d, 1H), 6.95 (m, 2H), 7.05 (d, 1H), 7.16 (m, 1H), 7.38 (dd, 1H) 7.56 (d, 1H), 7.82 (d, 1H), 8.53 (s, 1H).

### EXAMPLE 35

### rac-N-(3-Chloro-4-cyanophenyl)-2-cyclopentyl-2-hydroxy-3-(4-methoxyphenyl)-propionamide

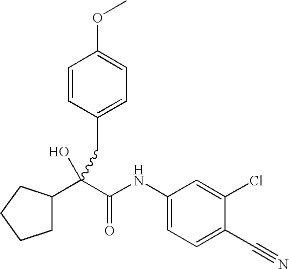

The preparation was effected analogously to Example 32 with 4-methoxybenzyl-magnesium chloride. ¹H NMR (ppm, CDCl₃, 400 MHz): 1.46-1.79 (m, 7H), 1.91 (m, 1H), 2.17 (s, 1H), 2.52 (m, 1H), 2.81 (d, 1H), 3.50 (d, 1H), 3.80 (s, 3H), 6.85 (m, 2H), 7.12 (m, 2H), 7.44 (dd, 1H), 7.61 (d, 1H), 7.89 (d, 1H), 8.61 (s, 1H).

### EXAMPLE 36

### rac-N-(3-Chloro-4-cyanophenyl)-2-cyclopentyl-2-hydroxy-3-(3-methoxyphenyl)-propionamide

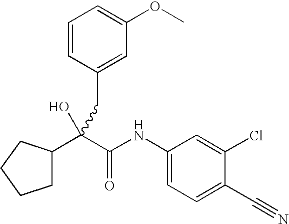

The preparation was effected analogously to Example 32 with 3-methoxybenzyl-magnesium chloride. ¹H NMR (ppm, CDCl₃, 400 MHz): 1.46-1.72 (m, 7H), 1.87 (m, 1H), 2.20 (s, 1H), 2.49 (m, 1H), 2.80 (d, 1H), 3.50 (d, 1H), 3.69 (s, 3H), 6.75 (m, 3H), 7.19 (m, 1H), 7.40 (dd, 1H), 7.56 (d, 1H), 7.84 (d, 1H), 8.58 (s, 1H).

### EXAMPLE 37

### rac-N-(3-Chloro-4-cyanophenyl)-2-cyclopentyl-2-hydroxy-3-(3-fluorophenyl)-propionamide

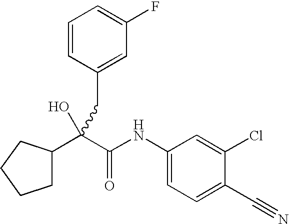

The preparation was effected analogously to Example 18, except using 3-fluorobenzyl-zinc chloride solution and N-(3-chloro-4-cyanophenyl)-2-cyclopentyl-2-oxoacetamide (see Example 32). ¹H NMR (ppm, CDCl₃, 400 MHz): 1.40-1.75 (m, 7H), 1.87 (m, 1H), 2.10 (s, 1H), 2.51 (m, 1H), 2.84 (d, 1H), 3.45 (d, 1H), 6.93 (m, 3H), 7.21 (m, 1H), 7.38 (m, 1H), 7.56 (d, 1H), 7.81 (d, 1H), 8.53 (s, 1H).

### EXAMPLE 38

### rac-N-(3-Chloro-4-cyanophenyl)-2-cyclopentyl-2-hydroxy-3-(3-chlorophenyl)-propionamide

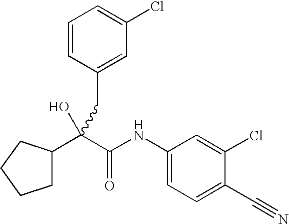

The preparation was effected analogously to Example 37 using 3-chlorobenzylzinc chloride solution. ¹H NMR (ppm, CDCl₃, 400 MHz): 1.41-1.75 (m, 7H), 1.89 (m, 1H), 2.09 (s, 1H), 2.52 (m, 1H), 2.81 (d, 1H), 3.40 (d, 1H), 7.04 (m, 1H), 7.19 (m, 3H), 7.39 (m, 1H), 7.56 (m, 1H), 7.80 (d, 1H), 8.51 (s, 1H).

### EXAMPLE 39

### rac-N-(4-Cyano-3-trifluoromethyl phenyl)-2-cyclopentyl-2-hydroxy-3-phenyl-propionamide

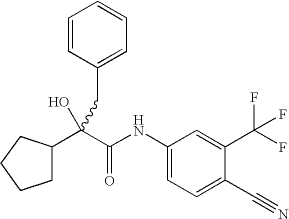

The compound was obtained analogously to Example 32 by reacting N-(4-cyano-3-trifluoromethylphenyl)-2-cyclopentyl-2-oxoacetamide (obtained analogously to the corresponding compound with Cl substitents, ¹H NMR (ppm, CDCl₃, 400 MHz): 1.60-1.87 (m, 7H), 1.99 (m, 1H), 3.84 (m, 1H), 7.84 (d, 1H), 7.99 (dd, 1H), 8.16 (d, 1H), 9.12 (s, 1H)) with benzylmagnesium chloride. ¹H NMR (ppm, CDCl₃, 400 MHz): 1.45-1.75 (m, 7H), 1.89 (m, 1H), 2.16 (s, 1H), 2.52 (m, 1H), 2.85 (d, 1H), 3.50 (d, 1H), 7.17 (dd, 2H), 7.28 (m, 3H), 7.74 (d, 1H), 7.82 (dd, 1H), 7.88 (d, 1H), 8.65 (s, 1H).

### EXAMPLE 40

### rac-N-(4-Cyano-3-trifluoromethyl phenyl)-2-cyclopentyl-2-hydroxy-3-p-tolyl-propionamide

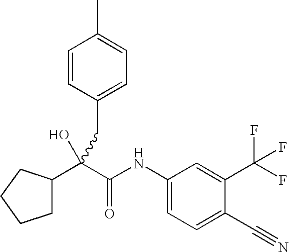

The preparation was effected analogously to Example 33. ¹H NMR (ppm, CDCl₃, 400 MHz): 1.48-1.72 (m, 7H), 1.89 (m, 1H), 2.18 (s, 1H), 2.28 (s, 3H), 2.49 (m, 1H), 2.81 (d, 1H), 3.48 (d, 1H), 7.06 (m, 4H), 7.75 (d, 1H), 8.86 (m, 2H), 8.67 (s, 1H).

### EXAMPLE 41

### rac-N-(4-Cyano-3-trifluoromethyl phenyl)-2-cyclopentyl-2-hydroxy-3-m-tolyl-propionamide

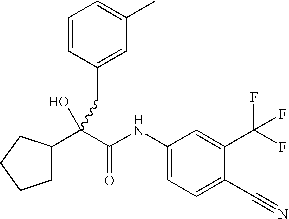

The preparation was effected analogously to Example 34. ¹H NMR (ppm, CDCl₃, 400 MHz): 1.45-1.75 (m, 7H), 1.89 (m, 1H), 2.19 (s, 1H), 2.24 (s, 3H), 2.51 (m, 1H), 2.80 (d, 1H), 3.47 (d, 1H), 6.96 (m, 2H), 7.05 (d, 1H), 7.16 (m, 1H), 7.75 (d, 1H) 7.83 (dd, 1H), 7.88 (d, 1H), 8.66 (s, 1H).

### EXAMPLE 42

### rac-N-(4-Cyano-3-trifluoromethyl phenyl)-2-cyclopentyl-2-hydroxy-3-(4-methoxy-phenyl)propionamide

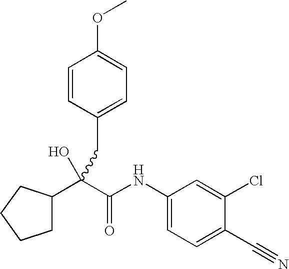

The preparation was effected analogously to Example 35. ¹H NMR (ppm, CDCl₃, 400 MHz): 1.50-1.75 (m, 7H), 1.93 (m, 1H), 2.21 (s, 1H), 2.53 (m, 1H), 2.83 (d, 1H), 3.51 (d, 1H), 3.79 (s, 3H), 6.85 (m, 2H), 7.13 (d, 2H), 7.79 (d, 1H), 7.90 (dd, 1H), 7.95 (d, 1H), 8.73 (s, 1H).

### EXAMPLE 43

### rac-N-(4-Cyano-3-trifluoromethyl phenyl)-2-cyclopentyl-2-hydroxy-3-(3-methoxy-phenyl)propionamide

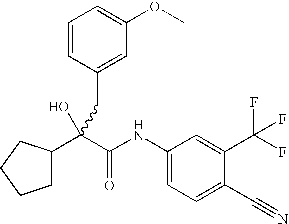

The preparation was effected analogously to Example 36. ¹H NMR (ppm, CDCl₃, 400 MHz): 1.46-1.73 (m, 7H), 1.88 (m, 1H), 2.23 (s, 1H), 2.50 (m, 1H), 2.81 (d, 1H), 3.49 (d, 1H), 3.68 (s, 3H), 6.69-6.80 (m, 3H), 7.19 (dd, 1H), 7.74 (d, 1H), 7.84 (dd, 1H), 7.91 (d, 1H), 8.70 (s, 1H).

### EXAMPLE 44

### rac-N-(4-Cyano-3-trifluoromethyl phenyl)-2-cyclopentyl-2-hydroxy-3-phenyl-propionamide

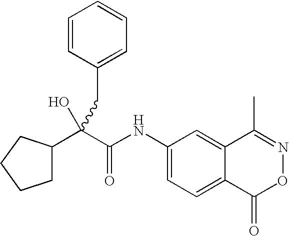

The compound was obtained analogously to Example 32 by reacting 2-cyclopentyl-N-(4-methyl-1-oxo-1H-benzo[d][1,2]oxazin-6-yl)-2-oxoacetamide (obtained analogously to the corresponding cyclohexyl compound from Example 15^{a}), ¹H NMR (ppm, CDCl₃, 400 MHz): 1.71 (m, 4H), 1.82 (m, 2H), 2.01 (m, 2H), 2.61 (s, 3H), 3.86 (m, 1H), 7.84 (dd, 1H), 8.35 (d, 1H), 8.38 (d, 1H), 9.21 (s, 1H)) with benzylmagnesium chloride. ¹H NMR (ppm, CDCl₃, 400 MHz): 1.50-1.80 (m, 7H), 1.96 (m, 1H), 2.25 (s, 1H), 2.59 (m, 1H), 2.63 (s, 3H), 2.93 (d, 1H), 3.57 (d, 1H), 7.21-7.43 (m, 5H), 7.61 (dd, 1H), 8.25 (d, 1H), 8.32 (d, 1H), 8.79 (s, 1H).

### EXAMPLE 45

### rac-N-(4-Cyano-3-trifluoromethyl phenyl)-2-hydroxy-2-hydroxy-3-phenyl-2-(tetra-hydropyran-4-yl)propionamide

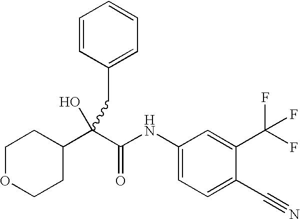

### a) Acetoxy(tetrahydropyran-4-ylidene)acetic acid ethyl ester

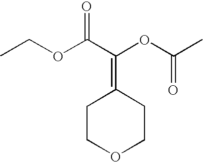

7.0 g of ethyl acetoxy(diethoxyphosphoryl)acetate were initially charged in 25 ml of THF, 1.06 g of lithium chloride were added and the mixture was cooled to 0° C. 3.1 ml of N,N,N′,N′-tetramethylguanidine were added dropwise and the mixture was stirred for 15 min. 2.46 g of tetrahydro-4H-pyran-4-one, dissolved in 10 ml of THF, were then added dropwise. The mixture was allowed to come to room temperature and stirred for 18 h. The mixture was partitioned between ethyl acetate and water, the phases were separated and the aqueous phase was extracted with ethyl acetate. The combined organic phases were dried over sodium sulphate and concentrated. The residue was chromatographed on silica gel. 3.37 g of the desired intermediate were obtained as a yellowish liquid. ¹H NMR (ppm, CDCl₃, 400 MHz): 1.28 (t, 3H), 2.21 (s, 3H), 2.36 (t, 2H), 2.98 (t, 2H), 3.75 (m, 4H), 4.21 (q, 2H).

### b) Oxo(tetrahydropyran-4-yl)acetic acid

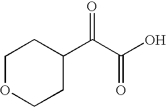

870 mg of acetoxy(tetrahydropyran-4-ylidene)acetic acid ethyl ester were added to 8 ml of 1M sodium hydroxide solution in 2:1 ethanol/water. The mixture was left to stir at room temperature for 15 min, then diluted with cold water, acidified with hydrochloric acid and extracted with ethyl acetate. The combined organic phases were dried over sodium sulphate and concentrated. 540 mg of oxo(tetrahydropyran-4-yl)acetic acid were obtained as a colourless crystalline solid. ¹H NMR (ppm, CDCl₃, 400 MHz): 1.77 (m, 2H), 1.89 (m, 2H), 3.45 (m, 1H), 3.56 (m, 2H), 4.07 (m, 2H), 8.76 (br s, 1H).

### c) N-(4-Cyano-3-trifluoromethylphenyl)-2-oxo-2-(tetrahydropyran-4-yl)acetamide

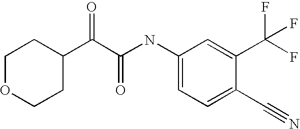

The preparation was effected analogously to the corresponding cyclohexyl compound (see above) from oxo(tetrahydropyran-4-yl)acetic acid, 4-cyano-3-trifluoromethylaniline and thionyl chloride. ¹H NMR (ppm, CDCl₃, 400 MHz): 1.75 (m, 2H), 1.85 (m, 2H), 3.55 (m, 2H), 3.68 (m, 1H), 4.05 (m, 2H), 7.86 (d, 1H), 7.98 (dd, 1H), 8.17 (d, 1H), 9.08 (s, 1H).

### d) rac-N-(4-Cyano-3-trifluoromethylphenyl)-2-hydroxy-2-hydroxy-3-phenyl-2-(tetrahydro-pyran-4-yl)propionamide

The preparation was effected analogously to Example 20 from N-(4-cyano-3-trifluoromethylphenyl)-2-oxo-2-(tetrahydropyran-4-yl)acetamide and benzyl-magnesium chloride. ¹H NMR (ppm, CDCl₃, 400 MHz): 1.45-1.87 (m, 4H), 2.19 (m, 1H), 2.25 (s, 1H), 2.88 (d, 1H), 3.40 (m, 2H), 3.46 (d, 1H), 4.01 (dd, 1H), 4.10 (dd, 1H), 7.17 (m, 2H), 7.28 (m, 3H), 7.75 (d, 1H), 7.82 (d, 1H), 7.88 (d, 1H), 8.64 (s, 1H).

### EXAMPLES 46 TO 55

Analogously to example 27, using the method used in example 19, the following compounds were prepared. Separation into the corresponding enantiomers was effected, where appropriate, by chiral HPLC:

| | | | MS (ESI+) |
|---|---|---|---|
| Example | Structure | Name | m/z (M + 1) |
| Example 46 | 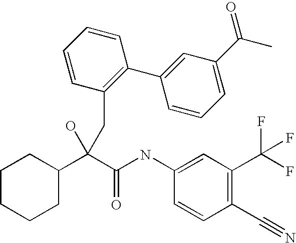 | 3-(3′-Acetylbiphenyl-2-yl)-N-(4- cyano-3-trifluoromethylphenyl)-2- cyclohexyl-2-hydroxypropionamide | 535 |
| Example 47 | 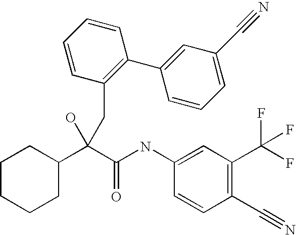 | 3-(3′-Cyanobiphenyl-2-yl)-N-(4- cyano-3-trifluoromethylphenyl)-2- cyclohexyl-2-hydroxypropionamide | 518 |
| Example 48 | 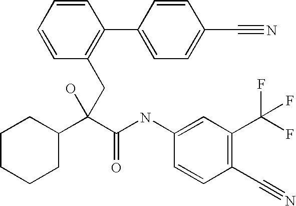 | 3-(4′-Cyanobiphenyl-2-yl)-N-(4- cyano-3-trifluoromethylphenyl)-2- cyclohexyl-2-hydroxypropionamide | 518 |
| Example 49 | 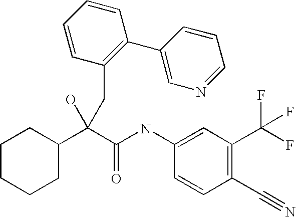 | N-(4-Cyano-3-trifluoromethylphenyl)- 2-cyclohexyl-2-hydroxy-3-(2-pyridin- 3-ylphenyl)propionamide | 494 |
| Example 50 | 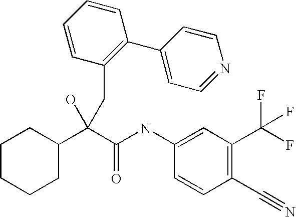 | N-(4-Cyano-3-trifluoromethyl- phenyl)-2-cyclohexyl-2-hydroxy- 3-(2-pyridin-4-ylphenyl)propionamide | 494 |
| Example 51 | 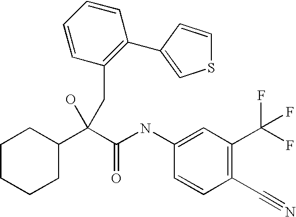 | N-(4-Cyano-3-trifluoromethyl- phenyl)-2-cyclohexyl-2-hydroxy- 3-(2-thiophen-3-ylphenyl)- propionamide | 499 |
| Example 52 | 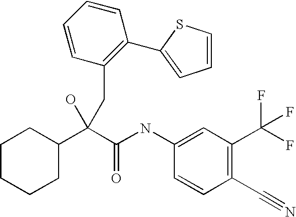 | N-(4-Cyano-3-trifluoromethylphenyl)- 2-cyclohexyl-2-hydroxy-3-(2-thiophen- 2-yl-phenyl)propionamide | 499 |
| Example 53 | 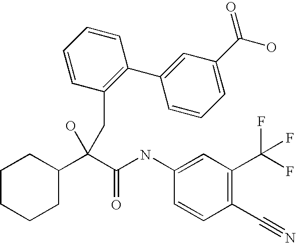 | 2′-[2-(4-Cyano-3-trifluoromethyl- phenylcarbamoyl)-2-cyclohexyl- 2-hydroxyethyl]biphenyl-3- carboxylic acid | 537 |
| Example 54 | 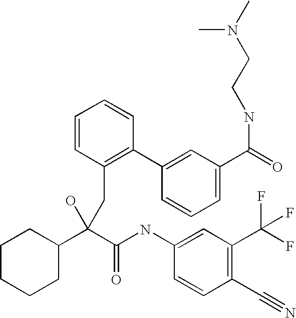 | N-(2-Dimethylaminoethyl)-2′- [2-(4-cyano-3-trifluoromethyl- phenylcarbamoyl)-2-cyclohexyl- 2-hydroxyethyl]biphenyl-3- carboxamide | 607 |
| Example 55 | 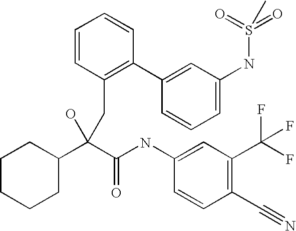 | N-(4-Cyano-3-trifluoromethylphenyl)- 2-cyclohexyl-2-hydroxy-3-(3′-methane- sulfonylaminobiphenyl-2-yl)propionamide | 586 |

### EXAMPLES 56 TO 78

Analogously to example 26, using the method used in example 28, the following compounds were prepared:

| | | | MS (ESI+) |
|---|---|---|---|
| Example | Structure | Name | m/z (M + 1) |
| Example 56 | 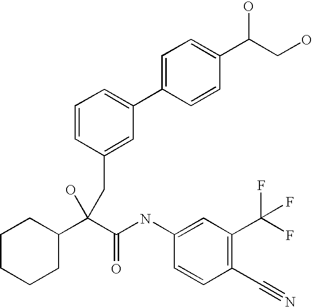 | N-(4-Cyano-3-trifluoromethylphenyl)- 2-cyclohexyl-3-[4′-(1,2-dihydroxyethyl)- biphenyl-3-yl]-2-hydroxypropionamide | 553 |
| Example 57 | 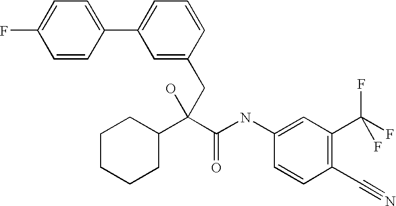 | N-(4-Cyano-3-trifluoromethylphenyl)- 2-cyclohexyl-3-(4′-fluorobiphenyl-3- yl)-2-hydroxypropionamide | 511 |
| Example 58 | 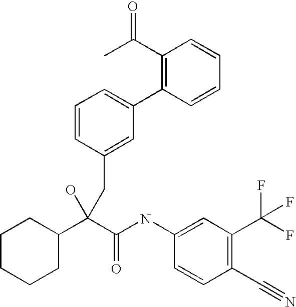 | 3-(2′-Acetylbiphenyl-3-yl)-N-(4- cyano-3-trifluoromethylphenyl)-2- cyclohexyl-2-hydroxypropionamide | 535 |
| Example 59 | 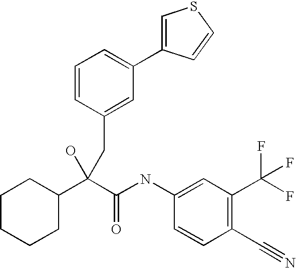 | N-(4-Cyano-3-trifluoromethylphenyl)- 2-cyclohexyl-2-hydroxy-3-(3-thiophen- 3-ylphenyl)propionamide | 499 |
| Example 60 | 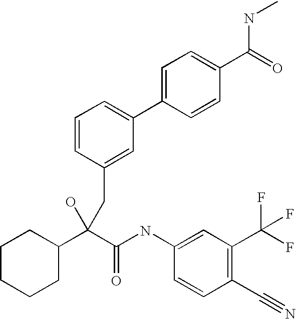 | N-Methyl-3′-[2-(4-cyano-3-trifluoro- methylphenylcarbamoyl)-2-cyclo- hexyl-2-hydroxyethyl]biphenyl-4- carboxamide | 550 |
| Example 61 | 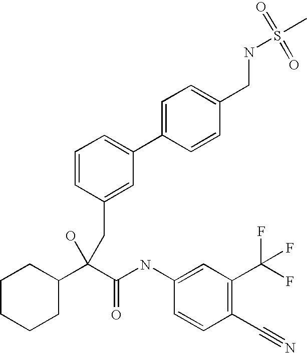 | N-(4-Cyano-3-trifluoromethylphenyl)- 2-cyclohexyl-2-hydroxy-3-[4′-(methane- sulfonylaminomethyl)biphenyl-3-yl]- propionamide | 600 |
| Example 62 | 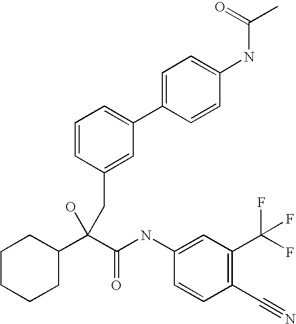 | 3-(4′-Acetylaminobiphenyl-3-yl)-N- (4-cyano-3-trifluoromethylphenyl)- 2-cyclohexyl-2-hydroxypropionamide | 550 |
| Example 63 | 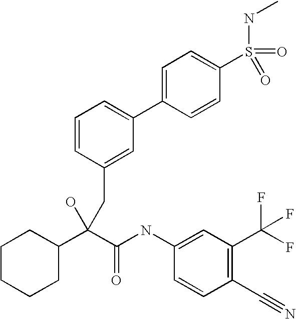 | N-(4-Cyano-3-trifluoromethylphenyl)- 2-cyclohexyl-2-hydroxy-3-(4′-methyl- sulfamoylbiphenyl-3-yl)propionamide | 586 |
| Example 64 | 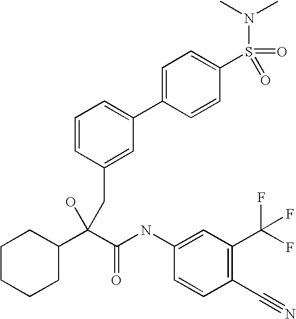 | N-(4-Cyano-3-trifluormethylphenyl)- 2-cyclohexyl-3-(4′-dimethylsulfamoyl- biphenyl-3-yl)-2-hydroxypropionamide | 600 |
| Example 65 | 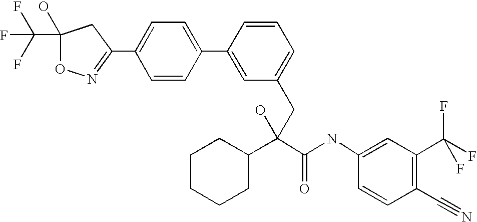 | N-(4-Cyano-3-trifluoromethylphenyl)-2- cyclohexyl-2-hydroxy-3-[4′-(5-hydroxy- 5-trifluoromethyl-4,5-dihydroisoxazol- 3-yl)biphenyl-3-yl]propionamide | 646 |
| Example 66 | 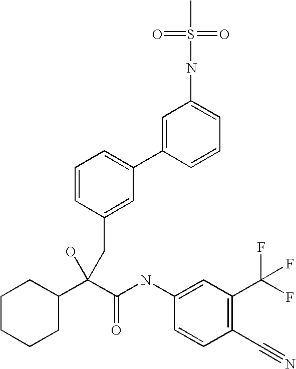 | N-(4-Cyano-3-trifluoromethylphenyl)- 2-cyclohexyl-2-hydroxy-3-(3′-methane- sulfonylaminobiphenyl-3-yl)propionamide | 586 |
| Example 67 | 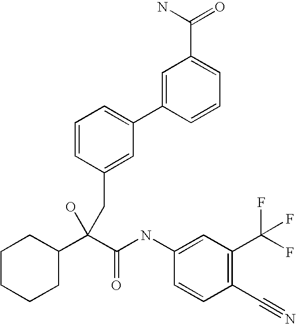 | 3′-[2-(4-Cyano-3-trifluoromethylphenyl- carbamoyl)-2-cyclohexyl-2-hydroxy- ethyl]biphenyl-3-carboxamide | 536 |
| Example 68 | 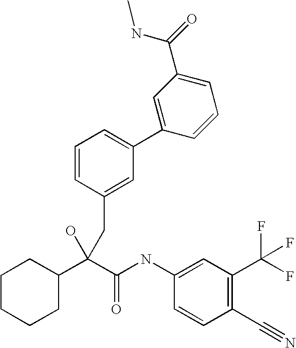 | N-Methyl-3′-[2-(4-cyano-3-trifluoro- methylphenylcarbamoyl)-2-cyclohexyl- 2-hydroxyethyl]biphenyl-3-carboxamide | 550 |
| Example 69 | 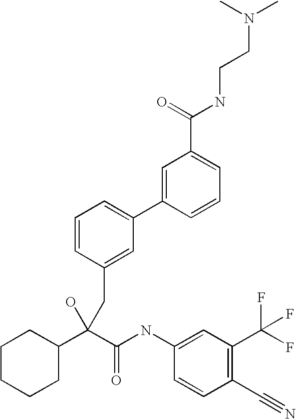 | N-(2-Dimethylaminoethyl)-3′-[2-(4- cyano-3-trifluoromethylphenyl- carbamoyl)-2-cyclohexyl-2-hydroxy- ethyl]biphenyl-3-carboxamide | 607 |
| Example 70 | 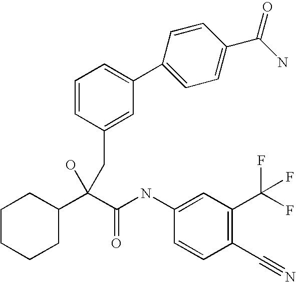 | 3′-[2-(4-Cyano-3-trifluoromethylphenyl- carbamoyl)-2-cyclohexyl-2-hydroxy- ethyl]biphenyl-4-carboxamide | 536 |
| Example 71 | 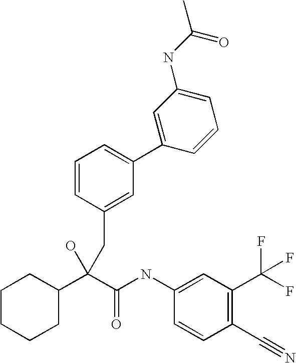 | 3-(3′-Acetylaminobiphenyl-3-yl)-N- (4-cyano-3-trifluoromethylphenyl)- 2-cyclohexyl-2-hydroxypropionamide | 550 MS (ESI+) m/z (M + 1) |
| Example 72 | 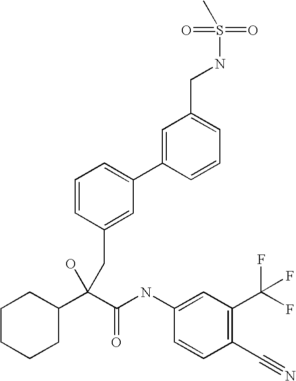 | N-(4-Cyano-3-trifluoromethylphenyl)- 2-cyclohexyl-2-hydroxy-3-[3′-(methane- sulfonylaminomethyl)biphenyl-3-yl]- propionamide | 600 |
| Example 73 | 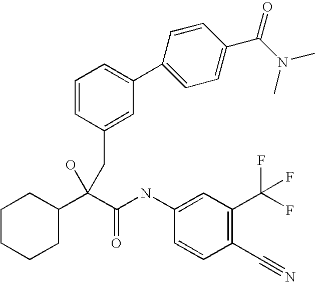 | N,N-Dimethyl-3′-[2-(4-Cyano-3-tri- fluoromethylphenylcarbamoyl)-2- cyclohexyl-2-hydroxyethyl]biphenyl-4- carboxamide | 564 |
| Example 74 | 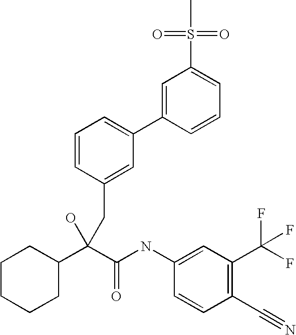 | N-(4-Cyano-3-trifluoromethylphenyl)- 2-cyclohexyl-2-hydroxy-3-(3′-methane- sulfonylbiphenyl-3-yl)propionamide | 571 |
| Example 75 | 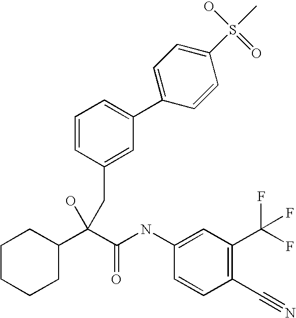 | N-(4-Cyano-3-trifluoromethylphenyl)- 2-cyclohexyl-2-hydroxy-3-(4′-methane- sulfonylbiphenyl-3-yl)propionamide | 571 |
| Example 76 | 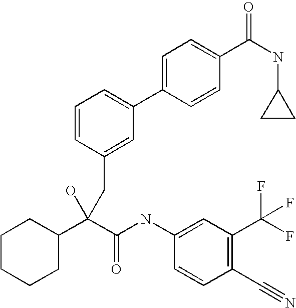 | N-Cyclopropyl-3′-[2-(4-cyano-3- trifluoromethylphenylcarbamoyl)- 2-cyclohexyl-2-hydroxyethyl]- biphenyl-4-carboxamide | 576 |
| Example 77 | 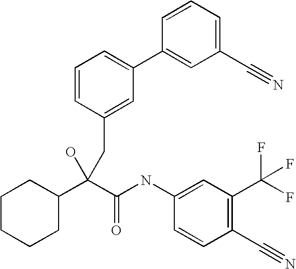 | 3-(3′-Cyanobiphenyl-3-yl)-N-(4- cyano-3-trifluoromethylphenyl)-2- cyclohexyl-2-hydroxypropionamide | 518 |
| Example 78 | 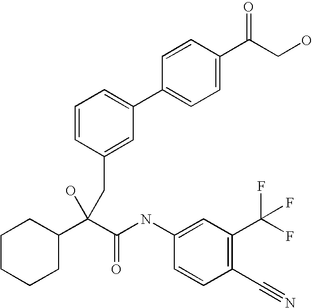 | N-(4-Cyano-3-trifluoromethylphenyl)- 2-cyclohexyl-2-hydroxy-3-[4′-(2-hydroxy- acetyl)biphenyl-3-yl]propionamide | 551 |

### EXAMPLE 79

### N-(4-Cyano-3-trifluoromethyl phenyl)-2-cyclohexyl-3-(3-{[(2-dimethylamino-ethyl)methylamino]methyl}phenyl)-2-hydroxypropionamide

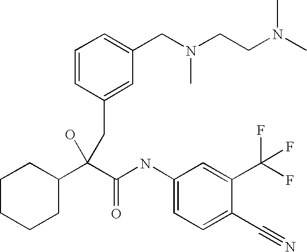

### a) 3-[2-(4-Cyano-3-trifluoromethylphenylcarbamoyl)-2-cyclohexyl-2-hydroxyethyl]-benzoic acid methyl ester

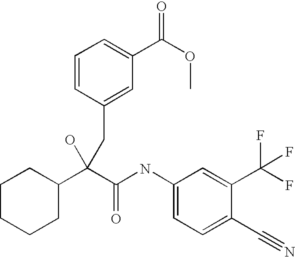

570 mg of N-(4-cyano-3-trifluoromethylphenyl)-2-cyclohexyl-2-hydroxy-3-(3-iodo-phenyl)propionamide (example 26) were suspended in a glass pressure tube together with 75 mg of dichlorobis(triphenylphosphine)palladium and 0.32 ml of triethylamine in 17 ml of methanol and 1 ml of DMSO. The mixture was degassed and placed under a carbon monoxide atmosphere. The pressure tube was then closed and heated to 100° C. The mixture was stirred for 16 h and cooled, and the pressure vessel was opened. The mixture was concentrated and purified by chromatography. This afforded 293 mg (59%) of the target product.

### b) N-(4-Cyano-3-trifluoromethylphenyl)-2-cyclohexyl-2-hydroxy-3-(3-hydroxymethyl-phenyl)propionamide

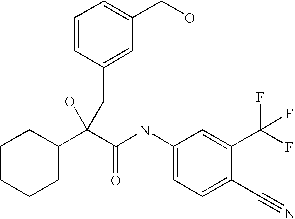

170 mg of the product obtained in step a) were initially charged in 6 ml of THF at RT and admixed with 0.25 ml of 1M lithium aluminum hydride solution in THF and stirred for 2 h. The reaction was ended by adding water. The mixture was extracted with ethyl acetate, washed with NaCl solution, dried with sodium sulfate and concentrated. This afforded 156 mg (98%) of the desired product.

### c) N-(4-Cyano-3-trifluoromethylphenyl)-2-cyclohexyl-3-(3-formylphenyl)-2-hydroxy-propionamide

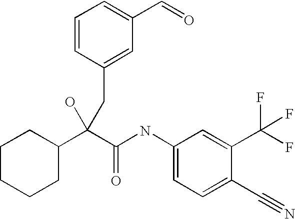

156 mg of the product obtained in step b) were dissolved in 5 ml of dichloromethane and, at 0° C., 222 mg of Dess-Martin periodinane were added. After stirring for 2 h, the mixture was added to 1:1 sodium hydrogencarbonate solution/sodium thiosulfate solution and extracted with ethyl acetate, and the organic phases were washed with NaCl solution, dried with sodium sulfate and concentrated. This afforded 160 mg of crude product, which was converted further directly.

### d) N-(4-Cyano-3-trifluoromethylphenyl)-2-cyclohexyl-3-(3-{[(2-dimethylaminoethyl)-methylamino]methyl}phenyl)-2-hydroxypropionamide

The product obtained in step c) was initially charged in 6 ml of dichloromethane, 60 μl of N,N,N′-trimethylethylenediamine were added and, after stirring at RT for 15 min, 170 mg of trisacetoxyborohydride were added in portions. After stirring for 16 h, the mixture was admixed with sodium hydrogencarbonate solution admixed (pH 9) and extracted with ethyl acetate, and the organic phases were washed with water and NaCl solution, dried with sodium sulfate and concentrated. This afforded 170 mg of crude product, which was purified by chromatography. The resulting purified racemate was separated into the enantiomers by means of chiral HPLC (Chiralcel OD-H 5μ, 250×20 mm, 99:1 hexane (0.1% DEA)/ethanol, 25 ml/min).

Example 79a: Rₜ=17.5-21.4 min

Example 79b: Rₜ=23.0-27.4 min

¹H NMR (ppm, d₆-DMSO, 400 MHz): 1.02-1.29 (m, 3H), 1.45 (m, 1H), 1.58 (m, 1H), 1.65 (m, 1H), 1.76 (m, 2H), 1.92 (m, 1H), 2.05 (m, 6H), 3.23 (m, 3H), 2.87 (d, 1H), 2.99 (d, 1H), 3.23 (m, 2H), 5.44 (s, 1H), 6.96 (m, 1H), 7.06 (m, 3H), 7.95 (d, 1H), 8.06 (dd, 1H), 8.80 (d, 1H), 9.89 (s, 1H). LCMS (ESI+): m/z=531 (M+1).

This reaction sequence can be carried out analogously beginning with the corresponding 2-iodo or 4-iodo compounds.

### EXAMPLE 80 TO 95

Analogously to example 79, a multitude of different amines can be used in the reductive amination in step d). The following example compounds were prepared:

| | | | MS (ESI+) |
|---|---|---|---|
| Example | Structure | Name | m/z (M + 1) |
| Example 80 | 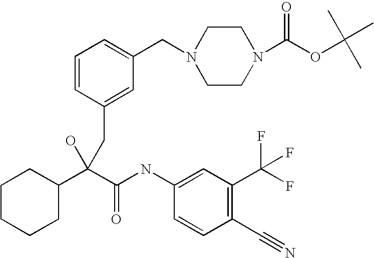 | tert-Butyl 4-{3-[2-(4-cyano-3-tri- fluoromethylphenylcarbamoyl)-2- cyclohexyl-2-hydroxyethyl]benzyl}- piperazine-1-carboxylate | 615 |
| Example 81 | 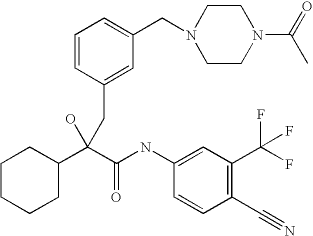 | 3-[3-(4-Acetylpiperazin-1-ylmethyl)- phenyl]-N-(4-cyano-3-trifluoromethyl- phenyl)-2-cyclohexyl-2-hydroxy- propionamide | 557 |
| Example 82 | 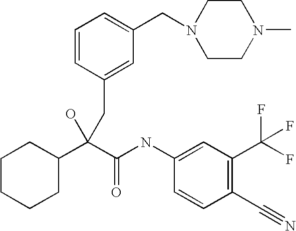 | N-(4-Cyano-3-trifluoromethylphenyl)- 2-cyclohexyl-2-hydroxy-3-[3-(4-methyl- piperazin-1-ylmethyl)phenyl]propionamide | 529 |
| Example 83 | 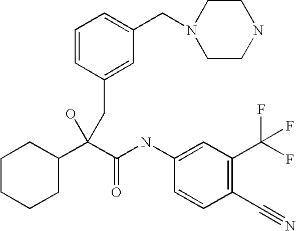 | N-(4-Cyano-3-trifluoromethylphenyl)- 2-cyclohexyl-2-hydroxy-3-(3-piperazin- 1-ylmethylphenyl)propionamide | 515 |
| Example 84 | 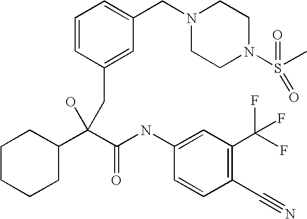 | N-(4-Cyano-3-trifluoromethylphenyl)- 2-cyclohexyl-2-hydroxy-3-[3-(4-methane- sulfonylpiperazin-1-ylmethyl)phenyl]- propionamide | 593 |
| Example 85 | 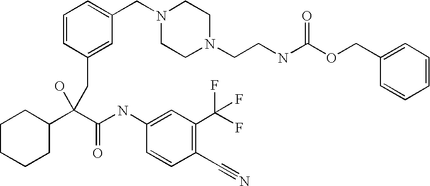 | Benzyl [2-(4-{3-[2-(4-cyano-3-trifluoro- methylphenylcarbamoyl)-2-cyclohexyl- 2-hydroxyethyl]benzyl}piperazin-1-yl)- ethyl]carbamate | 692 |
| Example 86 | 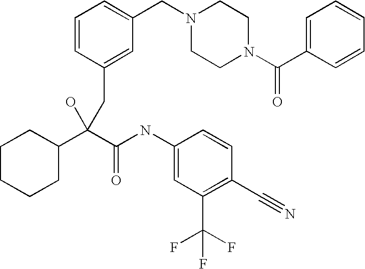 | 3-[3-(4-Benzoylpiperazin-1-ylmethyl)- phenyl]-N-(4-cyano-3-trifluoromethyl- phenyl)-2-cyclohexyl-2-hydroxy- propionamide | 619 |
| Example 87 | 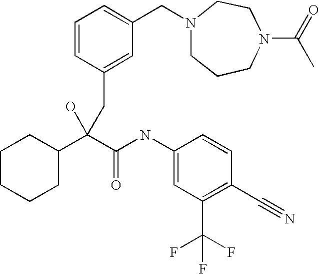 | 3-[3-(4-Acetyl-[1,4]diazepan-1-yl- methyl)phenyl]-N-(4-cyano-3- trifluoromethylphenyl)-2-cyclohexyl- 2-hydroxypropionamide | 571 |
| Example 88 | 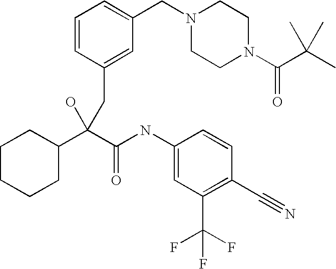 | N-(4-Cyano-3-trifluoromethylphenyl)- 2-cyclohexyl-3-{3-[4-(2,2-dimethyl- propionyl)piperazin-1-ylmethyl]- phenyl}-2-hydroxypropionamide | 599 |
| Example 89 | 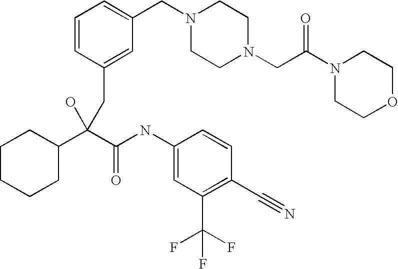 | N-(4-Cyano-3-trifluoromethylphenyl)- 2-cyclohexyl-2-hydroxy-3-{3-[4-(2- morpholin-4-yl-2-oxoethyl)piperazin- 1-ylmethyl]phenyl}propionamide | 642 |
| Example 90 | 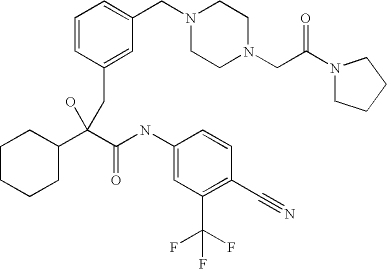 | N-(4-Cyano-3-trifluoromethylphenyl)- 2-cyclohexyl-2-hydroxy-3-{3-[4-(2- oxo-2-pyrrolidin-1-ylethyl)piperazin- 1-ylmethyl]phenyl}propionamide | 626 |
| Example 91 | 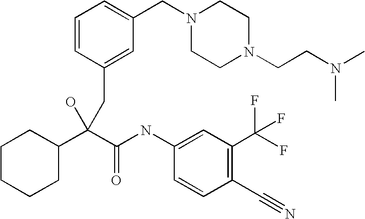 | N-(4-Cyano-3-trifluoromethylphenyl)- 2-cyclohexyl-3-{3-[4-(2-dimethylamino- ethyl)piperazin-1-ylmethyl]phenyl}- 2-hydroxypropionamide | 586 |
| Example 92 | 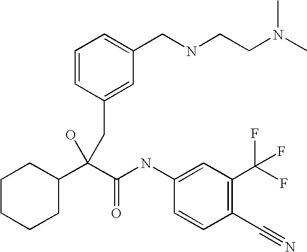 | N-(4-Cyano-3-trifluoromethylphenyl)- 2-cyclohexyl-3-{3-[(2-dimethylamino- ethylamino)methyl]phenyl}-2-hydroxy- propionamide | 517 |
| Example 93 | 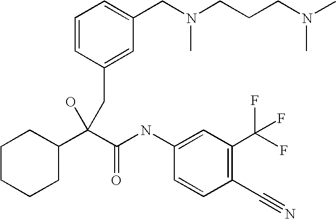 | N-(4-Cyano-3-trifluoromethylphenyl)- 2-cyclohexyl-3-(3-{[(3-dimethylamino- propyl)methylamino]methyl}phenyl)- 2-hydroxypropionamide | 545 |
| Example 94 | 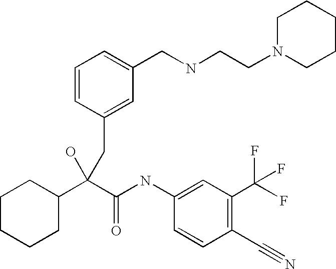 | N-(4-Cyano-3-trifluoromethylphenyl)- 2-cyclohexyl-2-hydroxy-3-{3-[(2-piperidin- 1-ylethylamino)methyl]phenyl}propionamide | 557 |
| Example 95 | 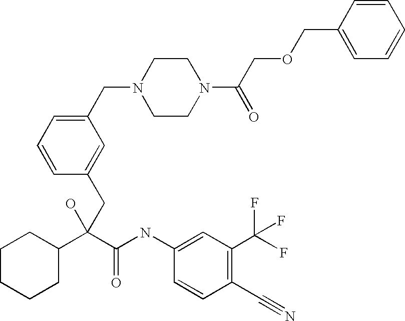 | 3-{3-[4-(2-Benzyloxyacetyl)piperazin- 1-ylmethyl]phenyl}-N-(4-cyano-3-tri- fluoromethylphenyl)-2-cyclohexyl-2- hydroxypropionamide | 663 |

### EXAMPLE 96

### 3-[3-(4-Benzoylpiperazine-1-carbonyl)-phenyl]-N-(4-cyano-3-trifluoromethyl-phenyl)-2-cyclohexyl-2-hydroxypropionamide

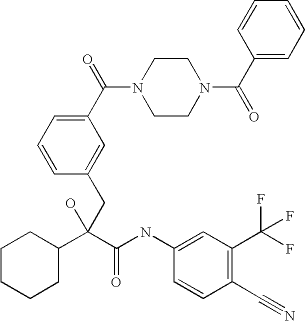

a) 250 mg of the methyl ester obtained in example 79a) were dissolved in 5 ml of 1:1 THF/water and 125 mg of LiOH were added. The mixture was stirred at RT for 16 h, water was added, and the mixture was adjusted to pH 4 with 2M HCl, extracted with ethyl acetate, washed with NaCl solution, dried with sodium sulfate and concentrated. The crude product was recrystallized from dichloromethane and converted further directly.

b) 50 mg of the acid obtained in a), 30 mg of benzoylpiperazine and 0.05 ml of triethylamine were dissolved in 6 ml of DMF, and 46 mg of the coupling reagent HATU were added. The mixture was stirred at RT for 16 h, water was added, and the mixture was extracted with ethyl acetate, washed with NaCl solution, dried with sodium sulfate and concentrated. The crude product was chromatographed on amino phase. This afforded 20 mg of the desired product.

LCMS: ES+: m/z=633 (M+1), ES−: m/z=631 (M−1)

Analogously, further examples can be prepared using various amine components.

### EXAMPLE 97

### N-(5-Cyano-1-methyl-1H-pyrrol-2-yl)-2-cyclohexyl-2-hydroxy-3-phenyl-propionamide

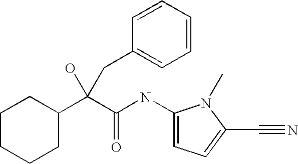

### a) 5-Nitro-1-methyl-1H-pyrrole-2-carbonitrile

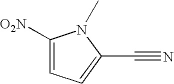

1.2 g of 1-methyl-1H-pyrrole-2-carbonitrile were dissolved in 7 ml of acetic anhydride. At 0° C., a mixture of 1 ml of fuming nitric acid and 2 ml of acetic anhydride was added dropwise at such a rate that the internal temperature did not rise above 1° C. After 2 h, the mixture was added to 25 ml of ice-water, extracted with diethyl ether and ethyl acetate, dried with sodium sulfate and concentrated. The crude product was purified by chromatography. This afforded 170 mg of the desired isomer: ¹H NMR (ppm, d₆-DMSO, 400 MHz): 3.98 (s, 3H), 7.12 (d, 1H), 7.27 (d, 1H); and 260 mg of 4-nitro-1-methyl-1H-pyrrole-2-carbonitrile ¹H NMR (ppm, d₆-DMSO, 400 MHz): 3.79 (s, 3H), 7.67 (s, 1H), 8.31 (s, 1H).

### b) 5-Amino-1-methyl-1H-pyrrole-2-carbonitrile

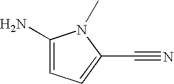

200 mg of 5-nitro-1-methyl-1H-pyrrole-2-carbonitrile were dissolved in 6 ml of ethyl acetate, 140 mg of palladium (10% on activated carbon) were added and hydrogenation was effected at RT and 1 atm of hydrogen pressure. The catalyst was filtered off and the solution was concentrated. This afforded 160 mg of the desired product. ¹H NMR (ppm, CDCl₃, 400 MHz): 2.85 (s br, 2H), 3.65 (s, 3H), 6.29 (d, 1H), 6.35 (d, 1H);

### c) N-(5-Cyano-1-methyl-1H-pyrrol-2-yl)-2-cyclohexyl-2-oxoacetamide

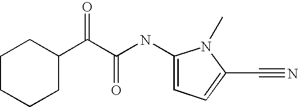

116 mg of cyclohexyloxoacetic acid and 90 mg of 5-amino-1-methyl-1H-pyrrole-2-carbonitrile were converted analogously to the preparation of N-(3-chloro-4-cyanophenyl)-2-oxo-2-cyclohexylacetamide (see above) to N-(5-cyano-1-methyl-1H-pyrrol-2-yl)-2-cyclohexyl-2-oxoacetamide. Yield 125 mg (65%).

### d) N-(5-Cyano-1-methyl-1H-pyrrol-2-yl)-2-cyclohexyl-2-hydroxy-3-phenyl-propionamide

107 mg of the keto amide obtained in step c) were reacted analogously to example 20 with benzylmagnesium chloride. This afforded 97 mg (90%) of the desired product. ¹H NMR (ppm, CDCl₃, 400 MHz): 1.10-1.36 (m, 5H), 1.65-1.86 (m, 5H), 1.99 (m, 1H), 2.87 (d, 1H), 3.42 (d, 1H), 3.73 (s, 3H), 6.42 (m, 1H), 7.16 (m, 2H), 7.26 (m, 3H), 7.41 (m, 1H), 8.13 (s, 1H).

### EXAMPLE 98

### N-(5-Cyano-1-methyl-1H-pyrrol-3-yl)-2-cyclohexyl-2-hydroxy-3-phenylpropionamide

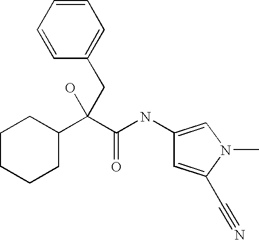

Analogously to example 97, the sequence can also be carried out with the 4-nitro-1-methyl-1H-pyrrole-2-carbonitrile obtained in step a) of example 97. ¹H NMR (ppm, CDCl₃, 400 MHz): 1.10-1.39 (m, 5H), 1.60-2.03 (m, 5H), 2.23 (m, 1H), 2.85 (d, 1H), 3.35 (d, 1H), 3.74 (s, 3H), 5.95 (m, 1H), 6.70 (d, 1H), 6.80 (m, 3H), 7.21 (m, 1H), 7.26 (m, 1H), 8.10 (s, 1H).

### EXAMPLE 99

### tert-Butyl rac-4-[1-(4-cyano-3-trifluoromethyl phenylcarbamoyl)-1-hydroxy-2-phenylethyl]piperidine-1-carboxylate

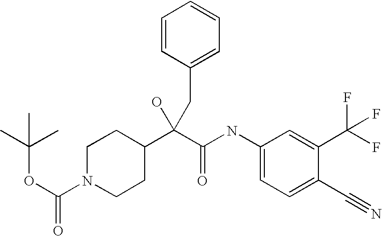

### a) tert-Butyl 4-(acetoxyethoxycarbonylmethylene)piperidine-1-carboxylate

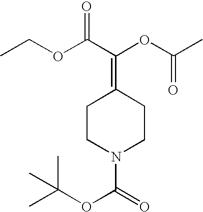

26.15 g of ethyl acetoxy(diethoxyphosphoryl)acetate were initially charged in 130 ml of THF, 3.93 g of lithium chloride were added and the mixture was cooled to 0° C. 12 ml of N,N,N′,N′-tetramethylguanidine were added dropwise and the mixture was stirred for 15 min. Then 14 g of 1-(tert-butyloxycarbonyl)-4-piperidinone, dissolved in 60 ml of THF, were added dropwise. The mixture was allowed to come to room temperature and was stirred for 18 h. The mixture was partitioned between ethyl acetate and water, and the phases were separated and extracted with ethyl acetate. The combined organic phases were dried with sodium sulfate and concentrated. The residue was chromatographed on silica gel. This afforded 22.5 g of the desired intermediate as a colorless oil. ¹H NMR (ppm, CDCl₃, 400 MHz): 1.27 (t, 3H), 1.45 (s, 9H), 2.20 (s, 3H), 2.32 (dt, 2H), 2.91 (dt, 2H), 3.47 (m, 4H), 4.20 (q, 2H).

### b) tert-Butyl 4-oxalylpiperidine-1-carboxylate

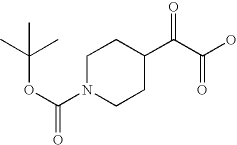

22.5 g of tert-butyl 4-(acetoxyethoxycarbonylmethylene)piperidine-1-carboxylate were added to 140 ml of 1M sodium hydroxide solution in 2:1 ethanol/water. The mixture was left to stir at room temperature for 30 min, then diluted with 1.4 l of cold water, acidified with hydrochloric acid (pH 3-4), extracted with ethyl acetate and with dichloromethane/methanol, then acidified to pH 2-3 and extracted again with dichloromethane/methanol. The combined organic phases were dried with sodium sulfate and concentrated. This afforded 16.9 g of tert-butyl 4-oxalylpiperidine-1-carboxylate as a colorless solid. LCMS: m/z (ES−)=256 (M−1).

### c) tert-Butyl 4-(4-cyano-3-trifluoromethylphenylaminooxalyl)piperidine-1-carboxylate

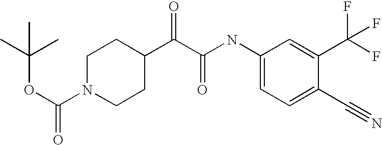

16.9 g of tert-butyl 4-oxalylpiperidine-1-carboxylate were initially charged in 500 ml of dimethylacetamide and 8.6 ml of thionyl chloride were added dropwise at 0° C. After 30 min, 12.23 g of 5-amino-2-cyanobenzotrifluoride were added and the mixture was stirred at RT for 18 h. The mixture was added slowly and with vigorous stirring to 1.8 l of water and stirred for a further 2.5 h. The crystals were filtered off and dried in a vacuum drying cabinet. This afforded 21.5 g of the desired keto amide. ¹H NMR (ppm, d₆-DMSO, 400 MHz): 1.30 (m, 4H), 1.36 (s, 9H), 1.82 (m, 2H), 3.45 (m, 1H), 3.92 (m, 2H), 8.13 (d, 1H), 8.24 (dd, 1H), 8.48 (d, 1H), 11.22 (s, 1H).

### d) tert-Butyl rac-4-[1-(4-cyano-3-trifluoromethylphenylcarbamoyl)-1-hydroxy-2-phenyl-ethyl]piperidine-1-carboxylate

4.2 g of tert-Butyl 4-(4-cyano-3-trifluoromethylphenylaminooxalyl)piperidine-1-carboxylate were initially charged in 129 ml of THF under Ar and, at 0° C., 14 ml of a 2 molar benzylmagnesium bromide solution in THF were added dropwise. The mixture was allowed to thaw to RT and stirred for a further 14 h. The reaction was ended by adding ammonium chloride solution. The mixture was partitioned between ethyl acetate and water, and the phases were separated and extracted with ethyl acetate. The combined organic phases were dried with sodium sulfate and concentrated. Recrystallization from dichloromethane afforded the desired product in approx. 70% yield. ¹H NMR (ppm, CDCl₃, 400 MHz): 1.45 (s, 9H), 1.57 (m, 2H), 1.91 (m, 1H), 2.29 (m, 1H), 2.70 (m, 2H), 2.88 (d, 1H), 3.48 (d, 1H), 4.22 (m, 2H), 4.71 (s, 1H), 7.15 (m, 3H), 7.28 (m, 2H), 7.74 (d, 1H), 7.79 (dd, 1H), 7.88 (d, 1H), 8.63 (s, 1H).

### EXAMPLE 100

### N-(4-Cyano-3-trifluoromethyl phenyl)-2-hydroxy-3-phenyl-2-piperidin-4-yl-propionamide

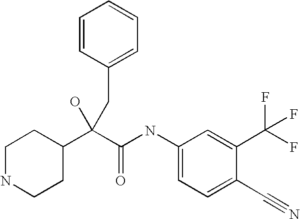

5 g of tert-butyl rac-4-[1-(4-cyano-3-trifluoromethylphenylcarbamoyl)-1-hydroxy-2-phenylethyl]piperidine-1-carboxylate were dissolved in 120 ml of dichloromethane, 20 ml of trifluoroacetic acid were added dropwise and the mixture was stirred for 16 h. The mixture was added to sodium carbonate solution/ice, adjusted to pH 8 with potassium carbonate and extracted with dichloromethane. The organic phases were dried with sodium sulfate and concentrated. This afforded 4.8 g of the racemic product, which was separated into the enantiomers by means of chiral HPLC (Chiralcel OD-H 5μ, 250×20 mm, 9:1 hexane/ethanol).

Example 100a: Rₜ=9.4-11.4 min; [α]^{D}₂₀=+70.50 (MeOH, c=0.46)

Example 100b: Rₜ=12.6-14.3 min; [α]^{D}₂₀=−74.0° (MeOH, c=0.47)

¹H NMR (ppm, d₆-DMSO, 400 MHz): 1.57 (m, 3H), 2.00 (m, 2H), 1.81 (m, 2H), 2.91 (d, 1H), 3.04 (d, 1H), 3.30 (m, 2H), 5.89 (s, 1H), 7.13 (m, 5H), 8.00 (d, 1H), 8.07 (dd, 1H), 8.27 (d, 1H), 10.02 (s, 1H).

### EXAMPLE 101

### 2-(1-Benzoylpiperidin-4-yl)-N-(4-cyano-3-trifluoromethyl phenyl)-2-hydroxy-3-phenylpropionamide

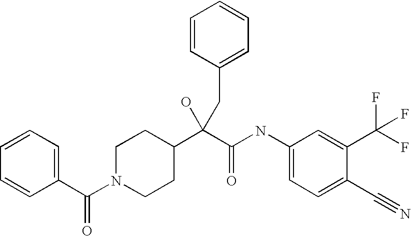

180 mg of rac-N-(4-cyano-3-trifluoromethylphenyl)-2-hydroxy-3-phenyl-2-piperidin-4-ylpropionamide were initially charged at 0° C. in 9 ml of dichloromethane and admixed with 0.12 ml of triethylamine, and 0.06 ml of benzoyl chloride was added dropwise. The mixture was allowed to come to RT and was stirred for approx. 14 h. The reaction was ended by adding sodium hydrogencarbonate solution, the phases were separated, the aqueous phase was extracted with dichloromethane and the combined organic phases were dried with sodium sulfate and concentrated. The crude product was recrystallized from diisopropyl ether/dichloromethane/hexane. The resulting 90 mg of racemate were separated by means of chiral HPLC into the enantiomers (Chiralpak IA 5μ 250×20 mm, 8:2 hexane/ethanol, 25 ml/min).

Example 101a: Rₜ=9.9-11.4 min

Example 101b: Rₜ=11.6-14.2 min

¹H NMR (ppm, CDCl₃, 400 MHz): 1.58 (m, 3H), 2.00 (m, 1H), 2.25 (m, 1H), 2.90 (d, 1H), 2.90 (m, 2H), 3.30 (m, 1H), 3.35 (d, 1H), 3.88 (m, 1H), 7.14 (m, 2H), 7.22 (m, 3H), 7.37 (m, 5H), 7.70 (m, 2H), 7.80 (s, 1H), 8.61 (s, 1H).

Analogously, by reaction of N-(4-cyano-3-trifluoromethylphenyl)-2-hydroxy-3-phenyl-2-piperidin-4-ylpropionamide with the appropriate carbonyl chloride or sulfonyl chloride, the following compound were prepared and, where appropriate, separated into the enantiomers:

| | | | MS (ESI+) |
|---|---|---|---|
| Example | Structure | Name | m/z (M + 1) |
| Example 102 | 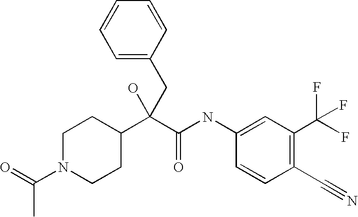 | 2-(1-Acetylpiperidin-4-yl)-N-(4- cyano-3-trifluoromethylphenyl)- 2-hydroxy-3-phenylpropionamide | 460 |
| Example 103 | 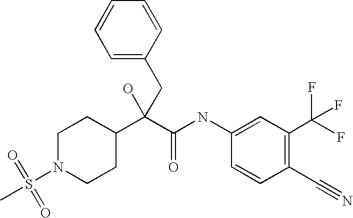 | N-(4-Cyano-3-trifluoromethylphenyl)- 2-hydroxy-2-(1-methanesulfonyl- piperidin-4-yl)-3-phenylpropionamide | 496 |
| Example 104 | 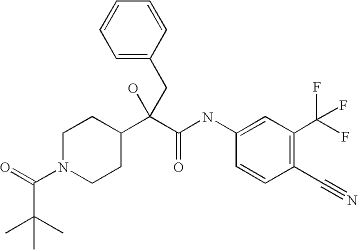 | N-(4-Cyano-3-trifluoromethylphenyl)- 2-[1-(2,2-dimethylpropionyl)- piperidin-4-yl]-2-hydroxy-3-phenyl- propionamide | 502 |
| Example 105 | 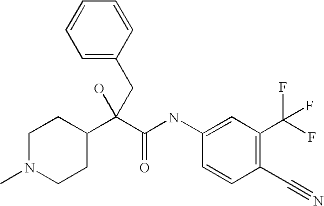 | N-(4-Cyano-3-trifluoromethylphenyl)- 2-hydroxy-2-(1-methylpiperidin-4-yl)- 3-phenylpropionamide | 432 |
| Example 106 | 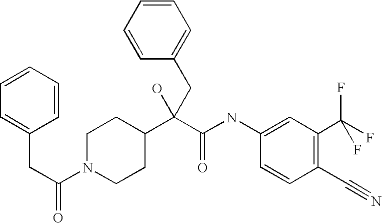 | N-(4-Cyano-3-trifluoromethylphenyl)- 2-hydroxy-3-phenyl-2-(1-phenylacetyl- piperidin-4-yl)propionamide | 536 |
| Example 107 | 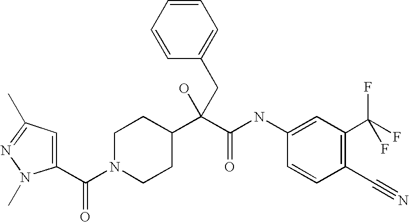 | N-(4-Cyano-3-trifluoromethylphenyl)- 2-[1-(2,5-dimethyl-2H-pyrazole-3- carbonyl)piperidin-4-yl]-2-hydroxy- 3-phenylpropionamide | 540 |
| Example 108 | 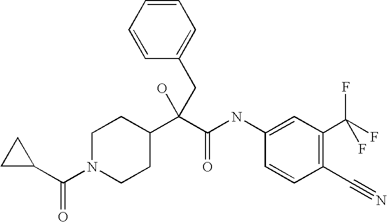 | N-(4-Cyano-3-trifluoromethylphenyl)- 2-(1-cyclopropanecarbonylpiperidin- 4-yl)-2-hydroxy-3-phenylpropion amide | 486 |
| Example 109 | 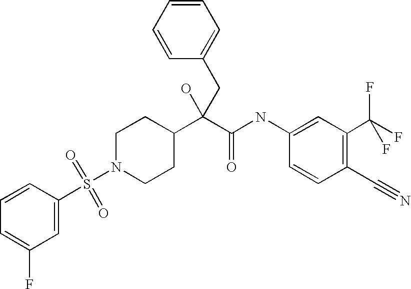 | N-(4-Cyano-3-trifluoromethylphenyl)- 2-[1-(3-fluorobenzenesulfonyl)- piperidin-4-yl]-2-hydroxy-3-phenyl- propionamide | 576 |
| Example 110 | 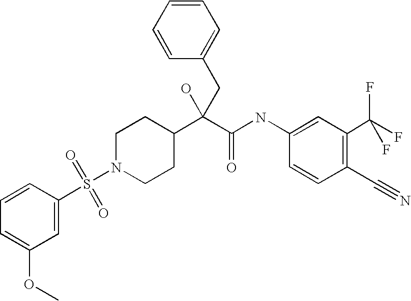 | N-(4-Cyano-3-trifluoromethylphenyl)- 2-hydroxy-2-[1-(3-methoxybenzene- sulfonyl)piperidin-4-yl]-3-phenyl- propionamide | 588 |
| Example 111 | 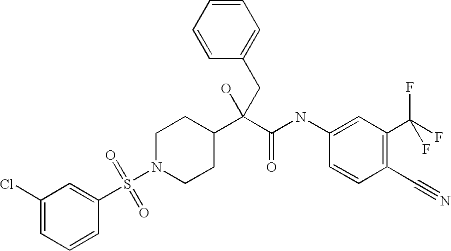 | 2-[1-(3-Chlorobenzenesulfonyl)- piperidin-4-yl]-N-(4-cyano-3-trifluoro- methylphenyl)-2-hydroxy-3-phenyl- propionamide | 592 |
| Example 112 | 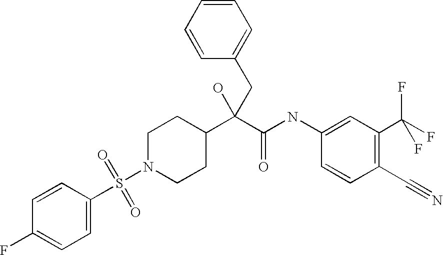 | N-(4-Cyano-3-trifluoromethylphenyl)- 2-[1-(4-fluorobenzenesulfonyl)- piperidin-4-yl]-2-hydroxy-3-phenyl- propionamide | 576 |
| Example 113 | 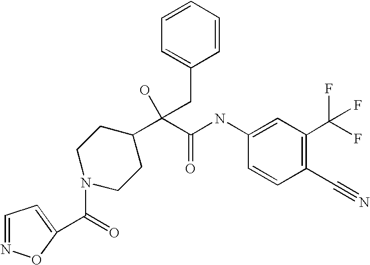 | N-(4-Cyano-3-trifluoromethylphenyl)- 2-hydroxy-2-[1-(isoxazole-5- carbonyl)piperidin-4-yl]-3-phenyl- propionamide | 513 |
| Example 114 | 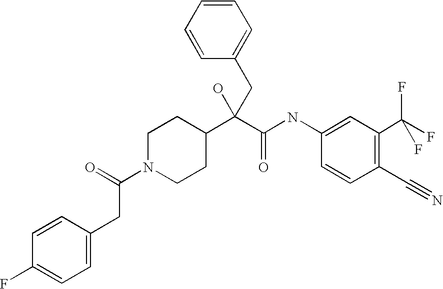 | N-(4-Cyano-3-trifluoromethylphenyl)- 2-{1-[2-(4-fluorophenyl)acetyl]- piperidin-4-yl}-2-hydroxy-3-phenyl- propionamide | 554 |
| Example 115 | 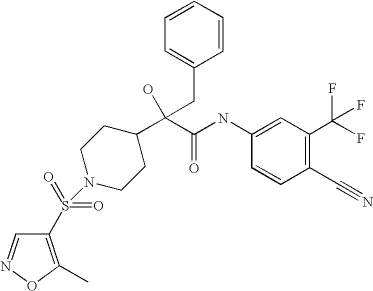 | N-(4-Cyano-3-trifluoromethylphenyl)- 2-hydroxy-2-[1-(5-methylisoxazole- 4-sulfonyl)piperidin-4-yl]-3-phenyl- propionamide | 563 |
| Example 116 | 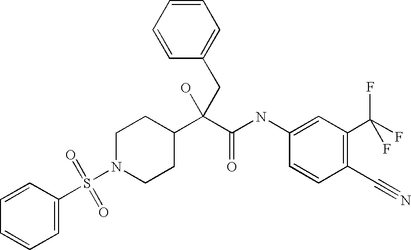 | 2-(1-Benzenesulfonylpiperidin-4-yl)- N-(4-cyano-3-trifluoromethylphenyl)- 2-hydroxy-3-phenylpropionamide | 558 |
| Example 117 | 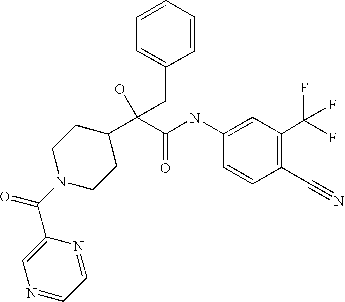 | N-(4-Cyano-3-trifluoromethylphenyl)- 2-hydroxy-3-phenyl-2-[1-(pyrazine- 2-carbonyl)piperidin-4-yl]propion- amide | 524 |
| Example 118 | 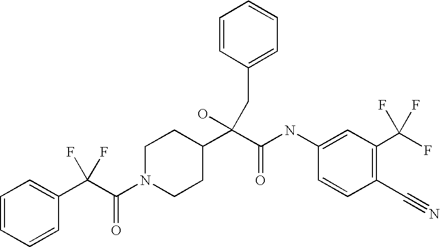 | N-(4-Cyano-3-trifluoromethylphenyl)- 2-[1-(2,2-difluoro-2-phenylacetyl)- piperidin-4-yl]-2-hydroxy-3-phenyl- propionamide | 572 |
| Example 119 | 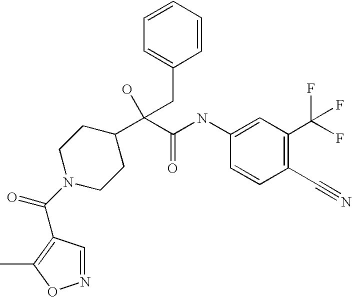 | N-(4-Cyano-3-trifluoromethylphenyl)- 2-hydroxy-2-[1-(5-methylisoxazole- 4-carbonyl)piperidin-4-yl]-3-phenyl- propionamide | 527 |
| Example 120 | 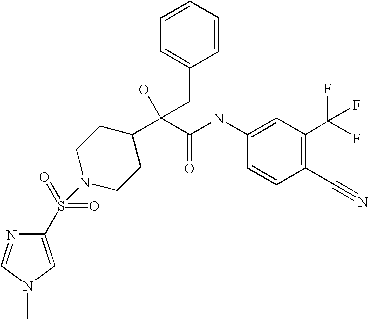 | N-(4-Cyano-3-trifluoromethylphenyl)- 2-hydroxy-2-[1-(1-methyl-1H-imid- azole-4-sulfonyl)piperidin-4-yl]-3- phenylpropionamide | 562 |
| Example 121 | 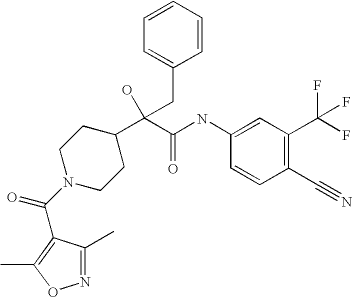 | N-(4-Cyano-3-trifluoromethylphenyl)- 2-[1-(3,5-dimethylisoxazole-4- carbonyl)piperidin-4-yl]-2-hydroxy-3- phenylpropionamide | 541 |
| Example 122 | 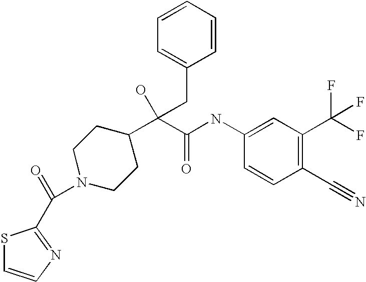 | N-(4-Cyano-3-trifluoromethylphenyl)- 2-hydroxy-3-phenyl-2-[1-(thiazole- 2-carbonyl)piperidin-4-yl]propion- amide | 529 |
| Example 123 | 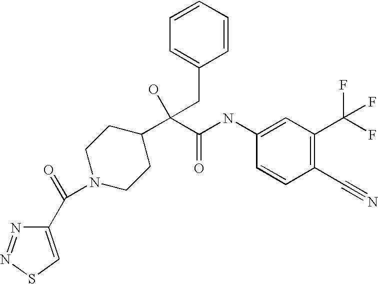 | N-(4-Cyano-3-trifluoromethylphenyl)- 2-hydroxy-3-phenyl-2-[1-([1,2,3]- thiadiazole-4-carbonyl)piperidin-4- yl]propionamide | 530 |
| Example 124 | 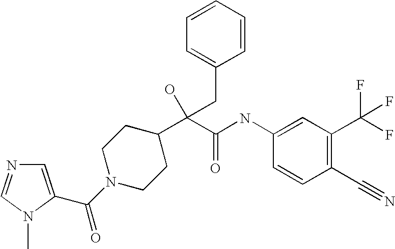 | N-(4-Cyano-3-trifluoromethylphenyl)- 2-hydroxy-2-[1-(3-methyl-3H-imid- azole-4-carbonyl)piperidin-4-yl]-3- phenylpropionamide | 526 |
| Example 125 | 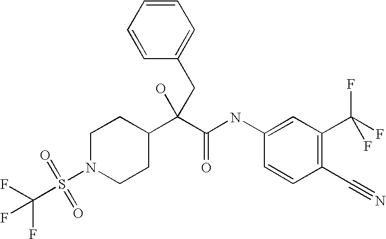 | N-(4-Cyano-3-trifluoromethylphenyl)- 2-hydroxy-3-phenyl-2-(1-trifluoro- methanesulfonylpiperidin-4-yl) propionamide | 550 |
| Example 126 | 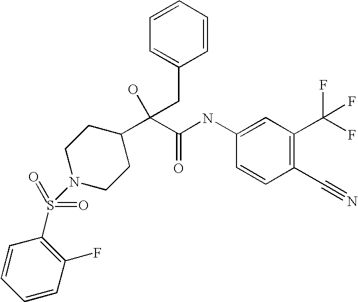 | N-(4-Cyano-3-trifluoromethylphenyl)- 2-[1-(2-fluorobenzenesulfonyl)- piperidin-4-yl]-2-hydroxy-3-phenyl- propionamide | 576 |

### EXAMPLE 127

### tert-Butyl 4-[1-(4-cyano-3-trifluoromethyl phenylcarbamoyl)-1-hydroxy-2-(3-iodophenyl)ethyl]piperidine-1-carboxylate

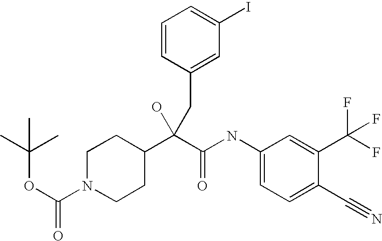

3.5 g of tert-butyl 4-(4-cyano-3-trifluoromethylphenylaminooxalyl)piperidine-1-carboxylate (for preparation see above) were dissolved under Ar at −75° C. in 150 ml of THF, then 50 ml of a 0.5 M solution of 3-iodobenzylzinc bromide in THF were added dropwise and the mixture was thawed overnight. The reaction was ended with ammonium chloride solution and diluted with ethyl acetate, the phases were separated, the aqueous phase was extracted with ethyl acetate, and the organic phases were washed with NaCl solution and dried with sodium sulfate. The crude product was purified by chromatography. This afforded 2.32 g of a pale yellowish solid, which was separated into the enantiomers by means of chiral HPLC (Chiralpak IA 5μ, 250×30 mm, 9:1 hexane/ethanol, 40 ml/min)

Example 127a: Rₜ=8.6-9.5 min; [α]^{D}₂₀=−38.0° (MeOH, c=1.03)

Example 127b: Rₜ=9.6-10.7 min; [α]^{D}₂₀=+19.4° (MeOH, c=0.48)

¹H NMR (ppm, d₆-DMSO, 400 MHz): 1.35 (s, 9H), 1.38 (m, 3H), 1.80 (m, 1H), 1.95 (m, 1H), 2.58 (m, 2H), 2.83 (d, 1H), 2.97 (d, 1H), 3.98 (m, 2H), 5.72 (s, 1H), 6.93 (m, 1H), 7.15 (d, 1H), 7.40 (d, 1H), 7.54 (s, 1H), 7.99 (d, 1H), 8.04 (d, 1H), 8.26 (s, 1H), 9.97 (s, 1H).

Analogously, by reaction with 2-iodobenzylzinc bromide or 4-iodobenzylzinc bromide, the corresponding ortho- and para-iodo compounds can be obtained:

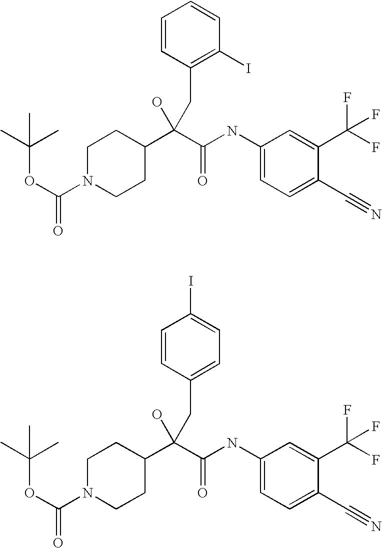

### EXAMPLE 130

### N-(4-Cyano-3-trifluoromethyl phenyl)-2-hydroxy-3-(3-iodophenyl)-2-piperidin-4-ylpropionamide

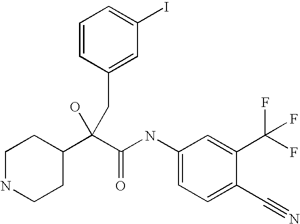

The reaction was effected analogously to example 100. One hour of reaction time was sufficient.

LCMS: m/z=544 (ES+, M+1); 542 (ES−, M−1)

The corresponding 2-iodo and 4-iodo compounds are obtained analogously.

### EXAMPLE 131

### 2-(1-Benzoylpiperidin-4-yl)-N-(4-cyano-3-trifluoromethyl phenyl)-2-hydroxy-3-(3-iodophenyl)propionamide

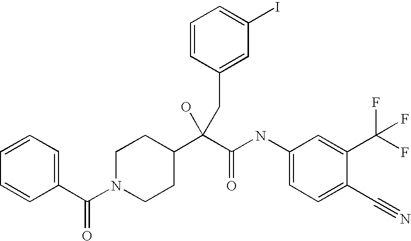

The reaction was effected analogously to example 101. 1.77 g of amine afforded 1.9 g (90%) of the desired product. 1.1 equivalents of benzoyl chloride were used.

LCMS: m/z=648 (ES+, M+1); 646 (ES−, M−1)

### EXAMPLE 132

### 3′-[2-(1-Benzoylpiperidin-4-yl)-2-(4-cyano-3-trifluoromethyl phenylcarbamoyl)-2-hydroxyethyl]biphenyl-3-carboxamide

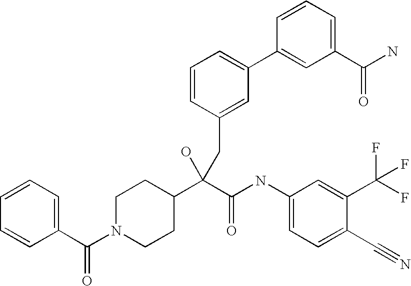

100 mg of 2-(1-benzoylpiperidin-4-yl)-N-(4-cyano-3-trifluoromethylphenyl)-2-hydroxy-3-(3-iodophenyl)propionamide and 26 mg of (3-aminocarbonylphenyl)boronic acid were initially charged in 2 ml of 1:1 toluene/ethanol, admixed with 0.15 ml of 2M sodium carbonate solution and 18 mg of tetrakis(triphenylphosphine)palladium and irradiated in a microwave at 150 W/120° C. for 30 min. Thereafter, the mixture was partitioned between water and ethyl acetate, filtered together and then the phases were separated. The aqueous phase was extracted with ethyl acetate, and the organic phases were washed with sat. NaCl solution, dried with sodium sulfate and concentrated. The crude product was purified by chromatography. This afforded 37 mg (38%) of the desired product as a colorless solid.

¹H NMR (ppm, CDCl₃, 400 MHz): 1.45-1.70 (m, 3H), 1.83-2.02 (m, 1H), 2.29-2.39 (m, 1H), 2.73-2.87 (m, 1H), 2.98 (d, 1H), 2.96-3.09 (m, 1H), 3.18 (d, 1H), 3.75-3.90 (m, 1H), 4.72-4.89 (m, 1H), 6.21 (s br, 1H), 6.77 (s br, 1H), 7.12-7.20 (m, 2H), 7.36 (m, 5H), 7.39-7.44 (m, 1H), 7.48-7.72 (m, 6H), 7.78 (s, 1H), 8.05 (s, 1H), 8.85 (s, 1H).

LCMS: m/z=641 (ES+, M+1); 639 (ES−, M−1)

By reaction of N-(4-cyano-3-trifluoromethylphenyl)-2-hydroxy-3-(3-iodophenyl)-2-piperidin-4-ylpropionamide (example 130) with a multitude of carbonyl chlorides and sulfonyl chlorides, it was possible using the process described in example 131 to prepare appropriate starting compounds for Suzuki reactions, which were converted analogously to example 132. Alternatively, a changeover of the reaction sequence was also possible. In this case, the Suzuki reaction (analogously to example 132) was carried out on the Boc-protected intermediate (analogously to example 127), which was followed by Boc-deprotection (analogously to example 130) and acylation or sulfonamide formation analogously to example 131. The enantiomers, which were pure in each case, were obtained by preparative chiral HPLC of the end compound or by performing the reaction sequence with material already separated at the stage of example 127.

The following further example compounds were prepared.

| | | | MS |
|---|---|---|---|
| | | | (ESI+) |
| | | | m/z |
| Example | Structure | Name | (M + 1) |
| Example 133 | 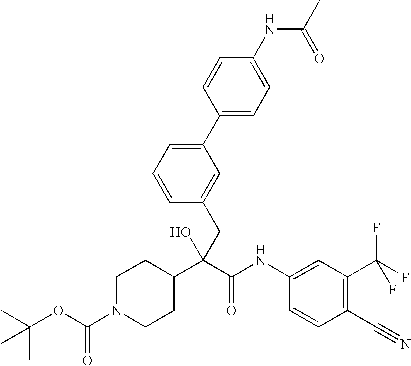 | tert-Butyl 4-[2-(4′-acetylamino- biphenyl-3-yl)-1-(4-cyano-3-trifluoro- methylphenylcarbamoyl)-1-hydroxy- ethyl]piperidine-1-carboxylate | 651 |
| Example 134 | 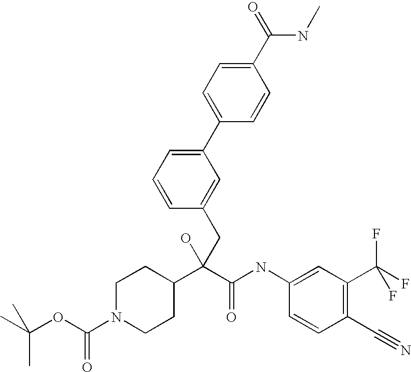 | tert-Butyl 4-[1-(4-cyano-3-trifluoromethyl- phenylcarbamoyl)-1-hydroxy-2-(4′- methylcarbamoylbiphenyl-3-yl)ethyl]- piperidine-1-carboxylate | 651 |
| Example 135 | 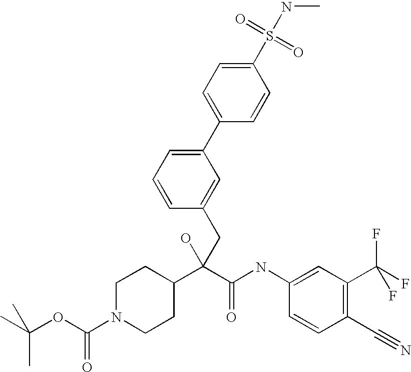 | tert-Butyl 4-[1-(4-Cyano-3-trifluoro- methylphenylcarbamoyl)-1-hydroxy- 2-(4′-methylsulfamoyl-biphenyl-3-yl)- ethyl]piperidine-1-carboxylate | 687 |
| Example 136 | 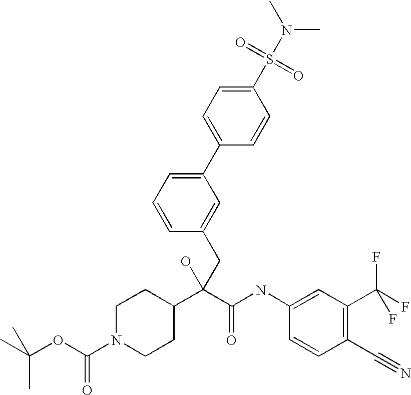 | tert-Butyl 4-[1-(4-cyano-3-trifluoro- methylphenylcarbamoyl)-2-(4′- dimethylsulfamoylbiphenyl-3-yl)- 1-hydroxyethyl]piperidin-1-carboxylate | 701 |
| Example 137 | 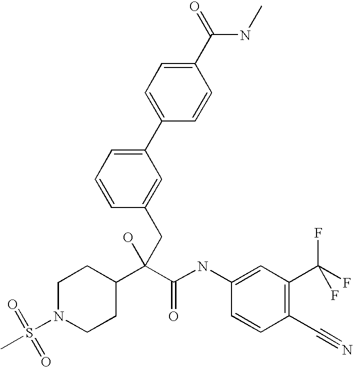 | N-Methyl-3′-[2-(4-cyano-3-trifluoro- methylphenylcarbamoyl)-2-hydroxy- 2-(1-methanesulfonyl-piperidin-4-yl)- ethyl]biphenyl-4-carboxamide | 629 |
| Example 138 | 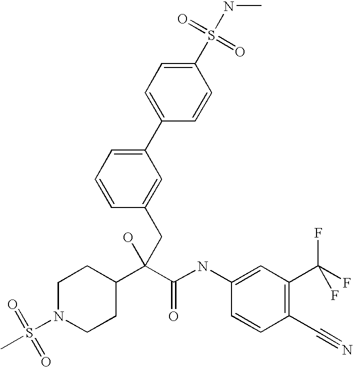 | N-(4-Cyano-3-trifluoromethylphenyl)- 2-hydroxy-2-(1-methanesulfonylpiperidin- 4-yl)-3-(4′-methylsulfamoylbiphenyl- 3-yl)propionamide | 665 |
| Example 139 | 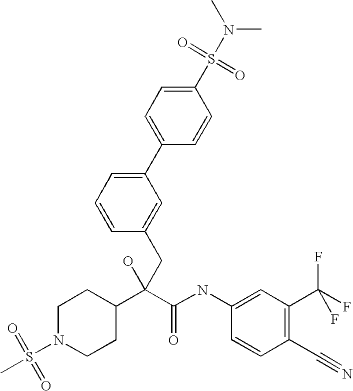 | N-(4-Cyano-3-trifluoromethylphenyl)- 3-(4′-dimethylsulfamoylbiphenyl- 3-yl)-2-hydroxy-2-(1-methanesulfonyl- piperidin-4-yl)propionamide | 679 |
| Example 140 | 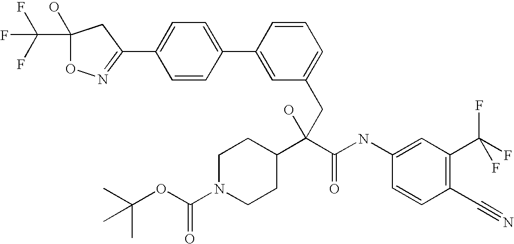 | tert-Butyl 4-{1-(4-cyano-3-trifluoromethyl- phenylcarbamoyl)-1-hydroxy-2-(4′-(5- hydroxy-5-trifluoromethyl-4,5-dihydro- isoxazol-3-yl)biphenyl-3- yl]ethyl}piperidine-1-carboxylate | 747 |
| Example 141 | 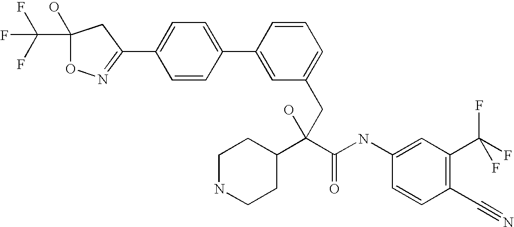 | N-(4-Cyano-3-trifluoromethylphenyl)- 2-hydroxy-3-[4′-(5-hydroxy-5-trifluoro- methyl-4,5-dihydroisoxazol-3-yl)- biphenyl-3-yl]-2-piperidin-4-yl- propionamide | 647 |
| Example 142 | 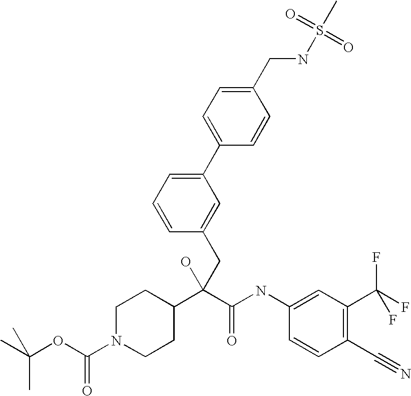 | tert-Butyl 4-{1-(4-cyano-3-trifluoromethyl- phenylcarbamoyl)-1-hydroxy-2-[4′- (methane-sulfonylaminomethyl)biphenyl- 3-yl]ethyl}-piperidine-1-carboxylate | 701 |
| Example 143 | 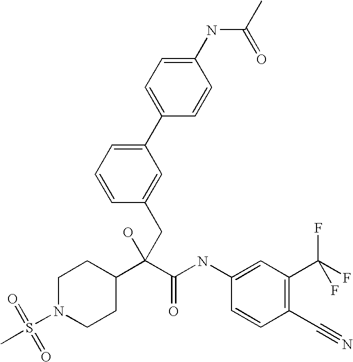 | 3-(4′-Acetylaminobiphenyl-3-yl)-N- (4-cyano-3-trifluoromethylphenyl)- 2-hydroxy-2-(1-methanesulfonyl- piperidin-4-yl)propionamide | 629 |
| Example 144 | 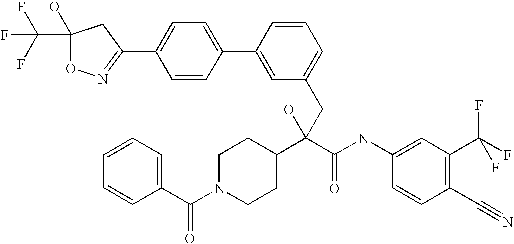 | 2-(1-Benzoylpiperidin-4-yl)-N-(4- cyano-3-trifluoromethylphenyl)-2- hydroxy-3-[4′-(5-hydroxy-5-trifluoro- methyl-4,5-dihydroisoxazol-3-yl)- biphenyl-3-yl]propionamide | 751 |
| Example 145 | 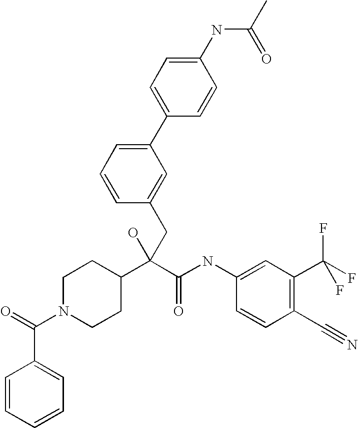 | 3-(4′-Acetylaminobiphenyl-3-yl)-2- (1-benzoylpiperidin-4-yl)-N-(4-cyano- 3-trifluoromethylphenyl)-2-hydroxy- propionamide | 655 |
| Example 146 | 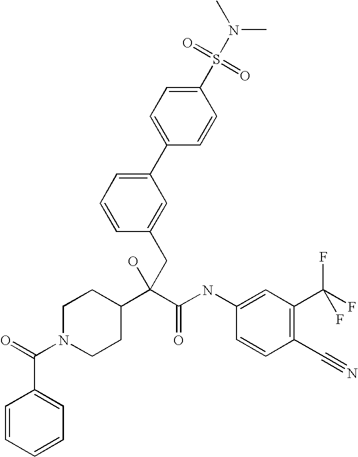 | 2-(1-Benzoylpiperidin-4-yl)-N-(4- cyano-3-trifluoromethylphenyl)-3- (4′-dimethylsulfamoylbiphenyl-3- yl)-2-hydroxypropionamide | 705 |
| Example 147 | 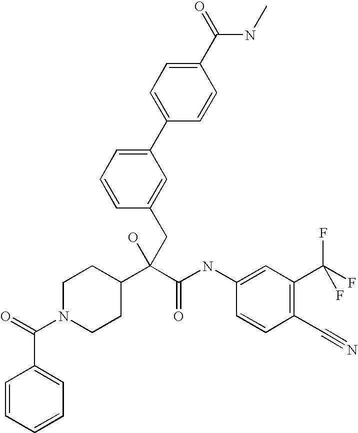 | N-Methyl-3′-[2-(1-benzoylpiperidin- 4-yl)-2-(4-cyano-3-trifluormethyl- phenylcarbamoyl)-2-hydroxyethyl]- biphenyl-4-carboxamide | 655 |
| Example 148 | 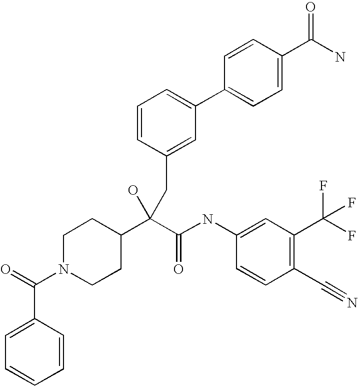 | 3′-[2-(1-Benzoylpiperidin-4-yl)-2- (4-cyano-3-trifluoromethylphenyl- carbamoyl)-2-hydroxyethyl]biphenyl- 4-carboxamide | 641 |
| Example 149 | 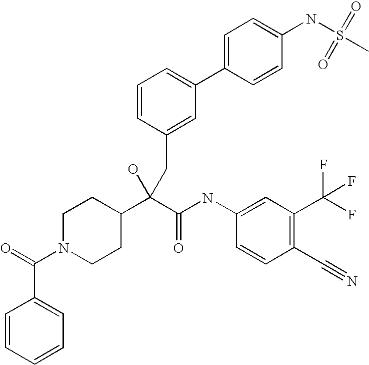 | 2-(1-Benzoylpiperidin-4-yl)-N-(4- cyano-3-trifluoromethylphenyl)-2- hydroxy-3-(4-methanesulfonylamino- biphenyl-3-yl)propionamide | 691 |
| Example 150 | 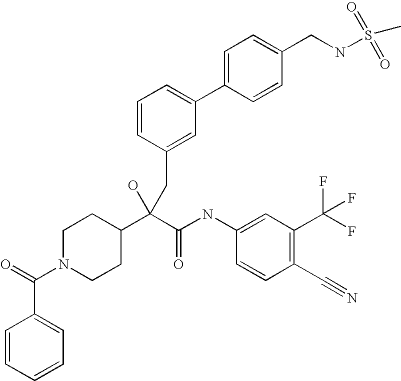 | 2-(1-Benzoylpiperidin-4-yl)-N-(4- cyano-3-trifluoromethylphenyl)-2- hydroxy-3-[4′-(methanesulfonylamino- methyl)biphenyl-3-yl]propionamide | 705 |
| Example 151 | 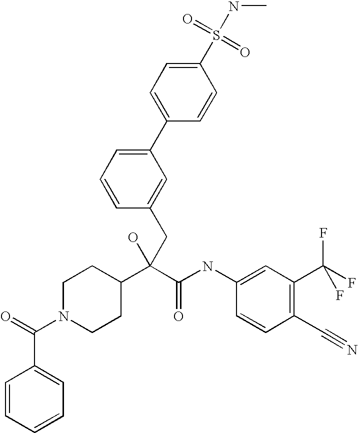 | 2-(1-Benzoylpiperidin-4-yl)-N-(4- cyano-3-trifluoromethylphenyl)-2- hydroxy-3-(4′-methylsulfamoyl- biphenyl-3-yl)propionamide | 691 |
| Example 152 | 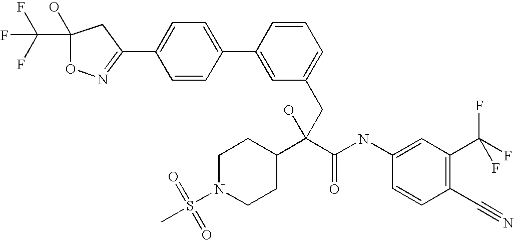 | N-(4-Cyano-3-trifluoromethylphenyl)- 2-hydroxy-3-[4′-(5-hydroxy-5-trifluoro- methyl-4,5-dihydroisoxazol-3-yl)- biphenyl-3-yl]-2-(1-methanesulfonyl- piperidin-4-yl)propionamide | 725 |
| Example 153 | 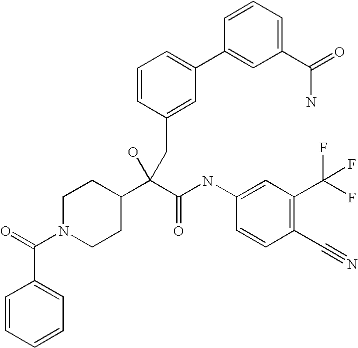 | 3′-[2-(1-Benzoylpiperidin-4-yl)-2- (4-cyano-3-trifluoromethylphenyl- carbamoyl)-2-hydroxyethyl]- biphenyl-3-carboxamide | 641 |
| Example 154 | 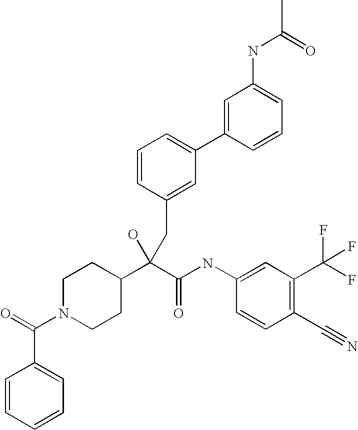 | 3-(3′-Acetylaminobiphenyl-3-yl)-2- (1-benzoylpiperidin-4-yl)-N-(4- cyano-3-trifluoromethylphenyl)-2- hydroxypropionamide | 655 |
| Example 155 | 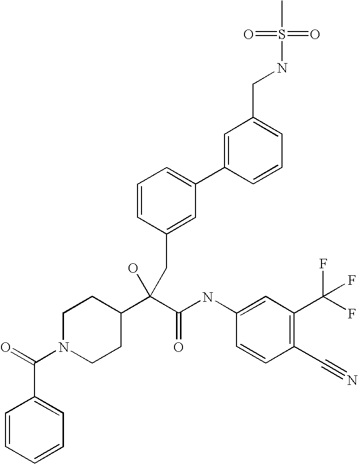 | 2-(1-Benzoylpiperidin-4-yl)-N-(4- cyano-3-trifluoromethylphenyl)-2- hydroxy-3-[3′-(methanesulfonylamino- methyl)biphenyl-3-yl]propionamide | 705 |
| Example 156 | 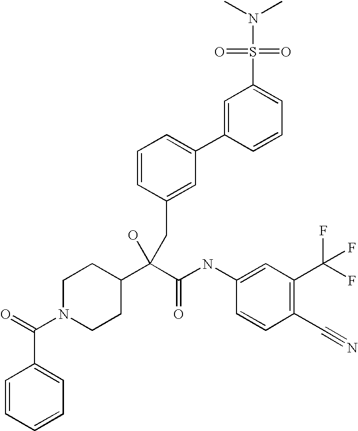 | 2-(1-Benzoylpiperidin-4-yl)-N-(4- cyano-3-trifluoromethylphenyl)-3- (3′-dimethylsulfamoylbiphenyl-3- yl)-2-hydroxypropionamide | 705 |
| Example 157 | 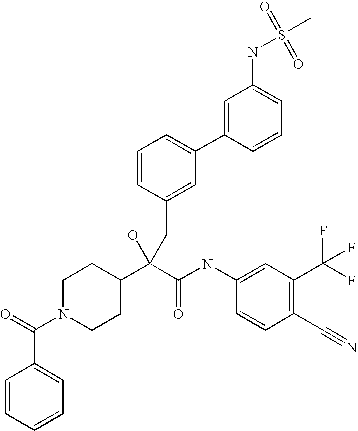 | 2-(1-Benzoylpiperidin-4-yl)-N-(4- cyano-3-trifluoromethylphenyl)-2- hydroxy-3-(3′-methanesulfonylamino- biphenyl-3-yl)propionamide | 691 |
| Example 158 | 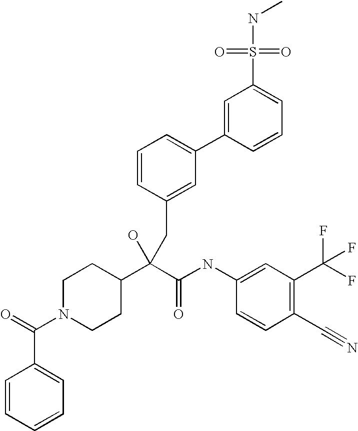 | 2-(1-Benzoylpiperidin-4-yl)-N-(4- cyano-3-trifluoromethylphenyl)-2- hydroxy-3-(3′-methylsulfamoyl- biphenyl-3-yl)propionamide | 691 |
| Example 159 | 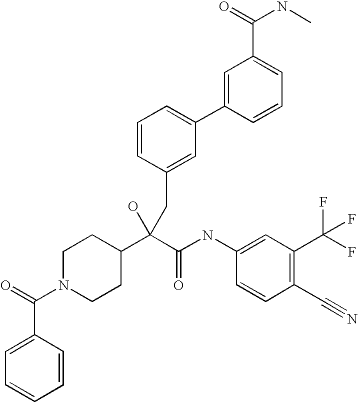 | N-Methyl-3′-[2-(1-benzoylpiperidin-4-yl)- 2-(4-cyano-3-trifluoromethylphenyl- carbamoyl)-2-hydroxyethyl]biphenyl- 3-carboxamide | 655 |
| Example 160 | 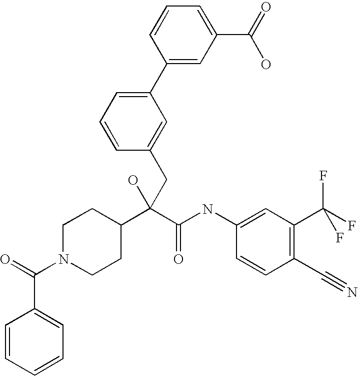 | 3′-[2-(1-Benzoylpiperidin-4-yl)-2- (4-cyano-3-trifluoromethylphenyl- carbamoyl)-2-hydroxyethyl]- biphenyl-3-carboxylic acid | 642 |
| Example 161 | 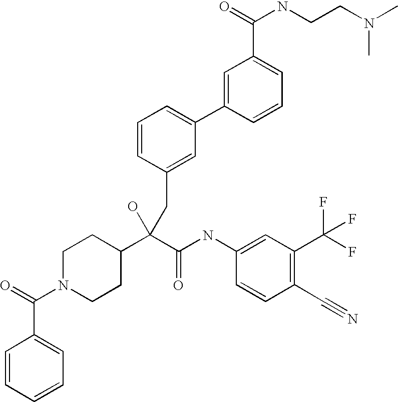 | N-(2-Dimethylaminoethyl)-3′-[2- (1-benzoylpiperidin-4-yl)-2- (4-cyano-3-trifluoromethylphenyl- carbamoyl)-2-hydroxyethyl]- biphenyl-3-carboxamide | 712 |
| Example 162 | 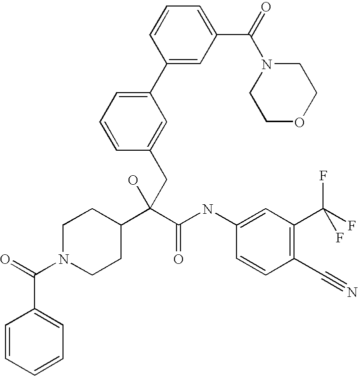 | 2-(1-Benzoylpiperidin-4-yl)-N-(4- cyano-3-trifluoromethylphenyl)-2- hydroxy-3-[3′-(morpholine-4-carbonyl)- biphenyl-3-yl]propionamide | 711 |
| Example 163 | 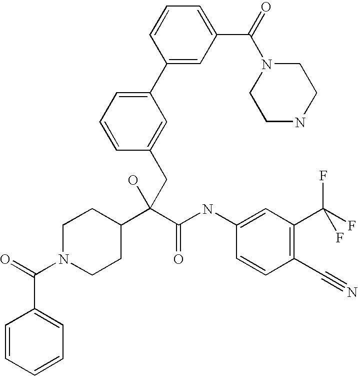 | 2-(1-Benzoylpiperidin-4-yl)-N-(4- cyano-3-trifluoromethylphenyl)-2- hydroxy-3-[3′-(piperazine-1-carbonyl)- biphenyl-3-yl]propionamide | 710 |
| Example 164 | 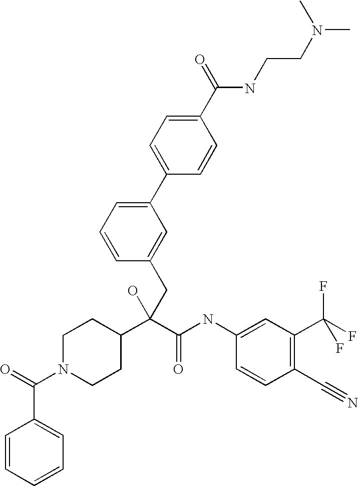 | N-(2-Dimethylaminoethyl)-3′-[2- (1-benzoylpiperidin-4-yl)-2- (4-cyano-3-trifluoromethylphenyl- carbamoyl)-2-hydroxyethyl]biphenyl- 4-carboxamide | 712 |
| Example 165 | 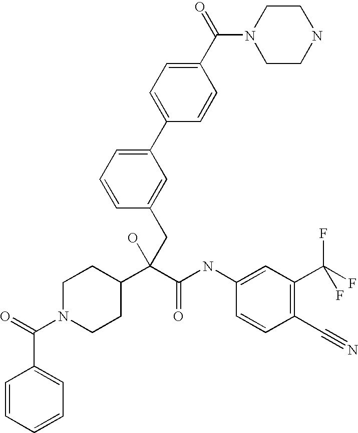 | 2-(1-Benzoylpiperidin-4-yl)-N-(4- cyano-3-trifluoromethylphenyl)-2- hydroxy-3-[4′-(piperazine-1-carbonyl)- biphenyl-3-yl]propionamide | 710 |
| Example 166 | 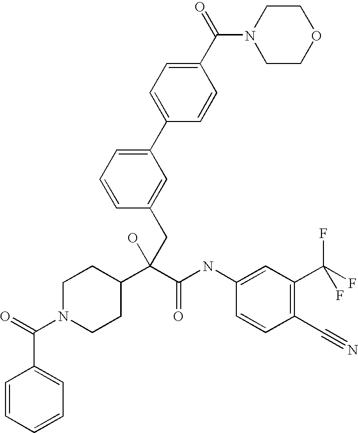 | 2-(1-Benzoylpiperidin-4-yl)-N-(4- cyano-3-trifluoromethylphenyl)-2- hydroxy-3-[4′-(morpholine-4-carbonyl)- biphenyl-3-yl]propionamide | 711 |
| Example 167 | 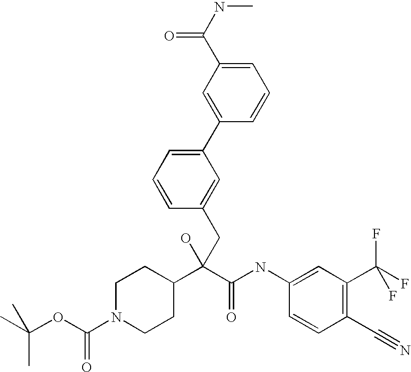 | tert-Butyl 4-[1-(4-cyano-3-trifluoro- methylphenylcarbamoyl)-1-hydroxy- 2-(3′-methylcarbamoylbiphenyl-3- yl)ethyl]piperidine-1-carboxylate | 651 |
| Example 168 | 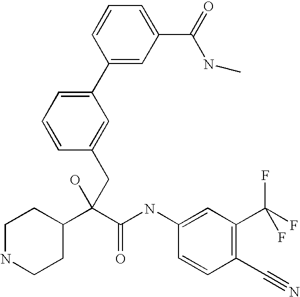 | N-Methyl-3′-[2-(4-cyano-3-trifluoro- methylphenylcarbamoyl)-2-hydroxy- 2-piperidin-4-ylethyl]biphenyl- 3-carboxamide | 551 |
| Example 169 | 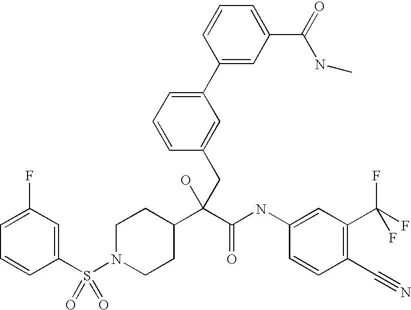 | N-Methyl-3′-{2-(4-cyano-3-trifluoro- methylphenylcarbamoyl)-2-[1-(3-fluoro- benzenesulfonyl)-piperidin-4-yl]-2- hydroxyethyl}biphenyl-3-carboxamide | 709 |
| Example 170 | 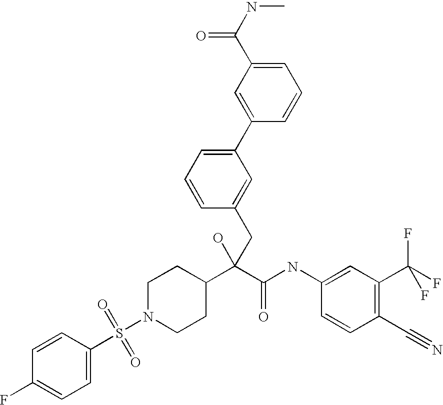 | N-Methyl-3′-{2-(4-cyano-3-trifluoro- methylphenylcarbamoyl)-2-[1-(4-fluoro- benzenesulfonyl)-piperidin-4-yl]-2- hydroxyethyl}biphenyl-3-carboxamide | 709 |
| Example 171 | 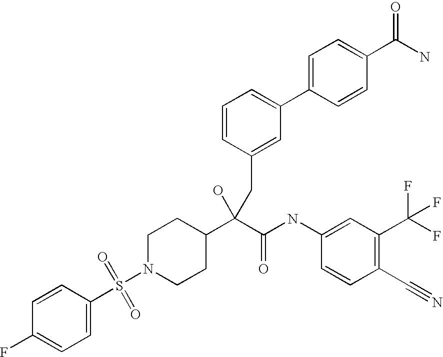 | 3′-{2-(4-Cyano-3-trifluoromethyl- phenylcarbamoyl)-2-[1-(4-fluoro- benzenesulfonyl)piperidin-4-yl]-2- hydroxyethyl}biphenyl-4- carboxamide | 695 |
| Example 172 | 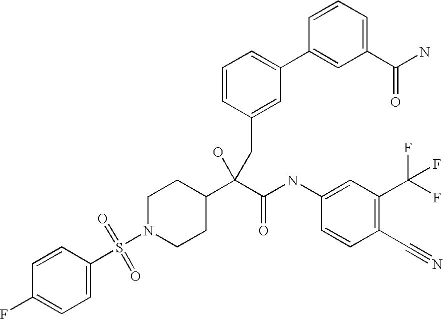 | 3′-{2-(4-Cyano-3-trifluoromethyl- phenylcarbamoyl)-2-[1-(4-fluoro- benzenesulfonyl)piperidin-4-yl]-2- hydroxyethyl}biphenyl-3- carboxamide | 695 |
| Example 173 | 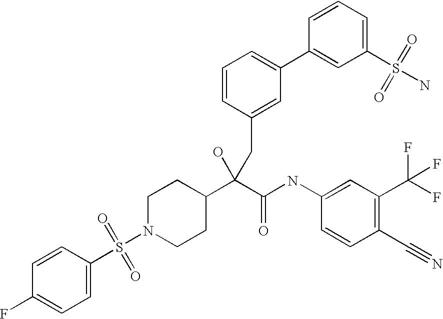 | N-(4-Cyano-3-trifluoromethyl- phenyl)-2-[1-(4-fluorobenzene- sulfonyl)piperidin-4-yl]-2- hydroxy-3-(3′-sulfamoylbiphenyl- 3-yl)propionamide | 731 |
| Example 174 | 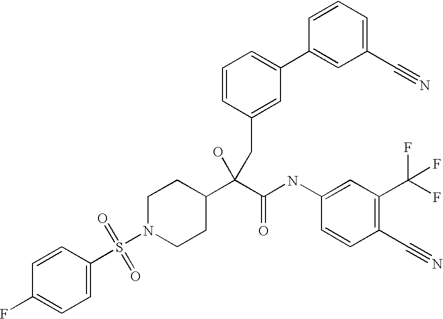 | 3-(3′-Cyanobiphenyl-3-yl)-N-(4- cyano-3-trifluoromethylphenyl)-2- [1-(4-fluorobenzenesulfonyl)piperidin- 4-yl]-2-hydroxypropionamide | 677 |
| Example 175 | 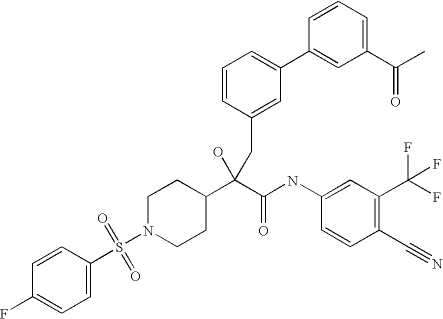 | 3-(3′-Acetylbiphenyl-3-yl)-N-(4- cyano-3-trifluoromethylphenyl)-2- [1-(4-fluorobenzenesulfonyl)- piperidin-4-yl]-2-hydroxypropionamide | 694 |
| Example 176 | 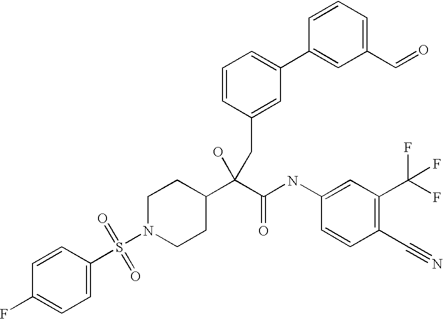 | N-(4-Cyano-3-trifluoromethyl- phenyl)-2-[1-(4-fluorobenzene- sulfonyl)piperidin-4-yl]-3-(3′- formylbiphenyl-3-yl)-2- hydroxypropionamide | 680 |
| Example 177 | 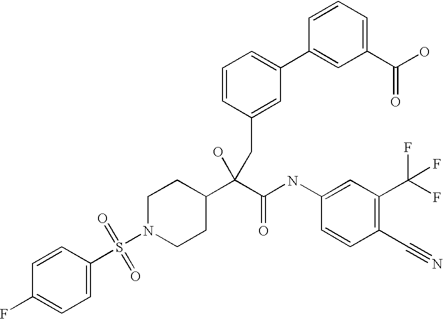 | 3′-{2-(4-Cyano-3-trifluoromethyl- phenylcarbamoyl)-2-[1-(4-fluoro- benzenesulfonyl)-piperidin-4-yl]-2- hydroxyethyl}biphenyl-3-carboxylic acid | 696 |
| Example 178 | 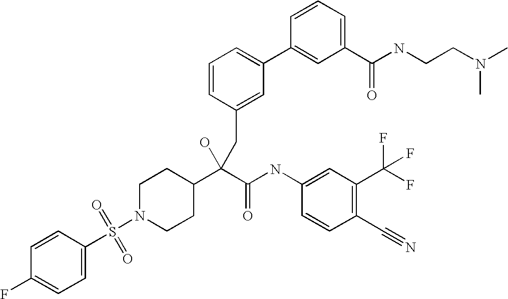 | N-(2-Dimethylaminoethyl)-3′-{2- (4-cyano-3-trifluoromethylphenyl- carbamoyl)-2-[1-(4-fluorobenzene- sulfonyl)piperidin-4-yl]-2-hydroxy- ethyl}biphenyl-3-carboxamide | 766 |
| Example 179 | 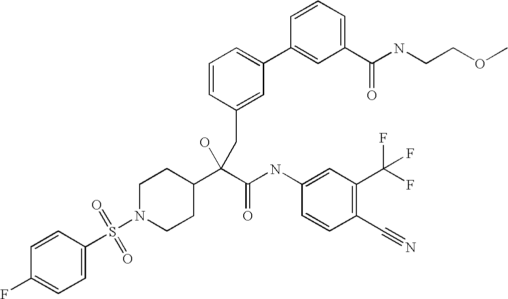 | N-(2-Methoxyethyl)-3′-{2-(4-cyano- 3-trifluoromethylphenylcarbamoyl)-2- [1-(4-fluorobenzenesulfonyl)-piperidin- 4-yl]-2-hydroxyethyl}biphenyl-3- carboxamide | 753 |
| Example 180 | 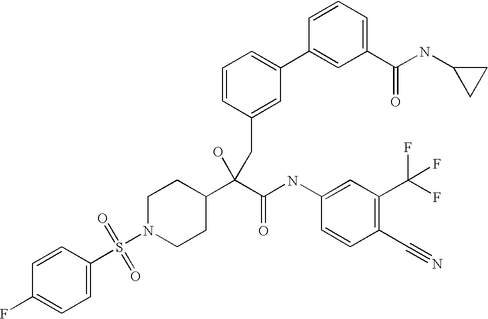 | N-Cyclopropyl-3′-{2-(4-cyano-3-trifluoro- methylphenylcarbamoyl)-2-[1-(4-fluoro- benzenesulfonyl)piperidin-4-yl]-2- hydroxyethyl}biphenyl-3-carboxamide | 735 |
| Example 181 | 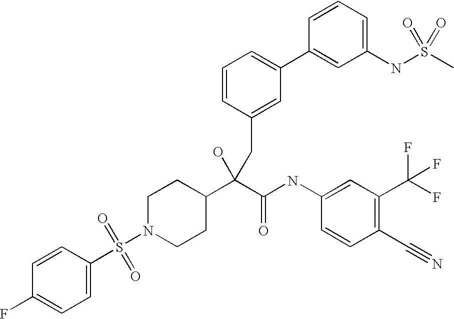 | N-(4-Cyano-3-trifluoromethylphenyl)- 2-[1-(4-fluorobenzenesulfonyl)- piperidin-4-yl]-2-hydroxy-3-(3′- methanesulfonylaminobiphenyl-3-yl)- propanamide | 745 |
| Example 182 | 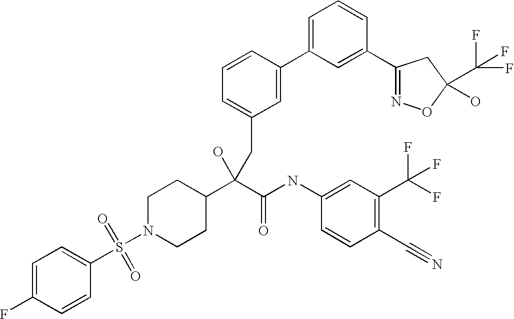 | N-(4-Cyano-3-trifluoromethylphenyl)- 2-[1-(4-fluorobenzenesulfonyl)piperidin- 4-yl]-2-hydroxy-3-[3′-(5-hydroxy-5- trifluoromethyl-4,5-dihydroisoxazol-3- yl)biphenyl-3-yl]propionamide | 805 |
| Example 183 | 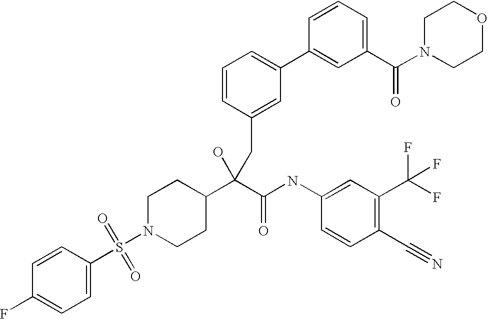 | N-(4-Cyano-3-trifluoromethylphenyl)- 2-[1-(4-fluorobenzenesulfonyl)piperidin- 4-yl]-2-hydroxy-3-[3′-(morpholine-4- carbonyl)biphenyl-3-yl]propionamide | 765 |
| Example 184 | 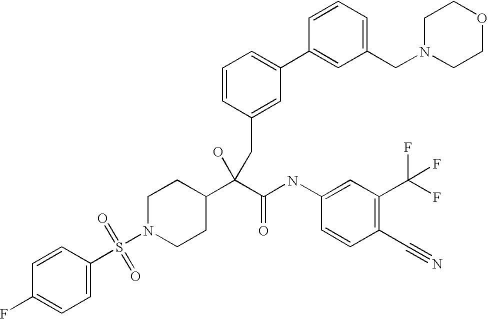 | N-(4-Cyano-3-trifluoromethylphenyl)- 2-[1-(4-fluorobenzenesulfonyl)piperidin- 4-yl]-2-hydroxy-3-(3′-morpholin-4- ylmethylbiphenyl-3-yl)propionamide | 751 |
| Example 185 | 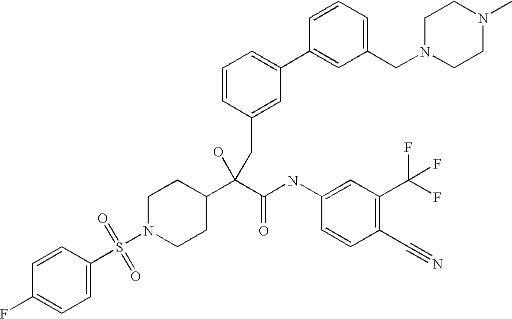 | N-(4-Cyano-3-trifluoromethylphenyl)- 2-[1-(4-fluorobenzenesulfonyl)piperidin- 4-yl]-2-hydroxy-3-[3′-(4-methylpiperazin- 1-ylmethyl)biphenyl-3-yl]propionamide | 764 |
| Example 186 | 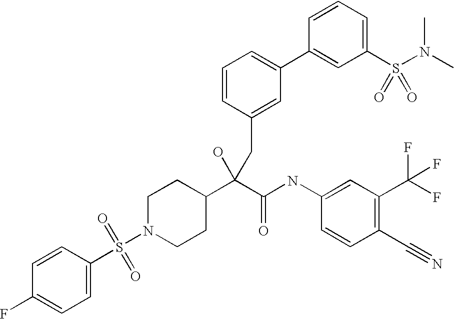 | N-(4-Cyano-3-trifluoromethylphenyl)- 3-(3′-dimethylsulfamoylbiphenyl- 3-yl)-2-[1-(4-fluorobenzenesulfonyl)- piperidin-4-yl]-2-hydroxypropionamide | 759 |
| Example 187 | 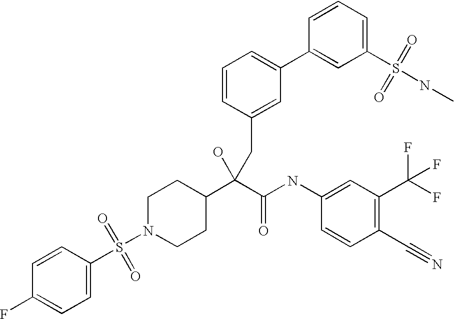 | N-(4-Cyano-3-trifluoromethylphenyl)- 2-[1-(4-fluorobenzenesulfonyl)piperidin- 4-yl]-2-hydroxy-3-(3′-methylsulfamoyl- biphenyl-3-yl)propionamide | 745 |
| Example 188 | 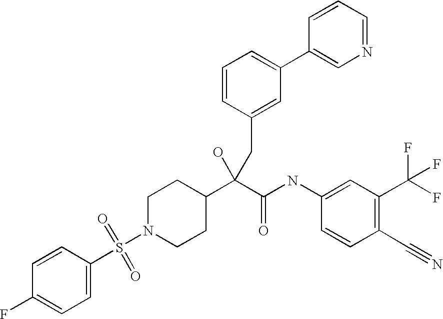 | N-(4-Cyano-3-trifluoromethylphenyl)- 2-[1-(4-fluorobenzenesulfonyl)piperidin- 4-yl]-2-hydroxy-3-(3-pyridin-3-yl- phenyl)propionamide | 653 |
| Example 189 | 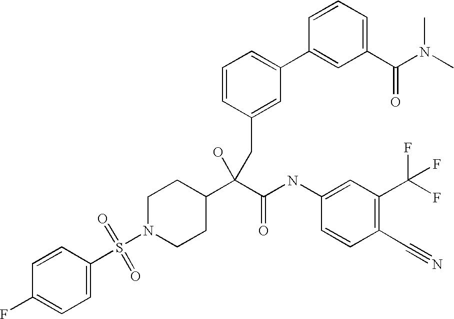 | N,N-Dimethyl-3′-{2-(4-cyano-3-trifluoro- methylphenylcarbamoyl)-2-[1-(4-fluoro- benzenesulfonyl)piperidin-4-yl]-2- hydroxyethyl}biphenyl-3-carboxamide | 723 |
| Example 190 | 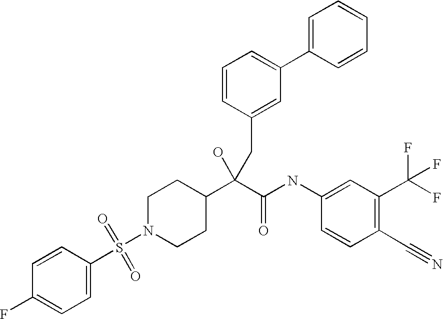 | 3-Biphenyl-3-yl-N-(4-cyano-3- trifluoromethylphenyl)-2-[1-(4- fluorobenzenesulfonyl)piperidin- 4-yl]-2-hydroxypropionamide | 652 |
| Example 191 | 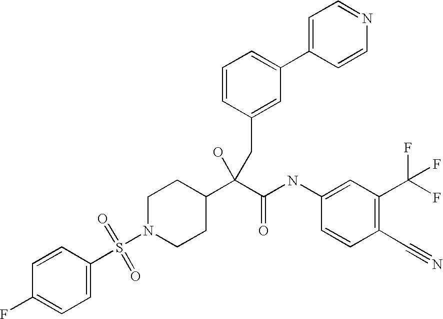 | N-(4-Cyano-3-trifluoromethylphenyl)- 2-[1-(4-fluorobenzenesulfonyl)piperidin- 4-yl]-2-hydroxy-3-(3-pyridin-4-yl- phenyl)propionamide | 653 |
| Example 192 | 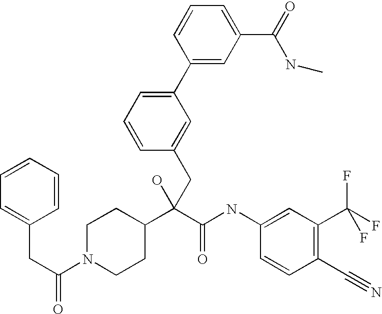 | N-Methyl-3′-[2-(4-cyano-3-trifluoro- methylphenylcarbamoyl)-2-hydroxy- 2-(1-phenylacetylpiperidin-4-yl)ethyl]- biphenyl-3-carboxamide | 669 |
| Example 193 | 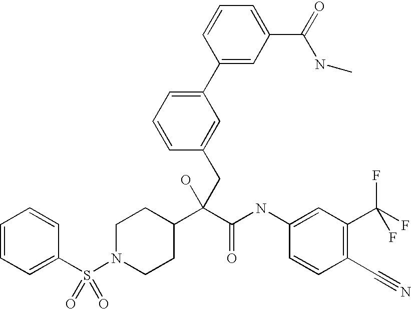 | N-Methyl-3′-[2-(1-benzenesulfonyl- piperidin-4-yl)-2-(4-cyano-3-trifluoro- methylphenylcarbamoyl)-2-hydroxy- ethyl]biphenyl-3-carboxamide | 691 |
| Example 194 | 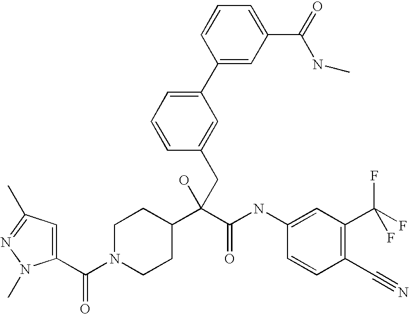 | N-Methyl-3′-{2-(4-cyano-3-trifluoro- methylphenylcarbamoyl)-2-[1-(2,5- dimethyl-2H-pyrazole-3-carbonyl)- piperidin-4-yl]-2-hydroxyethyl}- biphenyl-3-carboxamide | 673 |
| Example 195 | 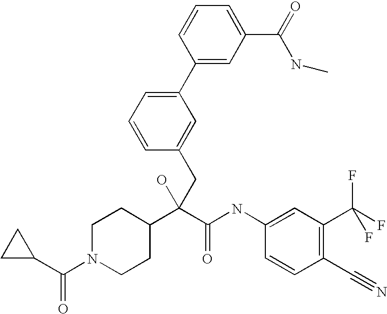 | N-Methyl-3′-[2-(4-cyano-3-trifluoro- methylphenylcarbamoyl)-2-(1-cyclo- propanecarbonylpiperidin-4-yl)-2- hydroxyethyl]biphenyl-3-carboxamide | 619 |
| Example 196 | 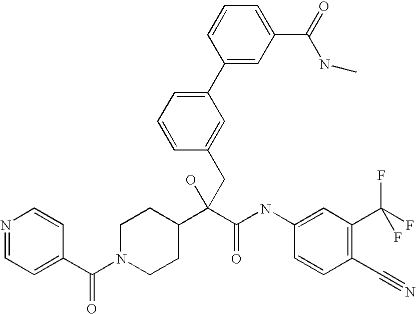 | N-Methyl-3′-{2-(4-cyano-3-trifluoro- methylphenylcarbamoyl)-2-hydroxy- 2-[1-(pyridine-4-carbonyl)piperidin- 4-yl]ethyl}biphenyl-3-carboxamide | 565 |
| Example 197 | 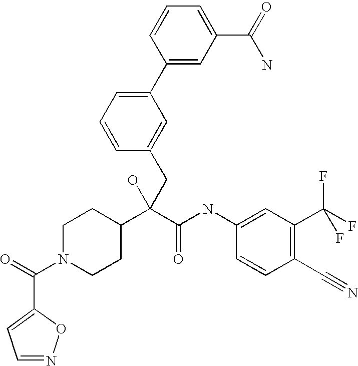 | 3′-{2-(4-Cyano-3-trifluoromethyl- phenylcarbamoyl)-2-hydroxy-2- [1-(isoxazole-5-carbonyl)piperidin- 4-yl]ethyl}biphenyl-3-carboxamide | 632 |

### EXAMPLE 198

### N-(4-Cyano-3-trifluoromethyl phenyl)-2-(4,4-difluorocyclohexyl)-2-hydroxy-3-phenylpropionamide

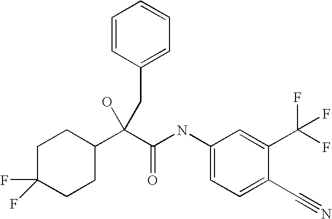

The compound was prepared analogously to example 99, beginning with 4,4-difluoro-cyclohexanone and separated into the enantiomers by means of chiral HPLC (Chiralpak AD-H 5μ, 250×20 mm, 8:2 hexane/ethanol, 25 ml/min).

Example 198a: Rₜ=5.1-6.1 min

Example 198b: Rₜ=7.1-8.1 min

¹H NMR (ppm, CDCl₃, 400 MHz): 1.57-2.25 (m, 9H), 2.90 (d, 1H), 3.48 (d, 1H), 7.16 (m, 2H), 7.29 (m, 3H), 7.75 (d, 1H), 7.80 (dd, 1H), 7.88 (d, 1H), 8.63 (s, 1H).

### EXAMPLE 199

### N-(4-Cyano-3-trifluoromethyl phenyl)-2-hydroxy-3-phenyl-2-(4-trifluoromethyl-cyclohexyl)propionamide

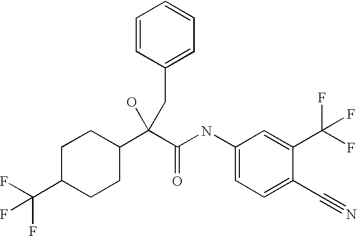

The compound was prepared analogously to example 99, beginning with 4-trifluoromethylcyclohexanone and separated into the isomers by means of chiral HPLC (Chiralpak IA 5μ, 250×20 mm, 9:1 hexane/ethanol, 25 ml/min). LCMS (ESI+) m/z=485 (M+1).

### EXAMPLE 200

### N-(4-Cyano-3-trifluoromethyl phenyl)-2-cyclohexyl-2-hydroxy-3-(3-hydroxy-phenyl)propionamide

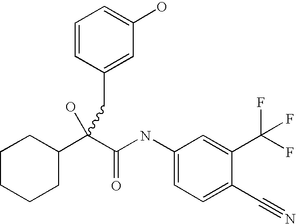

300 mg of N-(4-cyano-3-trifluoromethylphenyl)-2-cyclohexyl-2-hydroxy-3-(3-methoxy-phenyl)propionamide (example 24) were dissolved in 20 ml of dichloromethane and, at −10° C., 2 ml of tribromoborane were added dropwise. The mixture was left to stir at RT for 16 h, then the mixture was added to ice-water, extracted with ethyl acetate, washed with water and NaCl solution, dried with sodium sulfate and concentrated. The crude product was purified by chromatography (yield: 150 mg, 52%). The racemate was separated into the enantiomers by means of chiral HPLC (Chiralpak IA 5μ, 250×20 mm, 85:15 hexane/ethanol, 25 ml/min).

Example 200a: Rₜ=7.6-8.8 min; [α]^{D}₂₀=+116.5° (CHCl₃, c=0.34)

Example 200b: Rₜ=9.9-11.5 min; [α]^{D}₂₀=−122.5° (CHCl₃, c=0.43)

¹H NMR (ppm, CDCl₃, 400 MHz): 1.14 (m, 2H), 1.29 (m, 3H), 1.68 (m, 2H), 1.78 (m, 1H), 1.88 (m, 2H), 1.99 (m, 1H), 2.85 (d, 1H), 3.37 (d, 1H), 5.14 (s, 1H), 6.69 (m, 3H), 7.13 (dd, 1H), 7.78 (d, 1H), 7.82 (dd, 1H), 7.89 (d, 1H), 8.69 (s, 1H).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding European application No. 07076093.9, filed Dec. 14, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula (I) 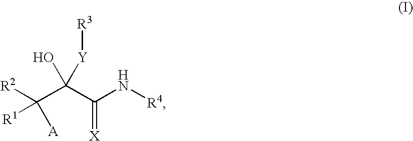
in which
A is hydrogen or a C₁-C₈-alkyl, C₂-C₈-alkenyl, C₂-C₈-alkynyl radical which is optionally mono- or polysubstituted identically or differently by Z, or else is Z itself, where Z is defined as follows:
cyano, halogen, hydroxyl, nitro, —C(O)R^{b}, C(O)CH₂R^{b}, —C(O)CF₂R^{b}, CO₂R^{b}, —O—R^{b},
—S—R^{b}, SO₂NR^{c}R^{d}, —C(O)—NR^{c}R^{d}, —OC(O)—NR^{c}R^{d}, —C═NOR^{b},
—NR^{c}R^{d}, PO₃(R^{b})₂, —NR^{e}COR^{b}, —NR^{e}CSR^{b}, —NR^{e}S(O)R^{b}, —NR^{e}S(O)₂R^{b},
NR^{e}CONR^{c}R^{d}, —NR^{e}COOR^{b},
—NR^{e}C(NH)NR^{c}R^{d}, —NR^{e}CSNR^{c}R^{d}, —NR^{e}S(O)NR^{c}R^{d},
—NR^{e}S(O)₂NR^{c}R^{d}, —S(O)R^{b}, —S(O)NR^{c}R^{d}, —S(O)₂R^{b}, —S(O)₂CH₂R^{b},
—S(O)₂CF₂R^{b}, —SO₂OR^{b},
—CSNR^{c}R^{d}, —CR^{b}(OH)—R^{b} where
R^{b} is hydrogen, a C₁-C₆-alkyl, hydroxy-C₁-C₃-alkyl, C₁-C₃-alkoxy-C₁-C₃-alkyl, C₂-C₈-alkenyl, C₂-C₈-alkynyl, —(CH₂)ₚC(O)₂H, —(CH₂)ₚC(O)₂C₁-C₃-alkyl, a 5- to 6-membered cycloalkyl or heterocycloalkyl radical which is optionally mono- or disubstituted by a halogen, a C₁-C₃-alkyl, C₁-C₃-alkoxy radical or a COOR^{b} radical and has 1, 2 or 3 heteroatoms or a phenyl or 3-12-membered heteroaryl radical which is optionally mono- or disubstituted by a halogen, a C₁-C₃-alkyl, C₁-C₃-alkoxy radical or a COOR^{b} radical and has 1, 2 or 3 heteroatoms, or a —(CH₂)ₚ—C₆-C₁₂-aryl radical which is optionally mono- or disubstituted by a halogen, a C₁-C₃-alkyl, C₁-C₃-alkoxy radical or a COOR^{b} radical or a partly or fully fluorinated C₁-C₃-fluoroalkyl radical
and
R^{c} and R^{d} are each independently hydrogen, a C₁-C₆-alkyl, C₂-C₈-alkenyl, C₂-C₈-alkynyl, C₃-C₁₀-cycloalkyl or C₆-C₁₂-aryl radical or a 5- to 12-membered heteroaryl radical optionally substituted by W, a C(O)R^{b} group with the definition of R^{b} specified above —S(O)₂C₁-C₃-alkyl, —C(O)C₁-C₃-alkyl or a hydroxyl group, or together including the nitrogen form a 3- to 7-membered ring which is optionally mono- or disubstituted by a trifluoromethyl and/or hydroxyl group and which is optionally extended by O, S or NR^{f} where
W is —NR^{g}R^{h} where
R^{g} is hydrogen or C₁-C₃-alkyl and
R^{h} is hydrogen or C₁-C₃-alkyl or
R^{g} and R^{h} together including the nitrogen form a 3- to 7-membered ring which is optionally extended by O, S or NR^{f} and
R^{f} is hydrogen, C₁-C₃-alkyl, C₁-C₃-acyl, C₁-C₃-alkylsulphonyl or C₁-C₃-alkoxycarbonyl,
and
where, when
R^{c} is a hydroxyl group, R^{d} can only be hydrogen, C₁-C₆-alkyl, C₂-C₈-alkenyl, C₂-C₈-alkynyl, C₃-C₁₀-cycloalkyl or C₆-C₁₂-aryl which is optionally substituted by W, and vice versa, and also
R^{e} is hydrogen, C₁-C₆-alkyl, C₂-C₈-alkenyl, C₂-C₈-alkynyl, C₃-C₁₀-cycloalkyl or C₆-C₁₂-aryl which is optionally substituted by W,
or
A is a C₃-C₁₀-cycloalkyl radical or 3-12-membered heterocycloalkyl radical which is optionally mono- or polysubstituted identically or differently by M and
M is C₁-C₆-alkyl or a —COR^{b}, CO₂R^{b}, —O—R^{b} or NR^{c}R^{d} group, where R^{b}, R^{c} and R^{d} are each as specified above, and
R¹ and R² are each independently an unbranched or branched C₁-C₅-alkyl group which is optionally substituted by Z or, together with the carbon atom of the chain, form a carbocyclic or heterocyclic ring which is optionally substituted by Z and has a total of 3-7 members,
where, when
A is hydrogen and R¹ is a methyl radical, R² cannot be a methyl radical or an ethyl radical,
A is hydrogen, R¹ and R² cannot together be a ring having 3-4 members,
A is a methyl radical, R¹ and R² cannot both be a methyl radical or, together with the carbon atom of the chain, form a cyclopropyl ring,
R³ is hydrogen or a C₁-C₈-alkyl, C₂-C₈-alkenyl, C₂-C₈-alkynyl, C₃-C₁₀-cycloalkyl, 3-12-membered heterocycloalkyl radical which is optionally mono- or polysubstituted identically or differently by K, or a C₆-C₁₂-aryl or 3-12-membered heteroaryl radical which is optionally mono-, di- or trisubstituted identically or differently by L, and
K is cyano, halogen, hydroxyl, nitro, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, —C═NOR^{b}, —NR^{c}R^{d} or a C₃-C₁₀-cycloalkyl, 3-12-membered hetero-cycloalkyl radical which is optionally mono- or polysubstituted identically or differently by M, or a C₆-C₁₂-aryl or 3-12-membered heteroaryl radical which is optionally mono-, di- or trisubstituted identically or differently by L, with the definition of M specified under A, and
L is C₁-C₈-alkyl, C₂-C₈-alkenyl, C₂-C₈-alkynyl, a partly or fully fluorinated C₁-C₆-fluoroalkyl, a partly or fully fluorinated C₁-C₆-fluoroalkoxy, C₁-C₆-alkoxy-C₁-C₆-alkyl, C₁-C₆-alkoxy-C₁-C₆-alkoxy, a mono- or bicyclic (CH₂)ₚ—C₃-C₁₀-cycloalkyl, a mono- or bicyclic 3-12-membered (CH₂)ₚ-heterocycloalkyl radical, (CH₂)ₚCN, (CH₂)ₚHal, (CH₂)ₚNO₂, a mono- or bicyclic (CH₂)ₚ—C₆-C₁₂-aryl radical which is optionally substituted by V, a mono- or bicyclic 3-12-membered (CH₂)ₚ-heteroaryl radical which is optionally substituted by V, or
—(CH₂)ₚPO₃(R^{b})₂, —(CH₂)ₚNR^{c}R^{d}, —(CH₂)ₚNR^{e}COR^{b},
—(CH₂)ₚNR^{e}CSR^{b}, —(CH₂)ₚNR^{e}S(O)R^{b}, —(CH₂)ₚNR^{e}S(O)₂R^{b},
—(CH₂)ₚNR^{e}CONR^{c}R^{d}, —(CH₂)ₚNR^{e}COOR^{b},
—(CH₂)ₚNR^{e}C(NH)NR^{c}R^{d}, —(CH₂)ₚNR^{e}CSNR^{c}R^{d},
—(CH₂)ₚNR^{e}S(O)NR^{c}R^{d}, —(CH₂)ₚNR^{e}S(O)₂NR^{c}R^{d},
—(CH₂)ₚCOR^{b}, —(CH₂)ₚCSR^{b}, —(CH₂)ₚS(O)R^{b},
—(CH₂)ₚS(O)(NH)R^{b}, —(CH₂)ₚS(O)₂R^{b}, —(CH₂)ₚS(O)₂NR^{c}R^{d},
—(CH₂)ₚSO₂OR^{b}, —(CH₂)ₚCO₂R^{b}, —(CH₂)ₚCONR^{c}R^{d},
—(CH₂)ₚCSNR^{c}R^{d}, —(CH₂)ₚOR^{b}, —(CH₂)ₚSR^{b},
—(CH₂)ₚCR^{b}(OH)—R^{b}, —(CH₂)ₚ—C═NOR^{b}, —O—(CH₂)ₙ—O—,
—O—(CH₂)ₙ—CH₂—, —OCH═CH— or —(CH₂)ₙ₊₂— and
the terminal oxygen atoms and/or carbon atoms are linked
to directly adjacent ring carbon atoms and
n is 1 or 2 and
p is 0, 1, 2, 3, 4, 5 or 6, and
V is cyano, halogen, nitro, —(CH₂)ₚOR^{b}, —(CH₂)ₚS(O)₂R^{b}, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, —C═NOR^{b}, —(CH₂)ₚNR^{c}R^{d}, partly or fully fluorinated C₁-C₆-fluoroalkyl or partly or fully fluorinated C₁-C₆-fluoroalkoxy,
X is one oxygen atom and two hydrogen atoms
Y is (CH₂)ₘ, —C≡C— or —CH═CH— where
m=0 or 1,
where, when Y is a CH₂ radical, R³ cannot be hydrogen, and
R⁴ is a mono- or bicyclic C₆-C₁₂-aryl which is optionally substituted identically or differently by 2 L, or one of the following groups mentioned under B or C:
B: 6-membered/6-membered ring systems: 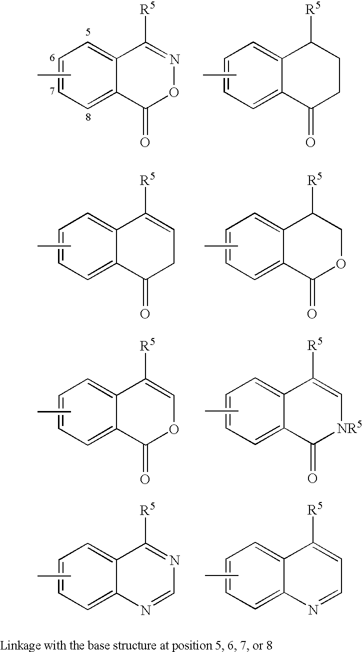
C: 6-membered/5-membered ring systems: 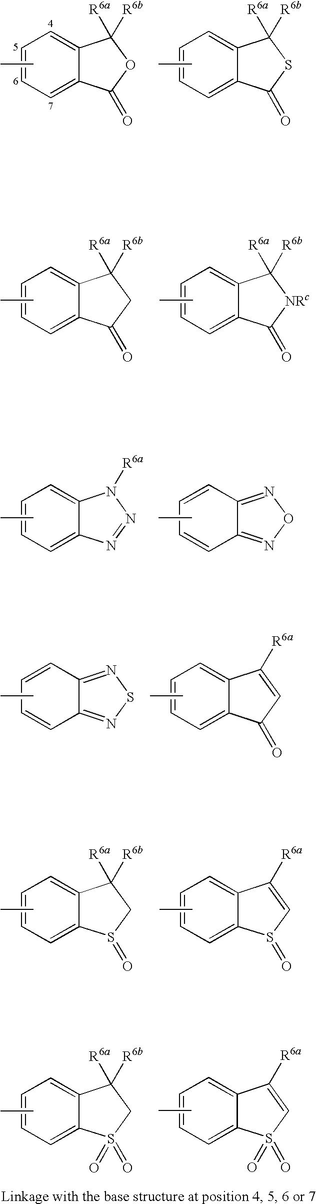
where
R⁵ is hydrogen or C₁-C₄-alkyl, or a partly or fully fluorinated C₁-C₄-fluoroalkyl,
R^{6a} and R^{6b} are each independently hydrogen, C₁-C₄-alkyl or a partly or fully fluorinated C₁-C₄-fluoroalkyl, or, together with the ring carbon atom, form a 3- to 6-membered ring,
and the pharmaceutically acceptable salts thereof.

2. Compounds according to claim 1, in which A is a hydrogen.

3. Compounds according to claim 2, in which Y is —C≡C—, R¹ and R² together with the carbon atom of the chain form a carbocyclic or heterocyclic 3-6-membered ring and R³ is optionally K-substituted C₁-C₈-alkyl, a C₆-C₁₂-aryl optionally mono-, di- or trisubstituted identically or differently by L, or 3- to 12-membered heteroaryl.

4. Compounds according to claim 2, in which Y is (CH₂)ₘ and R³ is a C₆-C₁₂-aryl optionally mono-, di- or trisubstituted identically or differently by L or 3- to 12-membered heteroaryl, and R⁴ is mono- or bicyclic aryl disubstituted identically or differently by L, or one of the B groups specified under R⁴ with linkage at position 6 or C with linkage at position 5.

5. Compounds according to claim 4, in which m=1.

6. Compounds according to claim 5, in which R⁴ is a phenyl ring disubstituted identically or differently by L.

7. Compounds according to claim 6, in which the phenyl ring is substituted by a cyano radical, by chlorine and/or by a trifluoromethyl radical.

8. Compounds according to claim 4, in which R⁴ has the following definition:

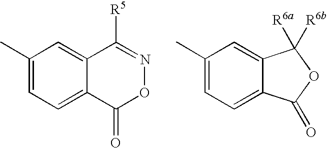

9. Compounds according to claim 8, in which
R⁵ is methyl or ethyl
R⁶ is hydrogen.

10. Compounds according to claim 2, in which
p is 0, 1 or 2, and
L is C₁-C₈-alkyl, C₂-C₈-alkenyl, C₂-C₈-alkynyl, partly or fully fluorinated C₁-C₆-fluoroalkyl, —(CH₂)ₚCN, (CH₂)ₚHal, (CH₂)ₚNO₂, (CH₂)ₚ—C₆-C₁₂-aryl, —(CH₂)ₚ-heteroaryl, —(CH₂)ₚNR^{c}R^{d}, —(CH₂)ₚNR^{e}COR^{b}, —(CH₂)ₚNR^{e}S(O)₂R^{b}, —(CH₂)ₚNR^{e}CONR^{c}R^{d}, —(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚS(O)R^{b}, —(CH₂)ₚS(O)₂R^{b}, —(CH₂)ₚS(O)₂NR^{c}R^{d}, —(CH₂)ₚCO₂R^{b}, —(CH₂)ₚCONR^{c}R^{d}, —(CH₂)ₚOR^{b}, —(CH₂)ₚCR^{b}(OH)—R^{b} and
Z is cyano, halogen, hydroxyl, nitro, —C(O)R^{b}, CO₂R^{b}, —O—R^{b}, —SO₂NR^{c}R^{d}, —C(O)—NR^{c}R^{d}, —NR^{c}R^{d}, —NR^{e}COR^{b}, —NR^{e}S(O)R^{b}, —NR^{e}S(O)₂R^{b}, —NR^{e}CONR^{c}R^{d}, —S(O)R^{b}, —S(O)NR^{c}R^{d}, —S(O)₂R^{b}, —CR^{b}(OH)—R^{b} or a C₃-C₁₀-cycloalkyl or heterocycloalkyl optionally mono- or polysubstituted identically or differently by M.

11. Compounds according to claim 2, in which R¹ and R² together with the carbon atom of the chain form a cyclopropyl, cyclopentyl or cyclohexyl ring.

12. Compounds according to claim 2, in which R¹ and R² together with the carbon atom of the chain form a tetrahydropyranyl, piperidinyl or tetrahydrothiopyranyl ring.

13. Compounds according to one of the preceding claims, specifically

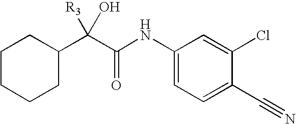

| No. | Racemic or enantiomer | R3 |
|---|---|---|
| 1 2 3 | rac + − | 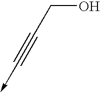 |
| 4 5 6 | rac + − | 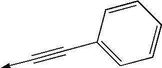 |
| 7 8 9 | rac + − | 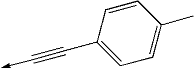 |
| 10 11 12 | rac + − | 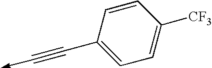 |
| 13 14 15 | rac + − | 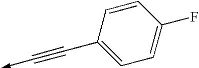 |
| 16 17 18 | rac + − | 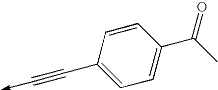 |
| 19 20 21 | rac + − | 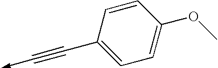 |
| 22 23 24 | rac + − | 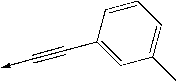 |
| 25 26 27 | rac + − | 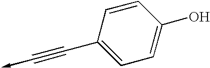 |
| 28 29 30 | rac + − | 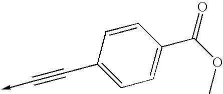 |
| 31 32 33 | rac + − | 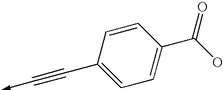 |
| 34 35 36 | rac + − | 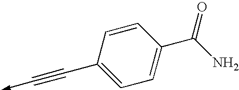 |
| 37 38 39 | rac + − | 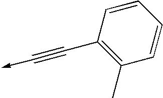 |
| 40 41 42 | rac + − | 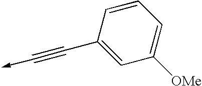 |
| 43 44 45 | rac + − | 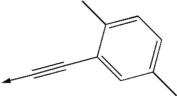 |
| 46 47 48 | rac + − | 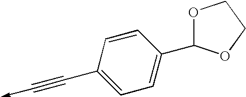 |
| 49 50 51 | rac + − | 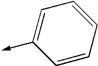 |
| 52 53 54 | rac + − | 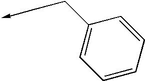 |
| 55 56 57 | rac + − | 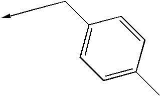 |
| 58 59 60 | rac + − | 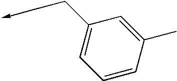 |
| 61 62 63 | rac + − | 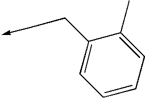 |
| 64 65 66 | rac + − | 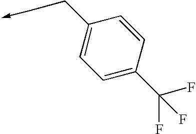 |
| 67 68 69 | rac + − | 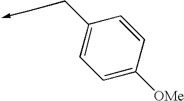 |
| 70 71 72 | rac + − | 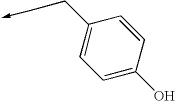 |
| 73 74 75 | rac + − | 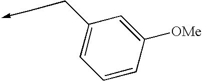 |
| 76 77 78 | rac + − | 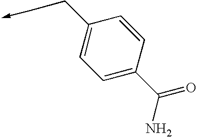 |
| 79 80 81 | rac + − | 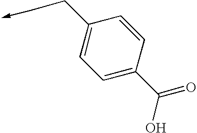 |
| 82 83 84 | rac + − | 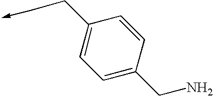 |
| 85 86 87 | rac + − | 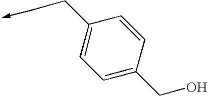 |
| 88 89 90 | rac + − | 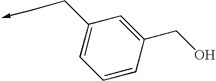 |
| 91 92 93 | rac + − | 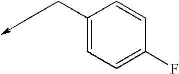 |
| 94 95 96 | rac + − | 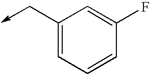 |
| 97 98 99 | rac + − | 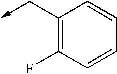 |
| 100 101 102 | rac + − | 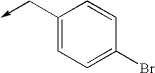 |
| 103 104 105 | rac + − | 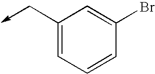 |
| 106 107 108 | rac + − | 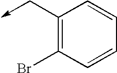 |
| 109 110 111 | rac + − | 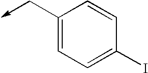 |
| 112 113 114 | rac + − | 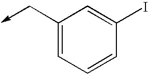 |
| 115 116 117 | rac + − | 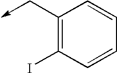 |
| 118 119 120 | rac + − | 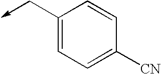 |
| 121 122 123 | rac + − | 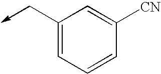 |
| 124 125 126 | rac + − | 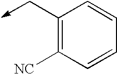 |
| 127 128 129 | rac + − | 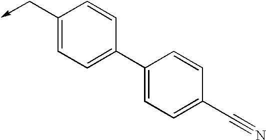 |
| 130 131 132 | rac + − | 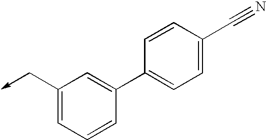 |
| 133 134 135 | rac + − | 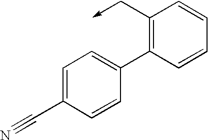 |
| 136 137 138 | rac + − | 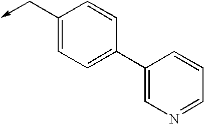 |
| 139 140 141 | rac + − | 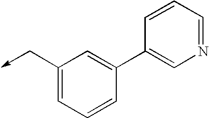 |
| 142 143 144 | rac + − | 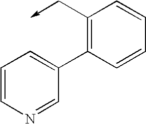 |
| 145 146 147 | rac + − | 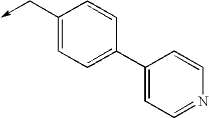 |
| 148 149 150 | rac + − | 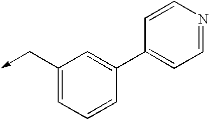 |
| 151 152 153 | rac + − | 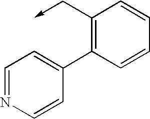 |
| 154 155 156 | rac + − | 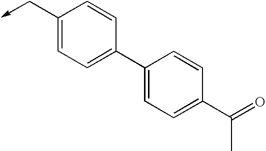 |
| 157 158 159 | rac + − | 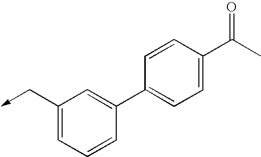 |
| 160 161 162 | rac + − | 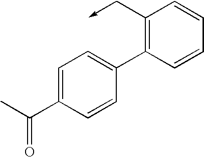 |
| 163 164 165 | rac + − | 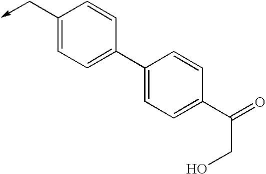 |
| 166 167 168 | rac + − | 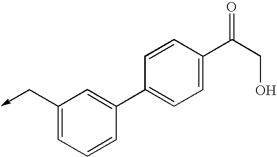 |
| 169 170 171 | rac + − | 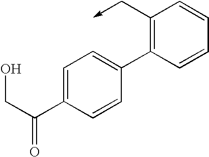 |
| 172 173 174 | rac + − | 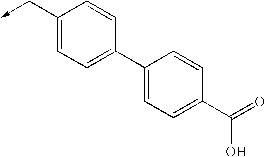 |
| 175 176 177 | rac + − | 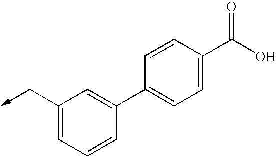 |
| 178 179 180 | rac + − | 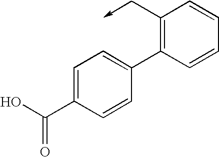 |
| 181 182 183 | rac + − | 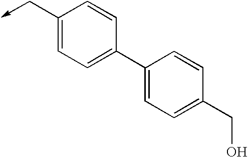 |
| 184 185 186 | rac + − | 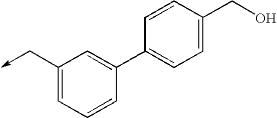 |
| 187 188 189 | rac + − | 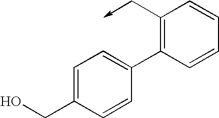 |
| 190 191 192 | rac + − | 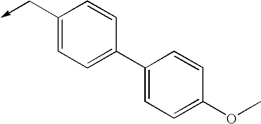 |
| 193 194 195 | rac + − | 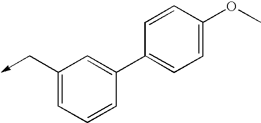 |
| 196 197 198 | rac + − | 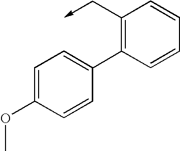 |
| 199 200 201 | rac + − | 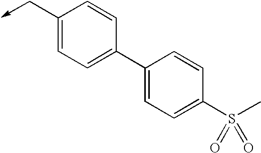 |
| 202 203 204 | rac + − | 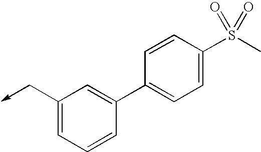 |
| 205 206 207 | rac + − | 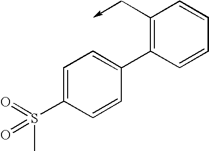 |
| 208 209 210 | rac + − | 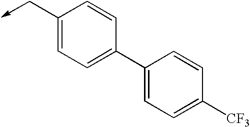 |
| 211 212 213 | rac + − | 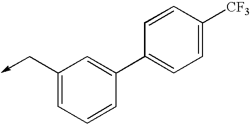 |
| 214 215 216 | rac + − | 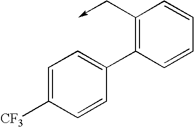 |
| 217 218 219 | rac + − | 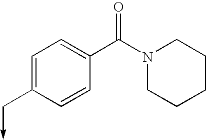 |
| 220 221 222 | rac + − | 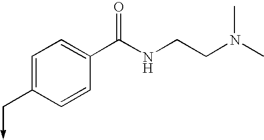 |
| 223 224 225 | rac + − | 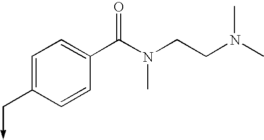 |
| 226 227 228 | rac + − | 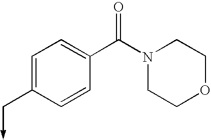 |
| 229 230 231 | rac + − | 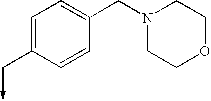 |
| 232 233 234 | rac + − | 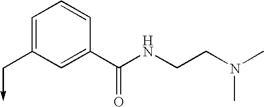 |
| 235 236 237 | rac + − | 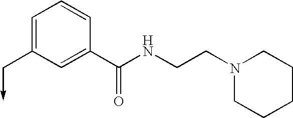 |
| 238 239 240 | rac + − | 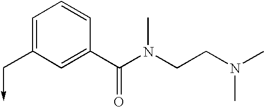 |
| 241 242 243 | rac + − | 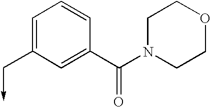 |
| 244 245 246 | rac + − | 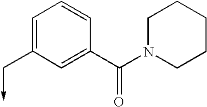 |
| 247 248 249 | rac + − | 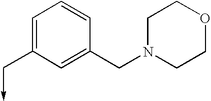 |
| 250 251 252 | rac + − | 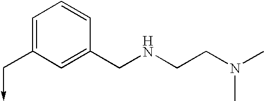 |
| 253 254 255 | rac + − | 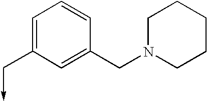 |
| 256 257 258 | rac + − | 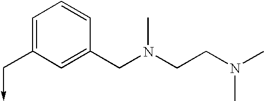 |
| 259 260 261 | rac + − | 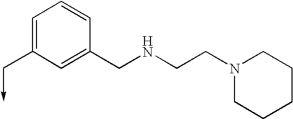 |

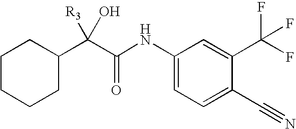
| No. | Racemic or enantiomer | R3 |
|---|---|---|
| 262 263 264 | rac + − | 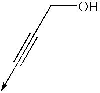 |
| 265 266 267 | rac + − | 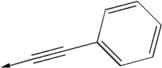 |
| 268 269 270 | rac + − | 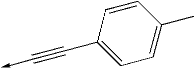 |
| 271 272 273 | rac + − | 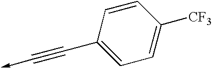 |
| 274 275 276 | rac + − | 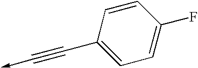 |
| 277 278 279 | rac + − | 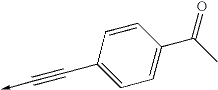 |
| 280 281 282 | rac + − | 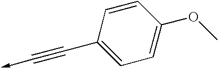 |
| 283 284 285 | rac + − | 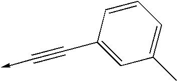 |
| 286 287 288 | rac + − | 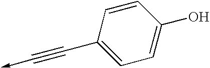 |
| 289 290 291 | rac + − | 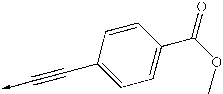 |
| 292 293 294 | rac + − | 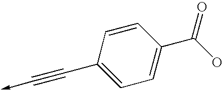 |
| 295 296 297 | rac + − | 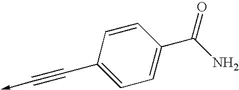 |
| 298 299 300 | rac + − | 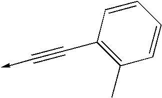 |
| 301 302 303 | rac + − | 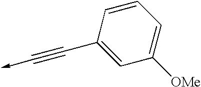 |
| 304 305 306 | rac + − | 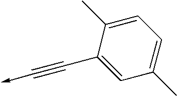 |
| 307 308 309 | rac + − | 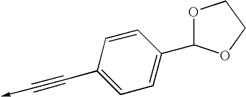 |
| 310 311 312 | rac + − | 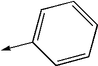 |
| 313 314 315 | rac + − | 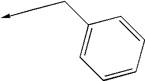 |
| 316 317 318 | rac + − | 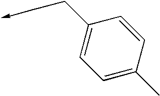 |
| 319 320 321 | rac + − | 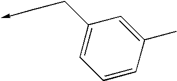 |
| 322 323 324 | rac + − | 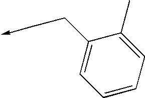 |
| 325 326 327 | rac + − | 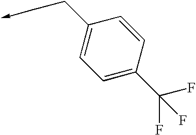 |
| 328 329 330 | rac + − | 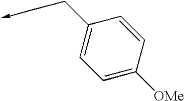 |
| 331 332 333 | rac + − | 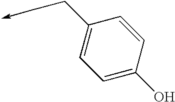 |
| 334 335 336 | rac + − | 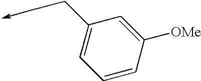 |
| 337 338 339 | rac + − | 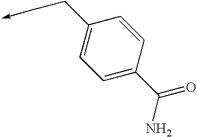 |
| 340 341 342 | rac + − | 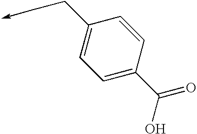 |
| 343 344 345 | rac + − | 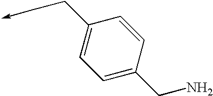 |
| 346 347 348 | rac + − | 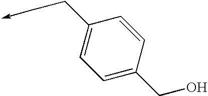 |
| 349 350 351 | rac + − | 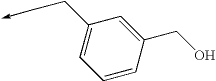 |
| 352 353 354 | rac + − | 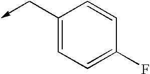 |
| 355 356 357 | rac + − | 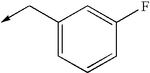 |
| 358 359 360 | rac + − | 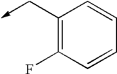 |
| 361 362 363 | rac + − | 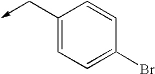 |
| 364 365 366 | rac + − | 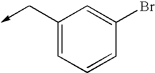 |
| 367 368 369 | rac + − | 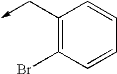 |
| 370 371 372 | rac + − | 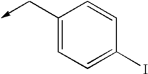 |
| 373 374 375 | rac + − | 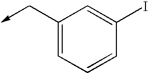 |
| 376 377 378 | rac + − | 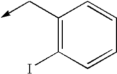 |
| 379 380 381 | rac + − | 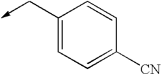 |
| 382 383 384 | rac + − | 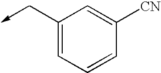 |
| 385 386 387 | rac + − | 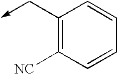 |
| 388 389 390 | rac + − | 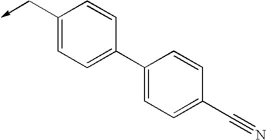 |
| 391 392 393 | rac + − | 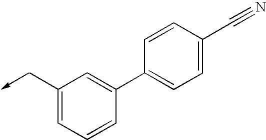 |
| 394 395 396 | rac + − | 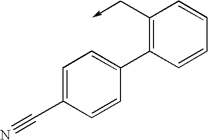 |
| 397 398 399 | rac + − | 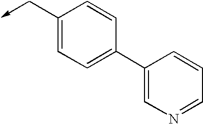 |
| 400 401 402 | rac + − | 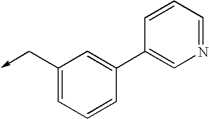 |
| 403 404 405 | rac + − | 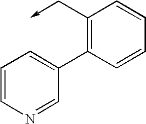 |
| 406 407 408 | rac + − | 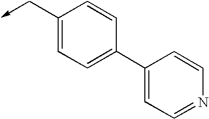 |
| 409 410 411 | rac + − | 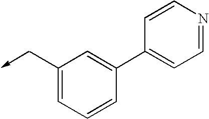 |
| 412 413 414 | rac + − | 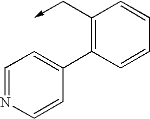 |
| 415 416 417 | rac + − | 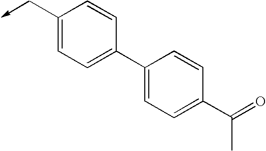 |
| 418 419 420 | rac + − | 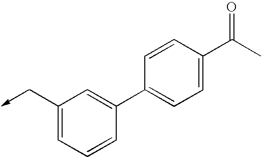 |
| 421 422 423 | rac + − | 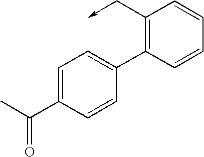 |
| 424 425 426 | rac + − | 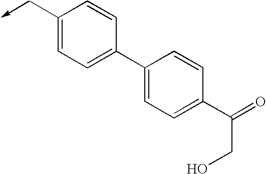 |
| 427 428 429 | rac + − | 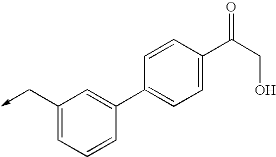 |
| 430 431 432 | rac + − | 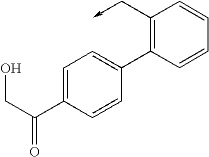 |
| 433 434 435 | rac + − | 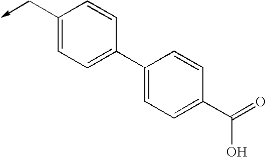 |
| 436 437 438 | rac + − | 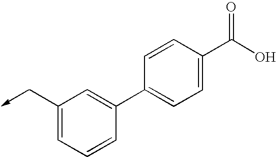 |
| 439 440 441 | rac + − | 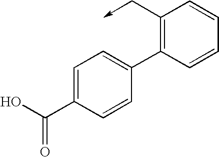 |
| 442 443 444 | rac + − | 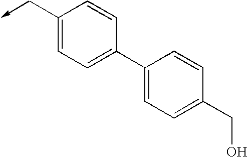 |
| 445 446 447 | rac + − | 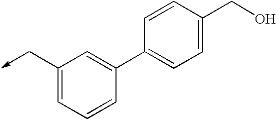 |
| 448 449 450 | rac + − | 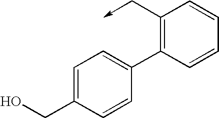 |
| 451 452 453 | rac + − | 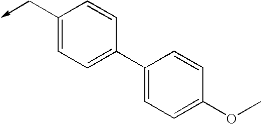 |
| 454 455 456 | rac + − | 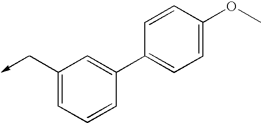 |
| 457 458 459 | rac + − | 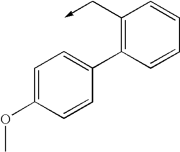 |
| 460 461 462 | rac + − | 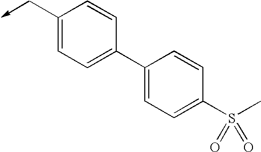 |
| 463 464 465 | rac + − | 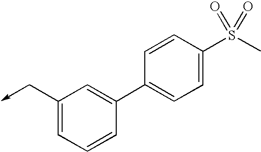 |
| 466 467 468 | rac + − | 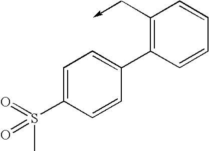 |
| 469 470 471 | rac + − | 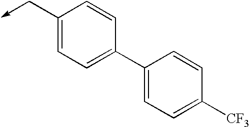 |
| 472 473 474 | rac + − | 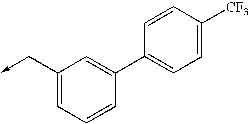 |
| 475 476 477 | rac + − | 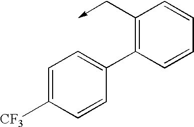 |
| 478 479 480 | rac + − | 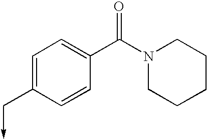 |
| 481 482 483 | rac + − | 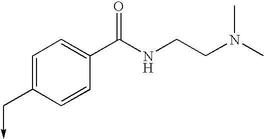 |
| 484 485 486 | rac + − | 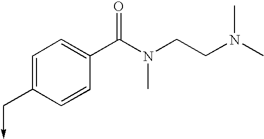 |
| 487 488 489 | rac + − | 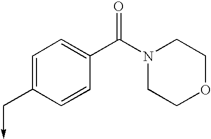 |
| 490 491 492 | rac + − | 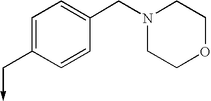 |
| 493 494 495 | rac + − | 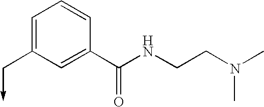 |
| 496 497 498 | rac + − | 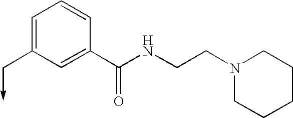 |
| 499 500 501 | rac + − | 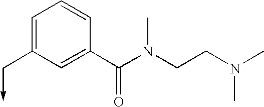 |
| 502 503 504 | rac + − | 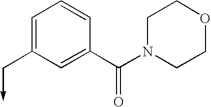 |
| 505 506 507 | rac + − | 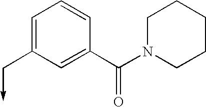 |
| 508 509 510 | rac + − | 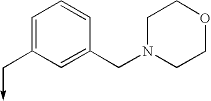 |
| 511 512 513 | rac + − | 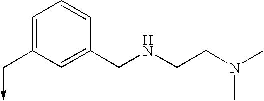 |
| 514 515 516 | rac + − | 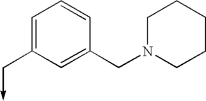 |
| 517 518 519 | rac + − | 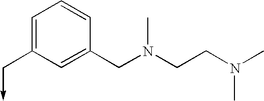 |
| 520 521 522 | rac + − | 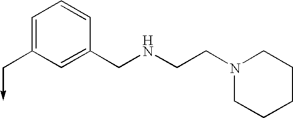 |

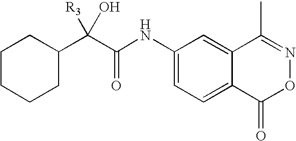
| No. | Racemic or enantiomer | R3 |
|---|---|---|
| 523 524 525 | rac + − | 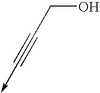 |
| 526 527 528 | rac + − | 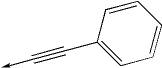 |
| 529 530 531 | rac + − | 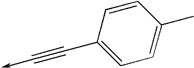 |
| 532 533 534 | rac + − | 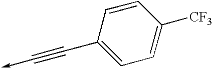 |
| 535 536 537 | rac + − | 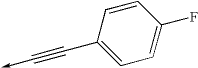 |
| 538 539 540 | rac + − | 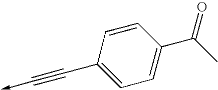 |
| 541 542 543 | rac + − | 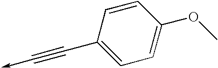 |
| 544 545 546 | rac + − | 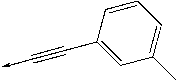 |
| 547 548 549 | rac + − | 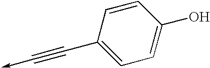 |
| 550 551 552 | rac + − | 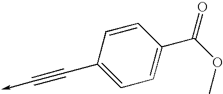 |
| 553 554 555 | rac + − | 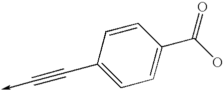 |
| 556 557 558 | rac + − | 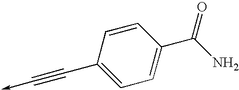 |
| 559 560 561 | rac + − | 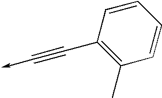 |
| 562 563 564 | rac + − | 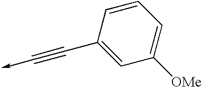 |
| 565 566 567 | rac + − | 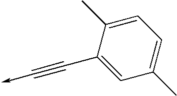 |
| 568 569 570 | rac + − | 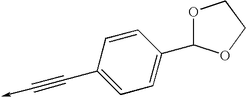 |
| 571 572 573 | rac + − | 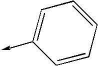 |
| 574 575 576 | rac + − | 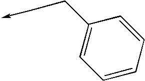 |
| 577 578 579 | rac + − | 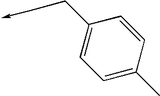 |
| 580 581 582 | rac + − | 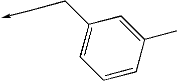 |
| 583 584 585 | rac + − | 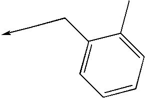 |
| 586 587 588 | rac + − | 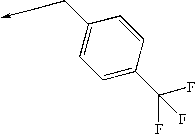 |
| 589 590 591 | rac + − | 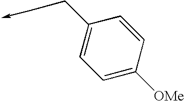 |
| 592 593 594 | rac + − | 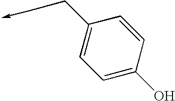 |
| 595 596 597 | rac + − | 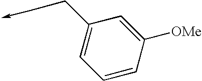 |
| 598 599 600 | rac + − | 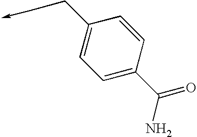 |
| 601 602 603 | rac + − | 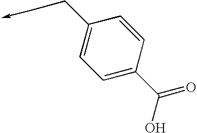 |
| 604 605 606 | rac + − | 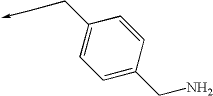 |
| 607 608 609 | rac + − | 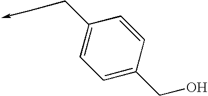 |
| 610 611 612 | rac + − | 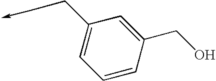 |
| 613 614 615 | rac + − | 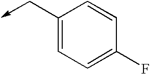 |
| 616 617 618 | rac + − | 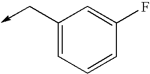 |
| 619 620 621 | rac + − | 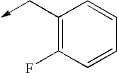 |
| 622 623 624 | rac + − | 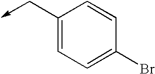 |
| 625 626 627 | rac + − | 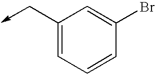 |
| 628 629 630 | rac + − | 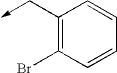 |
| 631 632 633 | rac + − | 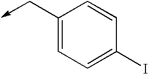 |
| 634 635 636 | rac + − | 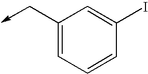 |
| 637 638 639 | rac + − | 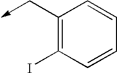 |
| 640 641 642 | rac + − | 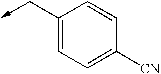 |
| 643 644 645 | rac + − | 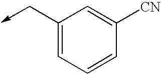 |
| 646 647 648 | rac + − | 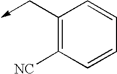 |
| 649 650 651 | rac + − | 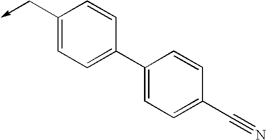 |
| 652 653 654 | rac + − | 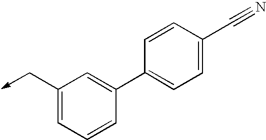 |
| 655 656 657 | rac + − | 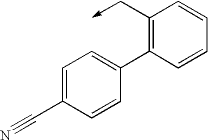 |
| 658 659 660 | rac + − | 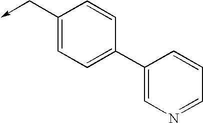 |
| 661 662 663 | rac + − | 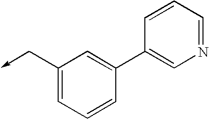 |
| 664 665 666 | rac + − | 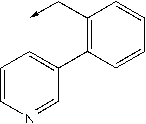 |
| 667 668 669 | rac + − | 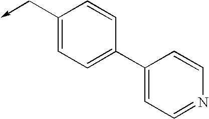 |
| 670 671 672 | rac + − | 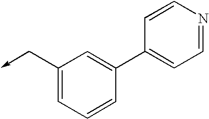 |
| 673 674 675 | rac + − | 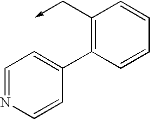 |
| 676 677 678 | rac + − | 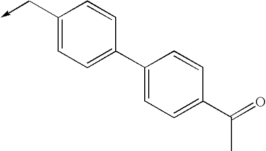 |
| 679 680 681 | rac + − | 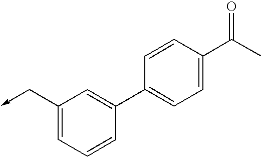 |
| 682 683 684 | rac + − | 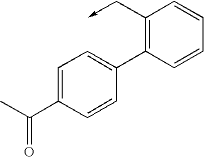 |
| 685 686 687 | rac + − | 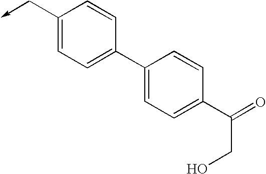 |
| 688 689 690 | rac + − | 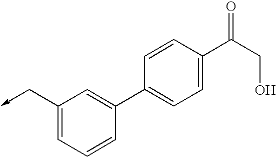 |
| 691 692 693 | rac + − | 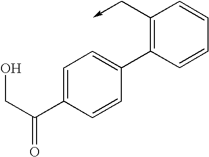 |
| 694 695 696 | rac + − | 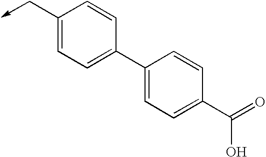 |
| 697 698 699 | rac + − | 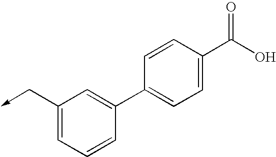 |
| 700 701 702 | rac + − | 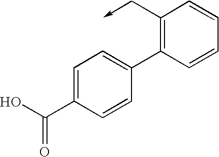 |
| 703 704 705 | rac + − | 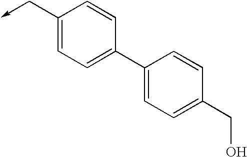 |
| 706 707 708 | rac + − | 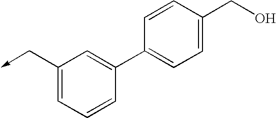 |
| 709 710 711 | rac + − | 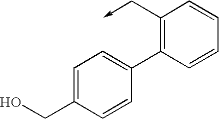 |
| 712 713 714 | rac + − | 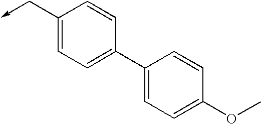 |
| 715 716 717 | rac + − | 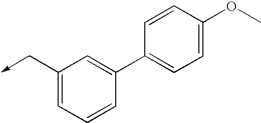 |
| 718 719 720 | rac + − | 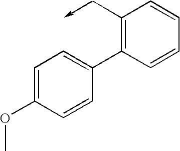 |
| 721 722 723 | rac + − | 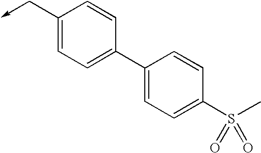 |
| 724 725 726 | rac + − | 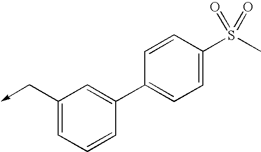 |
| 727 728 729 | rac + − | 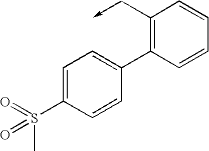 |
| 730 731 732 | rac + − | 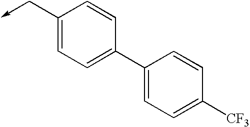 |
| 733 734 735 | rac + − | 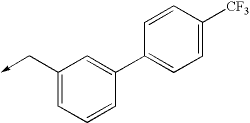 |
| 736 737 738 | rac + − | 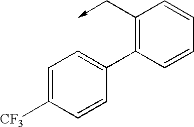 |
| 739 740 741 | rac + − | 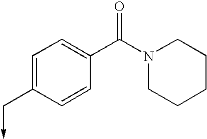 |
| 742 743 744 | rac + − | 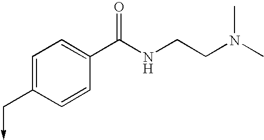 |
| 745 746 747 | rac + − | 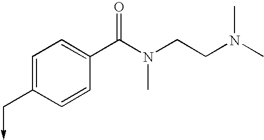 |
| 748 749 750 | rac + − | 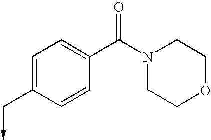 |
| 751 752 753 | rac + − | 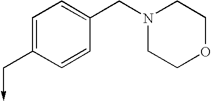 |
| 754 755 756 | rac + − | 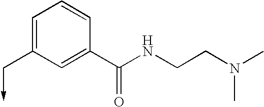 |
| 757 758 759 | rac + − | 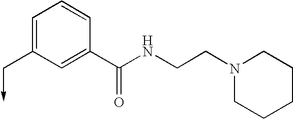 |
| 760 761 762 | rac + − | 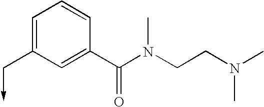 |
| 763 764 765 | rac + − | 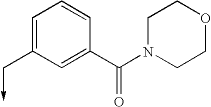 |
| 766 767 768 | rac + − | 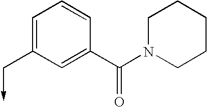 |
| 769 770 771 | rac + − | 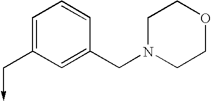 |
| 772 773 774 | rac + − | 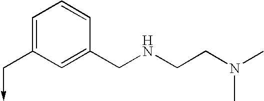 |
| 775 776 777 | rac + − | 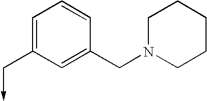 |
| 778 779 780 | rac + − | 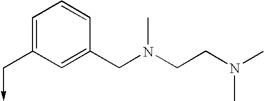 |
| 781 782 783 | rac + − | 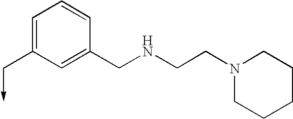 |

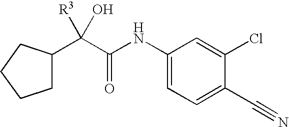
| No. | Racemic or enantiomer | R3 |
|---|---|---|
| 784 785 786 | rac + − | 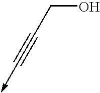 |
| 787 788 789 | rac + − | 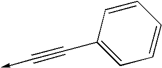 |
| 790 791 792 | rac + − | 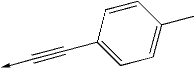 |
| 793 794 795 | rac + − | 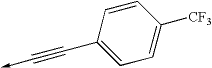 |
| 796 797 798 | rac + − | 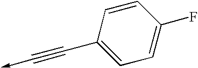 |
| 799 800 801 | rac + − | 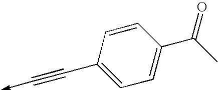 |
| 802 803 804 | rac + − | 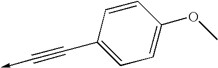 |
| 805 806 807 | rac + − | 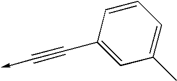 |
| 808 809 810 | rac + − | 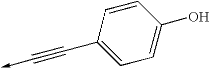 |
| 811 812 813 | rac + − | 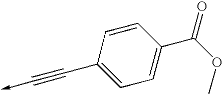 |
| 814 815 816 | rac + − | 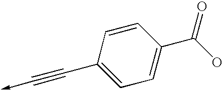 |
| 817 818 819 | rac + − | 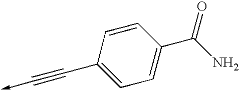 |
| 820 821 822 | rac + − | 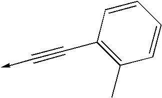 |
| 823 824 825 | rac + − | 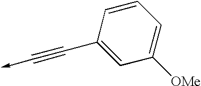 |
| 826 827 828 | rac + − | 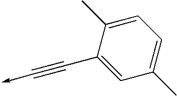 |
| 829 830 831 | rac + − | 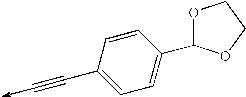 |
| 832 833 834 | rac + − | 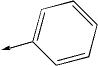 |
| 835 836 837 | rac + − | 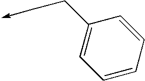 |
| 838 839 840 | rac + − | 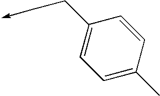 |
| 841 842 843 | rac + − | 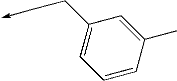 |
| 844 845 846 | rac + − | 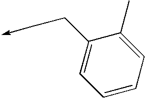 |
| 847 848 849 | rac + − | 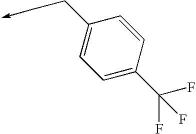 |
| 850 851 852 | rac + − | 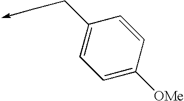 |
| 853 854 855 | rac + − | 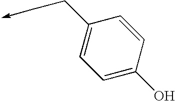 |
| 856 857 858 | rac + − | 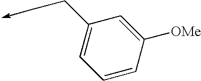 |
| 859 860 861 | rac + − | 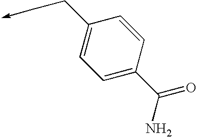 |
| 862 863 864 | rac + − | 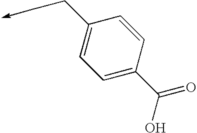 |
| 865 866 867 | rac + − | 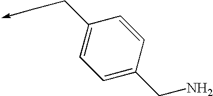 |
| 868 869 870 | rac + − | 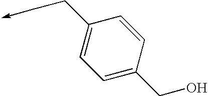 |
| 871 872 873 | rac + − | 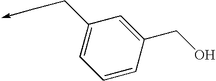 |
| 874 875 876 | rac + − | 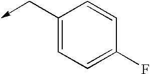 |
| 877 878 879 | rac + − | 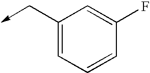 |
| 880 881 882 | rac + − | 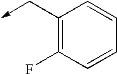 |
| 883 884 885 | rac + − | 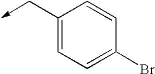 |
| 886 887 888 | rac + − | 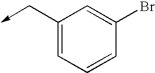 |
| 889 890 891 | rac + − | 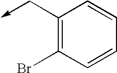 |
| 892 893 894 | rac + − | 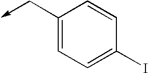 |
| 895 896 897 | rac + − | 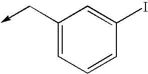 |
| 898 899 900 | rac + − | 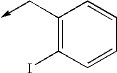 |
| 901 902 903 | rac + − | 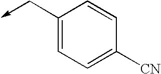 |
| 904 905 906 | rac + − | 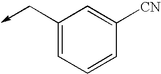 |
| 907 908 909 | rac + − | 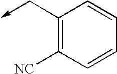 |
| 910 911 912 | rac + − | 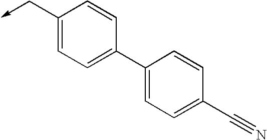 |
| 913 914 915 | rac + − | 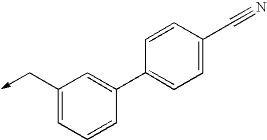 |
| 916 917 918 | rac + − | 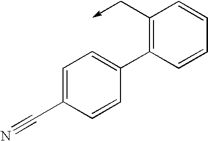 |
| 919 920 921 | rac + − | 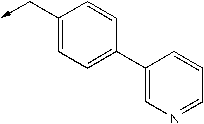 |
| 922 923 924 | rac + − | 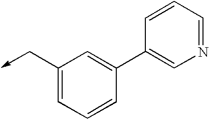 |
| 925 926 927 | rac + − | 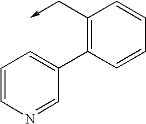 |
| 928 929 930 | rac + − | 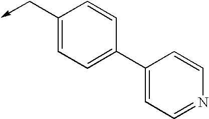 |
| 931 932 933 | rac + − | 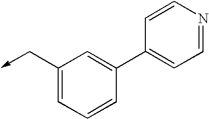 |
| 934 935 936 | rac + − | 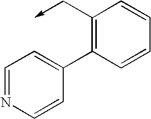 |
| 937 938 939 | rac + − | 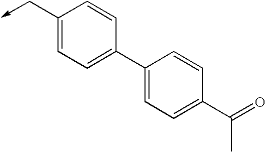 |
| 940 941 942 | rac + − | 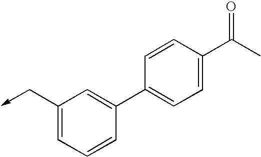 |
| 943 944 945 | rac + − | 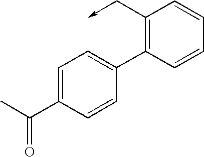 |
| 946 947 948 | rac + − | 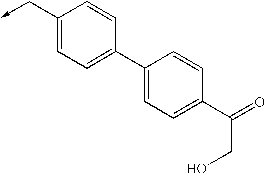 |
| 949 950 951 | rac + − | 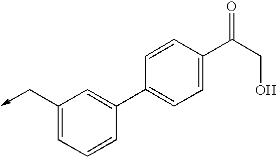 |
| 952 953 954 | rac + − | 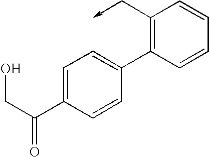 |
| 955 956 957 | rac + − | 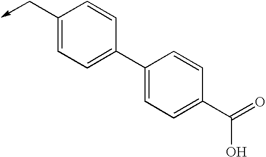 |
| 958 959 960 | rac + − | 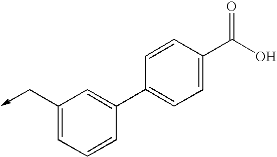 |
| 961 962 963 | rac + − | 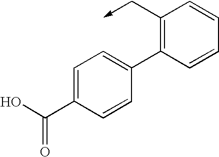 |
| 964 965 966 | rac + − | 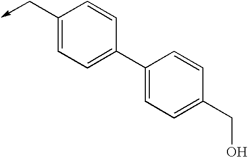 |
| 967 968 969 | rac + − | 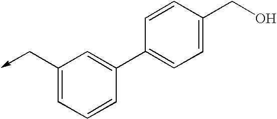 |
| 970 971 972 | rac + − | 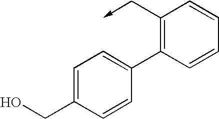 |
| 973 974 975 | rac + − | 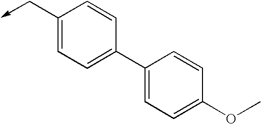 |
| 976 977 978 | rac + − | 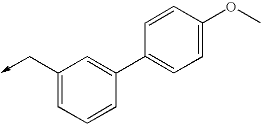 |
| 979 980 981 | rac + − | 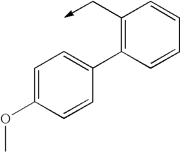 |
| 982 983 984 | rac + − | 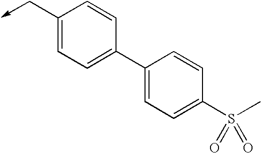 |
| 985 986 987 | rac + − | 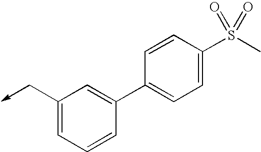 |
| 988 989 990 | rac + − | 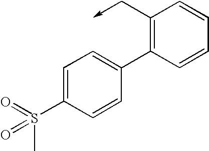 |
| 991 992 993 | rac + − | 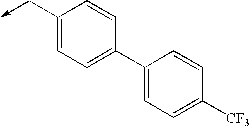 |
| 994 995 996 | rac + − | 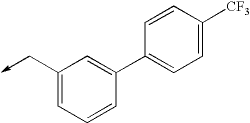 |
| 997 998 999 | rac + − | 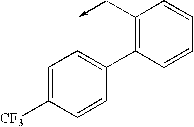 |
| 1000 1001 1002 | rac + − | 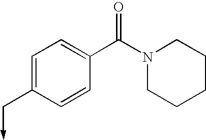 |
| 1003 1004 1005 | rac + − | 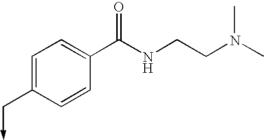 |
| 1006 1007 1008 | rac + − | 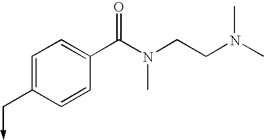 |
| 1009 1010 1011 | rac + − | 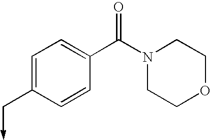 |
| 1012 1013 1014 | rac + − | 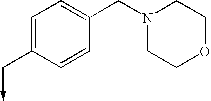 |
| 1015 1016 1017 | rac + − | 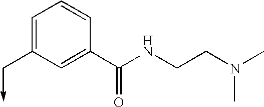 |
| 1018 1019 1020 | rac + − | 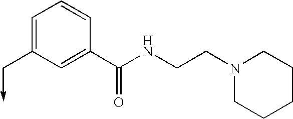 |
| 1021 1022 1023 | rac + − | 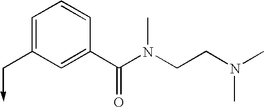 |
| 1024 1025 1026 | rac + − | 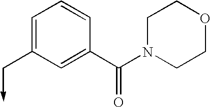 |
| 1027 1028 1029 | rac + − | 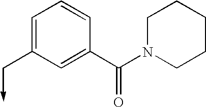 |
| 1030 1031 1032 | rac + − | 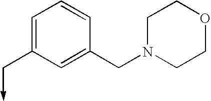 |
| 1033 1034 1035 | rac + − | 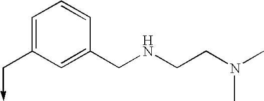 |
| 1036 1037 1038 | rac + − | 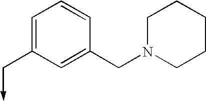 |
| 1039 1040 1041 | rac + − | 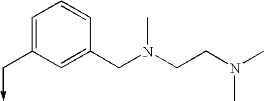 |
| 1042 1043 1044 | rac + − | 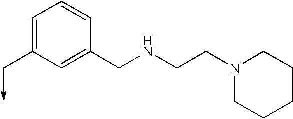 |

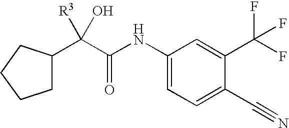
| No. | Racemic or enantiomer | R3 |
|---|---|---|
| 1045 1046 1047 | rac + − | 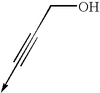 |
| 1048 1049 1050 | rac + − | 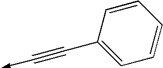 |
| 1051 1052 1053 | rac + − | 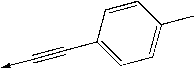 |
| 1054 1055 1056 | rac + − | 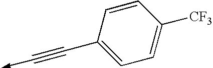 |
| 1057 1058 1059 | rac + − | 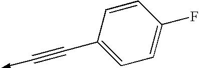 |
| 1060 1061 1062 | rac + − | 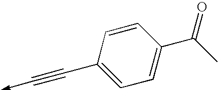 |
| 1063 1064 1065 | rac + − | 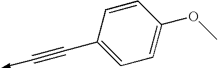 |
| 1066 1067 1068 | rac + − | 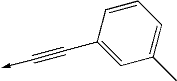 |
| 1069 1070 1071 | rac + − | 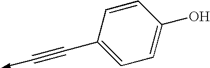 |
| 1072 1073 1074 | rac + − | 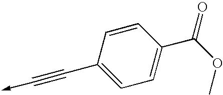 |
| 1075 1076 1077 | rac + − | 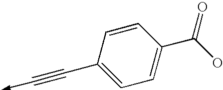 |
| 1078 1079 1080 | rac + − | 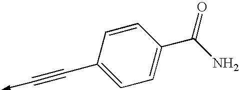 |
| 1081 1082 1083 | rac + − | 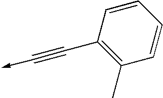 |
| 1084 1085 1086 | rac + − | 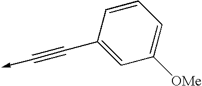 |
| 1087 1088 1089 | rac + − | 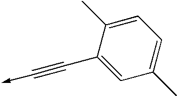 |
| 1090 1091 1092 | rac + − | 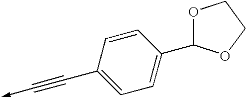 |
| 1093 1094 1095 | rac + − | 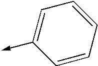 |
| 1096 1097 1098 | rac + − | 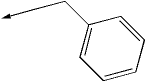 |
| 1099 1100 1101 | rac + − | 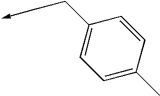 |
| 1102 1103 1104 | rac + − | 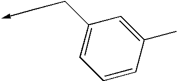 |
| 1105 1106 1107 | rac + − | 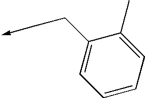 |
| 1108 1109 1110 | rac + − | 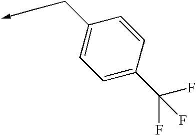 |
| 1111 1112 1113 | rac + − | 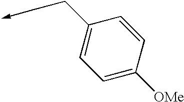 |
| 1114 1115 1116 | rac + − | 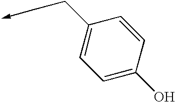 |
| 1117 1118 1119 | rac + − | 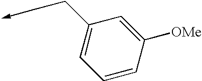 |
| 1120 1121 1122 | rac + − | 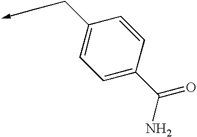 |
| 1123 1124 1125 | rac + − | 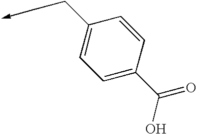 |
| 1126 1127 1128 | rac + − | 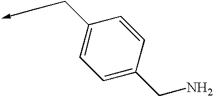 |
| 1129 1130 1131 | rac + − | 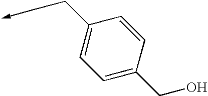 |
| 1132 1133 1134 | rac + − | 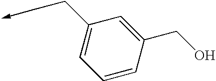 |
| 1135 1136 1137 | rac + − | 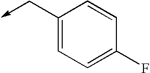 |
| 1138 1139 1140 | rac + − | 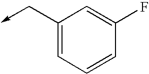 |
| 1141 1142 1143 | rac + − | 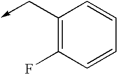 |
| 1144 1145 1146 | rac + − | 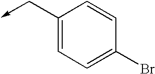 |
| 1147 1148 1149 | rac + − | 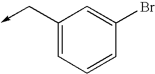 |
| 1150 1151 1152 | rac + − | 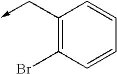 |
| 1153 1154 1155 | rac + − | 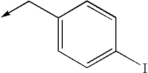 |
| 1156 1157 1158 | rac + − | 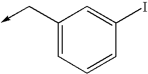 |
| 1159 1160 1161 | rac + − | 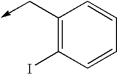 |
| 1162 1163 1164 | rac + − | 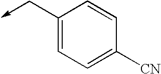 |
| 1165 1166 1167 | rac + − | 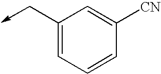 |
| 1168 1169 1170 | rac + − | 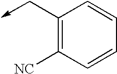 |
| 1171 1172 1173 | rac + − | 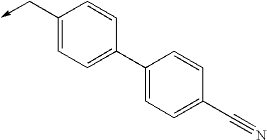 |
| 1174 1175 1176 | rac + − | 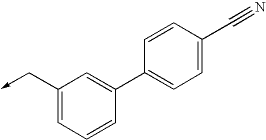 |
| 1177 1178 1179 | rac + − | 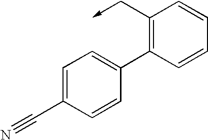 |
| 1180 1181 1182 | rac + − | 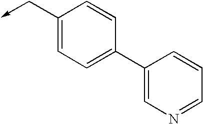 |
| 1183 1184 1185 | rac + − | 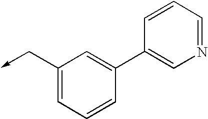 |
| 1186 1187 1188 | rac + − | 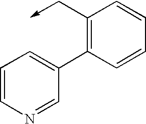 |
| 1189 1190 1191 | rac + − | 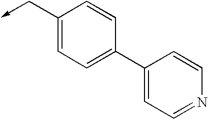 |
| 1192 1193 1194 | rac + − | 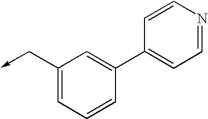 |
| 1195 1196 1197 | rac + − | 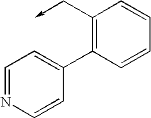 |
| 1198 1199 1200 | rac + − | 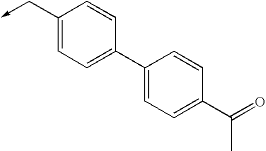 |
| 1201 1202 1203 | rac + − | 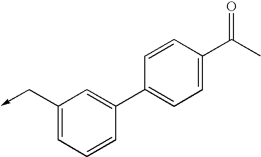 |
| 1204 1205 1206 | rac + − | 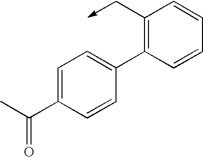 |
| 1207 1208 1209 | rac + − | 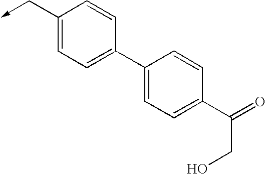 |
| 1210 1211 1212 | rac + − | 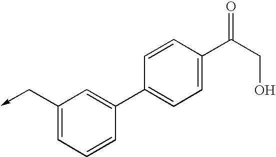 |
| 1213 1214 1215 | rac + − | 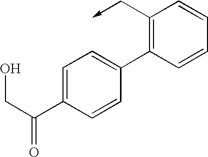 |
| 1216 1217 1218 | rac + − | 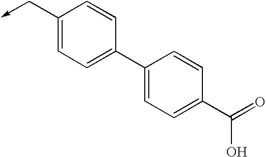 |
| 1219 1220 1221 | rac + − | 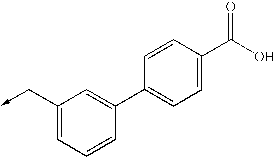 |
| 1222 1223 1224 | rac + − | 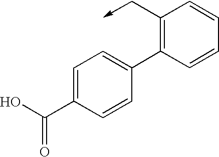 |
| 1225 1226 1227 | rac + − | 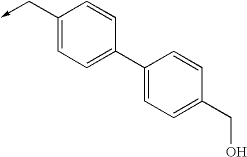 |
| 1228 1229 1230 | rac + − | 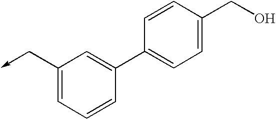 |
| 1231 1232 1233 | rac + − | 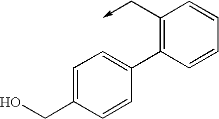 |
| 1234 1235 1236 | rac + − | 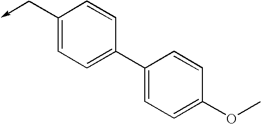 |
| 1237 1238 1239 | rac + − | 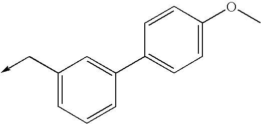 |
| 1240 1241 1242 | rac + − | 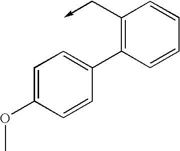 |
| 1243 1244 1245 | rac + − | 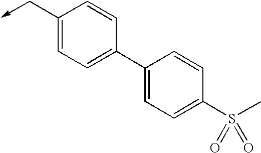 |
| 1246 1247 1248 | rac + − | 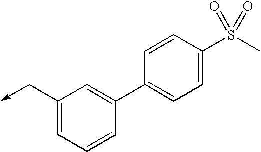 |
| 1249 1250 1251 | rac + − | 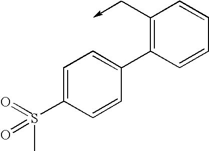 |
| 1252 1253 1254 | rac + − | 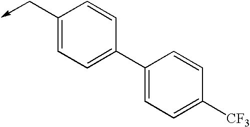 |
| 1255 1256 1257 | rac + − | 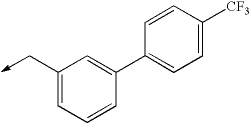 |
| 1258 1259 1260 | rac + − | 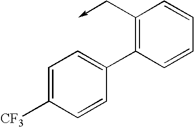 |
| 1261 1262 1263 | rac + − | 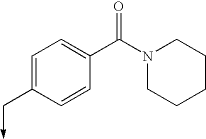 |
| 1264 1265 1266 | rac + − | 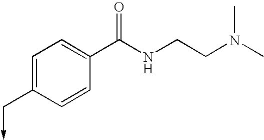 |
| 1267 1268 1269 | rac + − | 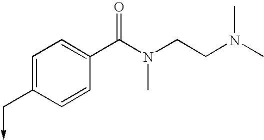 |
| 1270 1271 1272 | rac + − | 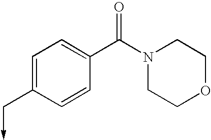 |
| 1273 1274 1275 | rac + − | 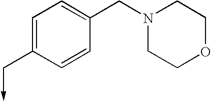 |
| 1276 1277 1278 | rac + − | 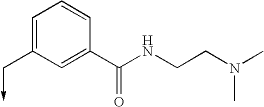 |
| 1279 1280 1281 | rac + − | 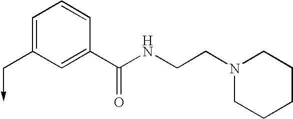 |
| 1282 1283 1284 | rac + − | 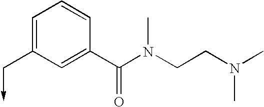 |
| 1285 1286 1287 | rac + − | 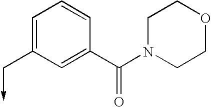 |
| 1288 1289 1290 | rac + − | 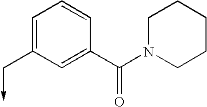 |
| 1291 1292 1293 | rac + − | 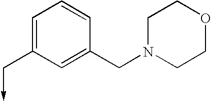 |
| 1294 1295 1296 | rac + − | 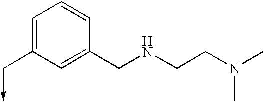 |
| 1297 1298 1299 | rac + − | 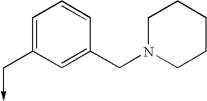 |
| 1300 1301 1302 | rac + − | 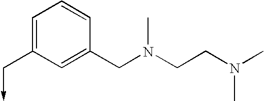 |
| 1303 1304 1305 | rac + − | 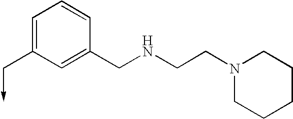 |

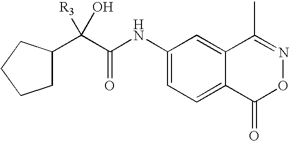
| No. | Racemic or enantiomer | R3 |
|---|---|---|
| 1306 1307 1308 | rac + − | 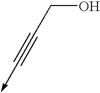 |
| 1309 1310 1311 | rac + − | 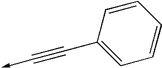 |
| 1312 1313 1314 | rac + − | 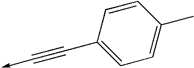 |
| 1315 1316 1317 | rac + − | 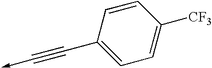 |
| 1318 1319 1320 | rac + − | 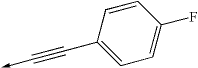 |
| 1321 1322 1323 | rac + − | 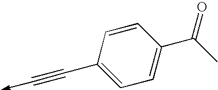 |
| 1324 1325 1326 | rac + − | 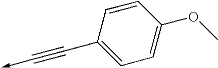 |
| 1327 1328 1329 | rac + − | 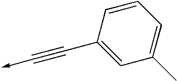 |
| 1330 1331 1332 | rac + − | 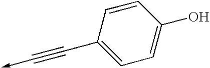 |
| 1333 1334 1335 | rac + − | 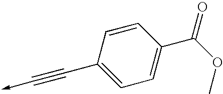 |
| 1336 1337 1338 | rac + − | 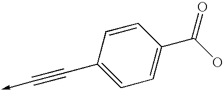 |
| 1339 1340 1341 | rac + − | 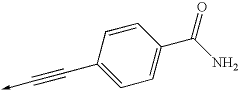 |
| 1342 1343 1344 | rac + − | 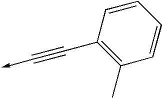 |
| 1345 1346 1347 | rac + − | 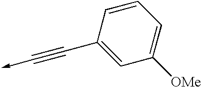 |
| 1348 1349 1350 | rac + − | 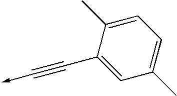 |
| 1351 1352 1353 | rac + − | 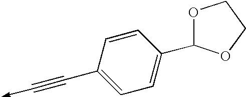 |
| 1354 1355 1356 | rac + − | 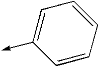 |
| 1357 1358 1359 | rac + − | 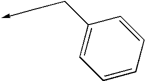 |
| 1360 1361 1362 | rac + − | 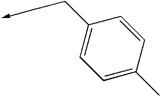 |
| 1363 1364 1365 | rac + − | 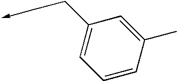 |
| 1366 1367 1368 | rac + − | 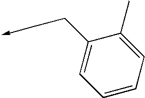 |
| 1369 1370 1371 | rac + − | 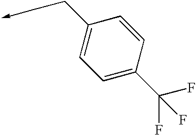 |
| 1372 1373 1374 | rac + − | 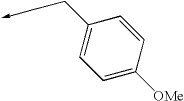 |
| 1375 1376 1377 | rac + − | 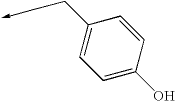 |
| 1378 1379 1380 | rac + − | 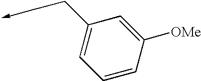 |
| 1381 1382 1383 | rac + − | 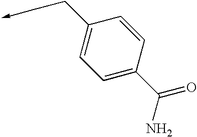 |
| 1384 1385 1386 | rac + − | 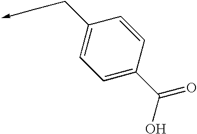 |
| 1387 1388 1389 | rac + − | 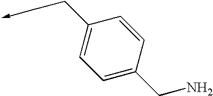 |
| 1390 1391 1392 | rac + − | 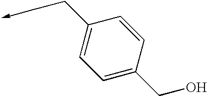 |
| 1393 1394 1395 | rac + − | 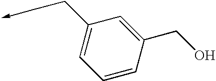 |
| 1396 1397 1398 | rac + − | 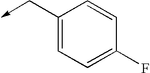 |
| 1399 1400 1401 | rac + − | 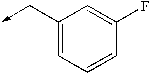 |
| 1402 1403 1404 | rac + − | 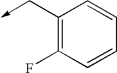 |
| 1405 1406 1407 | rac + − | 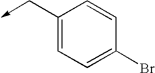 |
| 1408 1409 1410 | rac + − | 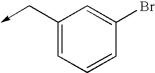 |
| 1411 1412 1413 | rac + − | 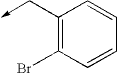 |
| 1414 1415 1416 | rac + − | 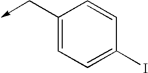 |
| 1417 1418 1419 | rac + − | 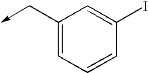 |
| 1420 1421 1422 | rac + − | 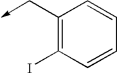 |
| 1423 1424 1425 | rac + − | 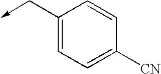 |
| 1426 1427 1428 | rac + − | 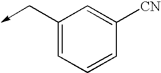 |
| 1429 1430 1431 | rac + − | 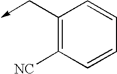 |
| 1432 1433 1434 | rac + − | 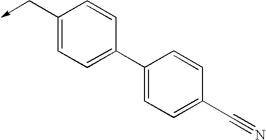 |
| 1435 1436 1437 | rac + − | 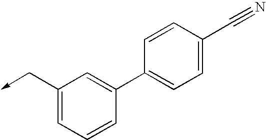 |
| 1438 1439 1440 | rac + − | 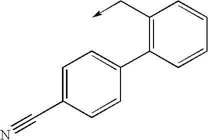 |
| 1441 1442 1443 | rac + − | 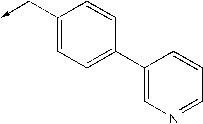 |
| 1444 1445 1446 | rac + − | 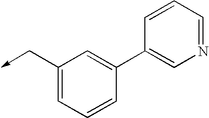 |
| 1447 1448 1449 | rac + − | 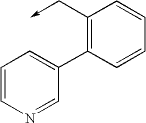 |
| 1450 1451 1452 | rac + − | 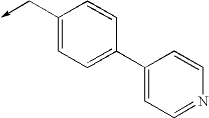 |
| 1453 1454 1455 | rac + − | 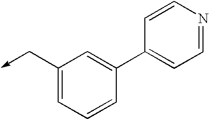 |
| 1456 1457 1458 | rac + − | 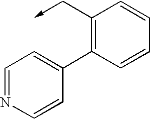 |
| 1459 1460 1461 | rac + − | 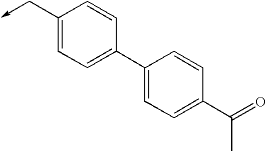 |
| 1462 1463 1464 | rac + − | 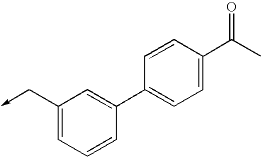 |
| 1465 1466 1467 | rac + − | 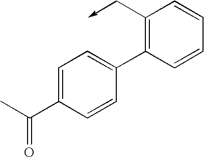 |
| 1468 1469 1470 | rac + − | 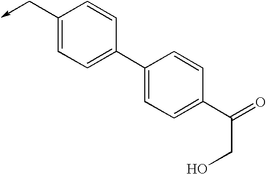 |
| 1471 1472 1473 | rac + − | 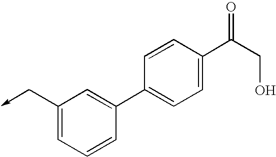 |
| 1474 1475 1476 | rac + − | 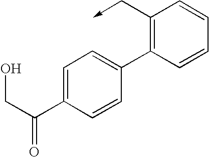 |
| 1477 1478 1479 | rac + − | 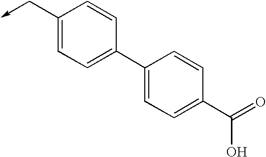 |
| 1480 1481 1482 | rac + − | 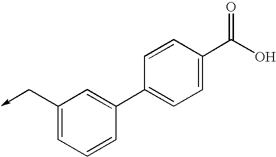 |
| 1483 1484 1485 | rac + − | 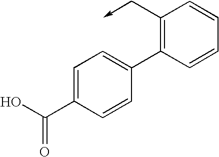 |
| 1486 1487 1488 | rac + − | 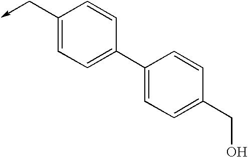 |
| 1489 1490 1491 | rac + − | 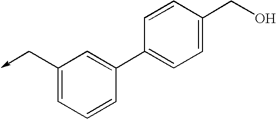 |
| 1492 1493 1494 | rac + − | 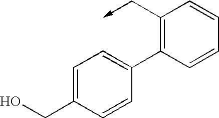 |
| 1495 1496 1497 | rac + − | 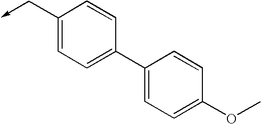 |
| 1498 1499 1500 | rac + − | 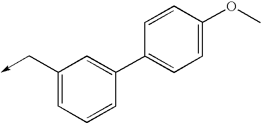 |
| 1501 1502 1503 | rac + − | 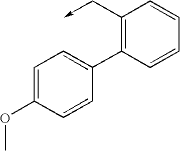 |
| 1504 1505 1506 | rac + − | 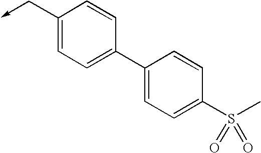 |
| 1507 1508 1509 | rac + − | 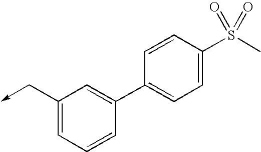 |
| 1510 1511 1512 | rac + − | 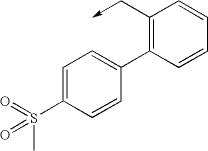 |
| 1513 1514 1515 | rac + − | 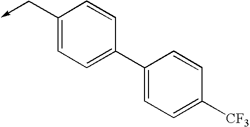 |
| 1516 1517 1518 | rac + − | 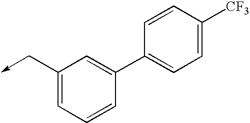 |
| 1519 1520 1521 | rac + − | 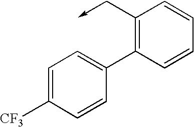 |
| 1522 1523 1524 | rac + − | 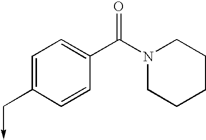 |
| 1525 1526 1527 | rac + − | 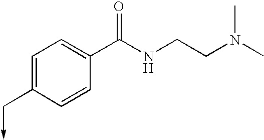 |
| 1528 1529 1530 | rac + − | 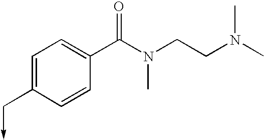 |
| 1531 1532 1533 | rac + − | 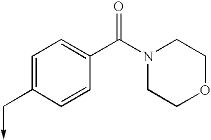 |
| 1534 1535 1536 | rac + − | 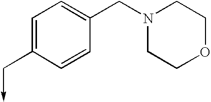 |
| 1537 1538 1539 | rac + − | 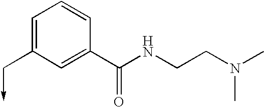 |
| 1540 1541 1542 | rac + − | 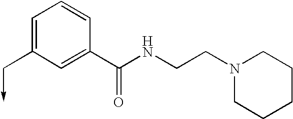 |
| 1543 1544 1545 | rac + − | 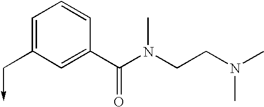 |
| 1546 1547 1548 | rac + − | 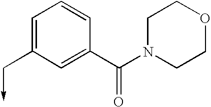 |
| 1549 1550 1551 | rac + − | 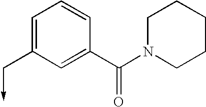 |
| 1552 1553 1554 | rac + − | 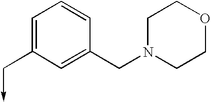 |
| 1555 1556 1557 | rac + − | 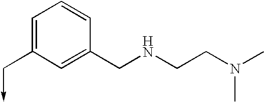 |
| 1558 1559 1560 | rac + − | 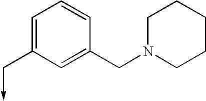 |
| 1561 1562 1563 | rac + − | 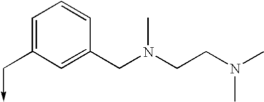 |
| 1564 1565 1566 | rac + − | 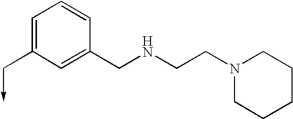 |

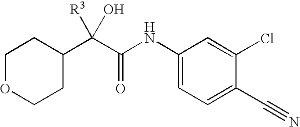
| No. | Racemic or enantiomer | R3 |
|---|---|---|
| 1567 1568 1569 | rac + − | 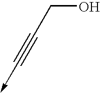 |
| 1570 1571 1572 | rac + − | 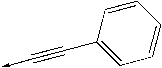 |
| 1573 1574 1575 | rac + − | 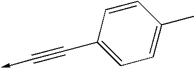 |
| 1576 1577 1578 | rac + − | 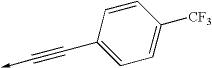 |
| 1579 1580 1581 | rac + − | 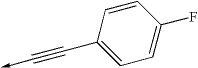 |
| 1582 1583 1584 | rac + − | 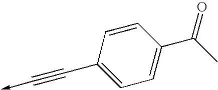 |
| 1585 1586 1587 | rac + − | 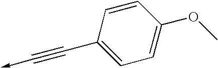 |
| 1588 1589 1590 | rac + − | 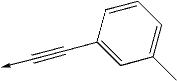 |
| 1591 1592 1593 | rac + − | 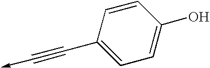 |
| 1594 1595 1596 | rac + − | 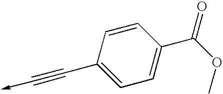 |
| 1597 1598 1599 | rac + − | 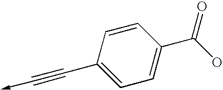 |
| 1600 1601 1602 | rac + − | 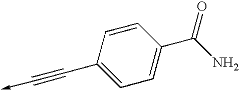 |
| 1603 1604 1605 | rac + − | 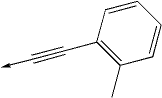 |
| 1606 1607 1608 | rac + − | 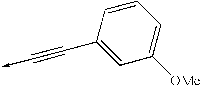 |
| 1609 1610 1611 | rac + − | 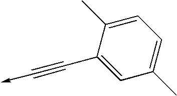 |
| 1612 1613 1614 | rac + − | 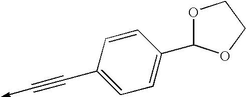 |
| 1615 1616 1617 | rac + − | 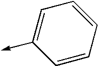 |
| 1618 1619 1620 | rac + − | 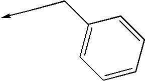 |
| 1621 1622 1623 | rac + − | 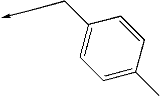 |
| 1624 1625 1626 | rac + − | 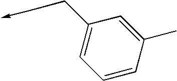 |
| 1627 1628 1629 | rac + − | 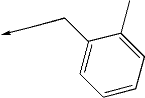 |
| 1630 1631 1632 | rac + − | 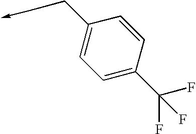 |
| 1633 1634 1635 | rac + − | 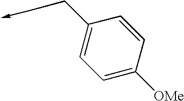 |
| 1636 1637 1638 | rac + − | 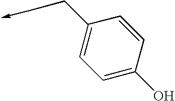 |
| 1639 1640 1641 | rac + − | 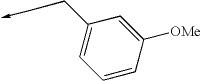 |
| 1642 1643 1644 | rac + − | 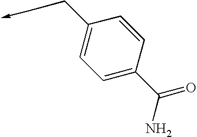 |
| 1645 1646 1647 | rac + − | 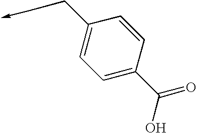 |
| 1648 1649 1650 | rac + − | 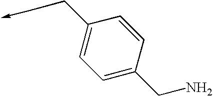 |
| 1651 1652 1653 | rac + − | 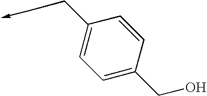 |
| 1654 1655 1656 | rac + − | 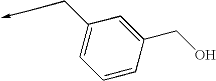 |
| 1657 1658 1659 | rac + − | 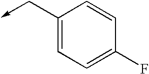 |
| 1660 1661 1662 | rac + − | 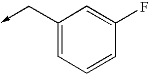 |
| 1663 1664 1665 | rac + − | 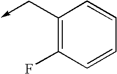 |
| 1666 1667 1668 | rac + − | 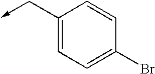 |
| 1669 1670 1671 | rac + − | 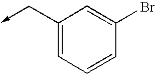 |
| 1672 1673 1674 | rac + − | 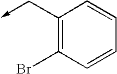 |
| 1675 1676 1677 | rac + − | 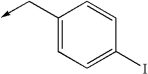 |
| 1678 1679 1680 | rac + − | 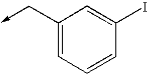 |
| 1681 1682 1683 | rac + − | 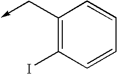 |
| 1684 1685 1686 | rac + − | 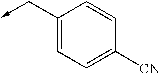 |
| 1687 1688 1689 | rac + − | 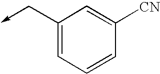 |
| 1690 1691 1692 | rac + − | 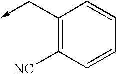 |
| 1693 1694 1695 | rac + − | 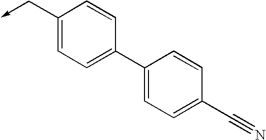 |
| 1696 1697 1698 | rac + − | 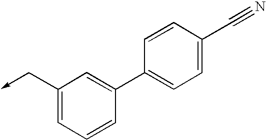 |
| 1699 1700 1701 | rac + − | 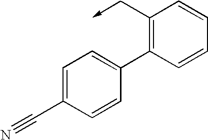 |
| 1702 1703 1704 | rac + − | 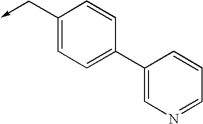 |
| 1705 1706 1707 | rac + − | 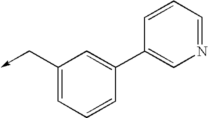 |
| 1708 1709 1710 | rac + − | 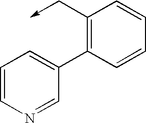 |
| 1711 1712 1713 | rac + − | 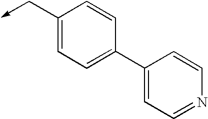 |
| 1714 1715 1716 | rac + − | 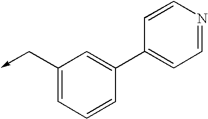 |
| 1717 1718 1719 | rac + − | 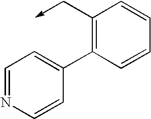 |
| 1720 1721 1722 | rac + − | 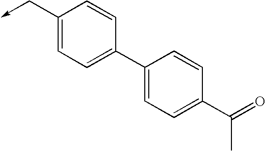 |
| 1723 1724 1725 | rac + − | 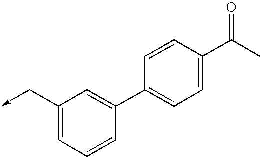 |
| 1726 1727 1728 | rac + − | 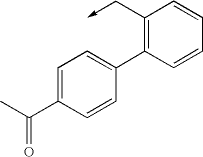 |
| 1729 1730 1731 | rac + − | 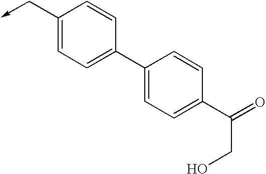 |
| 1732 1733 1734 | rac + − | 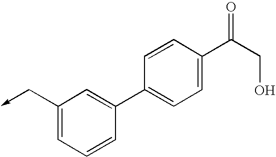 |
| 1735 1736 1737 | rac + − | 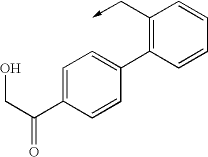 |
| 1738 1739 1740 | rac + − | 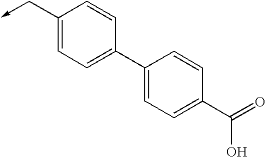 |
| 1741 1742 1743 | rac + − | 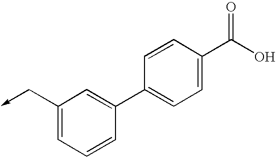 |
| 1744 1745 1746 | rac + − | 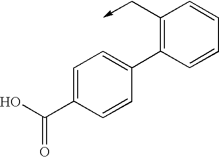 |
| 1747 1748 1749 | rac + − | 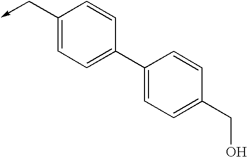 |
| 1750 1751 1752 | rac + − | 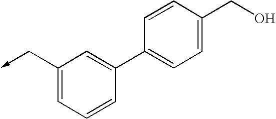 |
| 1753 1754 1755 | rac + − | 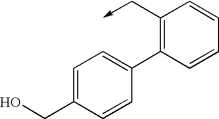 |
| 1756 1757 1758 | rac + − | 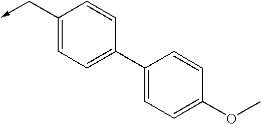 |
| 1759 1760 1761 | rac + − | 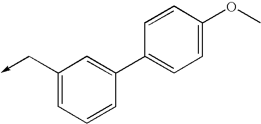 |
| 1762 1763 1764 | rac + − | 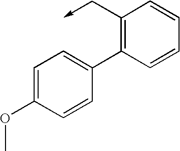 |
| 1765 1766 1767 | rac + − | 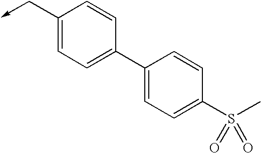 |
| 1768 1769 1770 | rac + − | 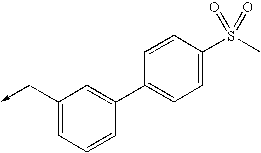 |
| 1771 1772 1773 | rac + − | 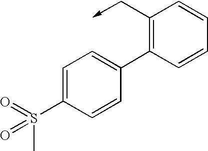 |
| 1774 1775 1776 | rac + − | 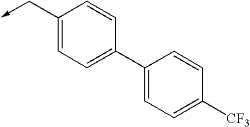 |
| 1777 1778 1779 | rac + − | 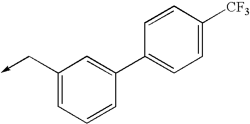 |
| 1780 1781 1782 | rac + − | 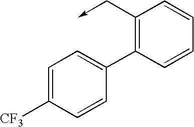 |
| 1783 1784 1785 | rac + − | 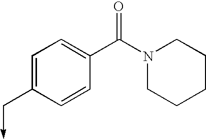 |
| 1786 1787 1788 | rac + − | 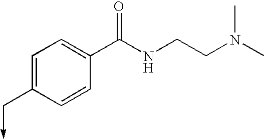 |
| 1789 1790 1791 | rac + − | 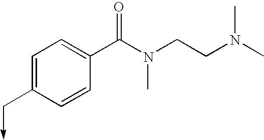 |
| 1792 1793 1794 | rac + − | 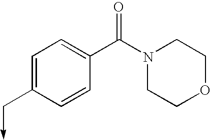 |
| 1795 1796 1797 | rac + − | 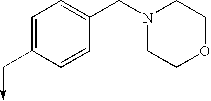 |
| 1798 1799 1800 | rac + − | 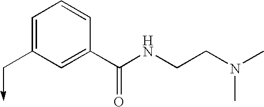 |
| 1801 1802 1803 | rac + − | 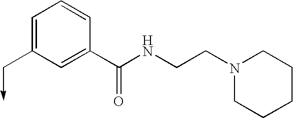 |
| 1804 1805 1806 | rac + − | 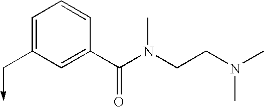 |
| 1807 1808 1809 | rac + − | 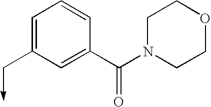 |
| 1810 1811 1812 | rac + − | 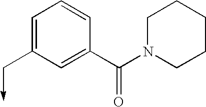 |
| 1813 1814 1815 | rac + − | 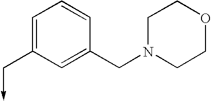 |
| 1816 1817 1818 | rac + − | 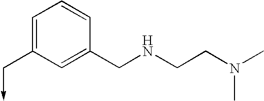 |
| 1819 1820 1821 | rac + − | 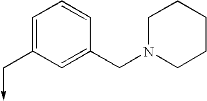 |
| 1822 1823 1824 | rac + − | 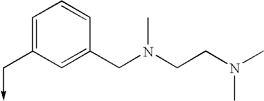 |
| 1825 1826 1827 | rac + − | 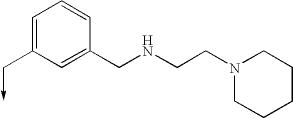 |

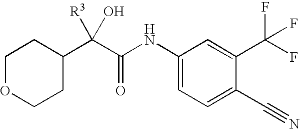
| No. | Racemic or enantiomer | R3 |
|---|---|---|
| 1828 1829 1830 | rac + − | 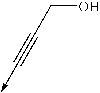 |
| 1831 1832 1833 | rac + − | 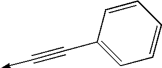 |
| 1834 1835 1836 | rac + − | 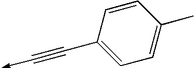 |
| 1837 1838 1839 | rac + − | 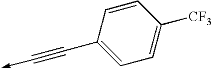 |
| 1840 1841 1842 | rac + − | 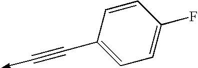 |
| 1843 1844 1845 | rac + − | 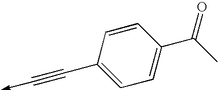 |
| 1846 1847 1848 | rac + − | 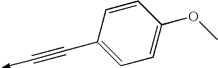 |
| 1849 1850 1851 | rac + − | 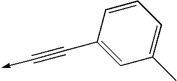 |
| 1852 1853 1854 | rac + − | 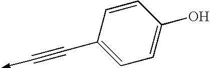 |
| 1855 1856 1857 | rac + − | 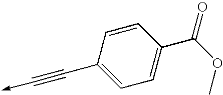 |
| 1858 1859 1860 | rac + − | 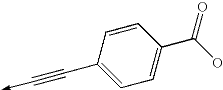 |
| 1861 1862 1863 | rac + − | 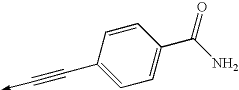 |
| 1864 1865 1866 | rac + − | 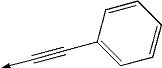 |
| 1867 1868 1869 | rac + − | 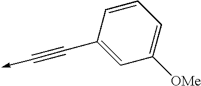 |
| 1870 1871 1872 | rac + − | 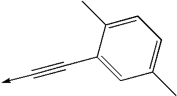 |
| 1873 1874 1875 | rac + − | 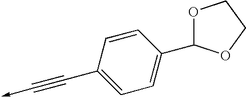 |
| 1876 1877 1878 | rac + − | 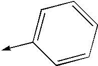 |
| 1879 1880 1881 | rac + − | 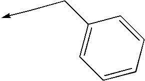 |
| 1882 1883 1884 | rac + − | 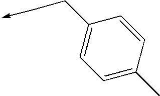 |
| 1885 1886 1887 | rac + − | 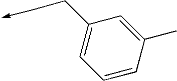 |
| 1888 1889 1890 | rac + − | 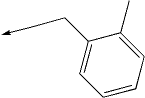 |
| 1891 1892 1893 | rac + − | 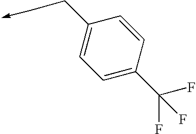 |
| 1894 1895 1896 | rac + − | 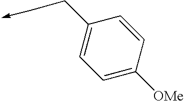 |
| 1897 1898 1899 | rac + − | 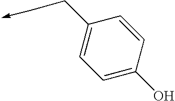 |
| 1900 1901 1902 | rac + − | 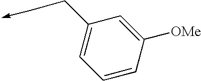 |
| 1903 1904 1905 | rac + − | 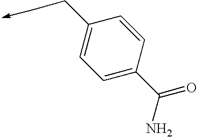 |
| 1906 1907 1908 | rac + − | 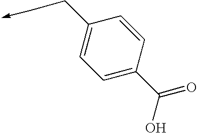 |
| 1909 1910 1911 | rac + − | 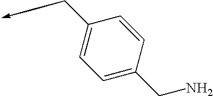 |
| 1912 1913 1914 | rac + − | 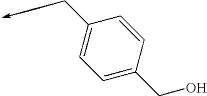 |
| 1915 1916 1917 | rac + − | 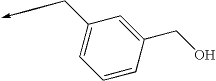 |
| 1918 1919 1920 | rac + − | 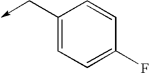 |
| 1921 1922 1923 | rac + − | 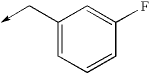 |
| 1924 1925 1926 | rac + − | 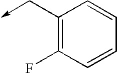 |
| 1927 1928 1929 | rac + − | 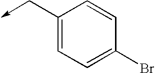 |
| 1930 1931 1932 | rac + − | 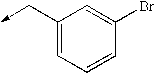 |
| 1933 1934 1935 | rac + − | 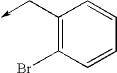 |
| 1936 1937 1938 | rac + − | 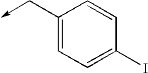 |
| 1939 1940 1941 | rac + − | 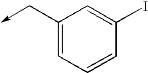 |
| 1942 1943 1944 | rac + − | 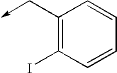 |
| 1945 1946 1947 | rac + − | 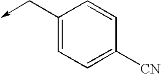 |
| 1948 1949 1950 | rac + − | 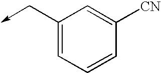 |
| 1951 1952 1953 | rac + − | 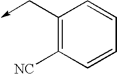 |
| 1954 1955 1956 | rac + − | 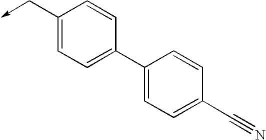 |
| 1957 1958 1959 | rac + − | 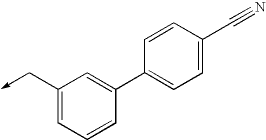 |
| 1960 1961 1962 | rac + − | 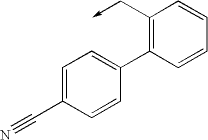 |
| 1963 1964 1965 | rac + − | 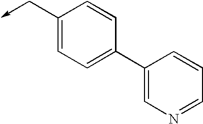 |
| 1966 1967 1968 | rac + − | 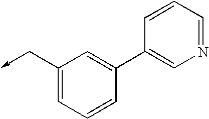 |
| 1969 1970 1971 | rac + − | 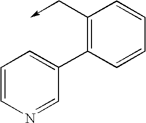 |
| 1972 1973 1974 | rac + − | 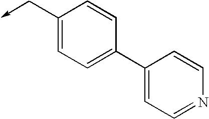 |
| 1975 1976 1977 | rac + − | 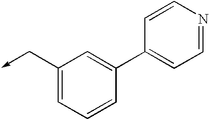 |
| 1978 1979 1980 | rac + − | 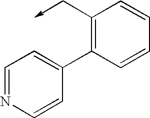 |
| 1981 1982 1983 | rac + − | 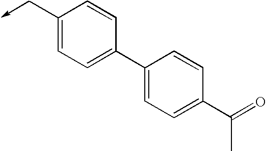 |
| 1984 1985 1986 | rac + − | 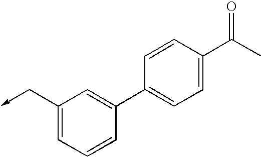 |
| 1987 1988 1989 | rac + − | 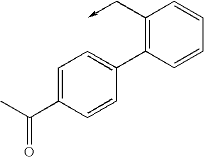 |
| 1990 1991 1992 | rac + − | 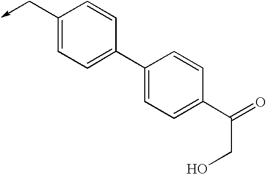 |
| 1993 1994 1995 | rac + − | 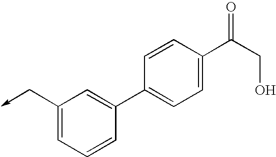 |
| 1996 1997 1998 | rac + − | 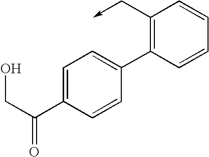 |
| 1999 2000 2001 | rac + − | 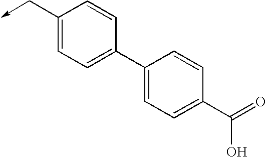 |
| 2002 2003 2004 | rac + − | 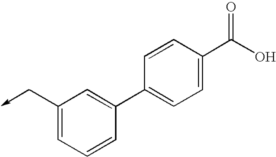 |
| 2005 2006 2007 | rac + − | 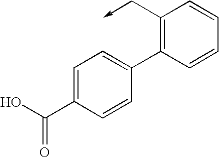 |
| 2008 2009 2010 | rac + − | 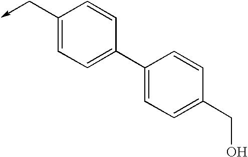 |
| 2011 2012 2013 | rac + − | 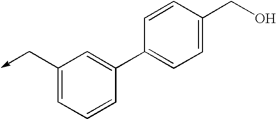 |
| 2014 2015 2016 | rac + − | 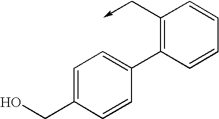 |
| 2017 2018 2019 | rac + − | 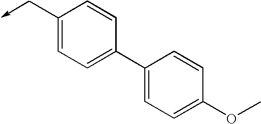 |
| 2020 2021 2022 | rac + − | 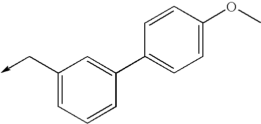 |
| 2023 2024 2025 | rac + − | 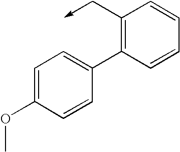 |
| 2026 2027 2028 | rac + − | 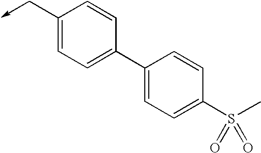 |
| 2029 2030 2031 | rac + − | 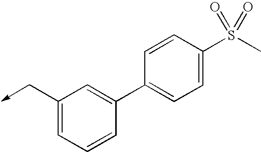 |
| 2032 2033 2034 | rac + − | 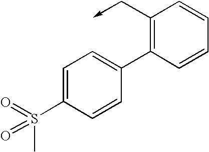 |
| 2035 2036 2037 | rac + − | 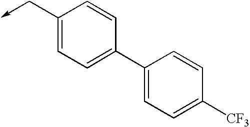 |
| 2038 2039 2040 | rac + − | 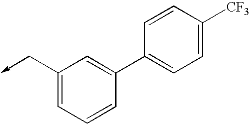 |
| 2041 2042 2043 | rac + − | 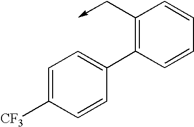 |
| 2044 2045 2046 | rac + − | 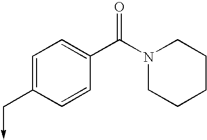 |
| 2047 2048 2049 | rac + − | 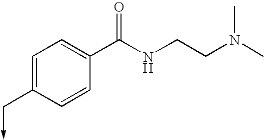 |
| 2050 2051 2052 | rac + − | 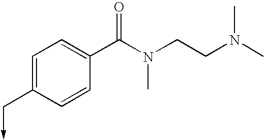 |
| 2053 2054 2055 | rac + − | 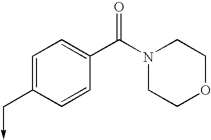 |
| 2056 2057 2058 | rac + − | 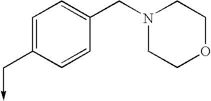 |
| 2059 2060 2061 | rac + − | 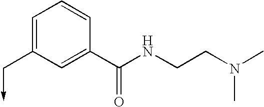 |
| 2062 2063 2064 | rac + − | 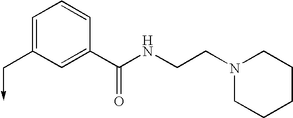 |
| 2065 2066 2067 | rac + − | 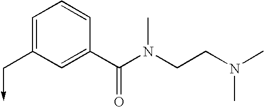 |
| 2068 2069 2070 | rac + − | 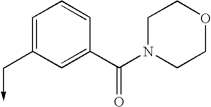 |
| 2071 2072 2073 | rac + − | 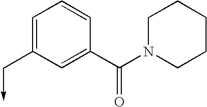 |
| 2074 2075 2076 | rac + − | 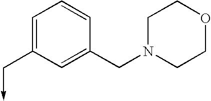 |
| 2077 2078 2079 | rac + − | 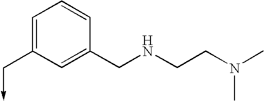 |
| 2080 2081 2082 | rac + − | 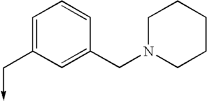 |
| 2083 2084 2085 | rac + − | 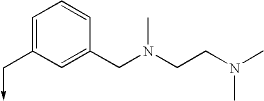 |
| 2086 2087 2088 | rac + − | 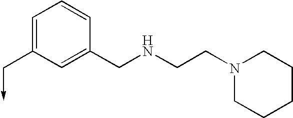 |

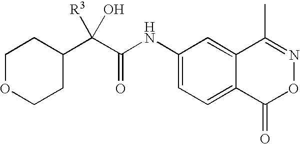
| No. | Racemic or enantiomer | R3 |
|---|---|---|
| 2089 2090 2091 | rac + − | 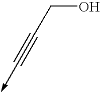 |
| 2092 2093 2094 | rac + − | 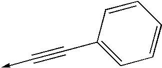 |
| 2095 2096 2097 | rac + − | 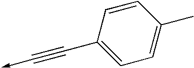 |
| 2098 2099 2100 | rac + − | 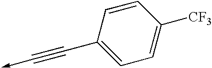 |
| 2101 2102 2103 | rac + − | 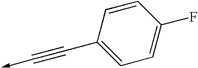 |
| 2104 2105 2106 | rac + − | 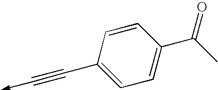 |
| 2107 2108 2109 | rac + − | 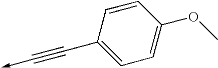 |
| 2110 2111 2112 | rac + − | 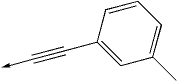 |
| 2113 2114 2115 | rac + − | 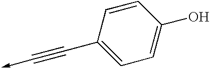 |
| 2116 2117 2118 | rac + − | 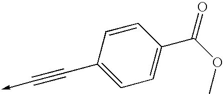 |
| 2119 2120 2121 | rac + − | 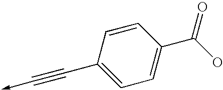 |
| 2122 2123 2124 | rac + − | 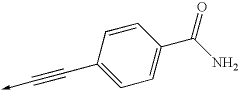 |
| 2125 2126 2127 | rac + − | 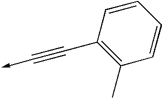 |
| 2128 2129 2130 | rac + − | 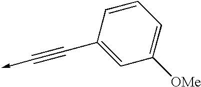 |
| 2131 2132 2133 | rac + − | 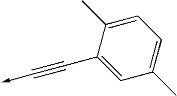 |
| 2134 2135 2136 | rac + − | 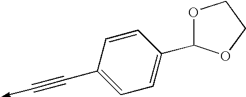 |
| 2137 2138 2139 | rac + − | 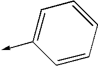 |
| 2140 2141 2142 | rac + − | 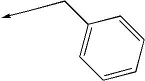 |
| 2143 2144 2145 | rac + − | 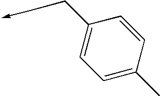 |
| 2146 2147 2148 | rac + − | 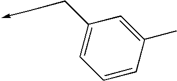 |
| 2149 2150 2151 | rac + − | 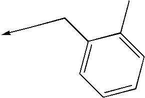 |
| 2152 2153 2154 | rac + − | 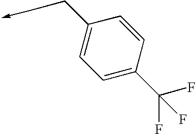 |
| 2155 2156 2157 | rac + − | 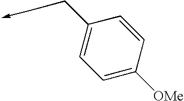 |
| 2158 2159 2160 | rac + − | 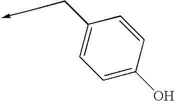 |
| 2161 2162 2163 | rac + − | 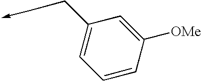 |
| 2164 2165 2166 | rac + − | 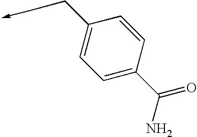 |
| 2167 2168 2169 | rac + − | 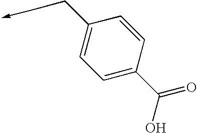 |
| 2170 2171 2172 | rac + − | 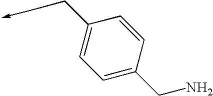 |
| 2173 2174 2175 | rac + − | 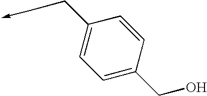 |
| 2176 2177 2178 | rac + − | 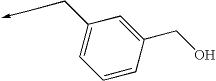 |
| 2179 2180 2181 | rac + − | 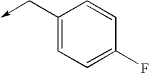 |
| 2182 2183 2184 | rac + − | 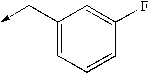 |
| 2185 2186 2187 | rac + − | 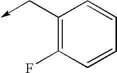 |
| 2188 2189 2190 | rac + − | 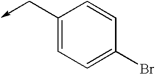 |
| 2191 2192 2193 | rac + − | 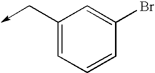 |
| 2194 2195 2196 | rac + − | 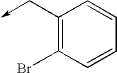 |
| 2197 2198 2199 | rac + − | 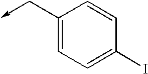 |
| 2200 2201 2202 | rac + − | 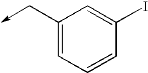 |
| 2203 2204 2205 | rac + − | 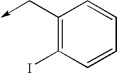 |
| 2206 2207 2208 | rac + − | 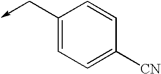 |
| 2209 2210 2211 | rac + − | 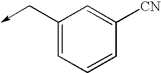 |
| 2212 2213 2214 | rac + − | 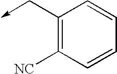 |
| 2215 2216 2217 | rac + − | 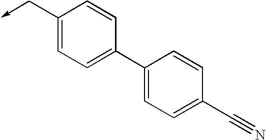 |
| 2218 2219 2220 | rac + − | 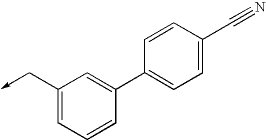 |
| 2221 2222 2223 | rac + − | 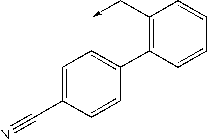 |
| 2224 2225 2226 | rac + − | 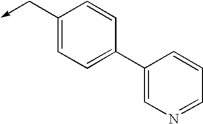 |
| 2227 2228 2229 | rac + − | 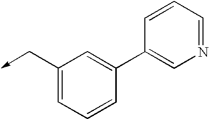 |
| 2230 2231 2232 | rac + − | 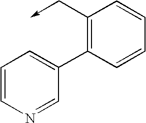 |
| 2233 2234 2235 | rac + − | 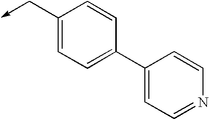 |
| 2236 2237 2238 | rac + − | 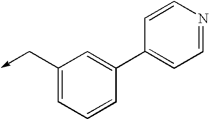 |
| 2239 2240 2241 | rac + − | 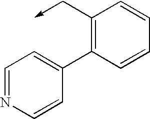 |
| 2242 2243 2244 | rac + − | 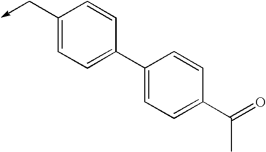 |
| 2245 2246 2247 | rac + − | 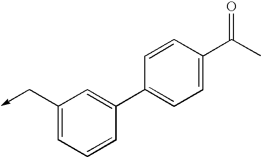 |
| 2248 2249 2250 | rac + − | 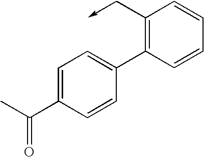 |
| 2251 2252 2253 | rac + − | 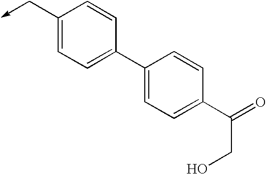 |
| 2254 2255 2256 | rac + − | 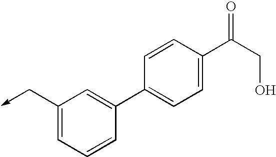 |
| 2257 2258 2259 | rac + − | 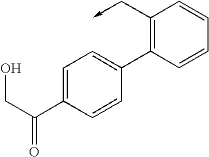 |
| 2260 2261 2262 | rac + − | 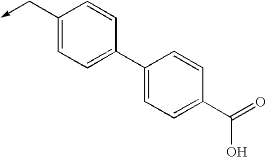 |
| 2263 2264 2265 | rac + − | 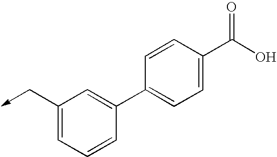 |
| 2266 2267 2268 | rac + − | 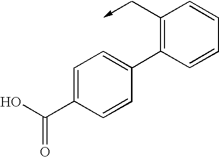 |
| 2269 2270 2271 | rac + − | 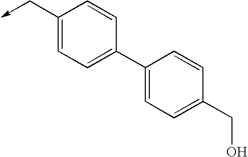 |
| 2272 2273 2274 | rac + − | 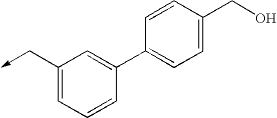 |
| 2275 2276 2277 | rac + − | 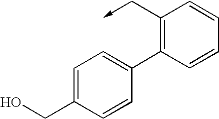 |
| 2278 2279 2280 | rac + − | 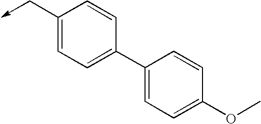 |
| 2281 2282 2283 | rac + − | 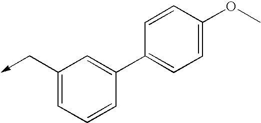 |
| 2284 2285 2286 | rac + − | 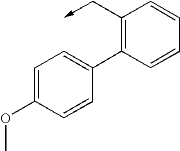 |
| 2287 2288 2289 | rac + − | 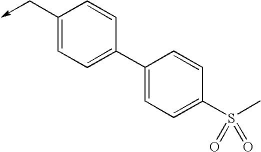 |
| 2290 2291 2292 | rac + − | 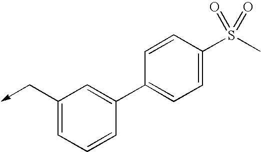 |
| 2293 2294 2295 | rac + − | 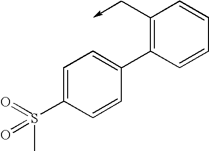 |
| 2296 2297 2298 | rac + − | 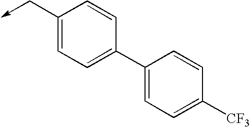 |
| 2299 2300 2301 | rac + − | 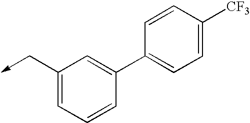 |
| 2302 2303 2304 | rac + − | 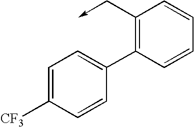 |
| 2305 2306 2307 | rac + − | 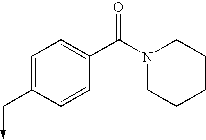 |
| 2308 2309 2310 | rac + − | 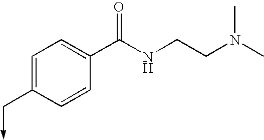 |
| 2311 2312 2313 | rac + − | 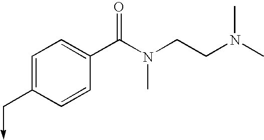 |
| 2314 2315 2316 | rac + − | 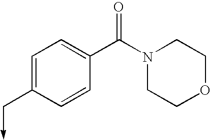 |
| 2317 2318 2319 | rac + − | 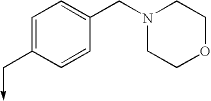 |
| 2320 2321 2322 | rac + − | 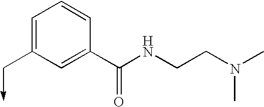 |
| 2323 2324 2325 | rac + − | 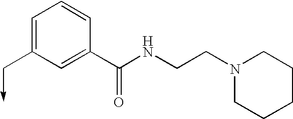 |
| 2326 2327 2328 | rac + − | 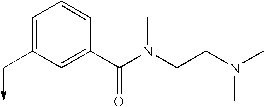 |
| 2329 2330 2331 | rac + − | 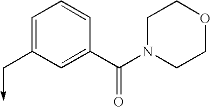 |
| 2332 2333 2334 | rac + − | 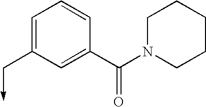 |
| 2335 2336 2337 | rac + − | 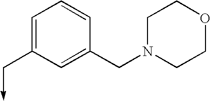 |
| 2338 2339 2340 | rac + − | 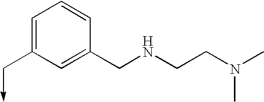 |
| 2341 2342 2343 | rac + − | 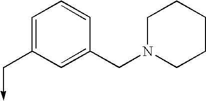 |
| 2344 2345 2346 | rac + − | 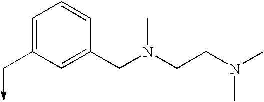 |
| 2347 2348 2349 | rac + − | 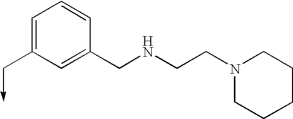 |

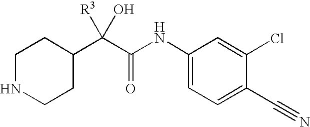
| No. | Racemic or enantiomer | R3 |
|---|---|---|
| 2350 2351 2352 | rac + − | 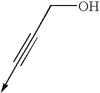 |
| 2353 2354 2355 | rac + − | 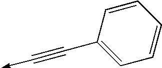 |
| 2356 2357 2358 | rac + − | 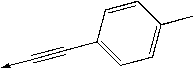 |
| 2359 2360 2361 | rac + − | 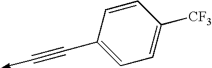 |
| 2362 2363 2364 | rac + − | 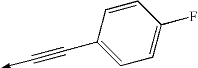 |
| 2365 2366 2367 | rac + − | 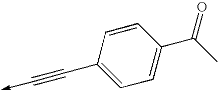 |
| 2368 2369 2370 | rac + − | 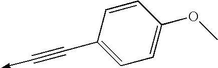 |
| 2371 2372 2373 | rac + − | 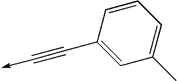 |
| 2374 2375 2376 | rac + − | 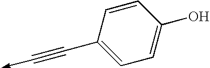 |
| 2377 2378 2379 | rac + − | 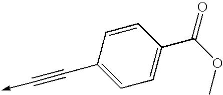 |
| 2380 2381 2382 | rac + − | 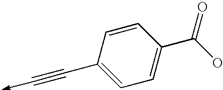 |
| 2383 2384 2385 | rac + − | 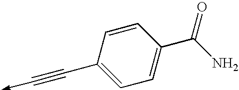 |
| 2386 2387 2388 | rac + − | 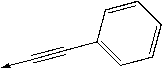 |
| 2389 2390 2391 | rac + − | 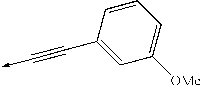 |
| 2392 2393 2394 | rac + − | 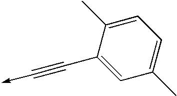 |
| 2395 2396 2397 | rac + − | 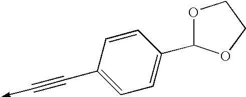 |
| 2398 2399 2400 | rac + − | 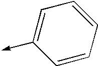 |
| 2401 2402 2403 | rac + − | 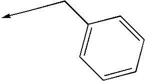 |
| 2404 2405 2406 | rac + − | 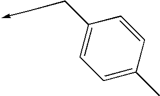 |
| 2407 2408 2409 | rac + − | 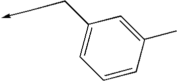 |
| 2410 2411 2412 | rac + − | 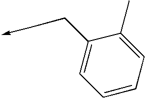 |
| 2413 2414 2415 | rac + − | 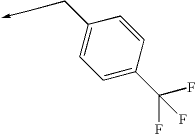 |
| 2416 2417 2418 | rac + − | 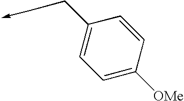 |
| 2419 2420 2421 | rac + − | 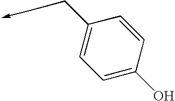 |
| 2422 2423 2424 | rac + − | 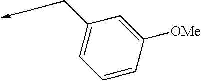 |
| 2425 2426 2427 | rac + − | 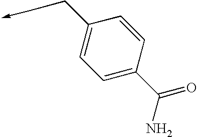 |
| 2428 2429 2430 | rac + − | 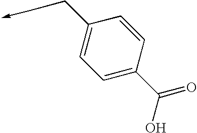 |
| 2431 2432 2433 | rac + − | 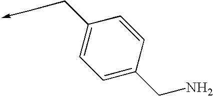 |
| 2434 2435 2436 | rac + − | 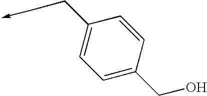 |
| 2437 2438 2439 | rac + − | 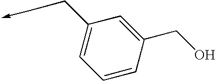 |
| 2440 2441 2442 | rac + − | 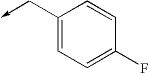 |
| 2443 2444 2445 | rac + − | 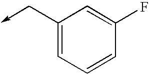 |
| 2446 2447 2448 | rac + − | 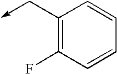 |
| 2449 2450 2451 | rac + − | 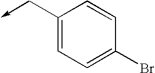 |
| 2452 2453 2454 | rac + − | 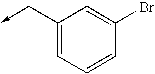 |
| 2455 2456 2457 | rac + − | 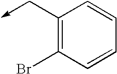 |
| 2458 2459 2460 | rac + − | 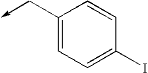 |
| 2461 2462 2463 | rac + − | 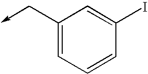 |
| 2464 2465 2466 | rac + − | 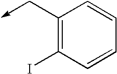 |
| 2467 2468 2469 | rac + − | 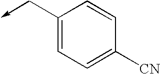 |
| 2470 2471 2472 | rac + − | 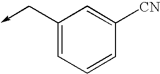 |
| 2473 2474 2475 | rac + − | 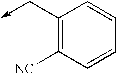 |
| 2476 2477 2478 | rac + − | 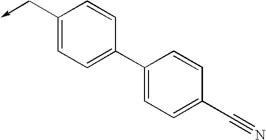 |
| 2479 2480 2481 | rac + − | 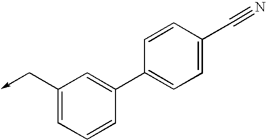 |
| 2482 2483 2484 | rac + − | 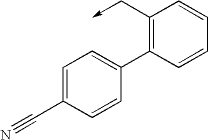 |
| 2485 2486 2487 | rac + − | 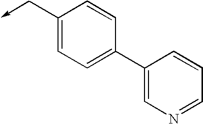 |
| 2488 2489 2490 | rac + − | 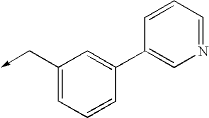 |
| 2491 2492 2493 | rac + − | 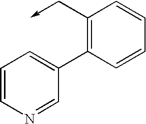 |
| 2494 2495 2496 | rac + − | 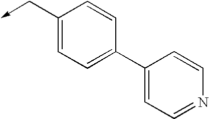 |
| 2497 2498 2499 | rac + − | 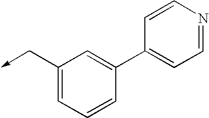 |
| 2500 2501 2502 | rac + − | 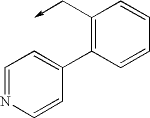 |
| 2503 2504 2505 | rac + − | 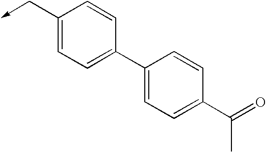 |
| 2506 2507 2508 | rac + − | 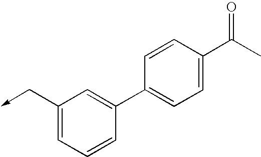 |
| 2509 2510 2511 | rac + − | 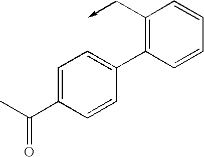 |
| 2512 2513 2514 | rac + − | 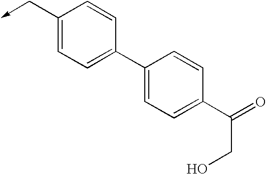 |
| 2515 2516 2517 | rac + − | 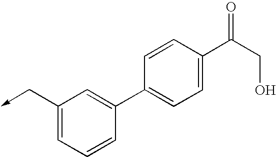 |
| 2518 2519 2520 | rac + − | 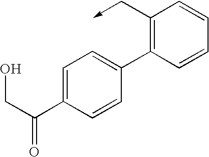 |
| 2521 2522 2523 | rac + − | 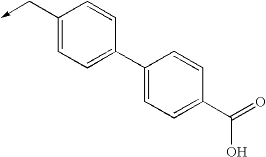 |
| 2524 2525 2526 | rac + − | 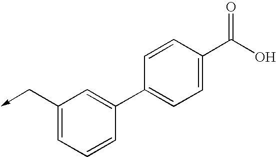 |
| 2527 2528 2529 | rac + − | 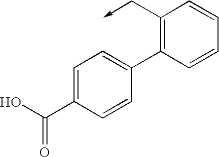 |
| 2530 2531 2532 | rac + − | 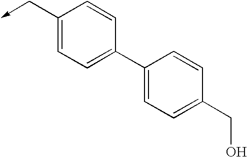 |
| 2533 2534 2535 | rac + − | 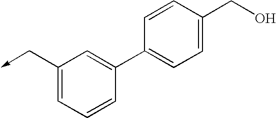 |
| 2536 2537 2538 | rac + − | 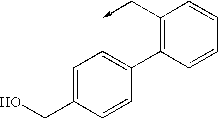 |
| 2539 2540 2541 | rac + − | 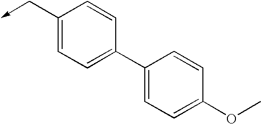 |
| 2542 2543 2544 | rac + − | 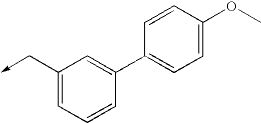 |
| 2545 2546 2547 | rac + − | 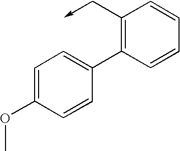 |
| 2548 2549 2550 | rac + − | 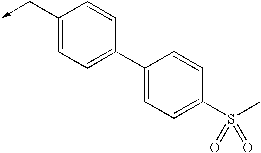 |
| 2551 2552 2553 | rac + − | 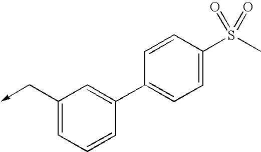 |
| 2554 2555 2556 | rac + − | 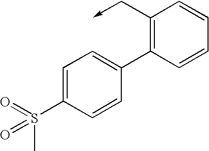 |
| 2557 2558 2559 | rac + − | 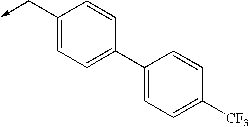 |
| 2560 2561 2562 | rac + − | 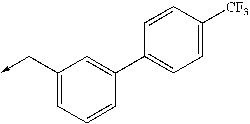 |
| 2563 2564 2565 | rac + − | 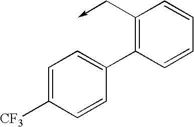 |
| 2566 2567 2568 | rac + − | 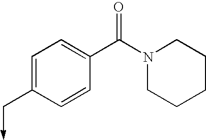 |
| 2569 2570 2571 | rac + − | 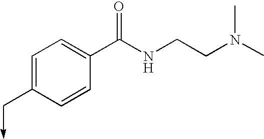 |
| 2572 2573 2574 | rac + − | 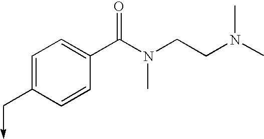 |
| 2575 2576 2577 | rac + − | 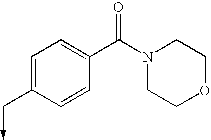 |
| 2578 2579 2580 | rac + − | 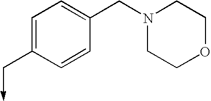 |
| 2581 2582 2583 | rac + − | 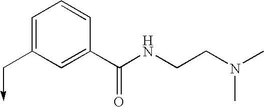 |
| 2584 2585 2586 | rac + − | 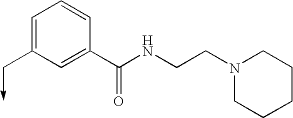 |
| 2587 2588 2589 | rac + − | 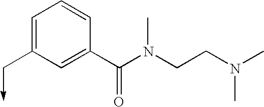 |
| 2590 2591 2592 | rac + − | 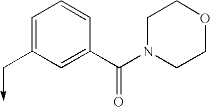 |
| 2593 2594 2595 | rac + − | 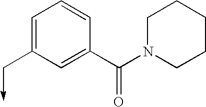 |
| 2596 2597 2598 | rac + − | 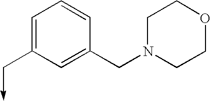 |
| 2599 2600 2601 | rac + − | 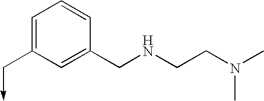 |
| 2602 2603 2604 | rac + − | 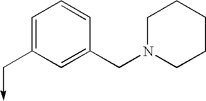 |
| 2605 2606 2607 | rac + − | 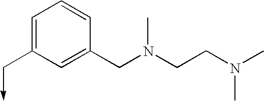 |
| 2608 2609 2610 | rac + − | 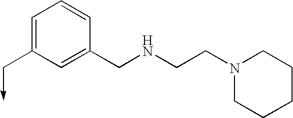 |

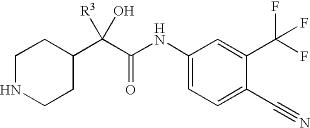
| No. | Racemic or enantiomer | R3 |
|---|---|---|
| 2611 2612 2613 | rac + − | 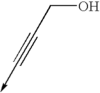 |
| 2614 2615 2616 | rac + − | 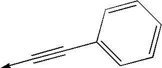 |
| 2617 2618 2619 | rac + − | 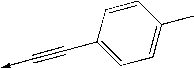 |
| 2620 2621 2622 | rac + − | 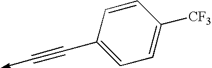 |
| 2623 2624 2625 | rac + − | 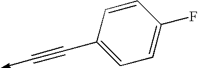 |
| 2626 2627 2628 | rac + − | 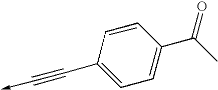 |
| 2629 2630 2631 | rac + − | 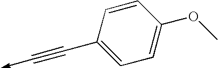 |
| 2632 2633 2634 | rac + − | 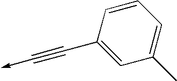 |
| 2635 2636 2637 | rac + − | 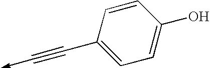 |
| 2638 2639 2640 | rac + − | 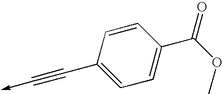 |
| 2641 2642 2643 | rac + − | 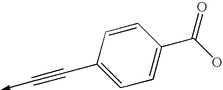 |
| 2644 2645 2646 | rac + − | 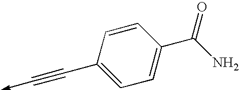 |
| 2647 2648 2649 | rac + − | 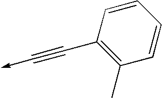 |
| 2650 2651 2652 | rac + − | 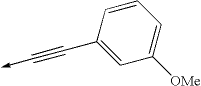 |
| 2653 2654 2655 | rac + − | 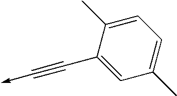 |
| 2656 2657 2658 | rac + − | 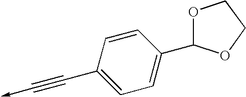 |
| 2659 2660 2661 | rac + − | 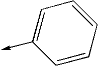 |
| 2662 2663 2664 | rac + − | 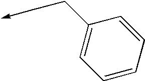 |
| 2665 2666 2667 | rac + − | 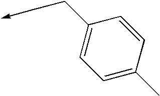 |
| 2668 2669 2670 | rac + − | 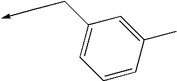 |
| 2671 2672 2673 | rac + − | 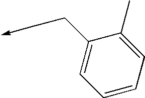 |
| 2674 2675 2676 | rac + − | 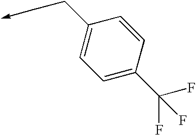 |
| 2677 2678 2679 | rac + − | 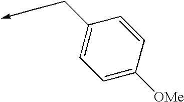 |
| 2680 2681 2682 | rac + − | 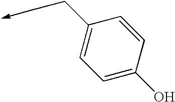 |
| 2683 2684 2685 | rac + − | 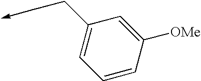 |
| 2686 2687 2688 | rac + − | 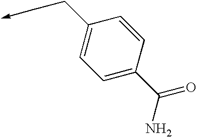 |
| 2689 2690 2691 | rac + − | 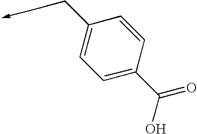 |
| 2692 2693 2694 | rac + − | 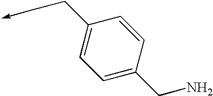 |
| 2695 2696 2697 | rac + − | 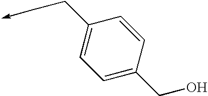 |
| 2698 2699 2700 | rac + − | 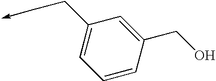 |
| 2701 2702 2703 | rac + − | 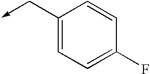 |
| 2704 2705 2706 | rac + − | 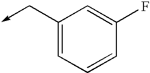 |
| 2707 2708 2709 | rac + − | 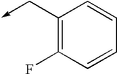 |
| 2710 2711 2712 | rac + − | 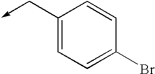 |
| 2713 2714 2715 | rac + − | 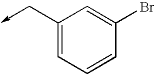 |
| 2716 2717 2718 | rac + − | 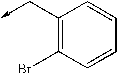 |
| 2719 2720 2721 | rac + − | 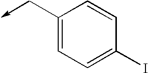 |
| 2722 2723 2724 | rac + − | 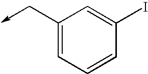 |
| 2725 2726 2727 | rac + − | 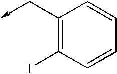 |
| 2728 2729 2730 | rac + − | 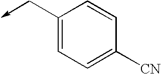 |
| 2731 2732 2733 | rac + − | 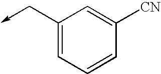 |
| 2734 2735 2736 | rac + − | 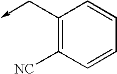 |
| 2737 2738 2739 | rac + − | 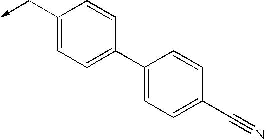 |
| 2740 2741 2742 | rac + − | 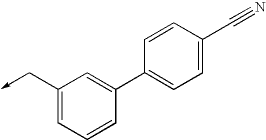 |
| 2743 2744 2745 | rac + − | 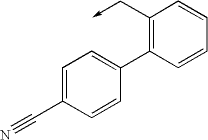 |
| 2746 2747 2748 | rac + − | 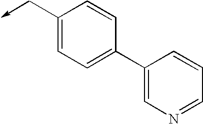 |
| 2749 2750 2751 | rac + − | 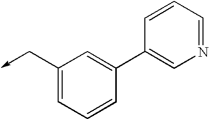 |
| 2752 2753 2754 | rac + − | 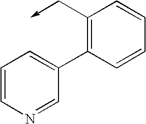 |
| 2755 2756 2757 | rac + − | 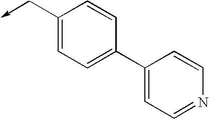 |
| 2758 2759 2760 | rac + − | 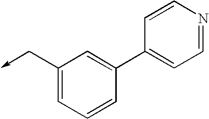 |
| 2761 2762 2763 | rac + − | 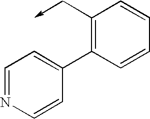 |
| 2764 2765 2766 | rac + − | 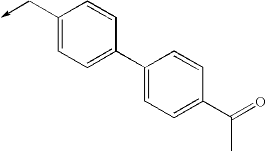 |
| 2767 2768 2769 | rac + − | 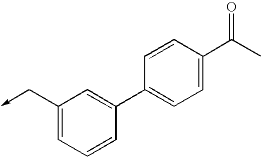 |
| 2770 2771 2772 | rac + − | 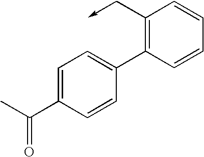 |
| 2773 2774 2775 | rac + − | 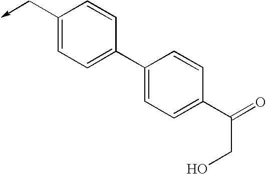 |
| 2776 2777 2778 | rac + − | 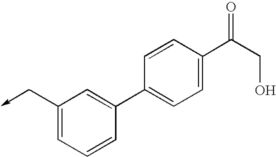 |
| 2779 2780 2781 | rac + − | 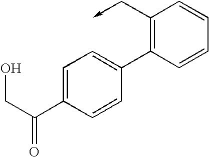 |
| 2782 2783 2784 | rac + − | 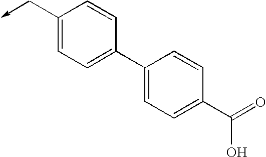 |
| 2785 2786 2787 | rac + − | 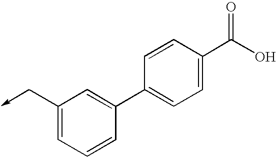 |
| 2788 2789 2790 | rac + − | 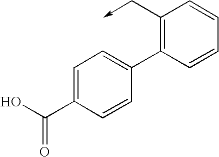 |
| 2791 2792 2793 | rac + − | 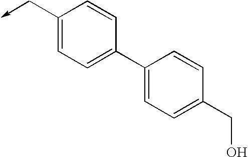 |
| 2794 2795 2796 | rac + − | 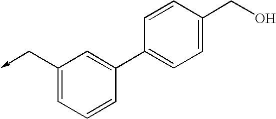 |
| 2797 2798 2799 | rac + − | 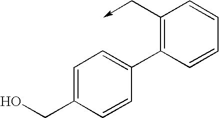 |
| 2800 2801 2802 | rac + − | 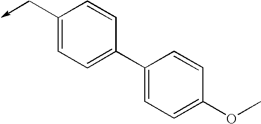 |
| 2803 2804 2805 | rac + − | 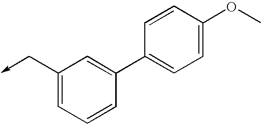 |
| 2806 2807 2808 | rac + − | 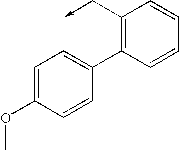 |
| 2809 2810 2811 | rac + − | 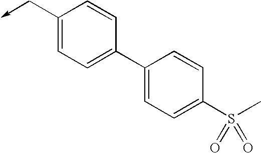 |
| 2812 2813 2814 | rac + − | 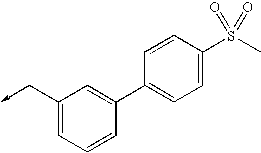 |
| 2815 2816 2817 | rac + − | 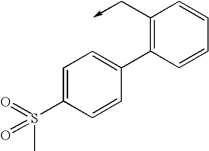 |
| 2818 2819 2820 | rac + − | 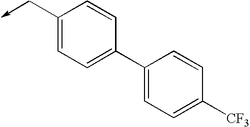 |
| 2821 2822 2823 | rac + − | 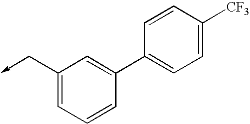 |
| 2824 2825 2826 | rac + − | 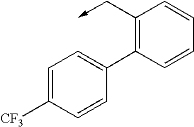 |
| 2827 2828 2829 | rac + − | 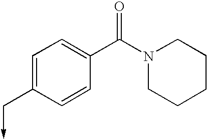 |
| 2830 2831 2832 | rac + − | 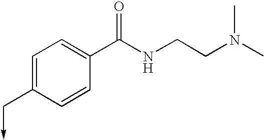 |
| 2833 2834 2835 | rac + − | 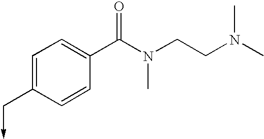 |
| 2836 2837 2838 | rac + − | 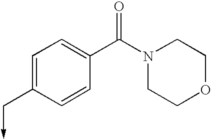 |
| 2839 2840 2841 | rac + − | 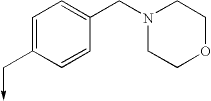 |
| 2842 2843 2844 | rac + − | 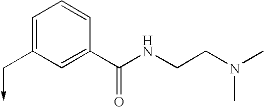 |
| 2845 2846 2847 | rac + − | 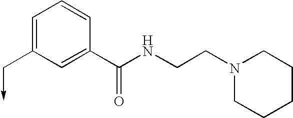 |
| 2848 2849 2850 | rac + − | 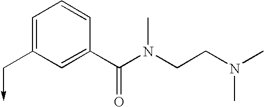 |
| 2851 2852 2853 | rac + − | 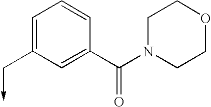 |
| 2854 2855 2856 | rac + − | 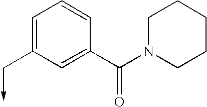 |
| 2857 2858 2859 | rac + − | 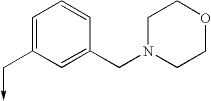 |
| 2860 2861 2862 | rac + − | 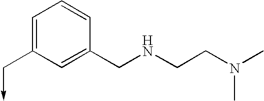 |
| 2863 2864 2865 | rac + − | 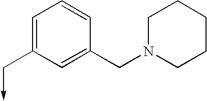 |
| 2866 2867 2868 | rac + − | 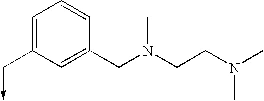 |
| 2869 2870 2871 | rac + − | 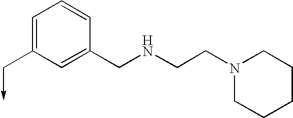 |

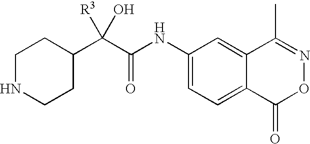
| No. | Racemic or enantiomer | R3 |
|---|---|---|
| 2872 2873 2874 | rac + − | 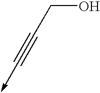 |
| 2875 2876 2877 | rac + − | 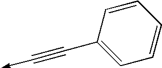 |
| 2878 2879 2880 | rac + − | 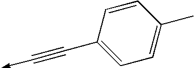 |
| 2881 2882 2883 | rac + − | 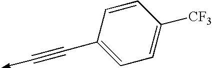 |
| 2884 2885 2886 | rac + − | 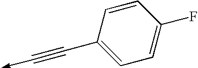 |
| 2887 2888 2889 | rac + − | 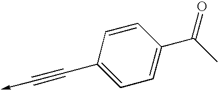 |
| 2890 2891 2892 | rac + − | 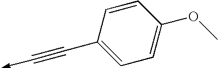 |
| 2893 2894 2895 | rac + − | 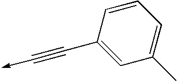 |
| 2896 2897 2898 | rac + − | 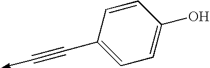 |
| 2899 2900 2901 | rac + − | 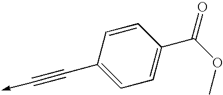 |
| 2902 2903 2904 | rac + − | 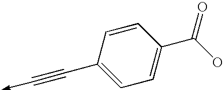 |
| 2905 2906 2907 | rac + − | 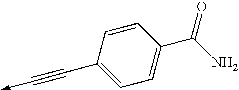 |
| 2908 2909 2910 | rac + − | 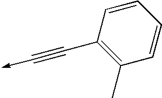 |
| 2911 2912 2913 | rac + − | 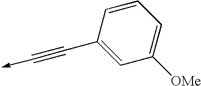 |
| 2914 2915 2916 | rac + − | 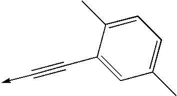 |
| 2917 2918 2919 | rac + − | 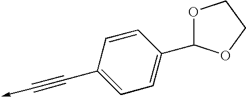 |
| 2920 2921 2922 | rac + − | 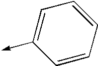 |
| 2923 2924 2925 | rac + − | 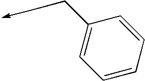 |
| 2926 2927 2928 | rac + − | 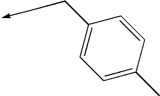 |
| 2929 2930 2931 | rac + − | 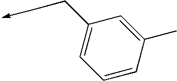 |
| 2932 2933 2934 | rac + − | 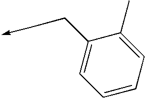 |
| 2935 2936 2937 | rac + − | 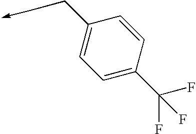 |
| 2938 2939 2940 | rac + − | 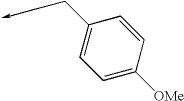 |
| 2941 2942 2943 | rac + − | 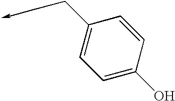 |
| 2944 2945 2946 | rac + − | 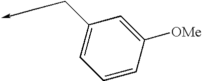 |
| 2947 2948 2949 | rac + − | 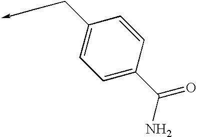 |
| 2950 2951 2952 | rac + − | 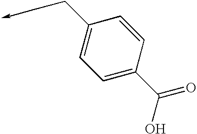 |
| 2953 2954 2955 | rac + − | 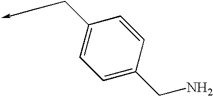 |
| 2956 2957 2958 | rac + − | 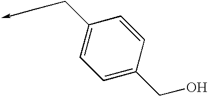 |
| 2959 2960 2961 | rac + − | 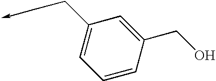 |
| 2962 2963 2964 | rac + − | 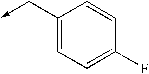 |
| 2965 2966 2967 | rac + − | 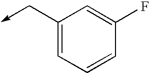 |
| 2968 2969 2970 | rac + − | 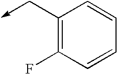 |
| 2971 2972 2973 | rac + − | 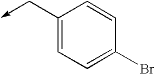 |
| 2974 2975 2976 | rac + − | 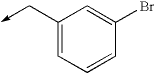 |
| 2977 2978 2979 | rac + − | 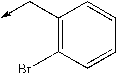 |
| 2980 2981 2982 | rac + − | 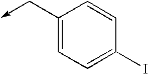 |
| 2983 2984 2985 | rac + − | 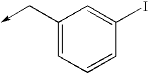 |
| 2986 2987 2988 | rac + − | 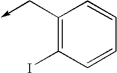 |
| 2989 2990 2991 | rac + − | 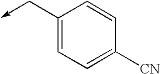 |
| 2992 2993 2994 | rac + − | 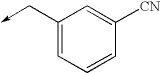 |
| 2995 2996 2997 | rac + − | 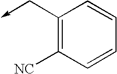 |
| 2998 2999 3000 | rac + − | 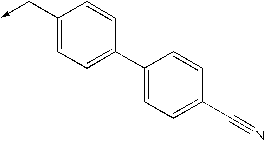 |
| 3001 3002 3003 | rac + − | 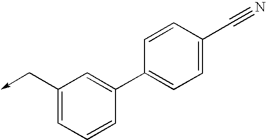 |
| 3004 3005 3006 | rac + − | 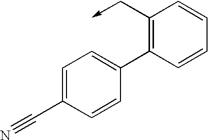 |
| 3007 3008 3009 | rac + − | 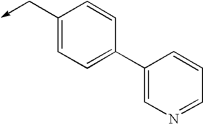 |
| 3010 3011 3012 | rac + − | 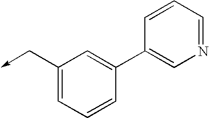 |
| 3013 3014 3015 | rac + − | 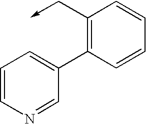 |
| 3016 3017 3018 | rac + − | 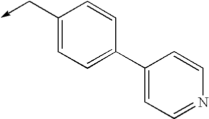 |
| 3019 3020 3021 | rac + − | 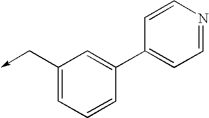 |
| 3022 3023 3024 | rac + − | 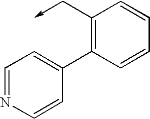 |
| 3025 3026 3027 | rac + − | 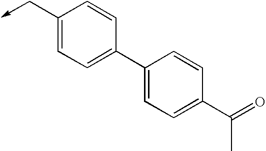 |
| 3028 3029 3030 | rac + − | 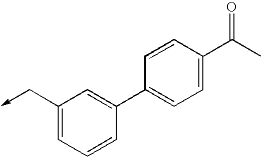 |
| 3031 3032 3033 | rac + − | 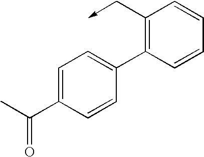 |
| 3034 3035 3036 | rac + − | 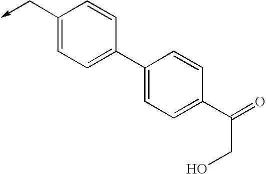 |
| 3037 3038 3039 | rac + − | 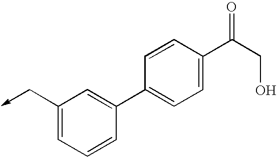 |
| 3040 3041 3042 | rac + − | 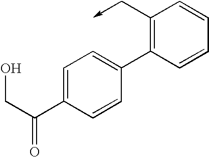 |
| 3043 3044 3045 | rac + − | 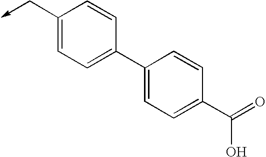 |
| 3046 3047 3048 | rac + − | 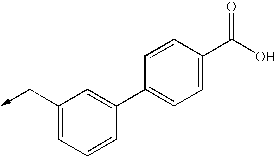 |
| 3049 3050 3051 | rac + − | 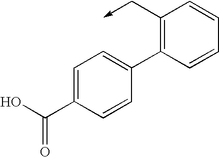 |
| 3052 3053 3054 | rac + − | 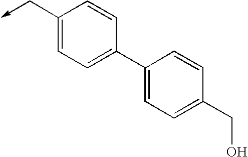 |
| 3055 3056 3057 | rac + − | 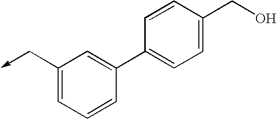 |
| 3058 3059 3060 | rac + − | 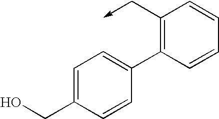 |
| 3061 3062 3063 | rac + − | 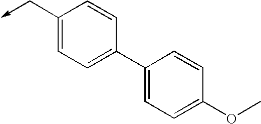 |
| 3064 3065 3066 | rac + − | 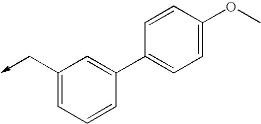 |
| 3067 3068 3069 | rac + − | 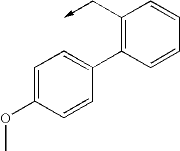 |
| 3070 3071 3072 | rac + − | 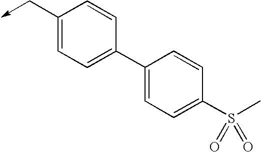 |
| 3073 3074 3075 | rac + − | 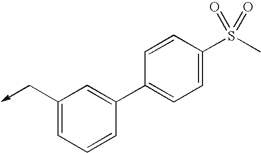 |
| 3076 3077 3078 | rac + − | 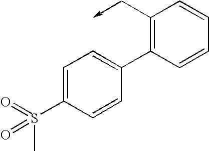 |
| 3079 3080 3081 | rac + − | 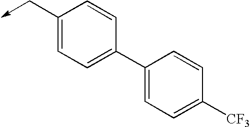 |
| 3082 3083 3084 | rac + − | 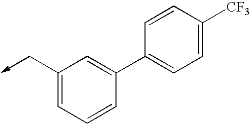 |
| 3085 3086 3087 | rac + − | 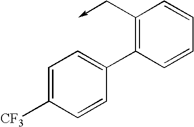 |
| 3088 3089 3090 | rac + − | 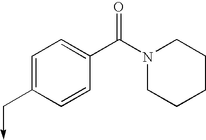 |
| 3091 3092 3093 | rac + − | 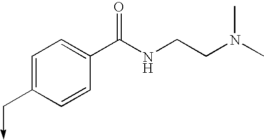 |
| 3094 3095 3096 | rac + − | 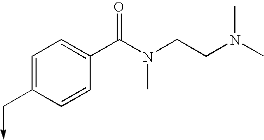 |
| 3097 3098 3099 | rac + − | 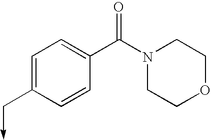 |
| 3100 3101 3102 | rac + − | 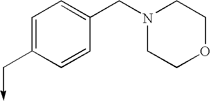 |
| 3103 3104 3105 | rac + − | 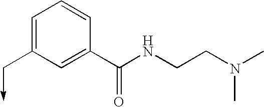 |
| 3106 3107 3108 | rac + − | 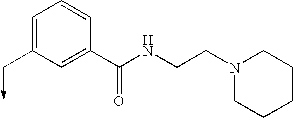 |
| 3109 3110 3111 | rac + − | 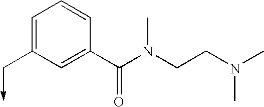 |
| 3112 3113 3114 | rac + − | 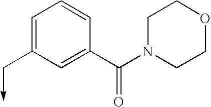 |
| 3115 3116 3117 | rac + − | 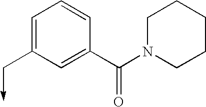 |
| 3118 3119 3120 | rac + − | 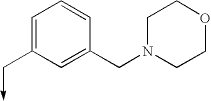 |
| 3121 3122 3123 | rac + − | 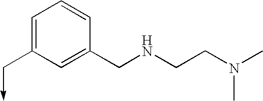 |
| 3124 3125 3126 | rac + − | 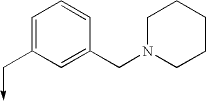 |
| 3127 3128 3129 | rac + − | 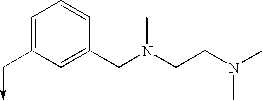 |
| 3130 3131 3132 | rac + − | 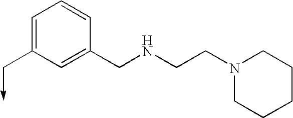 |

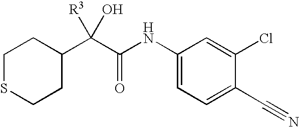
| No. | Racemic or enantiomer | R3 |
|---|---|---|
| 3133 3134 3135 | rac + − | 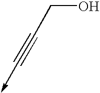 |
| 3136 3137 3138 | rac + − | 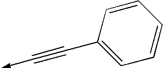 |
| 3139 3140 3141 | rac + − | 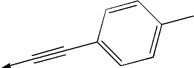 |
| 3142 3143 3144 | rac + − | 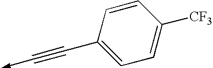 |
| 3145 3146 3147 | rac + − | 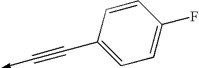 |
| 3148 3149 3150 | rac + − | 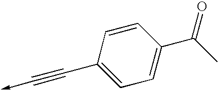 |
| 3151 3152 3153 | rac + − | 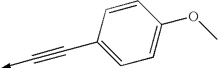 |
| 3154 3155 3156 | rac + − | 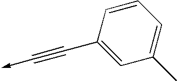 |
| 3157 3158 3159 | rac + − | 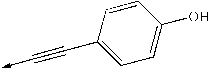 |
| 3160 3161 3162 | rac + − | 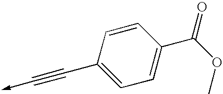 |
| 3163 3164 3165 | rac + − | 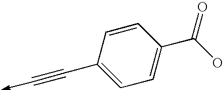 |
| 3166 3167 3168 | rac + − | 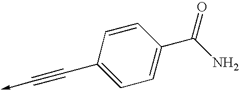 |
| 3169 3170 3171 | rac + − | 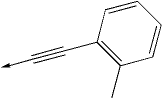 |
| 3172 3173 3174 | rac + − | 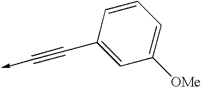 |
| 3175 3176 3177 | rac + − | 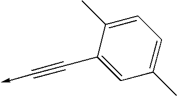 |
| 3178 3179 3180 | rac + − | 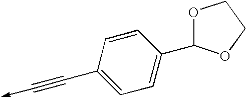 |
| 3181 3182 3183 | rac + − | 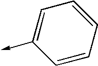 |
| 3184 3185 3186 | rac + − | 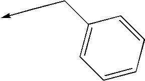 |
| 3187 3188 3189 | rac + − | 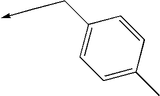 |
| 3190 3191 3192 | rac + − | 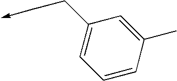 |
| 3193 3194 3195 | rac + − | 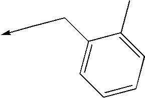 |
| 3196 3197 3198 | rac + − | 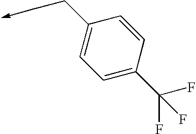 |
| 3199 3200 3201 | rac + − | 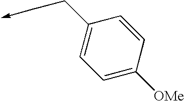 |
| 3202 3203 3204 | rac + − | 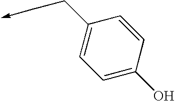 |
| 3205 3206 3207 | rac + − | 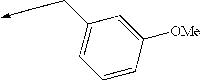 |
| 3208 3209 3210 | rac + − | 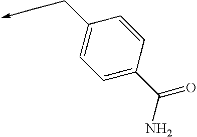 |
| 3211 3212 3213 | rac + − | 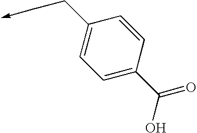 |
| 3214 3215 3216 | rac + − | 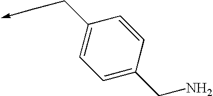 |
| 3217 3218 3219 | rac + − | 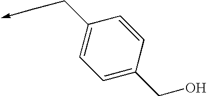 |
| 3220 3221 3222 | rac + − | 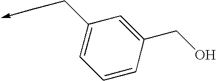 |
| 3223 3224 3225 | rac + − | 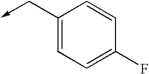 |
| 3226 3227 3228 | rac + − | 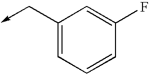 |
| 3229 3230 3231 | rac + − | 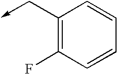 |
| 3232 3233 3234 | rac + − | 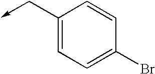 |
| 3235 3236 3237 | rac + − | 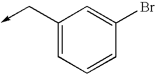 |
| 3238 3239 3240 | rac + − | 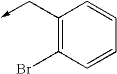 |
| 3241 3242 3243 | rac + − | 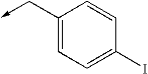 |
| 3244 3245 3246 | rac + − | 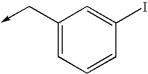 |
| 3247 3248 3249 | rac + − | 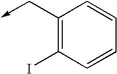 |
| 3250 3251 3252 | rac + − | 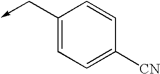 |
| 3253 3254 3255 | rac + − | 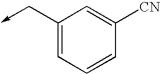 |
| 3256 3257 3258 | rac + − | 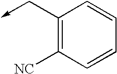 |
| 3259 3260 3261 | rac + − | 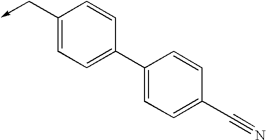 |
| 3262 3263 3264 | rac + − | 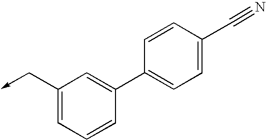 |
| 3265 3266 3267 | rac + − | 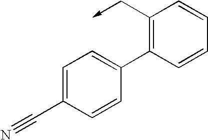 |
| 3268 3269 3270 | rac + − | 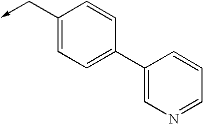 |
| 3271 3272 3273 | rac + − | 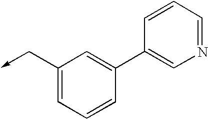 |
| 3274 3275 3276 | rac + − | 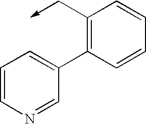 |
| 3277 3278 3279 | rac + − | 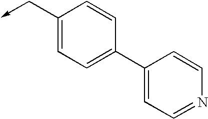 |
| 3280 3281 3282 | rac + − | 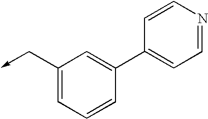 |
| 3283 3284 3285 | rac + − | 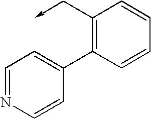 |
| 3286 3287 3288 | rac + − | 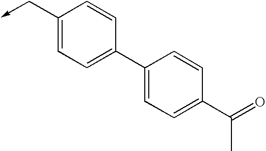 |
| 3289 3290 3291 | rac + − | 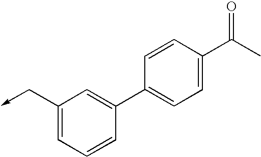 |
| 3292 3293 3294 | rac + − | 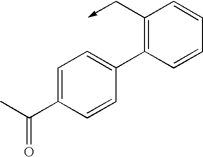 |
| 3295 3296 3297 | rac + − | 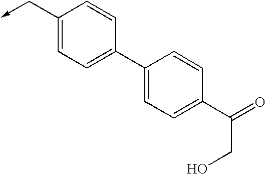 |
| 3298 3299 3300 | rac + − | 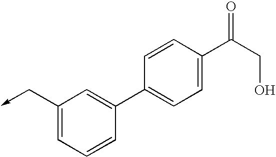 |
| 3301 3302 3303 | rac + − | 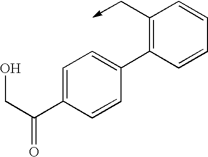 |
| 3304 3305 3306 | rac + − | 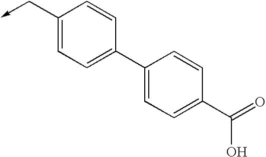 |
| 3307 3308 3309 | rac + − | 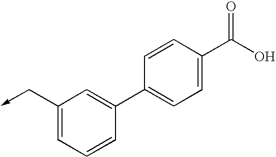 |
| 3310 3311 3312 | rac + − | 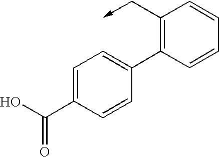 |
| 3313 3314 3315 | rac + − | 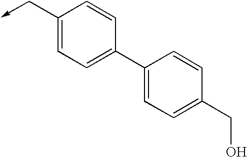 |
| 3316 3317 3318 | rac + − | 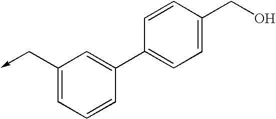 |
| 3319 3320 3321 | rac + − | 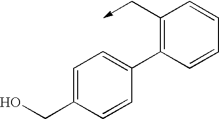 |
| 3322 3323 3324 | rac + − | 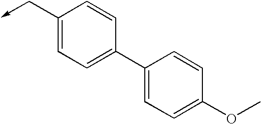 |
| 3325 3326 3327 | rac + − | 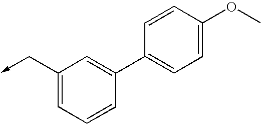 |
| 3328 3329 3330 | rac + − | 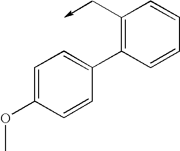 |
| 3331 3332 3333 | rac + − | 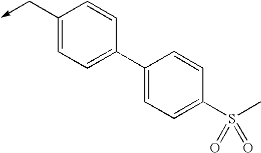 |
| 3334 3335 3336 | rac + − | 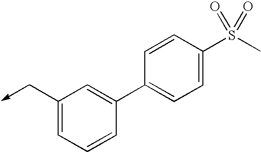 |
| 3337 3338 3339 | rac + − | 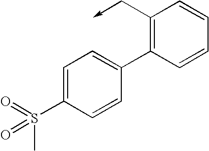 |
| 3340 3341 3342 | rac + − | 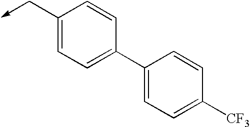 |
| 3343 3344 3345 | rac + − | 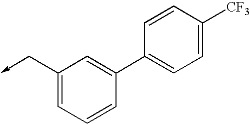 |
| 3346 3347 3348 | rac + − | 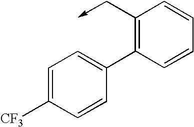 |
| 3349 3350 3351 | rac + − | 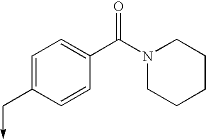 |
| 3352 3353 3354 | rac + − | 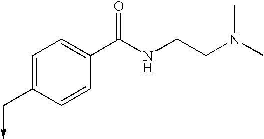 |
| 3355 3356 3357 | rac + − | 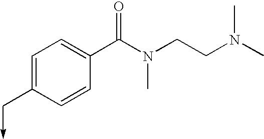 |
| 3358 3359 3360 | rac + − | 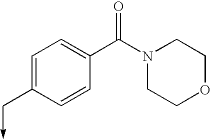 |
| 3361 3362 3363 | rac + − | 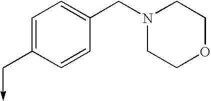 |
| 3364 3365 3366 | rac + − | 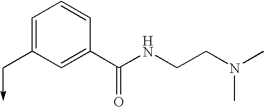 |
| 3367 3368 3369 | rac + − | 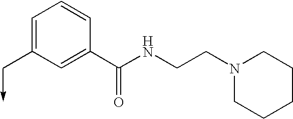 |
| 3370 3371 3372 | rac + − | 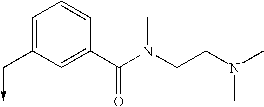 |
| 3373 3374 3375 | rac + − | 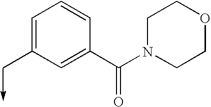 |
| 3376 3377 3378 | rac + − | 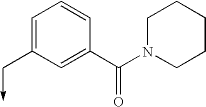 |
| 3379 3380 3381 | rac + − | 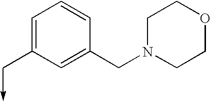 |
| 3382 3383 3384 | rac + − | 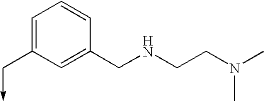 |
| 3385 3386 3387 | rac + − | 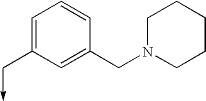 |
| 3388 3389 3390 | rac + − | 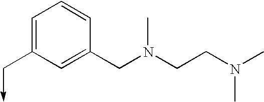 |
| 3391 3392 3393 | rac + − | 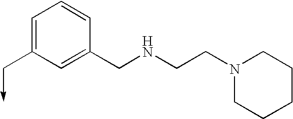 |

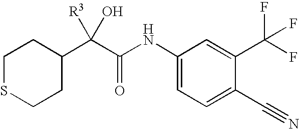
| No. | Racemic or enantiomer | R3 |
|---|---|---|
| 3394 3395 3396 | rac + − | 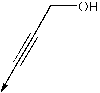 |
| 3397 3398 3399 | rac + − | 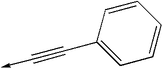 |
| 3400 3401 3402 | rac + − | 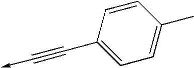 |
| 3403 3404 3405 | rac + − | 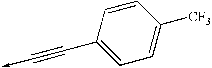 |
| 3406 3407 3408 | rac + − | 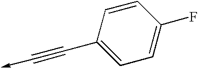 |
| 3409 3410 3411 | rac + − | 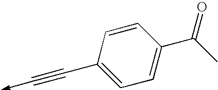 |
| 3412 3413 3414 | rac + − | 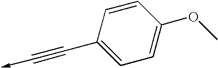 |
| 3415 3416 3417 | rac + − | 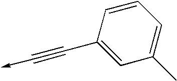 |
| 3418 3419 3420 | rac + − | 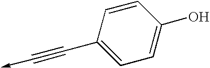 |
| 3421 3422 3423 | rac + − | 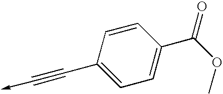 |
| 3424 3425 3426 | rac + − | 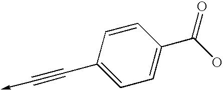 |
| 3427 3428 3429 | rac + − | 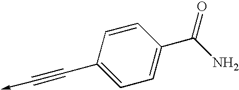 |
| 3430 3431 3432 | rac + − | 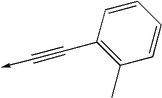 |
| 3433 3434 3435 | rac + − | 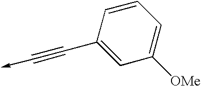 |
| 3436 3437 3438 | rac + − | 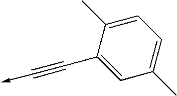 |
| 3439 3440 3441 | rac + − | 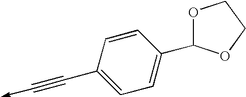 |
| 3442 3443 3444 | rac + − | 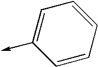 |
| 3445 3446 3447 | rac + − | 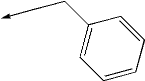 |
| 3448 3449 3450 | rac + − | 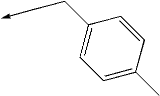 |
| 3451 3452 3453 | rac + − | 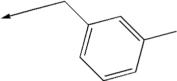 |
| 3454 3455 3456 | rac + − | 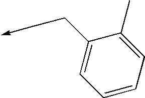 |
| 3457 3458 3459 | rac + − | 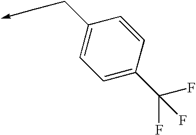 |
| 3460 3461 3462 | rac + − | 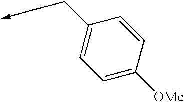 |
| 3463 3464 3465 | rac + − | 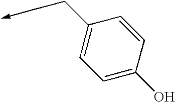 |
| 3466 3467 3468 | rac + − | 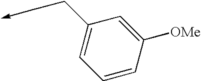 |
| 3469 3470 3471 | rac + − | 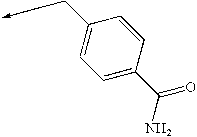 |
| 3472 3473 3474 | rac + − | 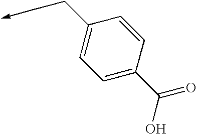 |
| 3475 3476 3477 | rac + − | 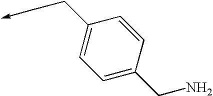 |
| 3478 3479 3480 | rac + − | 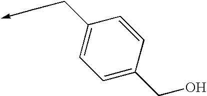 |
| 3481 3482 3483 | rac + − | 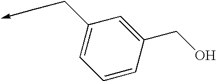 |
| 3484 3485 3486 | rac + − | 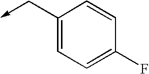 |
| 3487 3488 3489 | rac + − | 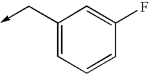 |
| 3490 3491 3492 | rac + − | 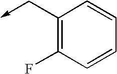 |
| 3493 3494 3495 | rac + − | 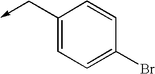 |
| 3496 3497 3498 | rac + − | 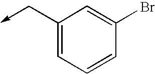 |
| 3499 3500 3501 | rac + − | 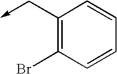 |
| 3502 3503 3504 | rac + − | 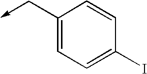 |
| 3505 3506 3507 | rac + − | 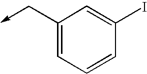 |
| 3508 3509 3510 | rac + − | 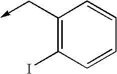 |
| 3511 3512 3513 | rac + − | 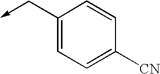 |
| 3514 3515 3516 | rac + − | 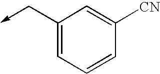 |
| 3517 3518 3519 | rac + − | 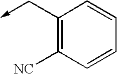 |
| 3520 3521 3522 | rac + − | 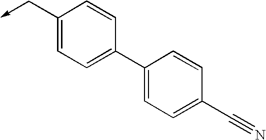 |
| 3523 3524 3525 | rac + − | 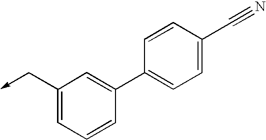 |
| 3526 3527 3528 | rac + − | 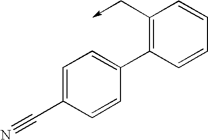 |
| 3529 3530 3531 | rac + − | 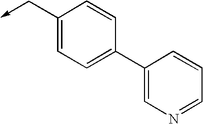 |
| 3532 3533 3534 | rac + − | 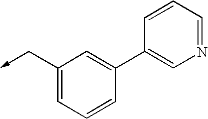 |
| 3535 3536 3537 | rac + − | 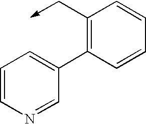 |
| 3538 3539 3540 | rac + − | 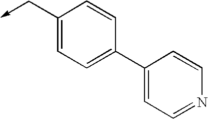 |
| 3541 3542 3543 | rac + − | 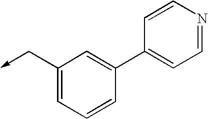 |
| 3544 3545 3546 | rac + − | 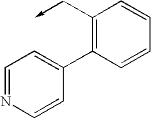 |
| 3547 3548 3549 | rac + − | 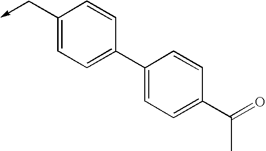 |
| 3550 3551 3552 | rac + − | 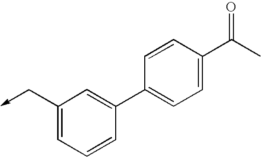 |
| 3553 3554 3555 | rac + − | 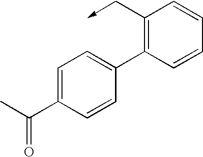 |
| 3556 3557 3558 | rac + − | 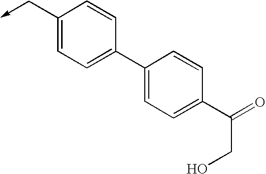 |
| 3559 3560 3561 | rac + − | 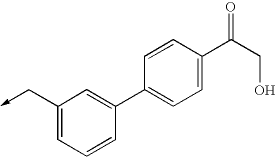 |
| 3562 3563 3564 | rac + − | 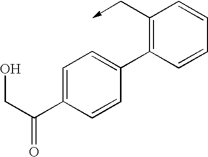 |
| 3565 3566 3567 | rac + − | 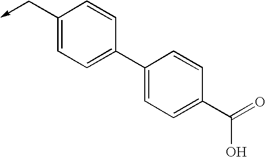 |
| 3568 3569 3570 | rac + − | 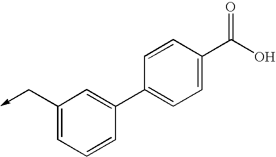 |
| 3571 3572 3573 | rac + − | 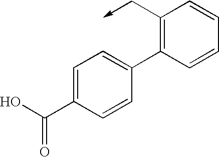 |
| 3574 3575 3576 | rac + − | 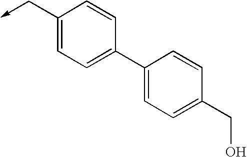 |
| 3577 3578 3579 | rac + − | 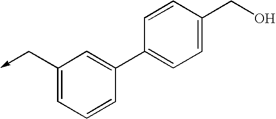 |
| 3580 3581 3582 | rac + − | 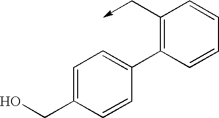 |
| 3583 3584 3585 | rac + − | 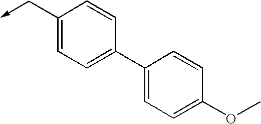 |
| 3586 3587 3588 | rac + − | 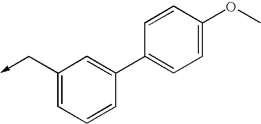 |
| 3589 3590 3591 | rac + − | 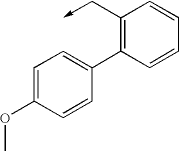 |
| 3592 3593 3594 | rac + − | 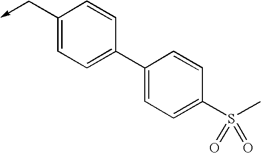 |
| 3595 3596 3597 | rac + − | 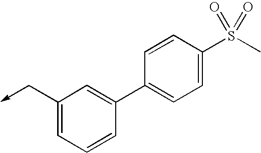 |
| 3598 3599 3600 | rac + − | 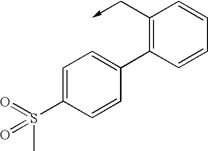 |
| 3601 3602 3603 | rac + − | 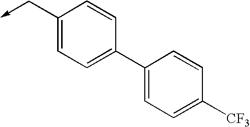 |
| 3604 3605 3606 | rac + − | 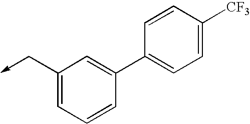 |
| 3607 3608 3609 | rac + − | 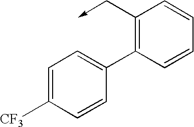 |
| 3610 3611 3612 | rac + − | 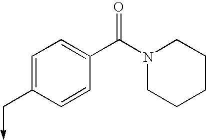 |
| 3613 3614 3615 | rac + − | 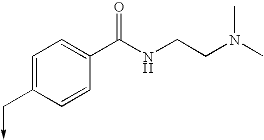 |
| 3616 3617 3618 | rac + − | 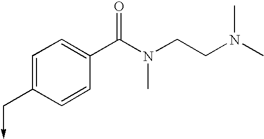 |
| 3619 3620 3621 | rac + − | 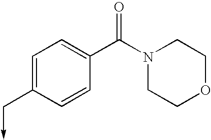 |
| 3622 3623 3624 | rac + − | 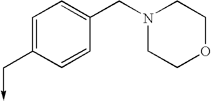 |
| 3625 3626 3627 | rac + − | 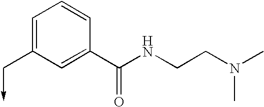 |
| 3628 3629 3630 | rac + − | 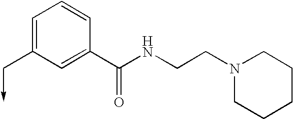 |
| 3631 3632 3633 | rac + − | 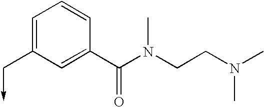 |
| 3634 3635 3636 | rac + − | 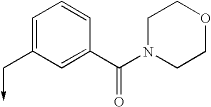 |
| 3637 3638 3639 | rac + − | 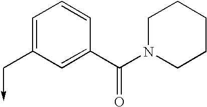 |
| 3640 3641 3642 | rac + − | 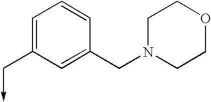 |
| 3643 3644 3645 | rac + − | 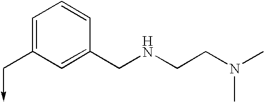 |
| 3646 3647 3648 | rac + − | 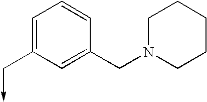 |
| 3649 3650 3651 | rac + − | 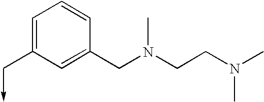 |
| 3652 3653 3654 | rac + − | 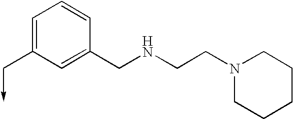 |

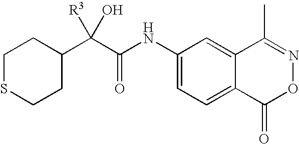
| No. | Racemic or enantiomer | R3 |
|---|---|---|
| 3655 3656 3657 | rac + − | 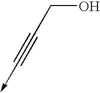 |
| 3658 3659 3660 | rac + − | 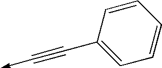 |
| 3661 3662 3663 | rac + − | 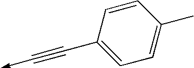 |
| 3664 3665 3666 | rac + − | 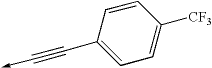 |
| 3667 3668 3669 | rac + − | 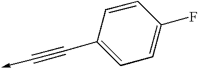 |
| 3670 3671 3672 | rac + − | 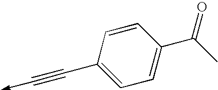 |
| 3673 3674 3675 | rac + − | 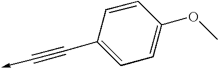 |
| 3676 3677 3678 | rac + − | 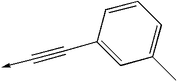 |
| 3679 3680 3681 | rac + − | 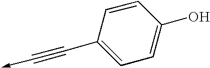 |
| 3682 3683 3684 | rac + − | 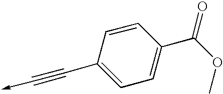 |
| 3685 3686 3687 | rac + − | 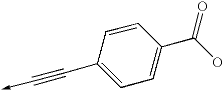 |
| 3688 3689 3690 | rac + − | 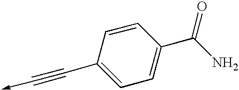 |
| 3691 3692 3693 | rac + − | 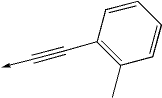 |
| 3694 3695 3696 | rac + − | 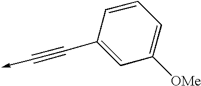 |
| 3697 3698 3699 | rac + − | 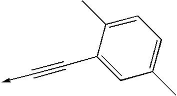 |
| 3700 3701 3702 | rac + − | 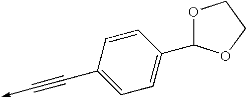 |
| 3703 3704 3705 | rac + − | 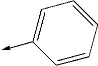 |
| 3706 3707 3708 | rac + − | 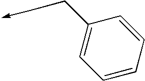 |
| 3709 3710 3711 | rac + − | 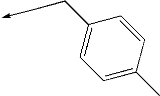 |
| 3712 3713 3714 | rac + − | 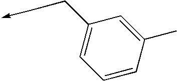 |
| 3715 3716 3717 | rac + − | 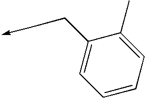 |
| 3718 3719 3720 | rac + − | 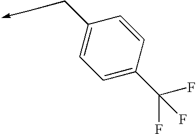 |
| 3721 3722 3723 | rac + − | 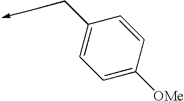 |
| 3724 3725 3726 | rac + − | 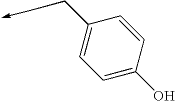 |
| 3727 3728 3729 | rac + − | 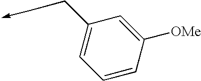 |
| 3730 3731 3732 | rac + − | 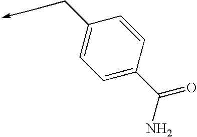 |
| 3733 3734 3735 | rac + − | 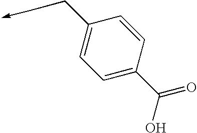 |
| 3736 3737 3738 | rac + − | 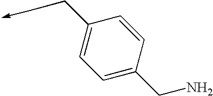 |
| 3739 3740 3741 | rac + − | 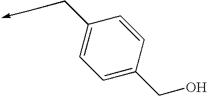 |
| 3742 3743 3744 | rac + − | 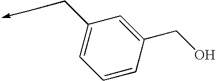 |
| 3745 3746 3747 | rac + − | 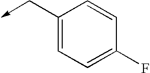 |
| 3748 3749 3750 | rac + − | 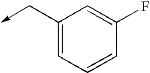 |
| 3751 3752 3753 | rac + − | 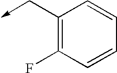 |
| 3754 3755 3756 | rac + − | 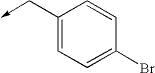 |
| 3757 3758 3759 | rac + − | 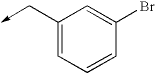 |
| 3760 3761 3762 | rac + − | 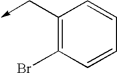 |
| 3763 3764 3765 | rac + − | 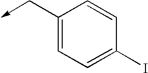 |
| 3766 3767 3768 | rac + − | 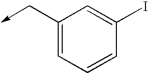 |
| 3769 3770 3771 | rac + − | 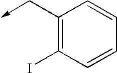 |
| 3772 3773 3774 | rac + − | 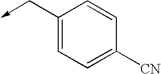 |
| 3775 3776 3777 | rac + − | 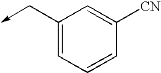 |
| 3778 3779 3780 | rac + − | 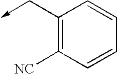 |
| 3781 3782 3783 | rac + − | 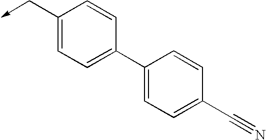 |
| 3784 3785 3786 | rac + − | 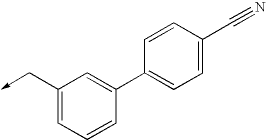 |
| 3787 3788 3789 | rac + − | 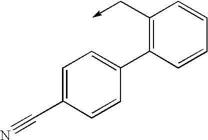 |
| 3790 3791 3792 | rac + − | 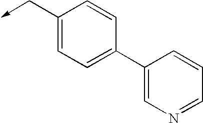 |
| 3793 3794 3795 | rac + − | 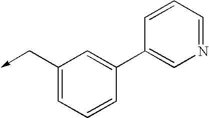 |
| 3796 3797 3798 | rac + − | 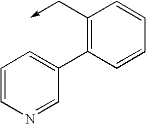 |
| 3799 3800 3801 | rac + − | 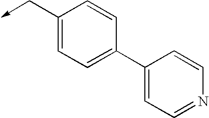 |
| 3802 3803 3804 | rac + − | 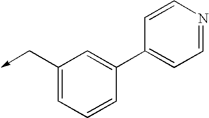 |
| 3805 3806 3807 | rac + − | 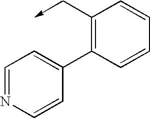 |
| 3808 3809 3810 | rac + − | 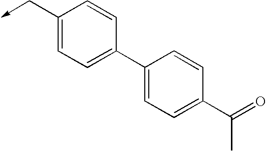 |
| 3811 3812 3813 | rac + − | 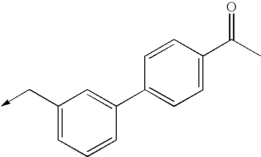 |
| 3814 3815 3816 | rac + − | 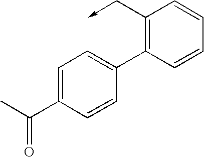 |
| 3817 3818 3819 | rac + − | 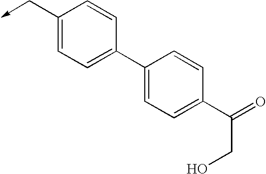 |
| 3820 3821 3822 | rac + − | 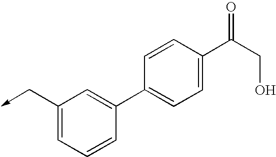 |
| 3823 3824 3825 | rac + − | 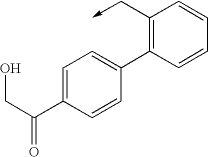 |
| 3826 3827 3828 | rac + − | 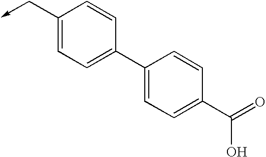 |
| 3829 3830 3831 | rac + − | 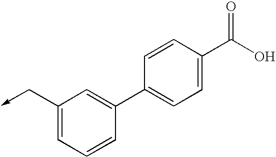 |
| 3832 3833 3834 | rac + − | 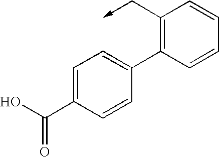 |
| 3835 3836 3837 | rac + − | 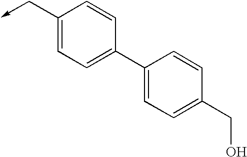 |
| 3838 3839 3840 | rac + − | 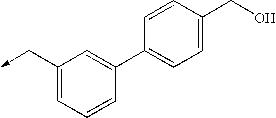 |
| 3841 3842 3843 | rac + − | 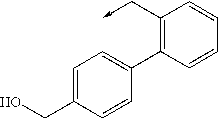 |
| 3844 3845 3846 | rac + − | 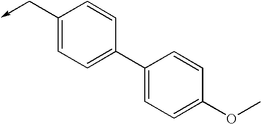 |
| 3847 3848 3849 | rac + − | 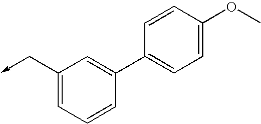 |
| 3850 3851 3852 | rac + − | 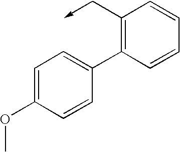 |
| 3853 3854 3855 | rac + − | 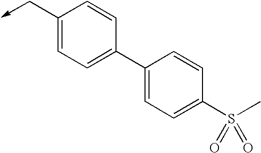 |
| 3856 3857 3858 | rac + − | 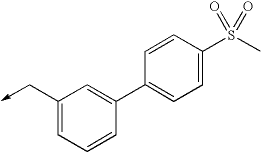 |
| 3859 3860 3861 | rac + − | 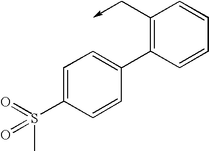 |
| 3862 3863 3864 | rac + − | 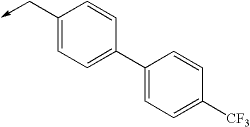 |
| 3865 3866 3867 | rac + − | 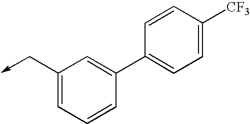 |
| 3868 3869 3870 | rac + − | 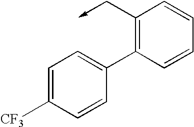 |
| 3871 3872 3873 | rac + − | 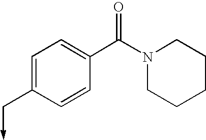 |
| 3874 3875 3876 | rac + − | 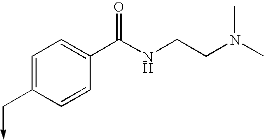 |
| 3877 3878 3879 | rac + − | 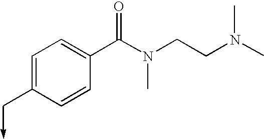 |
| 3880 3881 3882 | rac + − | 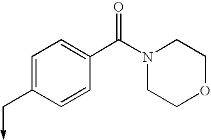 |
| 3883 3884 3885 | rac + − | 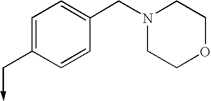 |
| 3886 3887 3888 | rac + − | 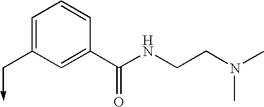 |
| 3889 3890 3891 | rac + − | 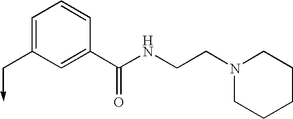 |
| 3892 3893 3894 | rac + − | 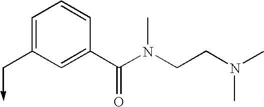 |
| 3895 3896 3897 | rac + − | 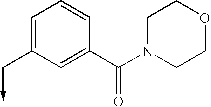 |
| 3898 3899 3900 | rac + − | 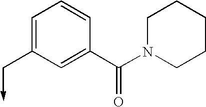 |
| 3901 3902 3903 | rac + − | 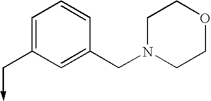 |
| 3904 3905 3906 | rac + − | 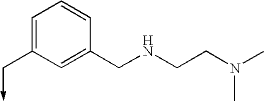 |
| 3907 3908 3909 | rac + − | 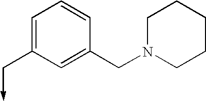 |
| 3910 3911 3912 | rac + − | 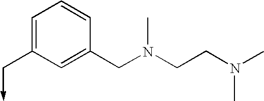 |
| 3913 3914 3915 | rac + − | 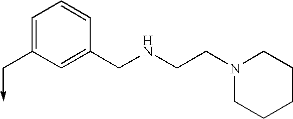 |
| No. | racemic or enantiomer or stereoisomer | structure |
| 3916 3917 3918 | rac + − | 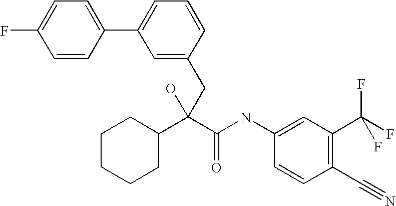 |
| 3919 3920 3921 3922 3923 | stereoisomer mixture (R, R) (S, S) (R, S) (S, R) | 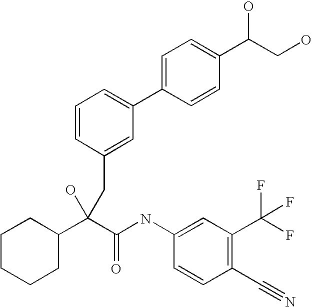 |
| 3924 3925 3926 | rac + − | 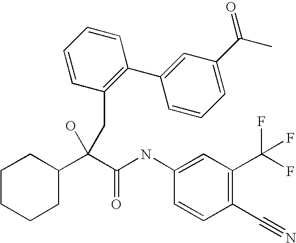 |
| 3927 3928 3929 | rac + − | 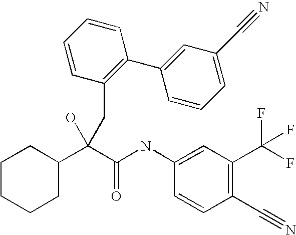 |
| 3930 3931 3932 | rac + − | 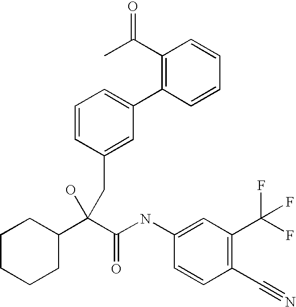 |
| 3933 3934 3935 | rac + − | 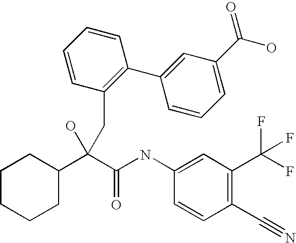 |
| 3936 3937 3938 | rac + − | 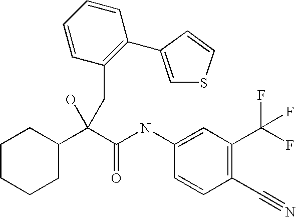 |
| 3939 3940 3941 | rac + − | 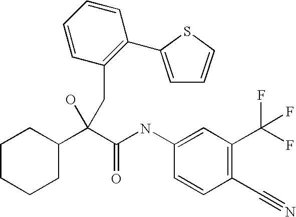 |
| 3942 3943 3944 | rac + − | 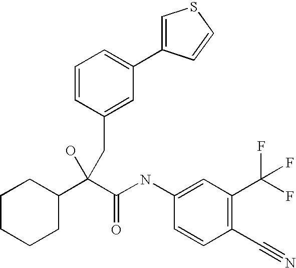 |
| 3945 3946 3947 | rac + − | 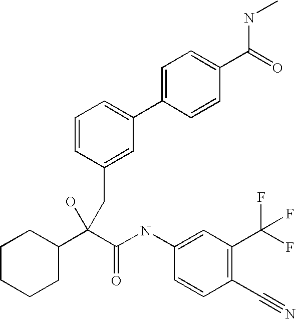 |
| 3948 3949 3950 | rac + − | 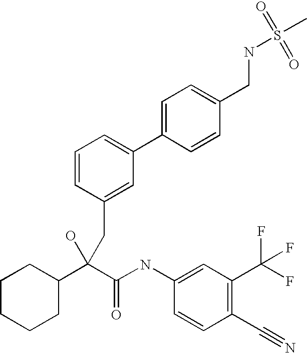 |
| 3951 3952 3953 | rac + − | 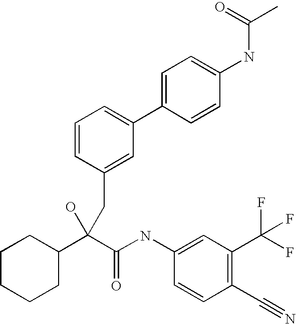 |
| 3954 3955 3956 | rac + − | 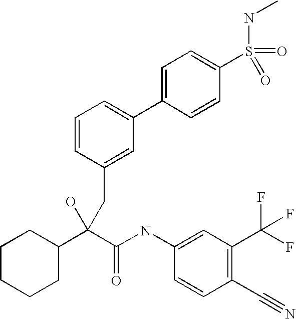 |
| 3957 3958 3959 | rac + − | 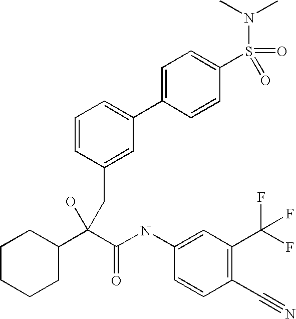 |
| 3960 3961 3962 | rac + − | 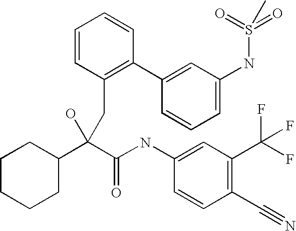 |
| 3963 3964 3965 | rac + − | 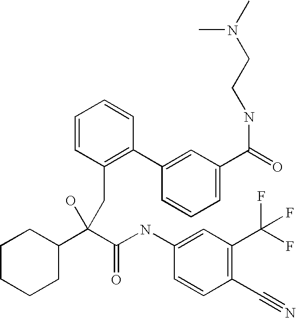 |
| 3966 3967 3968 3969 3970 | stereoisomer mixture (R, R) (S, S) (R, S) (S, R) | 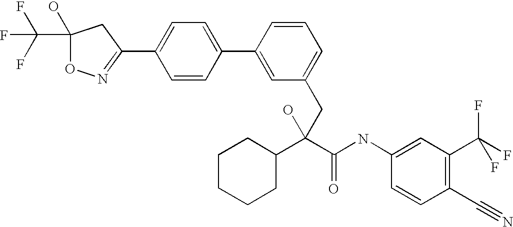 |
| 3971 3972 3973 | rac + − | 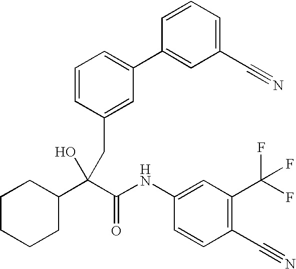 |
| 3974 3975 3976 | rac + − | 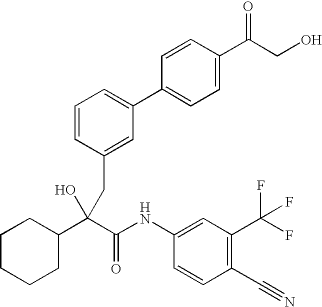 |
| 3977 3978 3979 | rac + − | 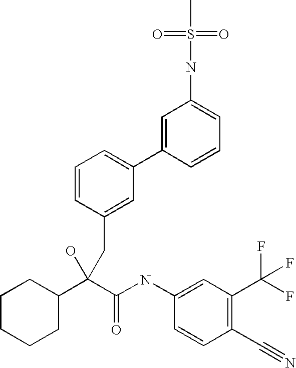 |
| 3980 3981 3982 | rac + − | 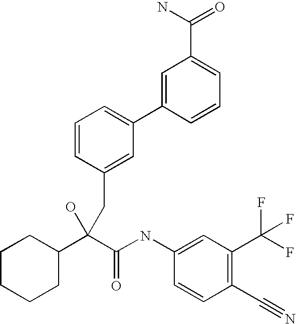 |
| 3983 3984 3985 | rac + − | 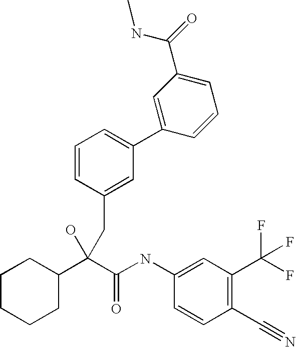 |
| 3986 3987 3988 | rac + − | 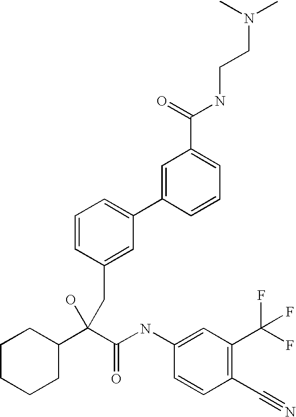 |
| 3989 3990 3991 | rac + − | 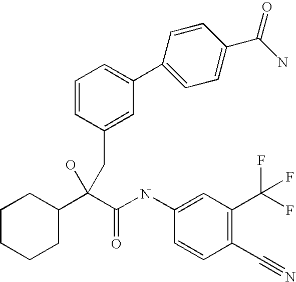 |
| 3992 3993 3994 | rac + − | 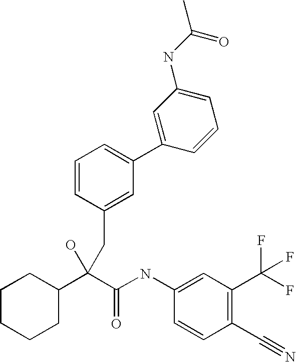 |
| 3995 3996 3997 | rac + − | 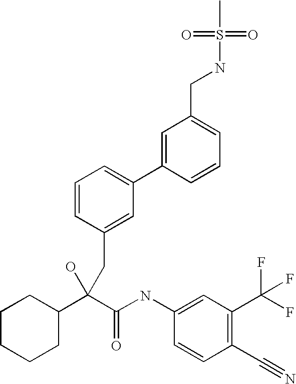 |
| 3998 3999 4000 4001 4002 | stereoisomer mixture (R, R) (S, S) (R, S) (S, R) | 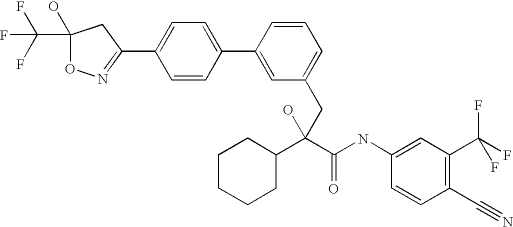 |
| 4003 4004 4005 | rac + − | 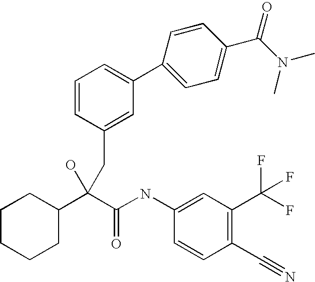 |
| 4006 4007 4008 | rac + − | 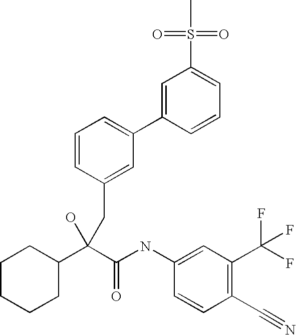 |
| 4009 4010 4011 | rac + − | 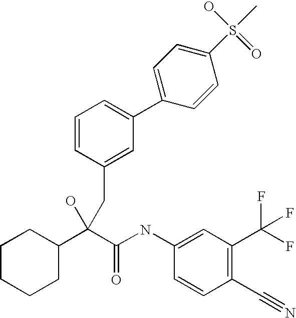 |
| 4012 4013 4014 | rac + − | 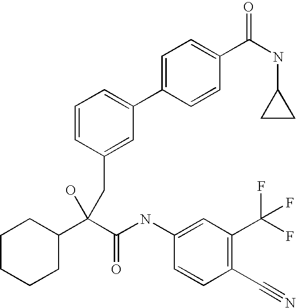 |
| 4015 4016 4017 | rac + − | 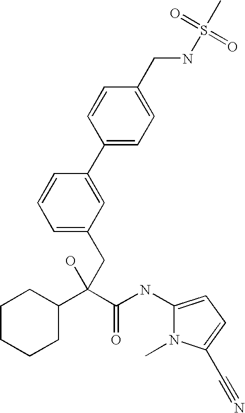 |
| 4018 4019 4020 | rac + − | 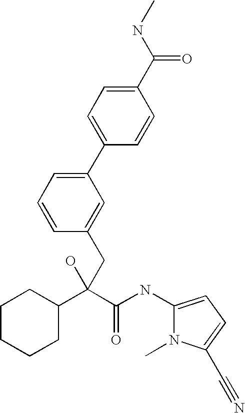 |
| 4021 4022 4023 | rac + − | 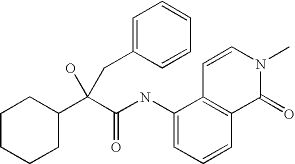 |
| 4024 4025 4026 | rac + − | 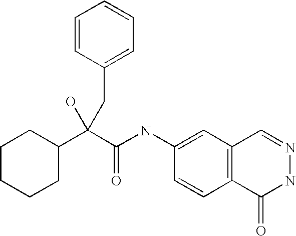 |
| 4027 4028 4029 | rac + − | 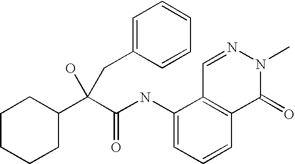 |
| 4030 4031 4032 | rac + − | 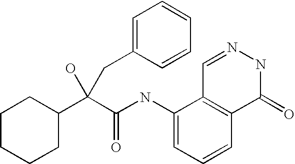 |
| 4033 4034 4035 | rac + − | 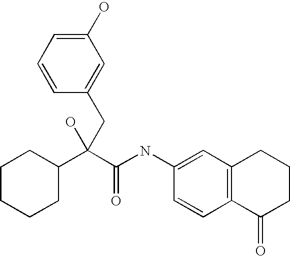 |
| 4036 4037 4038 | rac + − | 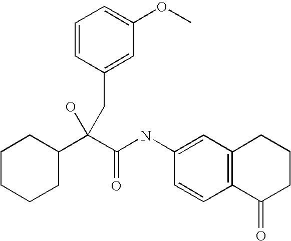 |
| 4039 4040 4041 | rac + − | 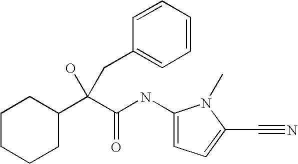 |
| 4042 4043 4044 | rac + − | 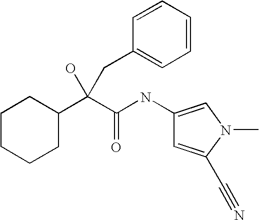 |
| 4045 4046 4047 | rac + − | 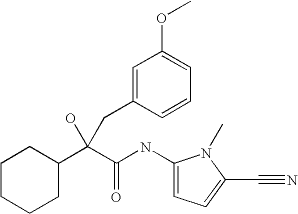 |
| 4048 4049 4050 | rac + − | 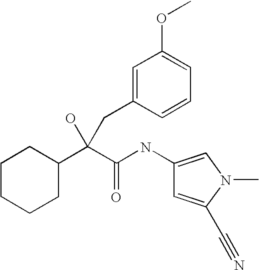 |
| 4051 4052 4053 | rac + − | 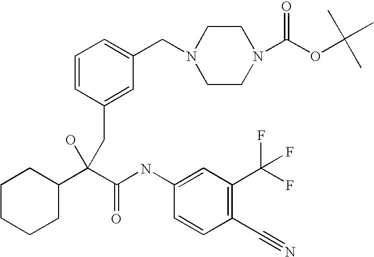 |
| 4054 4055 4056 | rac + − | 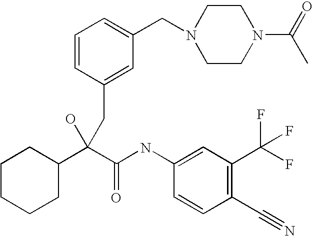 |
| 4057 4058 4059 | rac + − | 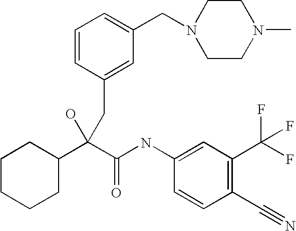 |
| 4060 4061 4062 | rac + − | 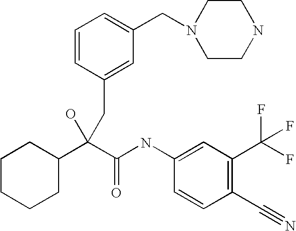 |
| 4063 4064 4065 | rac + − | 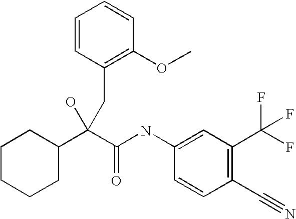 |
| 4066 4067 4068 | rac + − | 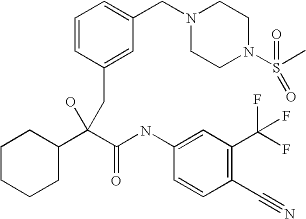 |
| 4069 4070 4071 | rac + − | 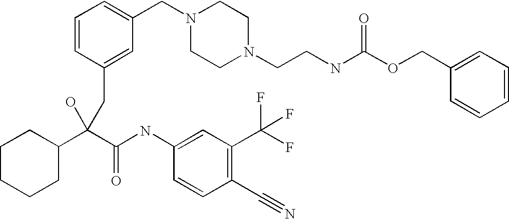 |
| 4072 4073 4074 | rac + − | 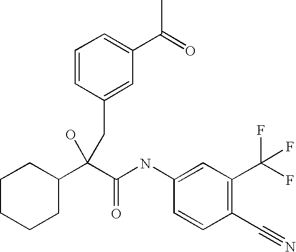 |
| 4075 4076 4077 | rac + − | 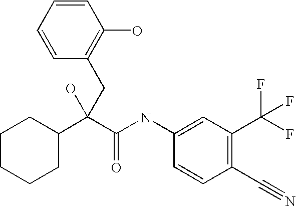 |
| 4078 4079 4080 | rac + − | 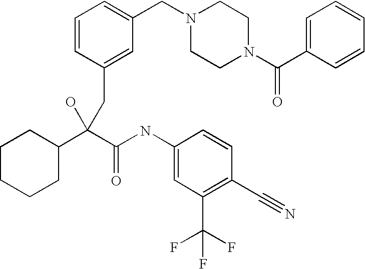 |
| 4081 4082 4083 | rac + − | 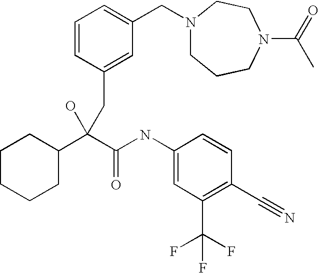 |
| 4084 4085 4086 | rac + − | 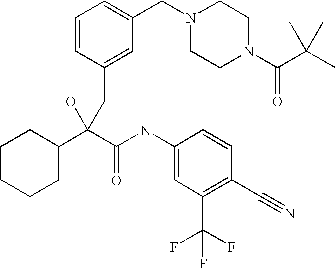 |
| 4087 4088 4089 | rac + − | 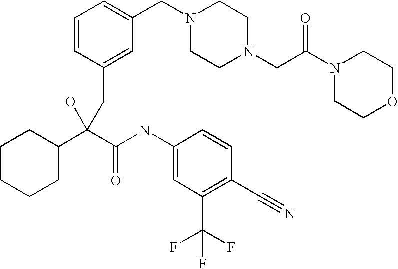 |
| 4090 4091 4092 | rac + − | 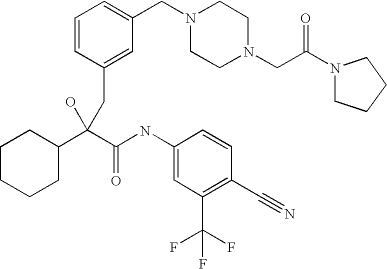 |
| 4093 4094 4095 | rac + − | 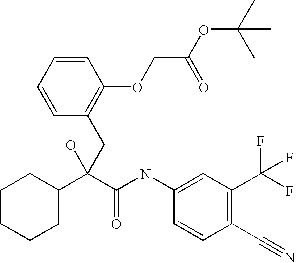 |
| 4096 4097 4098 | rac + − | 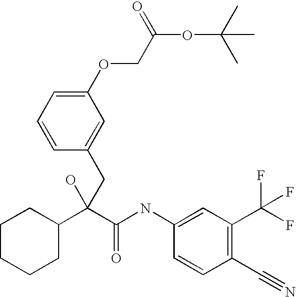 |
| 4099 4100 4101 | rac + − | 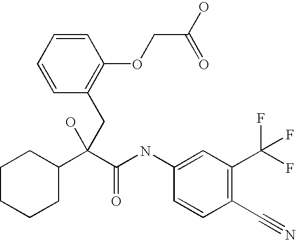 |
| 4102 4103 4104 | rac + − | 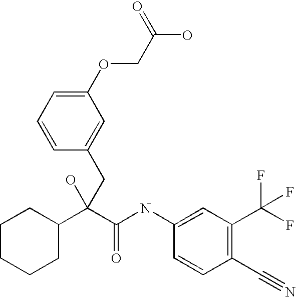 |
| 4105 4106 4107 | rac + − | 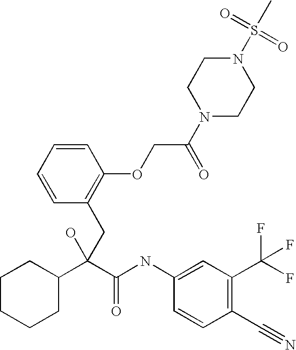 |
| 4108 4109 4110 | rac + − | 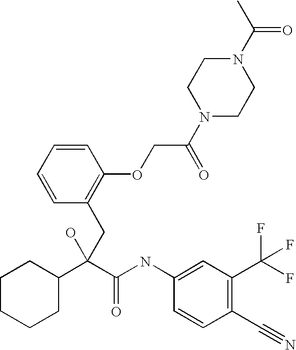 |
| 4111 4112 4113 | rac + − | 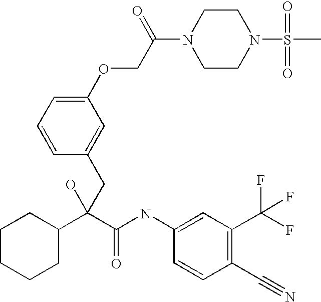 |
| 4114 4115 4116 | rac + − | 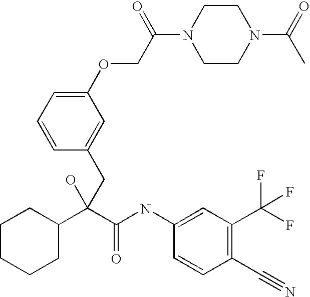 |
| 4117 4118 4119 | rac + − | 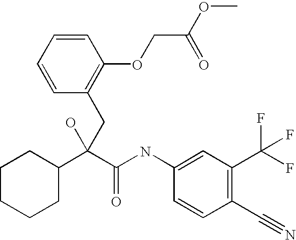 |
| 4120 4121 4122 | rac + − | 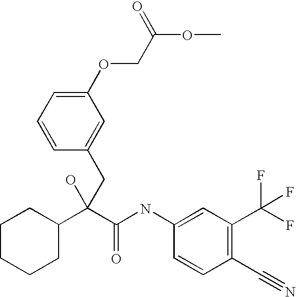 |
| 4123 4124 4125 | rac + − | 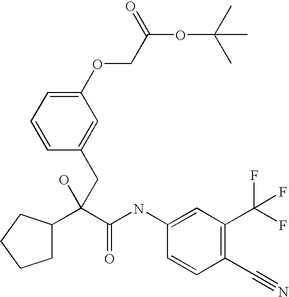 |
| 4126 4127 4128 | rac + − | 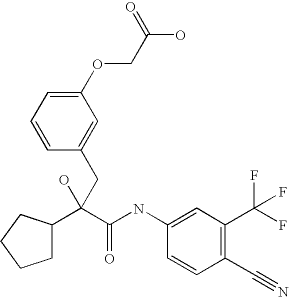 |
| 4129 4130 4131 | rac + − | 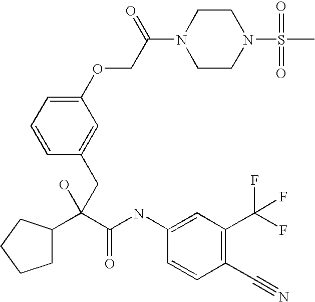 |
| 4132 4133 4134 | rac + − | 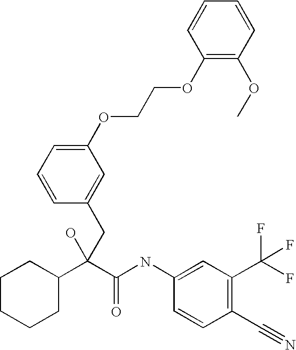 |
| 4135 4136 4137 | rac + − | 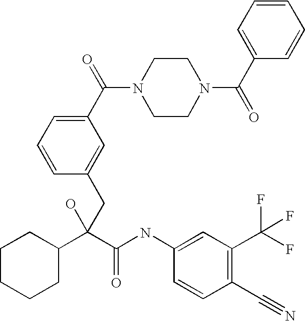 |
| 4138 4139 4140 | rac + − | 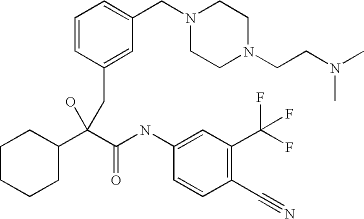 |
| 4141 4142 4143 | rac + − | 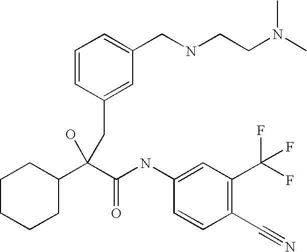 |
| 4144 4145 4146 | rac + − | 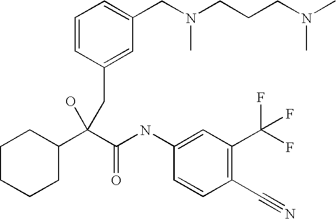 |
| 4147 4148 4149 | rac + − | 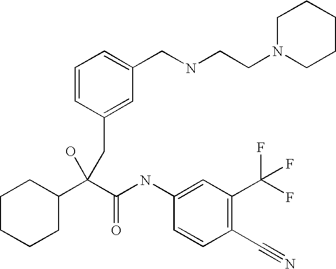 |
| 4150 4151 4152 | rac + − | 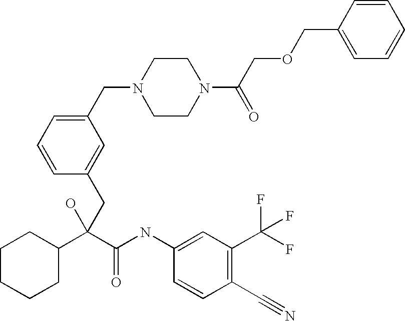 |
| 4153 4154 4155 | rac + − | 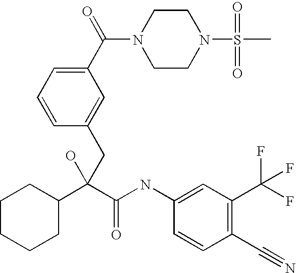 |
| 4156 4157 4158 | rac + − | 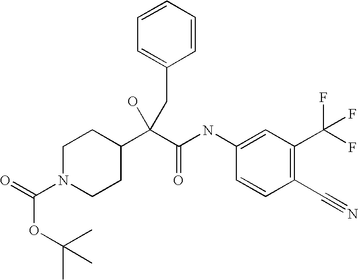 |
| 4159 4160 4161 | rac + − | 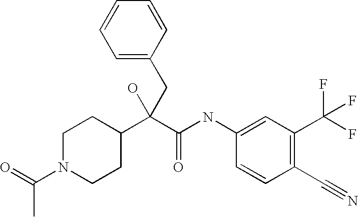 |
| 4162 4163 4164 | rac + − | 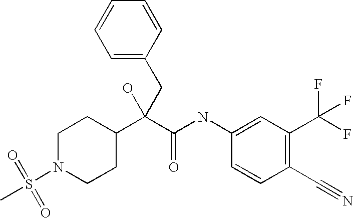 |
| 4165 4166 4167 | rac + − | 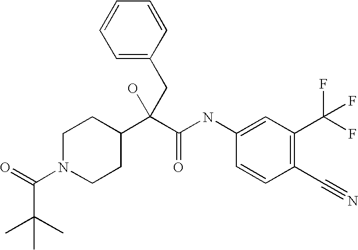 |
| 4168 4169 4170 | rac + − | 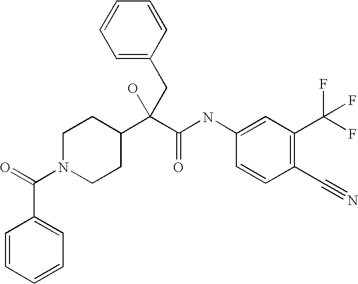 |
| 4171 4172 4173 | rac + − | 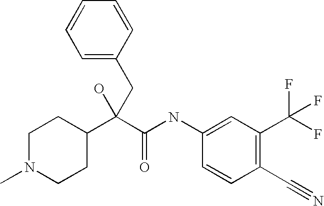 |
| 4174 4175 4176 | rac + − | 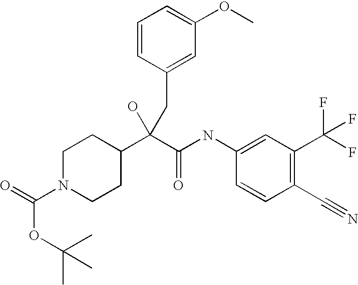 |
| 4177 4178 4179 | rac + − | 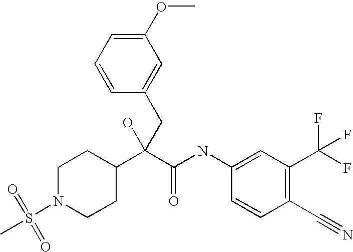 |
| 4180 4181 4182 | rac + − | 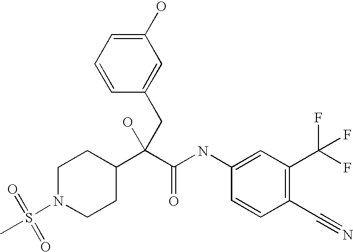 |
| 4183 4184 4185 | rac + − | 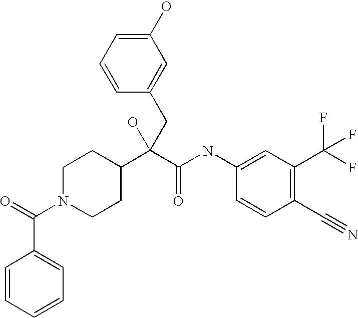 |
| 4186 4187 4188 | rac + − | 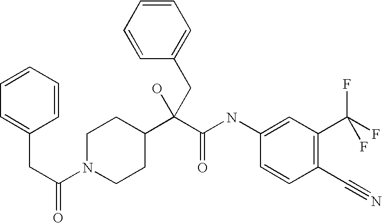 |
| 4189 4190 4191 | rac + − | 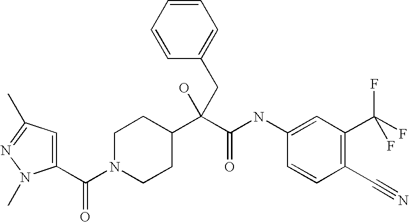 |
| 4192 4193 4194 | rac + − | 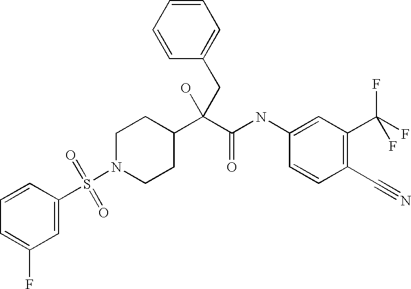 |
| 4195 4196 4197 | rac + − | 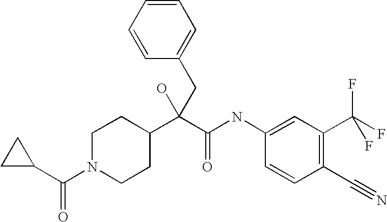 |
| 4198 4199 4200 | rac + − | 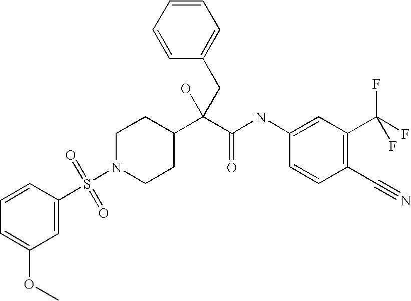 |
| 4201 4202 4203 | rac + − | 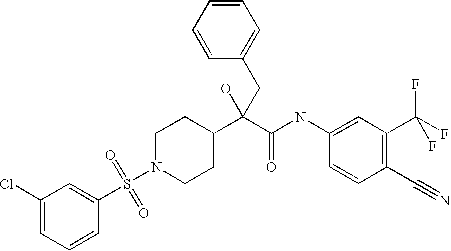 |
| 4204 4205 4206 | rac + − | 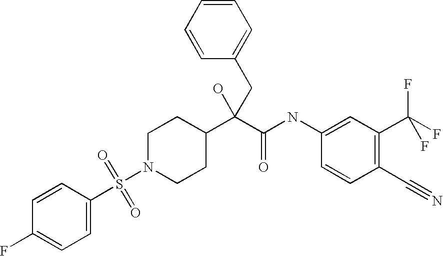 |
| 4207 4208 4209 | rac + − | 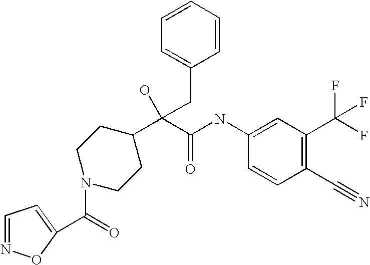 |
| 4210 4211 4212 | rac + − | 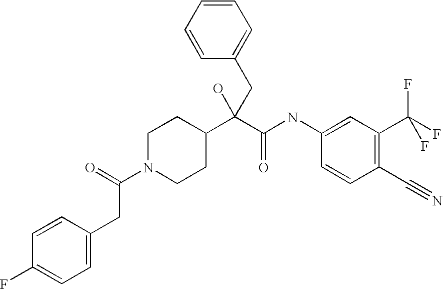 |
| 4213 4214 4215 | rac + − | 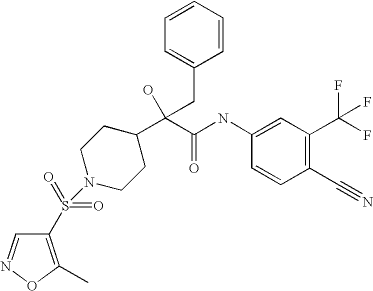 |
| 4216 4217 4218 | rac + − | 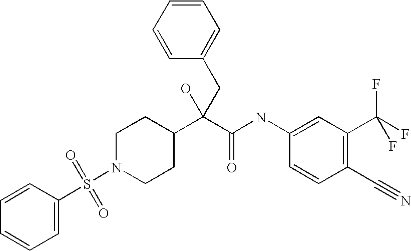 |
| 4219 4220 4221 | rac + − | 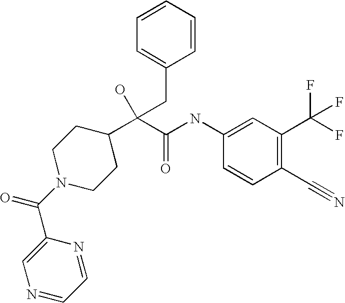 |
| 4222 4223 4224 | rac + − | 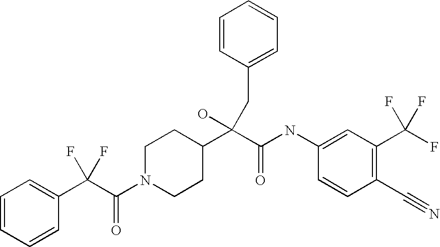 |
| 4225 4226 4227 | rac + − | 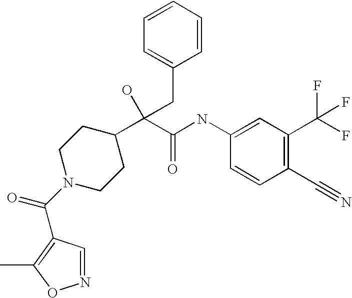 |
| 4228 4229 4230 | rac + − | 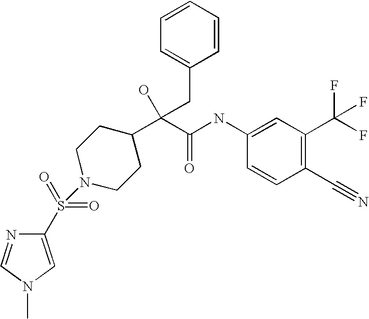 |
| 4231 4232 4233 | rac + − | 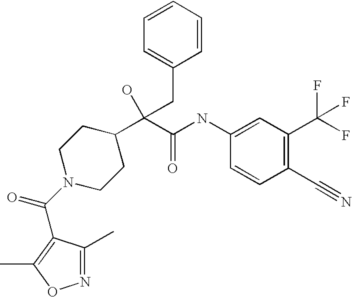 |
| 4234 4235 4236 | rac + − | 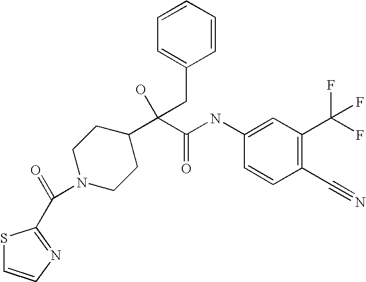 |
| 4237 4238 4239 | rac + − | 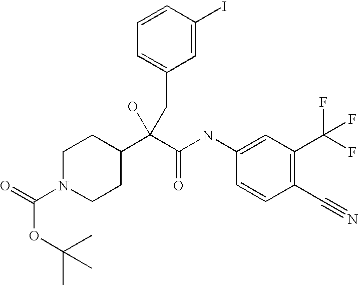 |
| 4240 4241 4242 | rac + − | 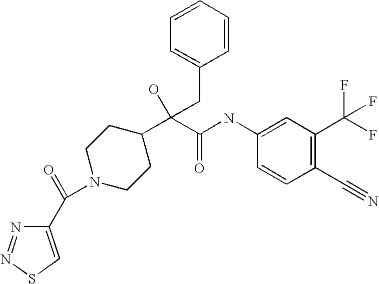 |
| 4243 4244 4245 | rac + − | 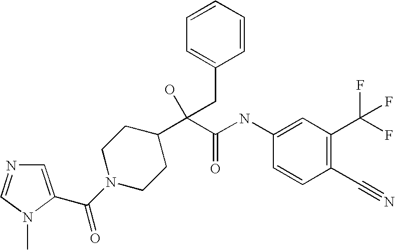 |
| 4246 4247 4248 | rac + − | 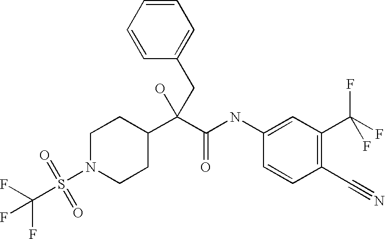 |
| 4249 4250 4251 | rac + − | 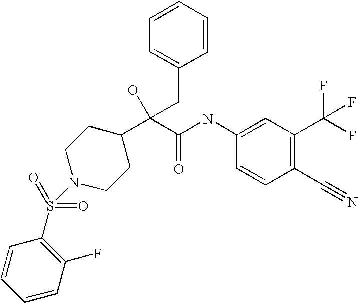 |
| 4252 4253 4254 | rac + − | 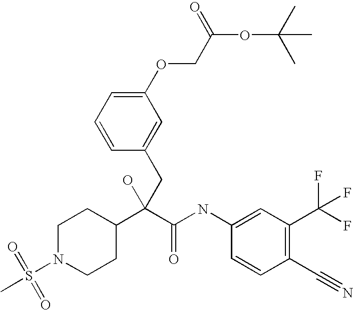 |
| 4255 4256 4257 | rac + − | 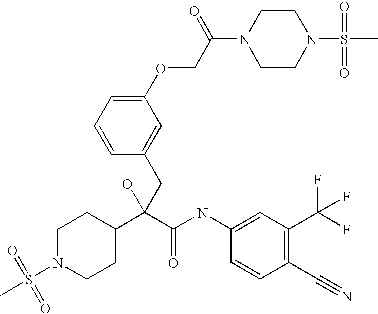 |
| 4258 4259 4260 | rac + − | 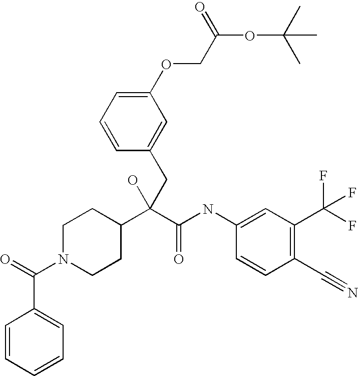 |
| 4261 4262 4263 | rac + − | 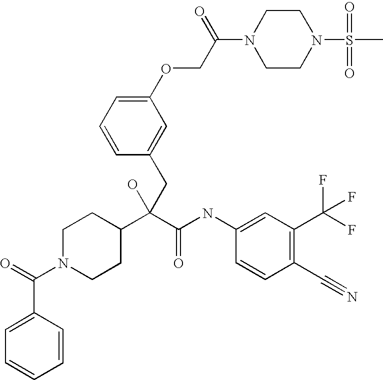 |
| 4264 4265 4266 | rac + − | 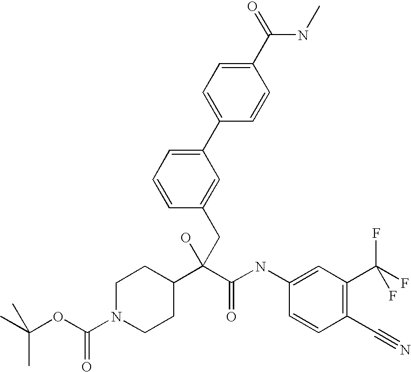 |
| 4267 4268 4269 | rac + − | 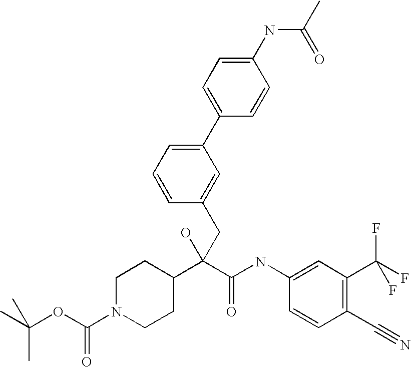 |
| 4270 4271 4272 | rac + − | 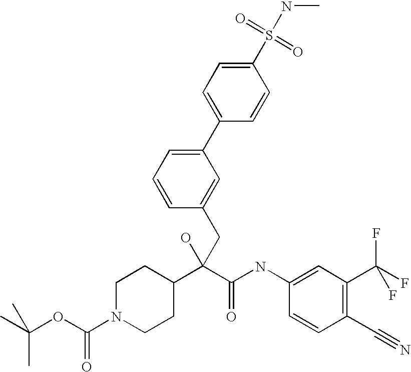 |
| 4273 4274 4275 | rac + − | 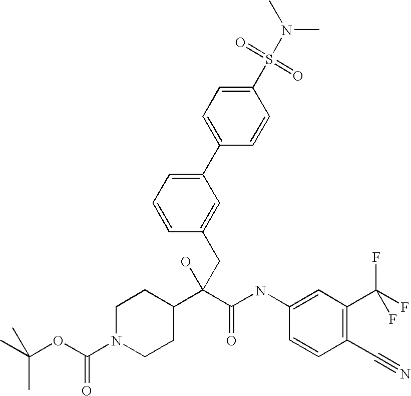 |
| 4276 4277 4278 | rac + − | 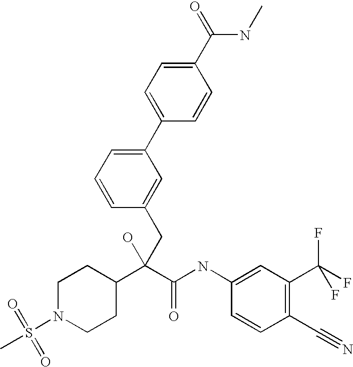 |
| 4279 4280 4281 | rac + − | 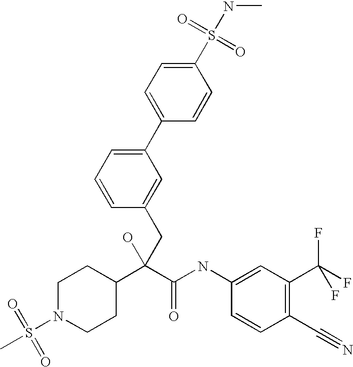 |
| 4282 4283 4284 | rac + − | 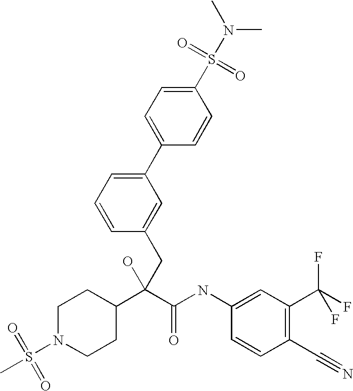 |
| 4285 4286 4287 4288 4289 | stereoisomer mixture (R, R) (S, S) (R, S) (S, R) | 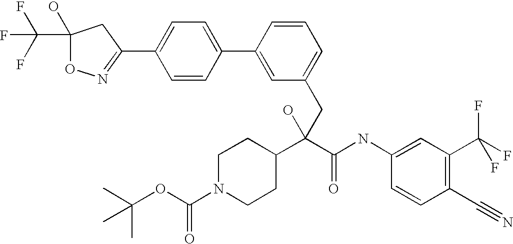 |
| 4290 4291 4292 4293 4294 | stereoisomer mixture (R, R) (S, S) (R, S) (S, R) | 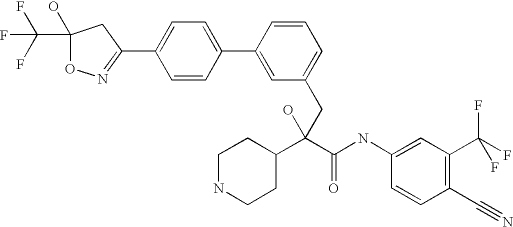 |
| 4295 4296 4297 | rac + − | 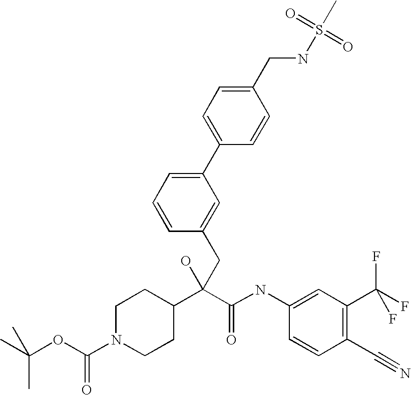 |
| 4298 4299 4300 | rac + − | 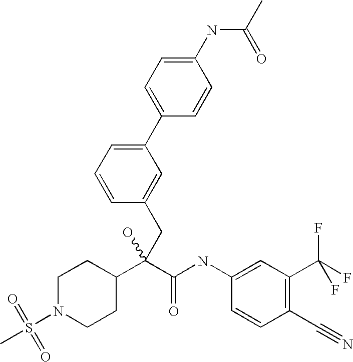 |
| 4301 4302 4303 4304 4305 | stereoisomer mixture (R, R) (S, S) (R, S) (S, R) | 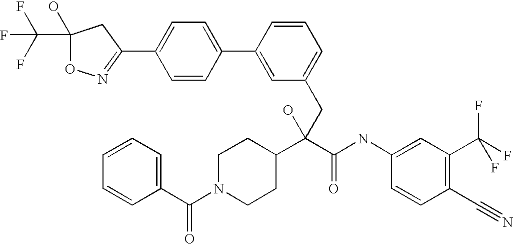 |
| 4306 4307 4308 | rac + − | 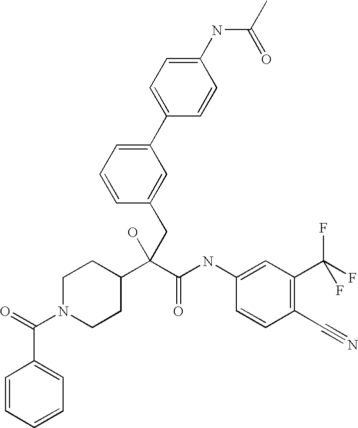 |
| 4309 4310 4311 | rac + − | 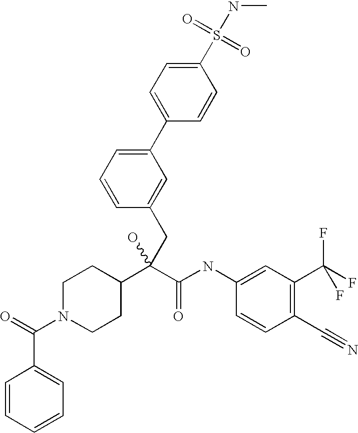 |
| 4312 4313 4314 | rac + − | 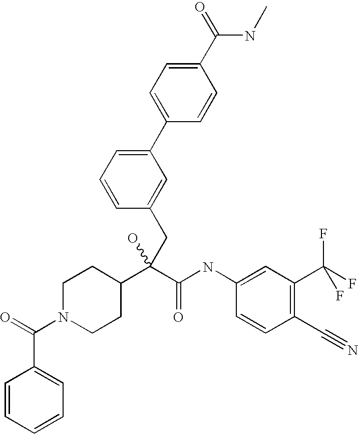 |
| 4315 4316 4317 | rac + − | 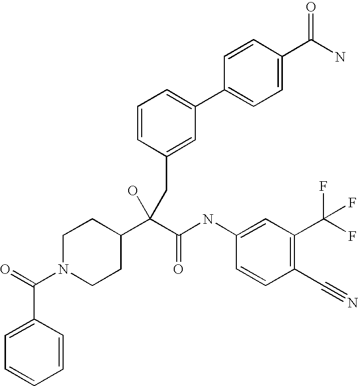 |
| 4318 4319 4320 | rac + − | 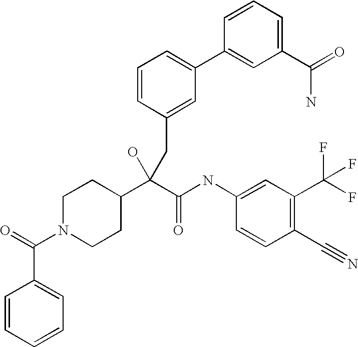 |
| 4321 4322 4323 | rac + − | 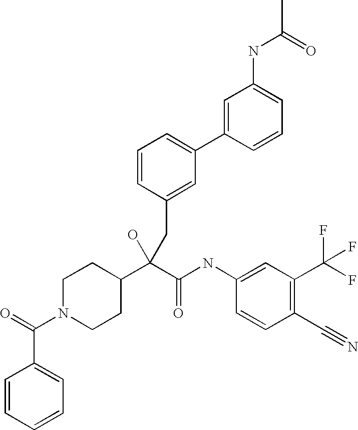 |
| 4324 4325 4326 | rac + − | 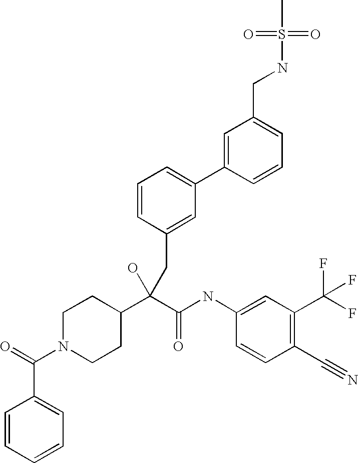 |
| 4327 4328 4329 | rac + − | 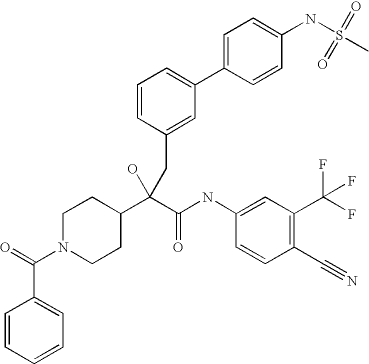 |
| 4330 4331 4332 | rac + − | 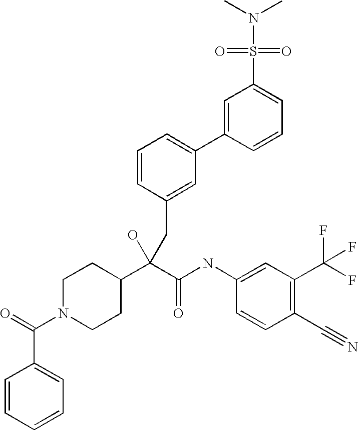 |
| 4333 4334 4335 | rac + − | 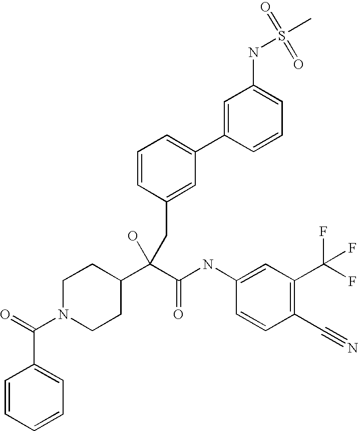 |
| 4336 4337 4338 | rac + − | 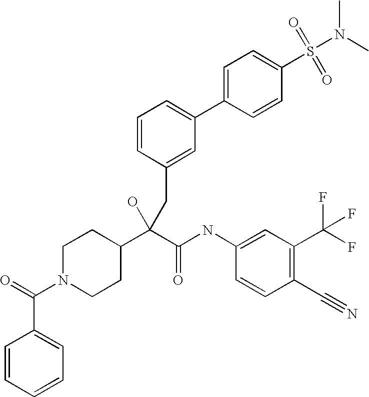 |
| 4339 4340 4341 | rac + − | 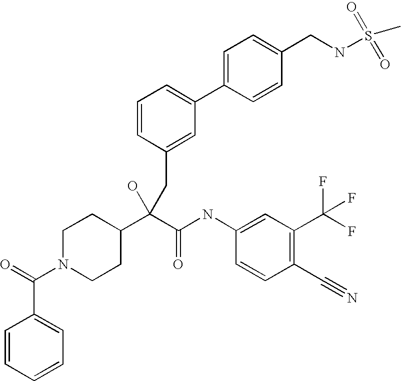 |
| 4342 4343 4344 | rac + − | 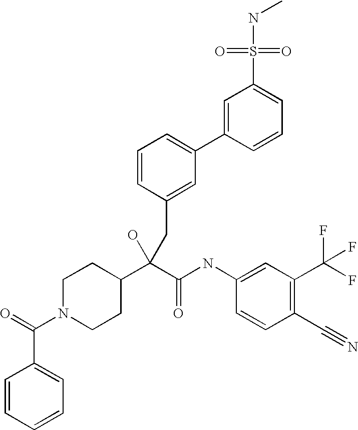 |
| 4345 4346 4347 | rac + − | 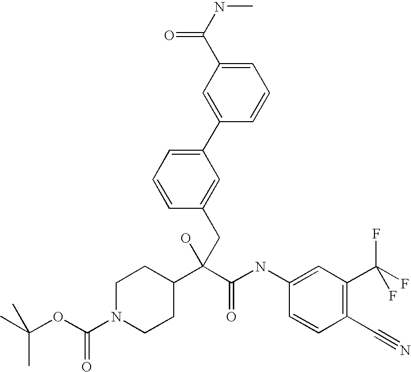 |
| 4348 4349 4350 | rac + − | 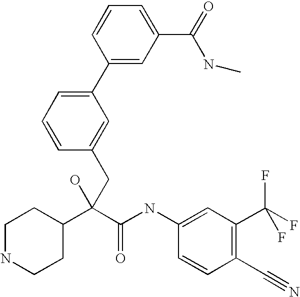 |
| 4351 4352 4353 | rac + − | 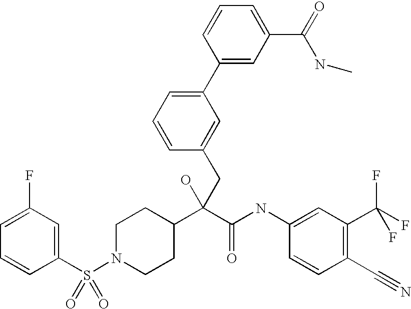 |
| 4354 4355 4356 | rac + − | 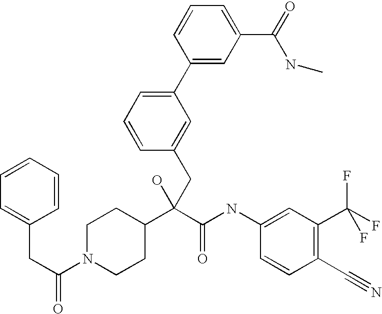 |
| 4357 4358 4359 | rac + − | 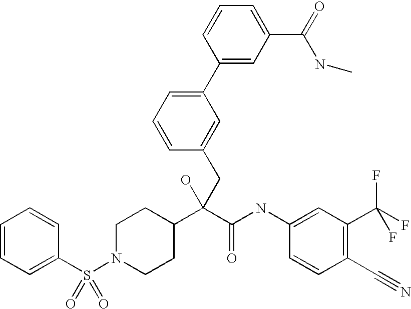 |
| 4360 4361 4362 | rac + − | 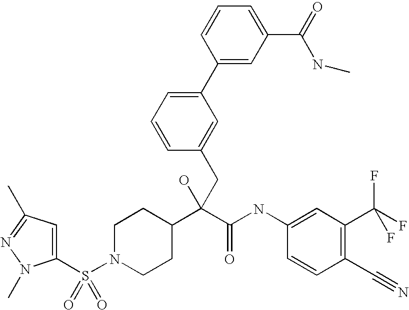 |
| 4363 4364 4365 | rac + − | 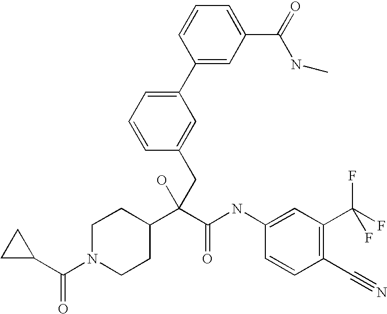 |
| 4366 4367 4368 | rac + − | 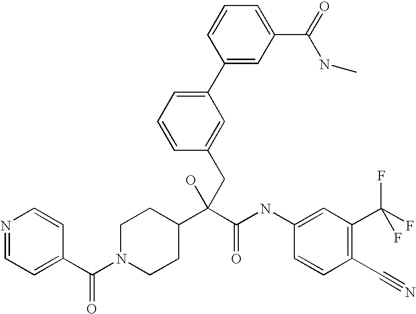 |
| 4369 4370 4371 4372 4373 | stereoisomer mixture (R, R) (S, S) (R, S) (S, R) | 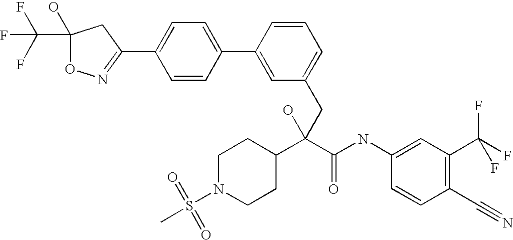 |
| 4374 4375 4376 | rac + − | 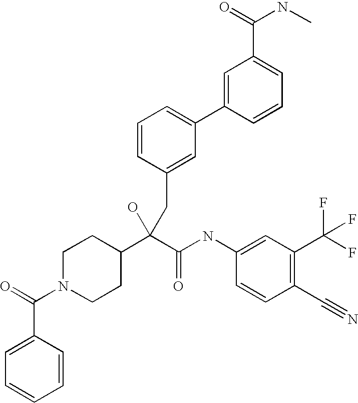 |
| 4377 4378 4379 | rac + − | 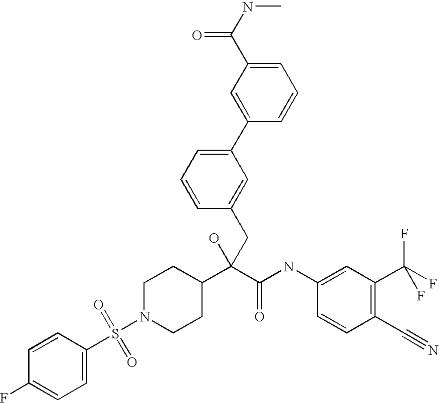 |
| 4380 4381 4382 | rac + − | 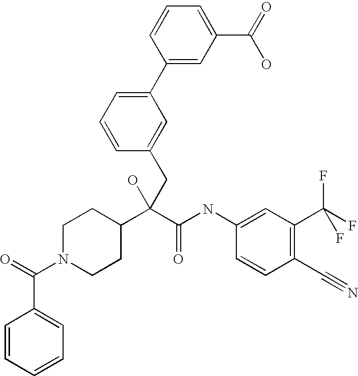 |
| 4383 4384 4385 | rac + − | 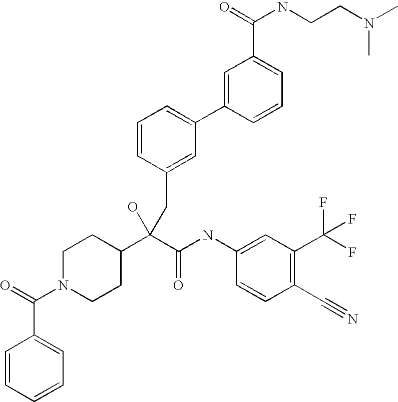 |
| 4386 4387 4388 | rac + − | 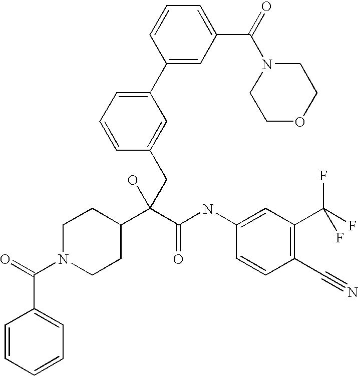 |
| 4389 4390 4391 | rac + − | 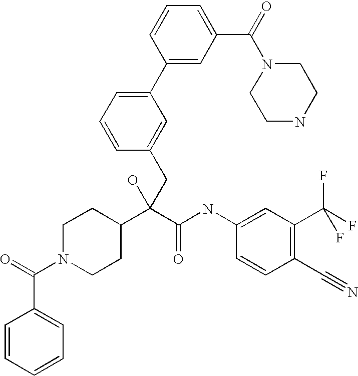 |
| 4392 4393 4394 | rac + − | 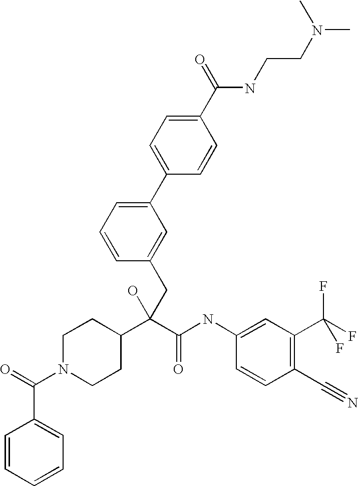 |
| 4395 4396 4397 | rac + − | 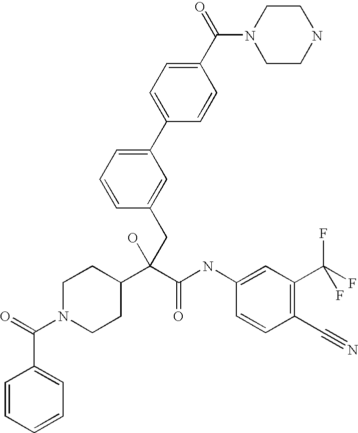 |
| 4398 4399 4400 | rac + − | 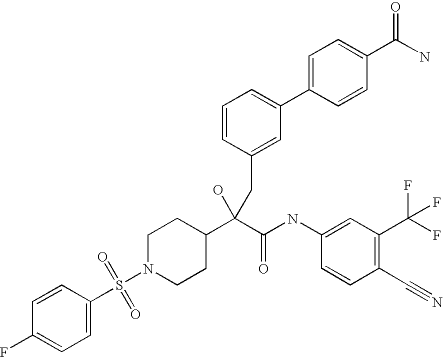 |
| 4401 4402 4403 | rac + − | 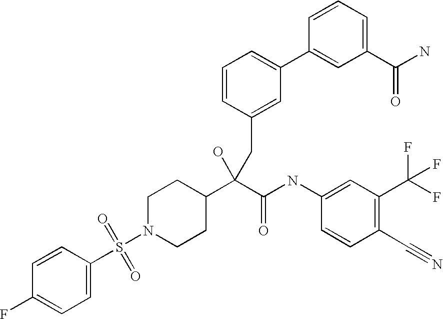 |
| 4404 4405 4406 | rac + − | 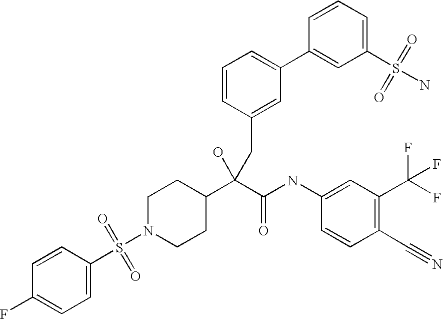 |
| 4407 4408 4409 | rac + − | 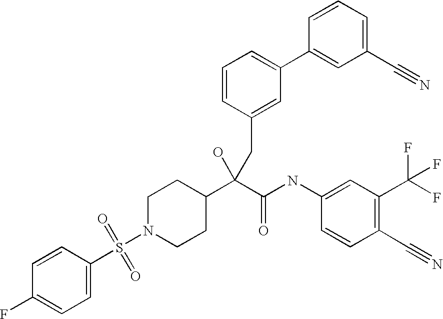 |
| 4410 4411 4412 | rac + − | 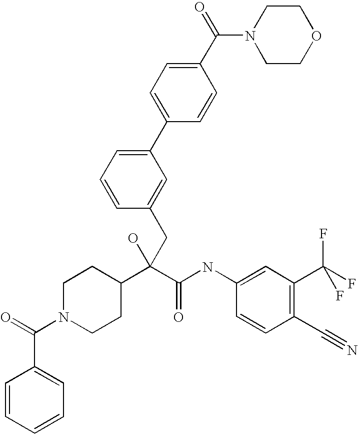 |
| 4413 4414 4415 | rac + − | 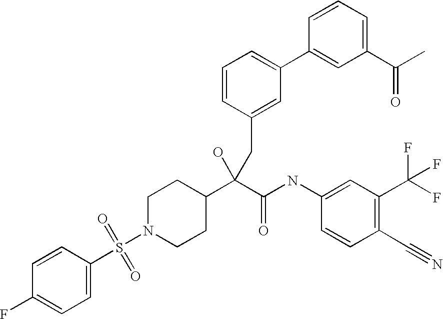 |
| 4416 4417 4418 | rac + − | 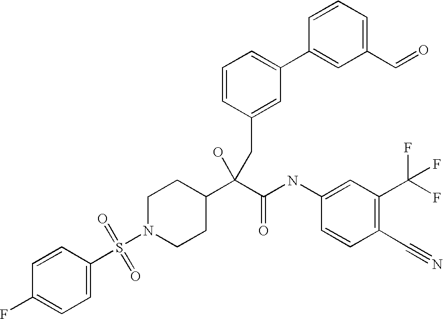 |
| 4419 4420 4421 | rac + − | 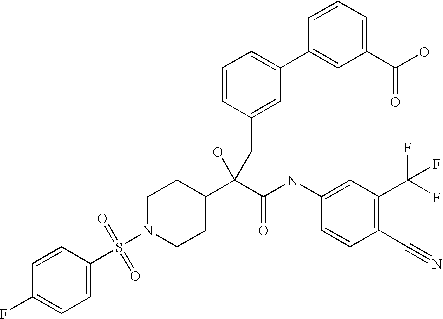 |
| 4422 4423 4424 | rac + − | 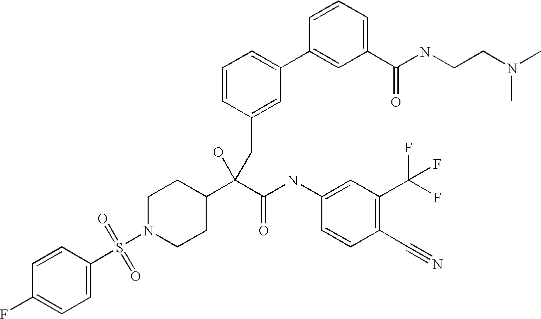 |
| 4425 4426 4427 | rac + − | 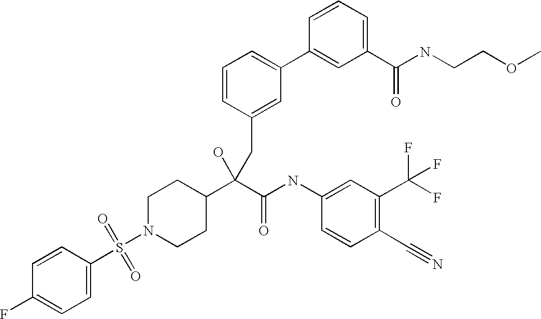 |
| 4428 4429 4430 | rac + − | 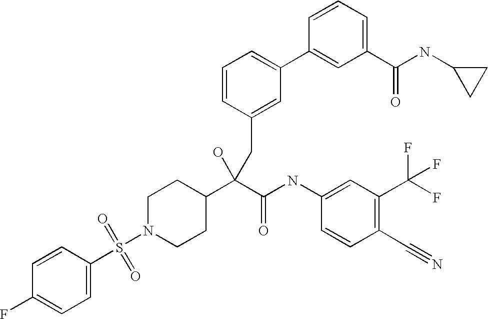 |
| 4431 4432 4433 | rac + − | 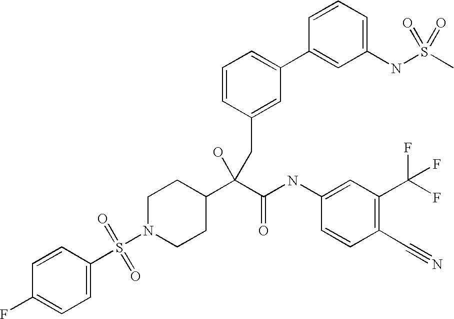 |
| 4434 4435 4436 4437 4438 | stereoisomer mixture (R, R) (S, S) (R, S) (S, R) | 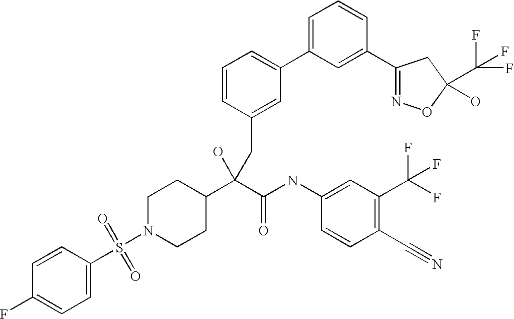 |
| 4439 4440 4441 | rac + − | 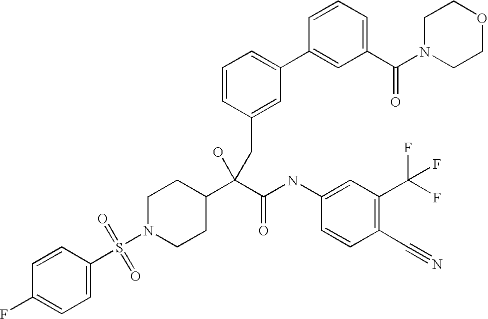 |
| 4442 4443 4444 | rac + − | 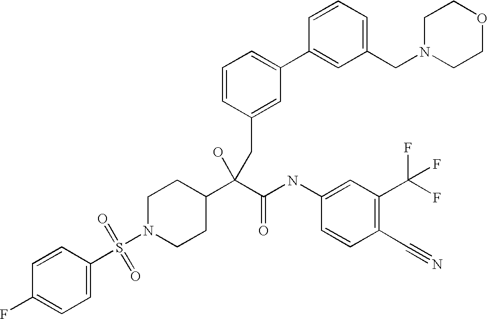 |
| 4445 4446 4447 | rac + − | 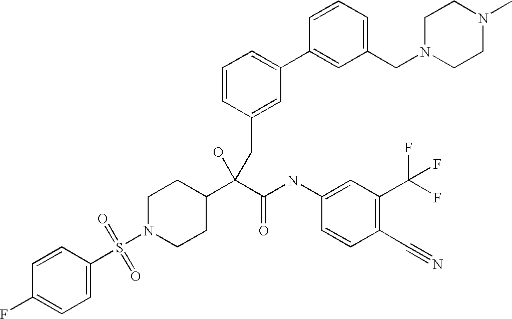 |
| 4448 4449 4450 | rac + − | 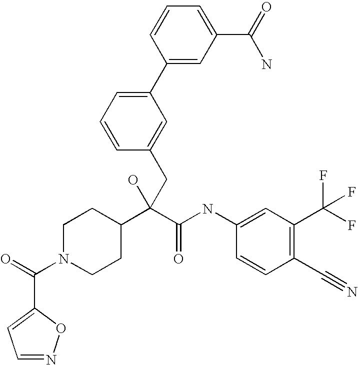 |
| 4451 4452 4453 | rac + − | 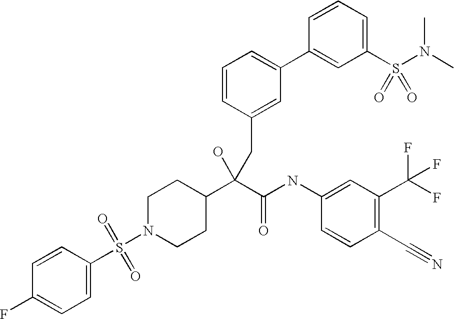 |
| 4454 4455 4456 | rac + − | 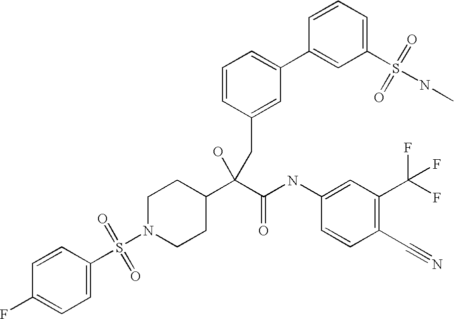 |
| 4457 4458 4459 | rac + − | 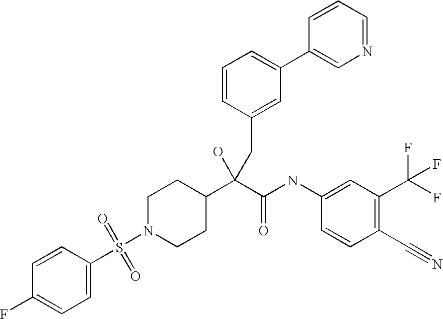 |
| 4460 4461 4462 | rac + − | 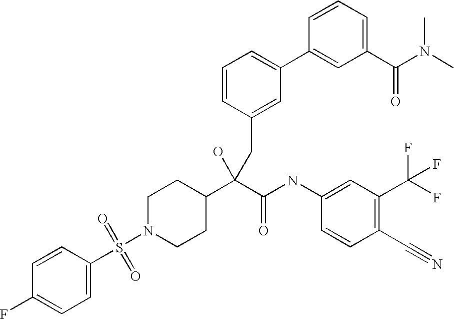 |
| 4463 4464 4465 | rac + − | 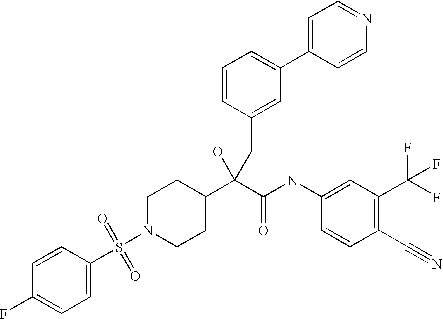 |
| 4466 4467 4468 | rac + − | 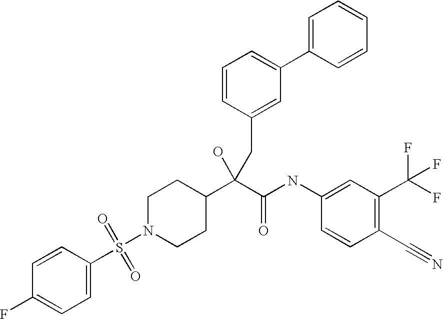 |
| 4469 4470 4471 4472 4473 | stereoisomer mixture (R, R) (S, S) (R, S) (S, R) | 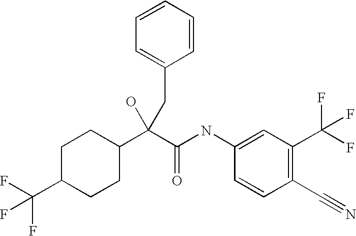 |
| 4474 4475 4476 4477 4478 4479 4480 4481 4482 | stereoisomer mixture (R, R, R) (S, S, S) (R, R, S) (R, S, S) (R, S, R) (S, S, R) (S, R, R) (S, R, S) | 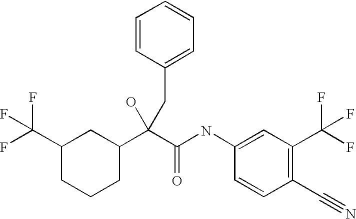 |
| 4483 4484 4485 4486 4487 | stereoisomer mixture (R, R) (S, S) (R, S) (S, R) | 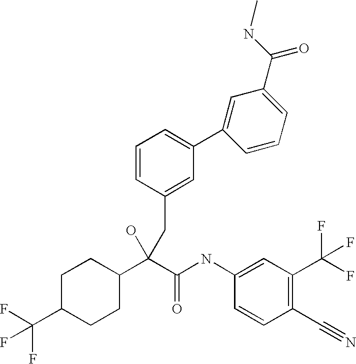 |
| 4488 4489 4490 | rac + − | 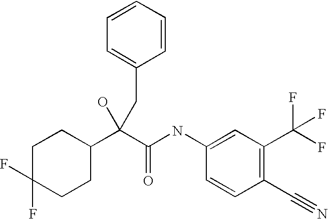 |
| 4491 4492 4493 | rac + − | 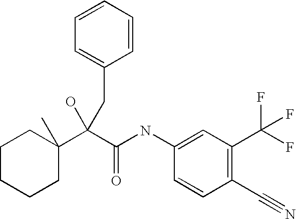 |

14. Pharmaceutical composition comprising at least one compound of the general formula I according to any of claims 1 to 12 and, where appropriate, at least one further active ingredient together with pharmaceutically suitable excipients and/or carriers.

15. Pharmaceutical composition according to claim 14, where the further active ingredient is a SERM (selective oestrogen receptor modulator), an aromatase inhibitor, antioestrogen or a prostaglandin.

16. Pharmaceutical composition according to claim 15, where the further active ingredients may be tamoxifen, 5-(4-{5-[(RS)-(4,4,5,5,5-pentafluoropentyl)sulphinyl]pentyloxy}phenyl)-6-phenyl-8,9-dihydro-7H-benzocyclohepten-2-ol, ICI 182 780 (7alpha-[9-(4,4,5,5-pentafluoropentylsulphinyl)nonyl]estra-1,3,5(10)-triene-3,17beta-diol), 11beta-fluoro-7alpha-[5-(methyl{3-[(4,4,5,5,5-pentafluoropentyl)sulphanyl]-propyl}amino)pentyl]estra-1,3,5(10)-triene-3,17beta-diol, 11beta-fluoro-7alpha-{5-[methyl(7,7,8,8,9,9,10,10,10-nonafluorodecyl)amino]pentyl}estra-1,3,5(10)-triene-3,17beta-diol, 11beta-fluoro-17alpha-methyl-7alpha-{5-[methyl(8,8,9,9,9-pentafluorononyl)amino]pentyl}estra-1,3,5(10)-triene-3,17beta-diol, clomifene, raloxifene, fadrozole, formestane, letrozole, anastrozole or atamestane.

17. Compounds according to any of claims 1 to 13 for the manufacture of a medicament.

18. Use of compounds according to any of claims 1 to 13 for the manufacture of a medicament for the therapy and/or prophylaxis of gynaecological disorders such as endometriosis, leiomyomas of the uterus, dysfunctional bleeding and dysmenorrhoea.

19. Use of compounds according to any of claims 1 to 13 for the manufacture of a medicament for the therapy and/or prophylaxis of hormone-dependent tumours.

20. Use of compounds according to any of claims 1 to 13 for the manufacture of a medicament for the therapy and/or prophylaxis of breast carcinomas.

21. Use of compounds according to any of claims 1 to 13 for the manufacture of a medicament for the therapy and/or prophylaxis of endometrial carcinoma.

22. Use of compounds according to any of claims 1 to 13 for the manufacture of a medicament for the therapy and/or prophylaxis of ovarian carcinomas.

23. Use of compounds according to any of claims 1 to 13 for the manufacture of a medicament for the therapy and/or prophylaxis of prostate carcinomas.

24. Use of compounds according to any of claims 1 to 13 for the manufacture of a medicament for female hormone replacement therapy.

25. Use of compounds according to any of claims 1 to 13 for female fertility control.
